(19) United States
(12) Patent Application Publication
(10) Pub. No.: US 2024/0124465 A1
(43) Pub. Date: Apr. 18, 2024

(54) MOLECULES AND METHODS RELATED TO TREATMENT OF DISORDERS ASSOCIATED WITH JAK-2 SIGNALING DYSFUNCTION

(71) Applicant: ST. JUDE CHILDREN'S RESEARCH HOSPITAL, Memphis, TN (US)

(72) Inventors: Jaeki Min, Canoga Park, CA (US); Jamie Jarusiewicz, Memphis, TN (US); Marisa Actis, Bartlett, TN (US); Yunchao Chang, Memphis, TN (US); Lisa Alcock, Memphis, TN (US); Charles Mulligan, Memphis, TN (US); Zoran Rankovic, Memphis, TN (US)

(21) Appl. No.: 18/256,857

(22) PCT Filed: Dec. 17, 2021
(86) PCT No.: PCT/US2021/064157

§ 371 Date: Jun. 9, 2023

Related U.S. Application Data
(60) Provisional application No. 63/127,677, filed on Dec. 18, 2020.

(51) Int. Cl. C07D 487/04; C07D 519/00

(52) U.S. Cl. CPC C07D 487/04; C07D 519/00

(57) ABSTRACT

The present disclosure relates to chemical compounds that modulate JAK-2 signaling, which are useful in the treatment of disorders associated with JAK-2 signaling dysfunction such as, for example, disorders of cellular proliferation (e.g., cancer) and autoimmune and inflammatory disorders, pharmaceutical compositions containing such compounds, and their use in treatment. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/127,677, filed on Dec. 18, 2020, the contents of which are hereby incorporated by reference in their entirety.

### BACKGROUND

The JAK-STAT (Janus associated kinase-signal transducer and activator of transcription) pathway is one of the critical intracellular signaling cascades in transduction of extracellular signals to the nucleus to control gene expression. A variety of cytokines and growth factors complete their physiological tasks through the JAK-STAT pathway, including hematopoiesis, immune-regulation, fertility, lactation, growth and embryogenesis. See, e.g., Ferrajoli et al. (2006) The JAK-STAT pathway: a therapeutic target in hematological malignancies. Curr Cancer Drug Targets. 6(8):671-679; Moriggl et al. (1999) Stat5 activation is uniquely associated with cytokine signaling in peripheral T cells. Immunity. 11(2):225-230; Friedrich et al. (1999) Activation of STAT5 by IL-4 relies on Janus kinase function but not on receptor tyrosine phosphorylation, and can contribute to both cell proliferation and gene regulation. Int Immunol. 11(8):1283-1294; Moriggl et al. (1999) Stat5 is required for IL-2-induced cell cycle progression of peripheral T cells. Immunity. 10(2):249-259; Bradley et al. (2002) Cell intrinsic defects in cytokine responsiveness of STAT5-deficient hematopoietic stem cells. Blood. 100(12):3983-3989; Bunting et al. (2002) Reduced lymphomyeloid repopulating activity from adult bone marrow and fetal liver of mice lacking expression of STAT5. Blood. 99(2):479-487).

Dysregulation in the JAK-STAT pathway has been described in a variety of cancers, especially in hematological malignancies including myeloid disorders. Cancer is a disease characterized primarily by an uncontrolled division of abnormal cells (uncontrolled cellular proliferation) derived from a given normal tissue and the invasion of adjacent tissues by these malignant cells. Blood or lymphatic transportation can spread cancer cells to other parts of the body leading to regional lymph nodes and to distant sites (metastasis). Cancer is a complex, multistep process that begins with minor preneoplastic changes, which may under certain conditions progress to neoplasia. There are more than 100 different types of cancer, which can be grouped into broader categories. The main categories include carcinoma, sarcoma, leukemia, lymphoma and myeloma, and central nervous system cancers. The incidence of cancer continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer.

Hematologic or hematopoietic malignancies are cancers of the blood or bone marrow, including leukemia and lymphoma. Leukemia is a type of cancer of the blood characterized by abnormal accumulation of immature white blood cells. There are four types of leukemia: acute lymphocytic leukemia (ALL), acute myelogenous leukemia (AML), chronic lymphocytic leukemia (CLL), and chronic myelogenous leukemia (CML). Acute leukemia is a rapidly progressing disease that results in the accumulation of immature, functionless cells in the marrow and blood. The marrow often stops producing enough normal red cells, which include cells and platelets. On the other hand, chronic leukemia progresses more slowly and allows greater numbers of more mature, functional cells to be made.

Leukemia can affect people at any age. The cause of most cases of leukemia is not known. Extraordinary doses of radiation and certain cancer therapies are possible causes. About 90% of leukemia cases are diagnosed in adults. Cases of chronic leukemia account for 4.5 percent more cases that acute leukemia.

The dramatic improvement in blood cancer treatment in the latter part of the twentieth century is largely the result of chemotherapy. In addition, there are more than fifty drugs individually used to treat these disorders and a number of potential new therapies are under investigation in clinical trials.

Dysregulation of the JAK-STAT pathway has also been identified in a variety of autoimmune and inflammatory diseases such as rheumatoid arthritis, psoriasis, pruritus, atopic dermatitis, and Crohn's disease. Indeed, the discovery of the numerous cytokines underlying the pathogenesis of allergic, inflammatory and autoimmune disorders has provided a basis for the development of highly successful therapeutic monoclonal antibodies and recombinant proteins that target several such cytokines and their receptors (Schwartz et al. (2016) Type I/II cytokines, JAKs, and new strategies for treating autoimmune diseases. Nat Rev Rheumatol 12: 25-36). Such therapies have dramatically altered outcomes for a range of diseases, including rheumatoid arthritis (RA), psoriasis and Inflammatory Bowel Disease (IBD) (Schwartz et al. (2016) Type I/II cytokines, JAKs, and new strategies for treating autoimmune diseases. Nat Rev Rheumatol 12: 25-36). However, even for a disorder like rheumatoid arthritis (RA) in which much progress has been made, most patients do not respond completely to currently available therapies, and there are relatively few examples of long-term remissions after cessation of therapy (Singh et al. (2016) Biologics or tofacitinib for rheumatoid arthritis in incomplete responders to methotrexate or other traditional disease-modifying anti-rheumatic drugs: a systematic review and network meta-analysis. Cochrane Database Syst Rev, CD012183). For other disorders, there has been even less progress, especially diseases in which fibrosis and tissue destruction are major features, such as systemic sclerosis.

Thus, despite the advances, there remains a need for methods of treating disorders impacted by the JAK-STAT pathway, including disorders of uncontrolled cellular proliferation and autoimmune and inflammatory disorders. The following disclosure describes a group of such compounds, as well as methods for making and using them.

### SUMMARY

In accordance with the purpose(s) of the invention, as embodied and broadly described herein, the invention, in one aspect, relates to compositions and methods for use in the prevention and treatment of disorders associated with JAK-2 signaling dysfunction such as, for example, a disorder associated with uncontrolled cellular proliferation, (e.g., cancer), and an autoimmune and inflammatory disorder (e.g., psoriasis, rheumatoid arthritis, pruritus, atopic dermatitis, Crohn's disease, inflammatory bowel disease (IBD), human immunodeficiency virus type-I (HIV)).

Disclosed are compounds having a structure represented by a formula:

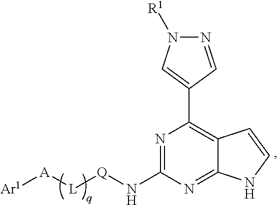

wherein q is selected from 0 and 1; wherein A is selected from —O—, —S—, —NH—, —NHCH₂—, —OCH₂C(O)NH—, —CH₂—, and a structure selected from:

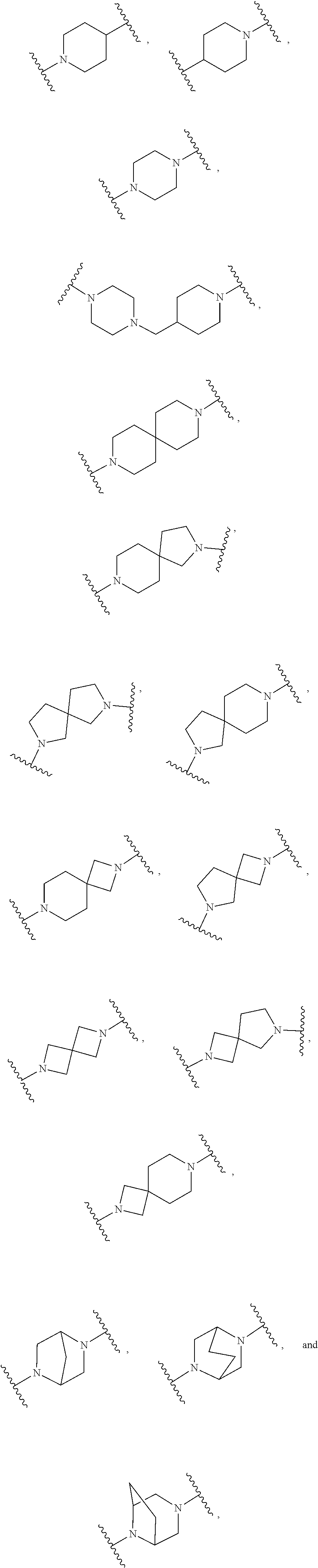

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

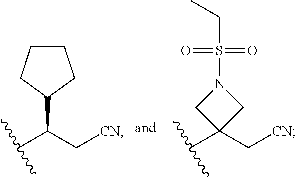 [SMILES: *[C@H](CC#N)C1CCCC1 ; *C1(CC#N)CN(S(=O)(=O)CC)C1]

and
wherein Ar¹ is a structure selected from:

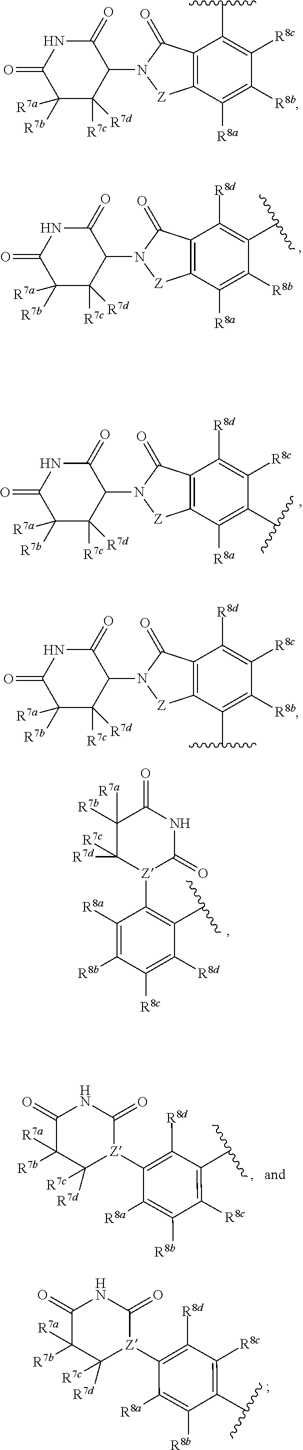

wherein Z is selected from —CH₂— and C(O); wherein Z′ is selected from —CH— and —N—; wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

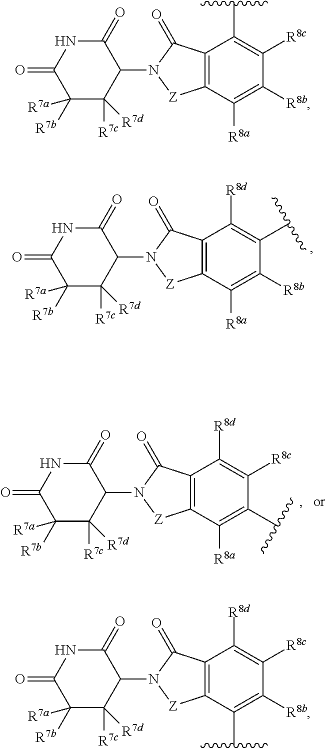

and q is 1, then A is a structure selected from:

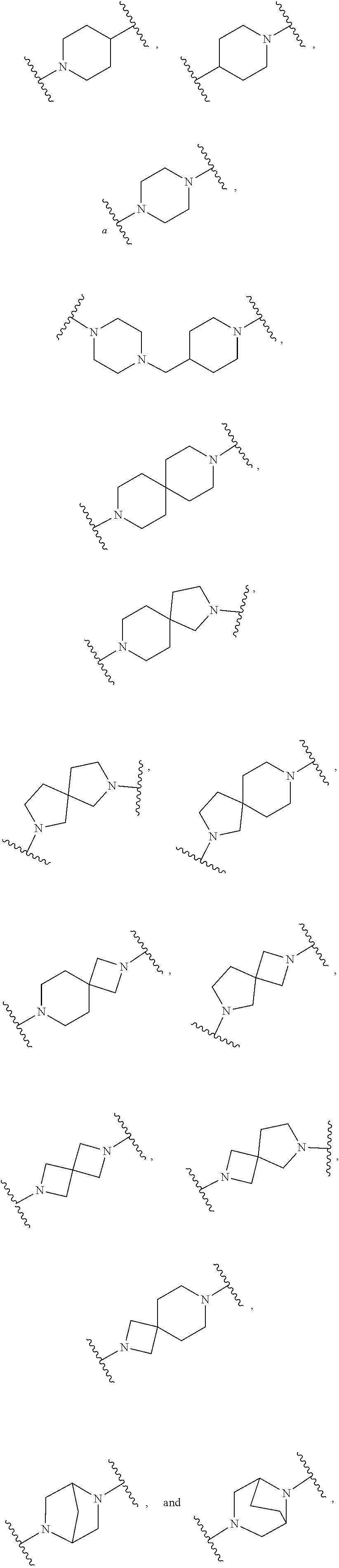

or a pharmaceutically acceptable salt thereof.

Also disclosed are compounds having a structure represented by a formula:

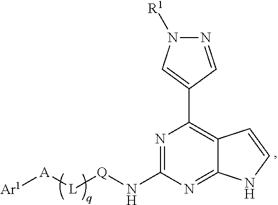

wherein q is selected from 0 and 1; wherein A is selected from —O—, —S—, —NH—, —NHCH₂—, —OCH₂C(O)NH—, —CH₂—, and a structure selected from:

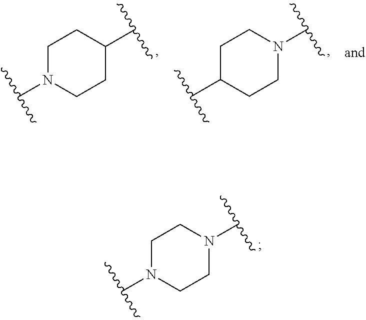

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

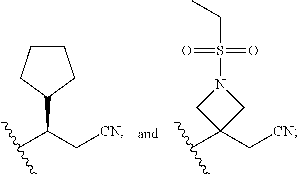 [SMILES: *[C@H](CC#N)C1CCCC1 ; *C1(CC#N)CN(S(=O)(=O)CC)C1]

and
wherein Ar¹ is a structure selected from:

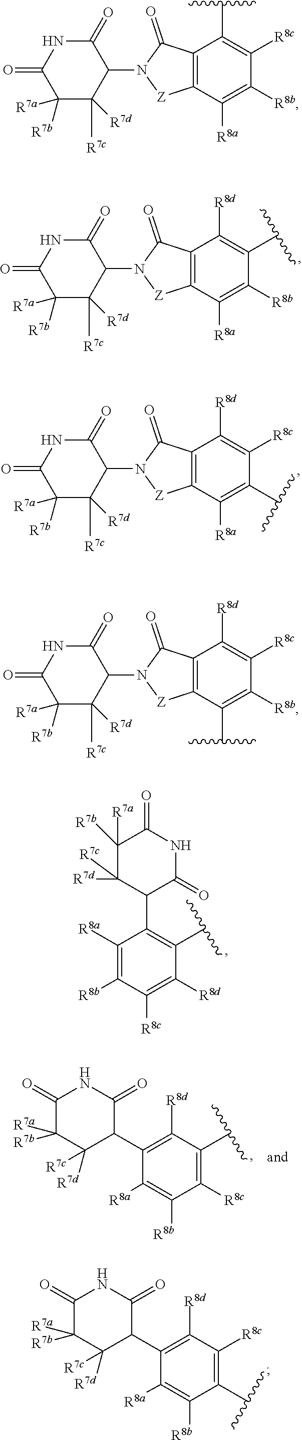

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

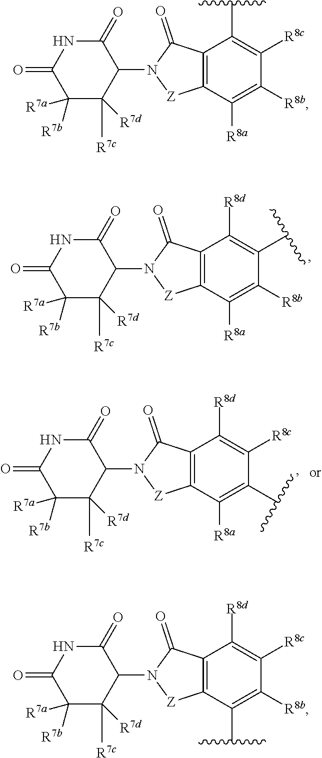

and q is 1, then A is a structure selected from:

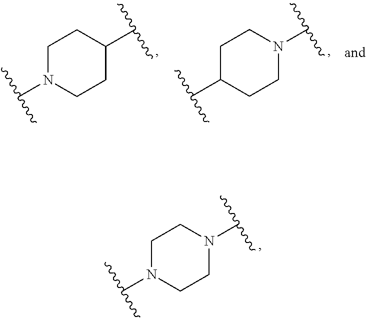 [SMILES: *C1CCN(*)CC1 ; *C1CCN(*)CC1 ; *N1CCN(*)CC1]

or a pharmaceutically acceptable salt thereof.

Also disclosed are pharmaceutical compositions comprising a therapeutically effective amount of a disclosed compound and a pharmaceutically acceptable carrier.

Also disclosed are methods of treating a disorder associated with JAK-2 signaling dysfunction in a subject, the method comprising administering to the subject an effective amount of a disclosed compound.

Also disclosed are methods of treating a disorder of uncontrolled cellular proliferation in a subject, the method comprising administering to the subject an effective amount of a disclosed compound.

Also disclosed are kits comprising a disclosed compound, and one or more of: (a) an agent known to treat a disorder associated with JAK-2 signaling dysfunction; (b) instructions for administering the compound in connection with treating a disorder associated with JAK-2 signaling dysfunction; and (c) instructions for treating a disorder associated with JAK-2 signaling dysfunction.

Also disclosed are kits comprising a disclosed compound, and one or more of: (a) an agent known to treat a disorder of uncontrolled cellular proliferation; (b) instructions for administering the compound in connection with treating a disorder of uncontrolled cellular proliferation; and (c) instructions for treating a disorder of uncontrolled cellular proliferation.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative JAK-CRBN conjugates.

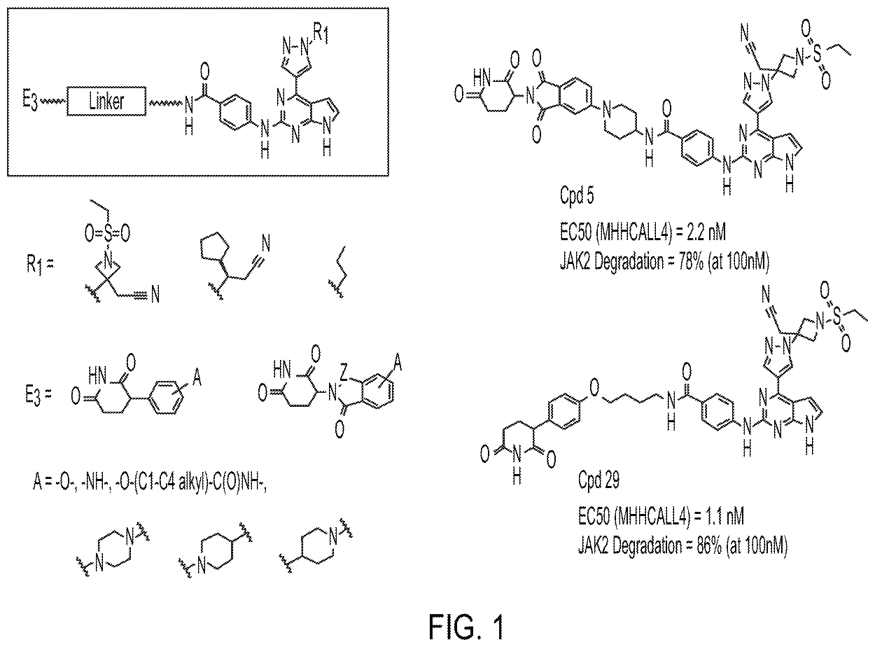

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage form can comprise a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions, which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. In a further aspect, the alkyl group can be substituted. For example, the alkyl group can be substituted with one or more groups including, but not limited to, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. In a further aspect, the cycloalkyl group and heterocycloalkyl group can be substituted. For example, the cycloalkyl group and heterocycloalkyl group can be substituted with 0, 1, 2, 3, or 4 groups independently selected from C1-C4 alkyl, C3-C7 cycloalkyl, C1-C4 alkoxy, —NH₂, (C1-C4) alkylamino, (C1-C4)(C1-C4) dialkylamino, ether, halogen, —OH, C1-C4 hydroxyalkyl, —NO₂, silyl, sulfo-oxo, —SH, and C1-C4 thioalkyl, as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. In a further aspect, the alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. In a further aspect, the cycloalkenyl group and heterocycloalkenyl group can be substituted. For example, the cycloalkenyl group and heterocycloalkenyl group can be substituted with 0, 1, 2, 3, or 4 groups independently selected from C1-C4 alkyl, C3-C7 cycloalkyl, C1-C4 alkoxy, C2-C4 alkenyl, C3-C6 cycloalkenyl, C2-C4 alkynyl, aryl, heteroaryl, aldeyhyde, —NH₂, (C1-C4) alkylamino, (C1-C4)(C1-C4) dialkylamino, carboxylic acid, ester, ether, halogen, —OH, C1-C4 hydroxyalkyl, ketone, azide, —NO₂, silyl, sulfo-oxo, —SH, and C1-C4 thioalkyl, as described herein.

The term “alkynyl” as used herein is a hydrocarbon group of 2 to 24 carbon atoms with a structural formula containing at least one carbon-carbon triple bond. In a further aspect, the alkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. In a further aspect, the cycloalkynyl group and heterocycloalkynyl group can be substituted. For example, the cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. In a further aspect, the aryl group can be substituted. For example, the aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” or “CO” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. In a further aspect, the heteroaryl group can be substituted. For example, the heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group, which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group, which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxy” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN or —C≡N.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

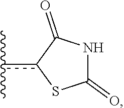

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically labeled or isotopically substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules that owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

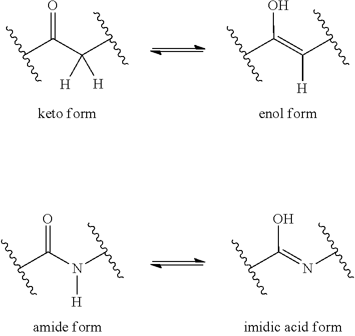 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

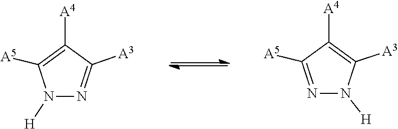

Unless stated to the contrary, the invention includes all such possible tautomers.

It is known that chemical substances form solids that are present in different states of order that are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

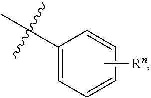

which is understood to be equivalent to a formula:

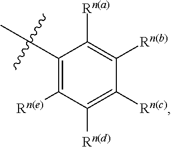

wherein n is typically an integer. That is, R is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance Rⁿ(a) is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, disclosed are compounds useful in treating or preventing a disorder associated with JAK-2 signaling dysfunction such as, for example, a disorder associated with uncontrolled cellular proliferation, (e.g., cancer), and an autoimmune and inflammatory disorder (e.g., psoriasis, rheumatoid arthritis, pruritus, atopic dermatitis, Crohn's disease, inflammatory bowel disease (IBD), human immunodeficiency virus type-I (HIV)). Exemplary cancers include, but are not limited to, CRLF2-rearranged (CRLF2r) and JAK-STAT signaling driven acute lymphocytic leukemia. CRLF2-rearranged ALL comprises up to 60% of Philadelphia-like (Ph-like, BCR ABL1-like) acute lymphoblastic leukemia (ALL), and up to 15% of B-ALL overall, each of which is associated with high risk features and poor outcome. In a further aspect, the disclosed compounds exhibit modulation of JAK-2 signaling. In a still further aspect, the compounds exhibit inhibition of JAK-2 signaling.

In a further aspect, the disclosed compounds exhibit modulation of uncontrolled cellular proliferation. In still a further aspect, the disclosed compounds exhibit inhibition of uncontrolled cellular proliferation.

In one aspect, the compounds of the invention are useful in the treatment or prevention of disorders associated with JAK-2 signaling dysfunction, as further described herein.

In one aspect, the compounds of the invention are useful in the treatment or prevention of disorders associated with uncontrolled cellular proliferation, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, disclosed are compounds having a structure represented by a formula:

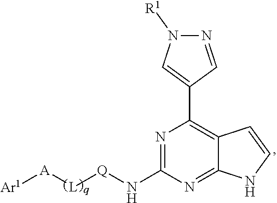

wherein q is selected from 0 and 1; wherein A is selected from —O—, —S—, —NH—, —NHCH₂—, —OCH₂C(O)NH—, —CH₂—, and a structure selected from:

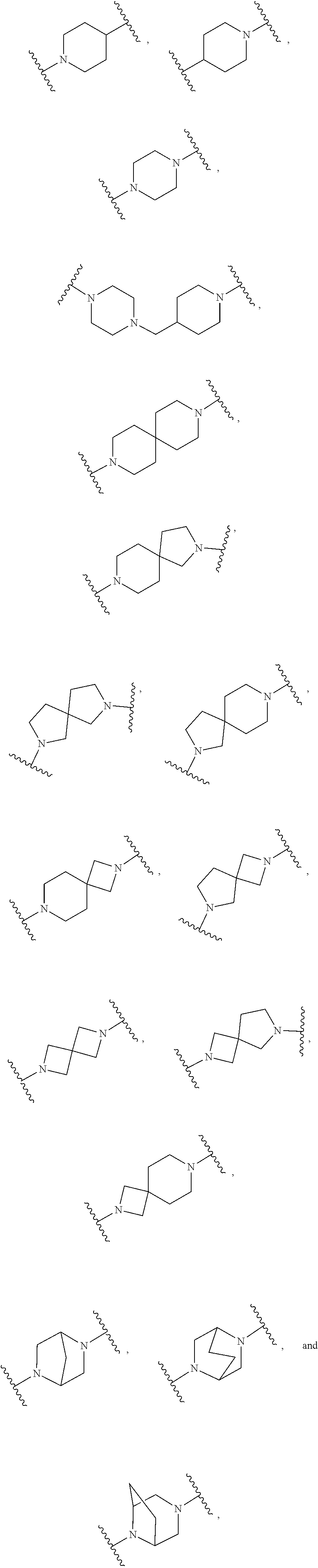

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

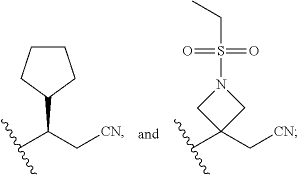 [SMILES: *[C@H](CC#N)C1CCCC1 ; *C1(CC#N)CN(S(=O)(=O)CC)C1]

and
wherein Ar¹ is a structure selected from:

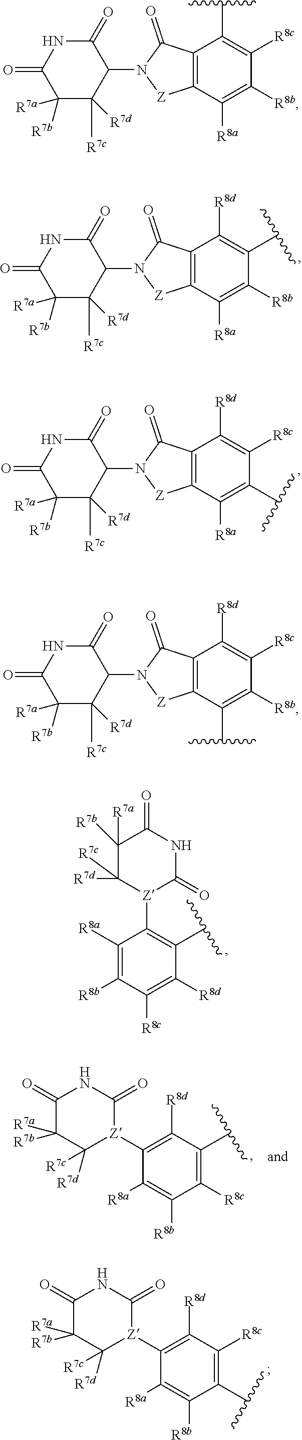

wherein Z is selected from —CH₂— and C(O); wherein Z′ is selected from —CH— and —N—; wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

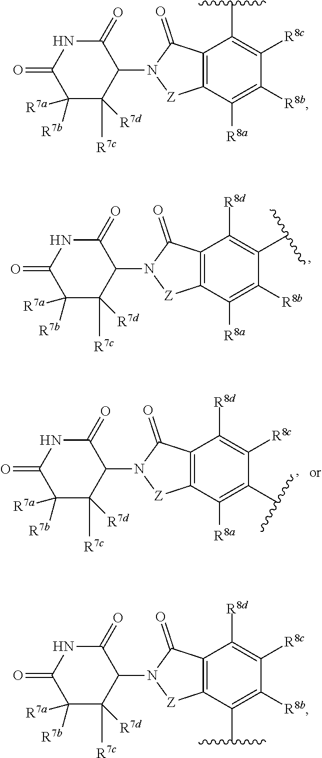

and q is 1, then A is a structure selected from:

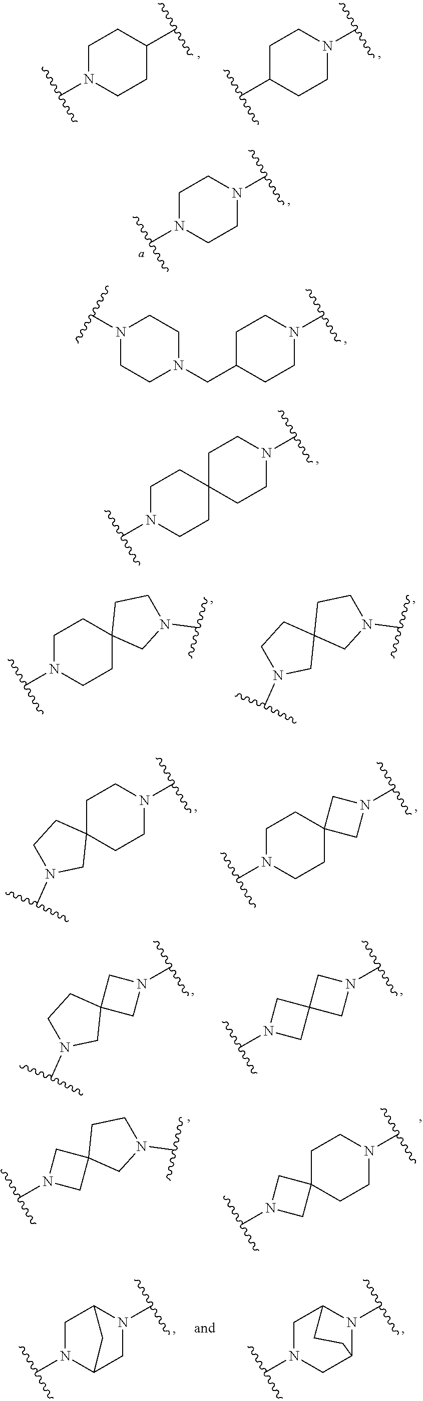

or a pharmaceutically acceptable salt thereof. In a further aspect, when Ar¹ is:

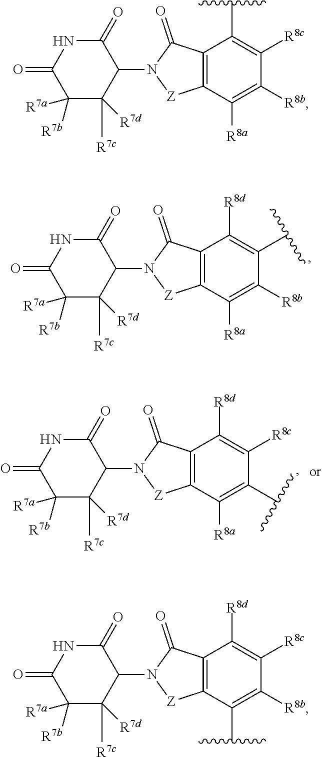

and q is 1, then A is a structure selected from:

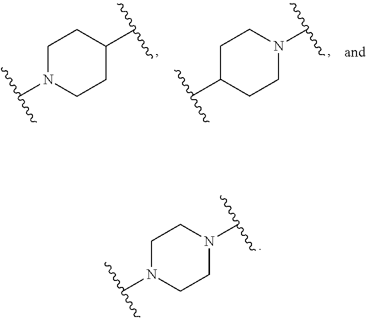 [SMILES: *C1CCN(*)CC1 ; *C1CCN(*)CC1 ; *N1CCN(*)CC1]

In one aspect, disclosed are compounds having a structure represented by a formula:

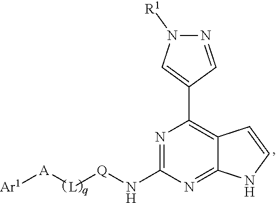

wherein q is selected from 0 and 1; wherein A is a structure selected from:

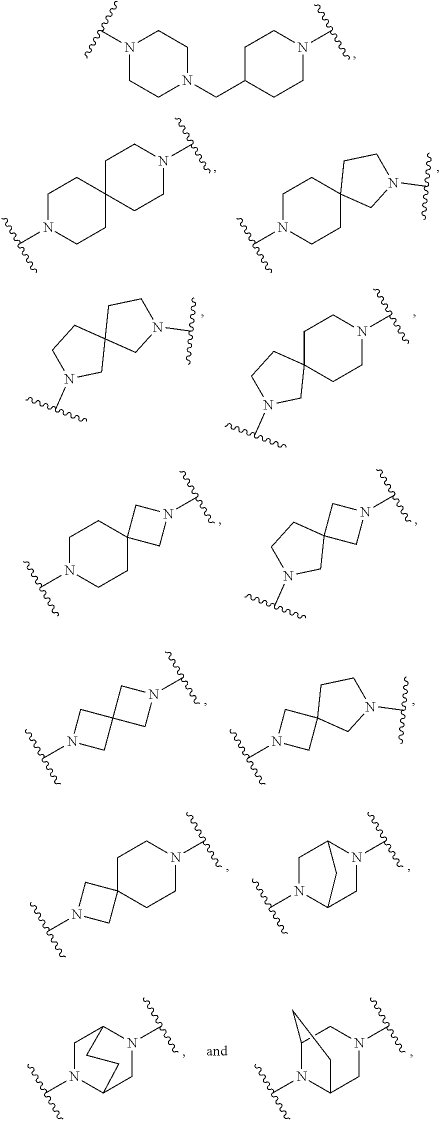

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

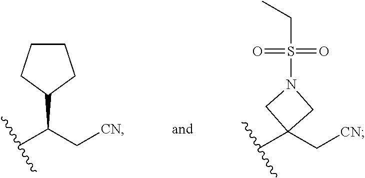 [SMILES: *[C@H](CC#N)C1CCCC1 ; "and" ; *C1(CC#N)CN(S(=O)(=O)CC)C1]

and
wherein Ar¹ is a structure selected from:

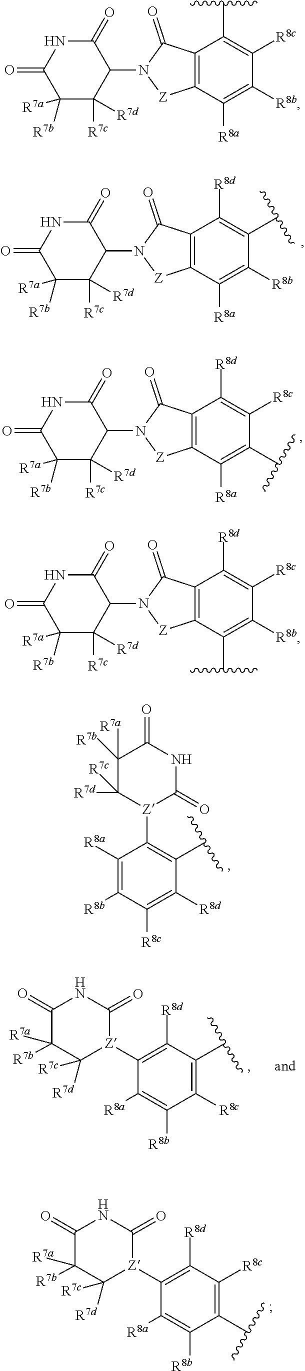

wherein Z is selected from —CH₂— and C(O); wherein Z′ is selected from —CH— and —N—; wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In one aspect, disclosed are compounds having a structure represented by a formula:

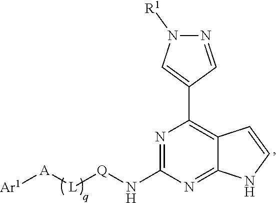

wherein q is selected from 0 and 1; wherein A is selected from —O—, —S—, —NH—, —NHCH₂—, —OCH₂C(O)NH—, —CH₂—, and a structure selected from:

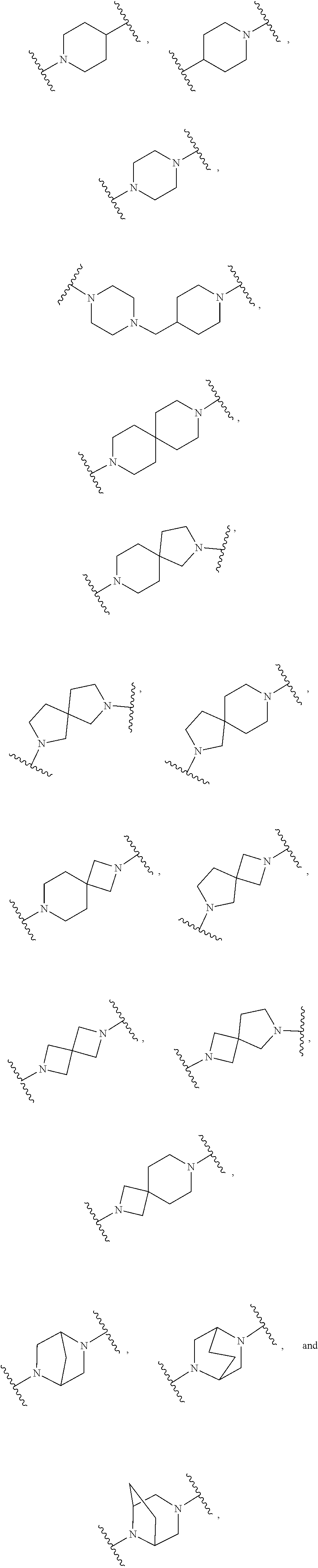

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

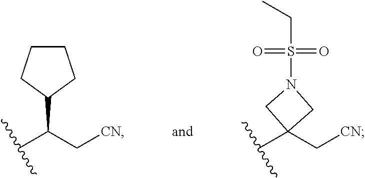 [SMILES: *[C@H](CC#N)C1CCCC1 ; "and" ; *C1(CC#N)CN(S(=O)(=O)CC)C1]

and
wherein Ar¹ is a structure selected from:

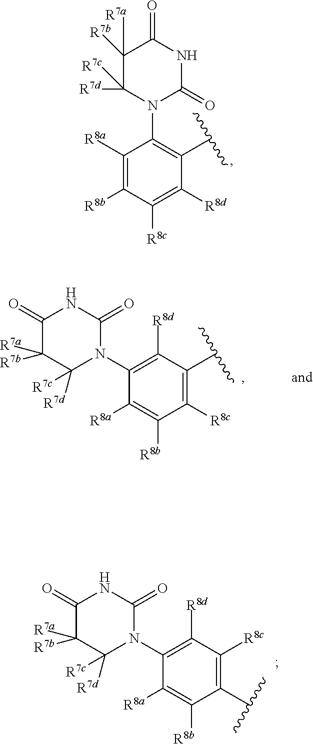

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, or a pharmaceutically acceptable salt thereof.

In one aspect, disclosed are compounds compound having a structure represented by a formula:

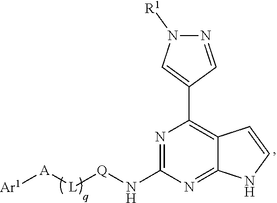

wherein q is selected from 0 and 1; wherein A is selected from —O—, —S—, —NH—, —NHCH₂—, —OCH₂C(O)NH—, —CH₂—, and a structure selected from:

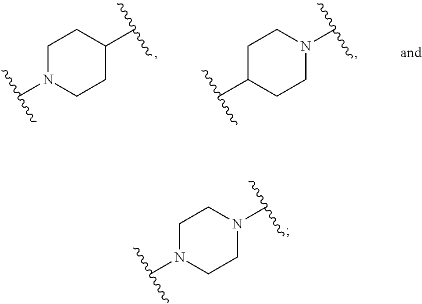

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

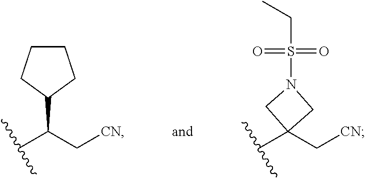 [SMILES: *[C@H](CC#N)C1CCCC1 ; "and" ; *C1(CC#N)CN(S(=O)(=O)CC)C1]

and
wherein Ar¹ is a structure selected from:

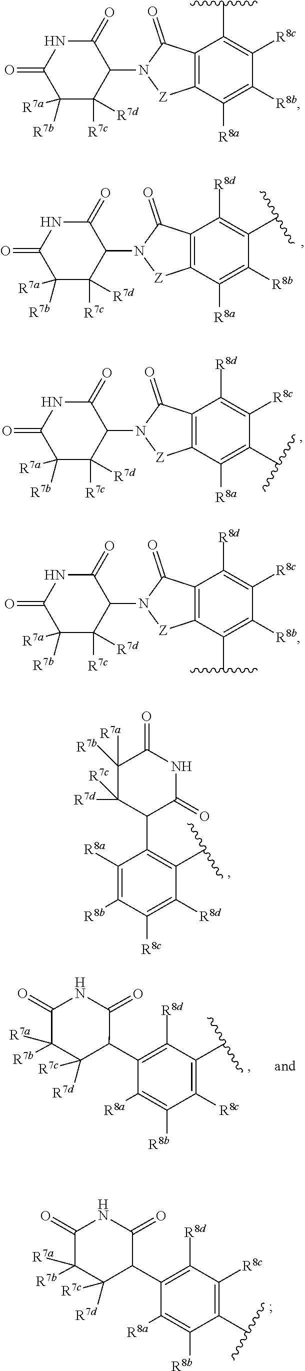

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

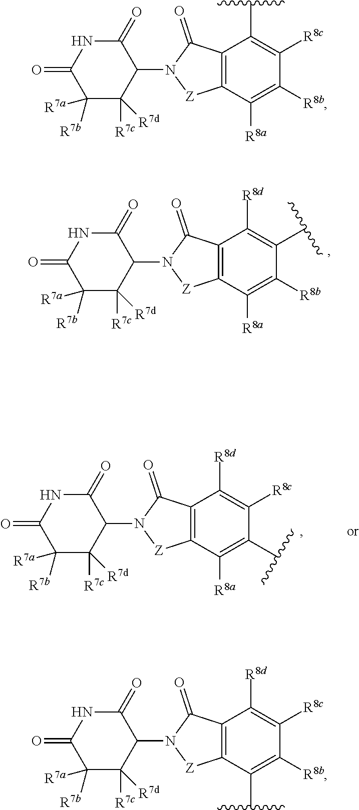

and q is 1, then A is a structure selected from:

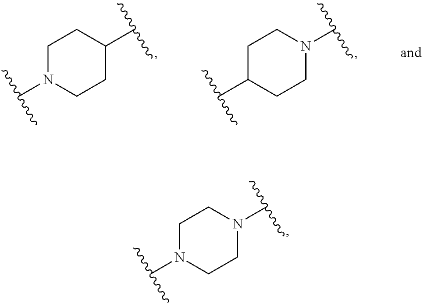 [SMILES: *C1CCN(*)CC1 ; *C1CCN(*)CC1 ; *N1CCN(*)CC1]

or a pharmaceutically acceptable salt thereof.

In one aspect, disclosed are compounds having a structure represented by a formula:

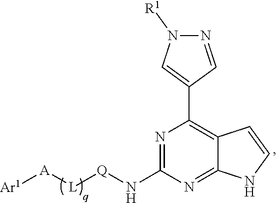

wherein q is selected from 0 and 1; wherein A is selected from —O—, —S—, —NH—, —NHCH₂—, —CH₂—, and a structure selected from:

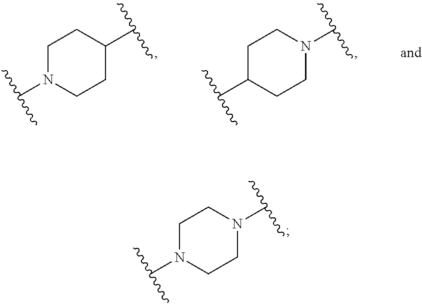

wherein L is selected from C2-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

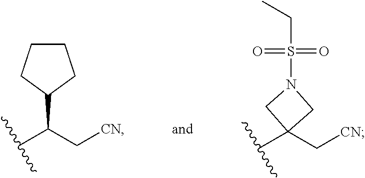 [SMILES: *[C@H](CC#N)C1CCCC1 ; "and" ; *C1(CC#N)CN(S(=O)(=O)CC)C1]

and
wherein Ar¹ is a structure selected from:

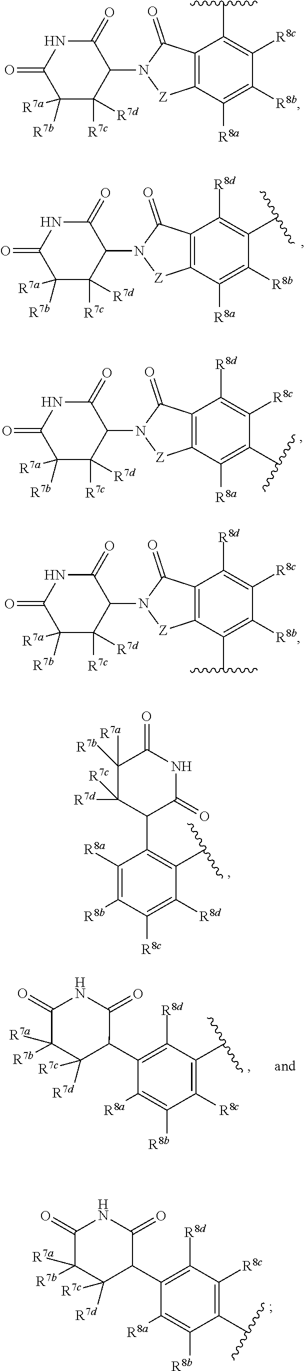

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

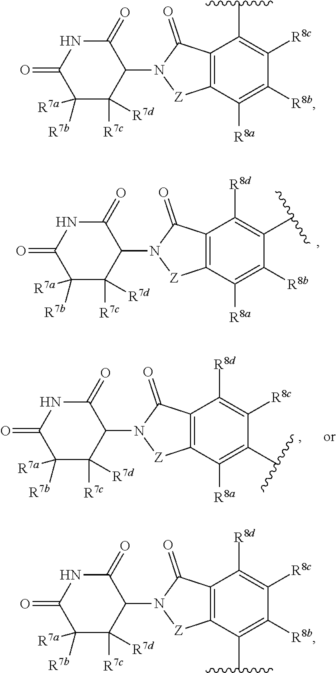

and q is 1, then A is a structure selected from:

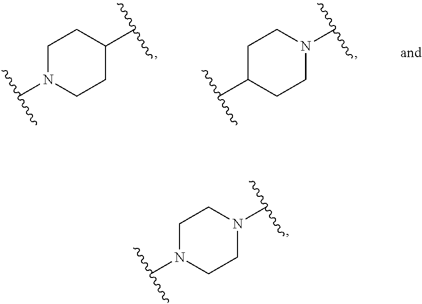 [SMILES: *C1CCN(*)CC1 ; *C1CCN(*)CC1 ; *N1CCN(*)CC1]

or a pharmaceutically acceptable salt thereof.

In a further aspect, the compound has a structure represented by a formula:

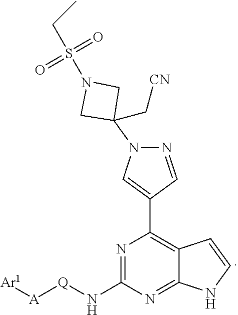

In a further aspect, the compound has a structure represented by a formula:

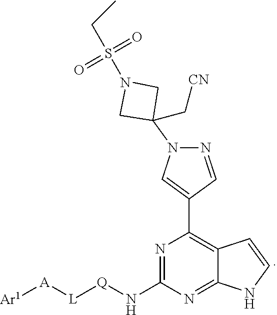

In a further aspect, the compound has a structure represented by a formula:

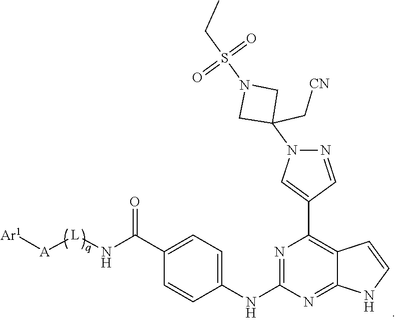

In a further aspect, the compound has a structure represented by a formula:

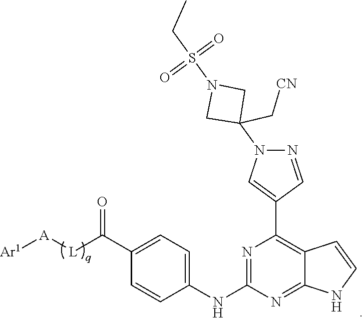

In a further aspect, the compound has a structure represented by a formula:

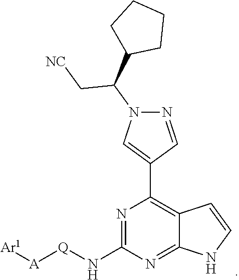

In a further aspect, the compound has a structure represented by a formula:

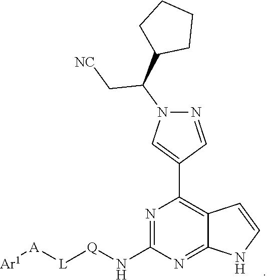

In a further aspect, the compound has a structure represented by a formula:

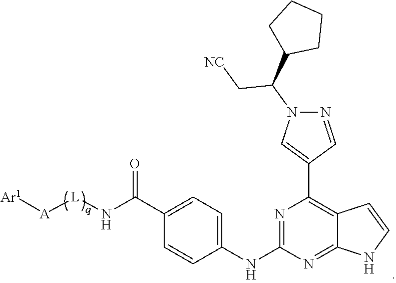

In a further aspect, the compound has a structure represented by a formula:

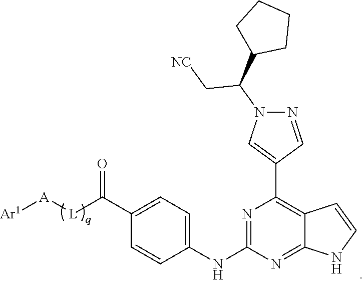

In a further aspect, the compound has a structure represented by a formula:

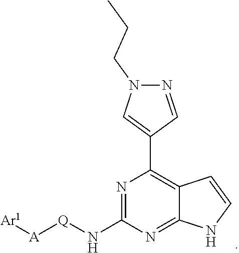

In a further aspect, the compound has a structure represented by a formula:

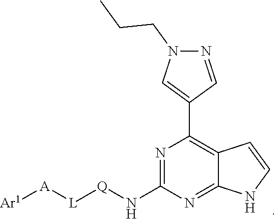

In a further aspect, the compound has a structure represented by a formula:

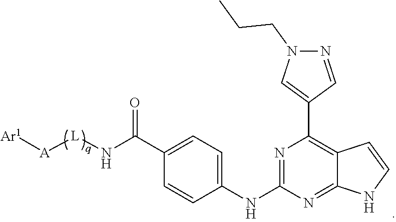

In a further aspect, the compound has a structure represented by a formula:

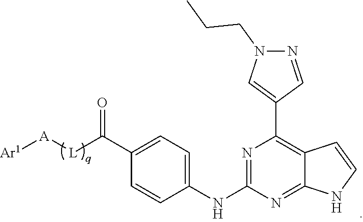

In a further aspect, the compound is selected from:

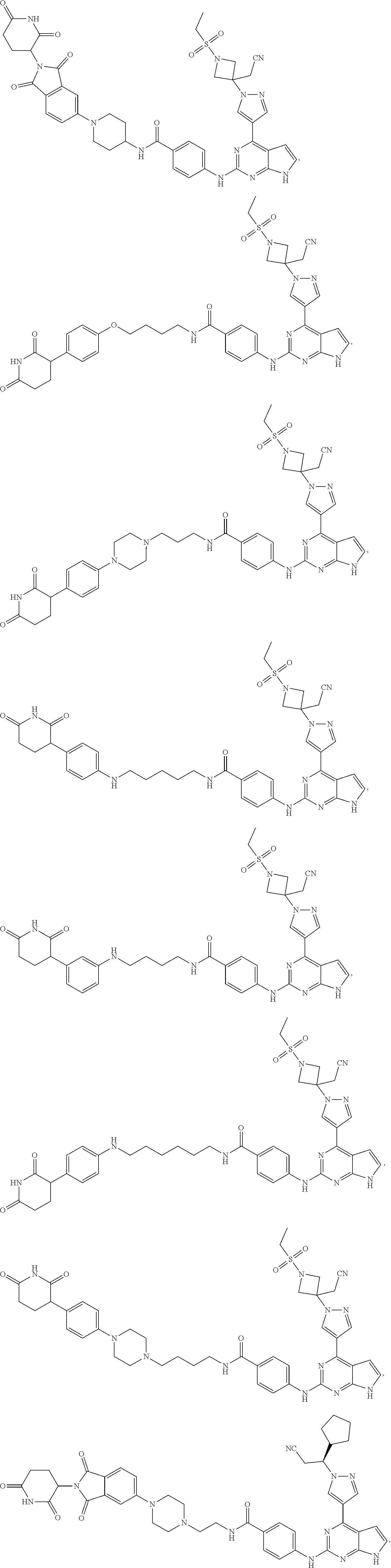

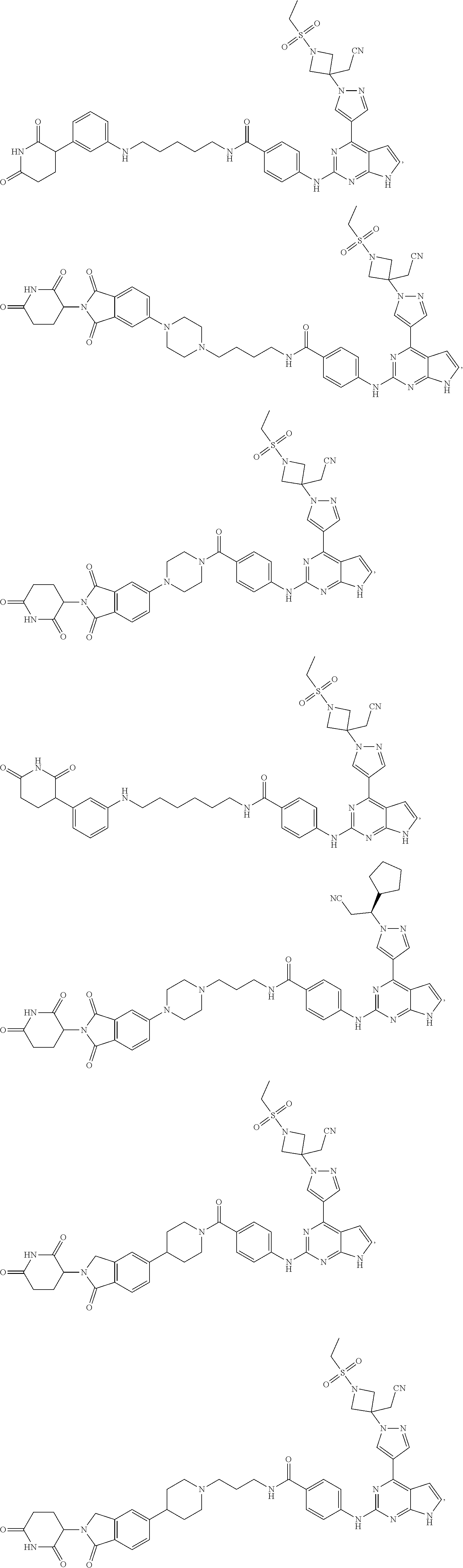

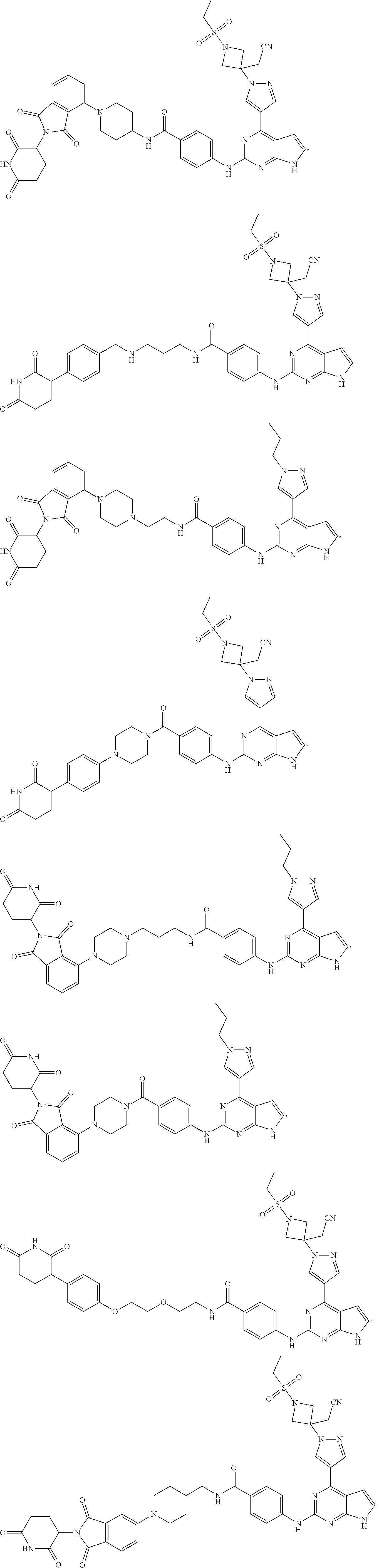

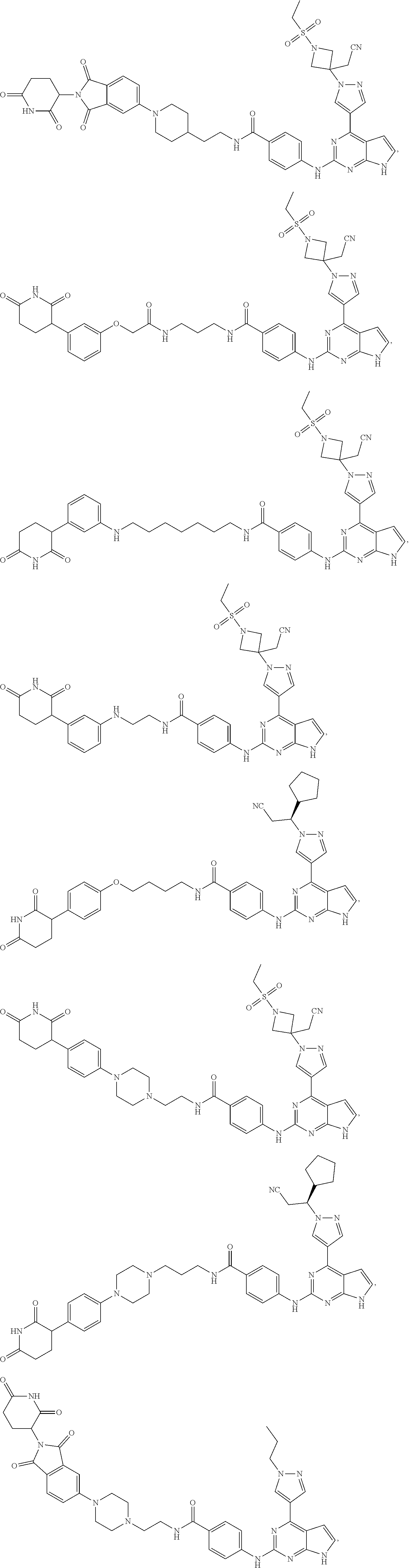

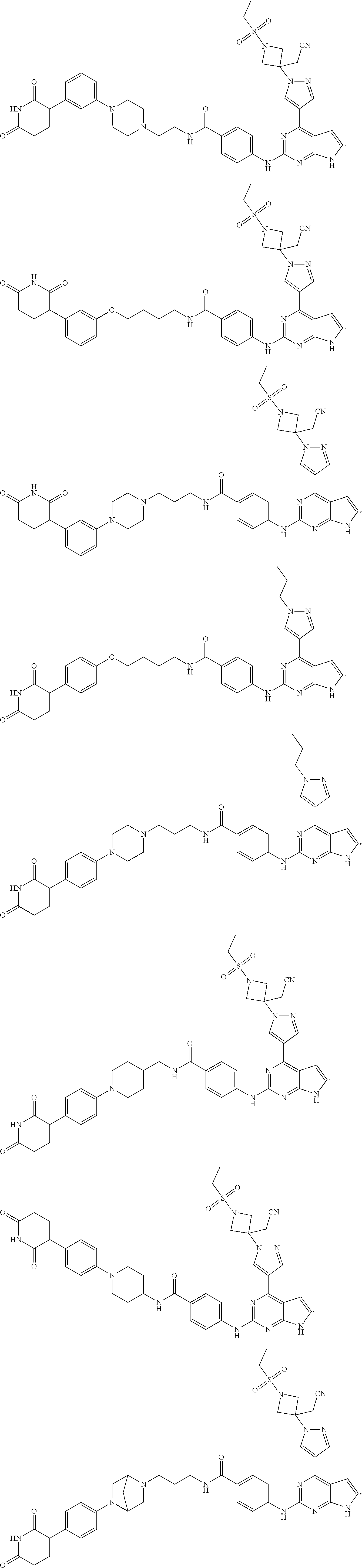

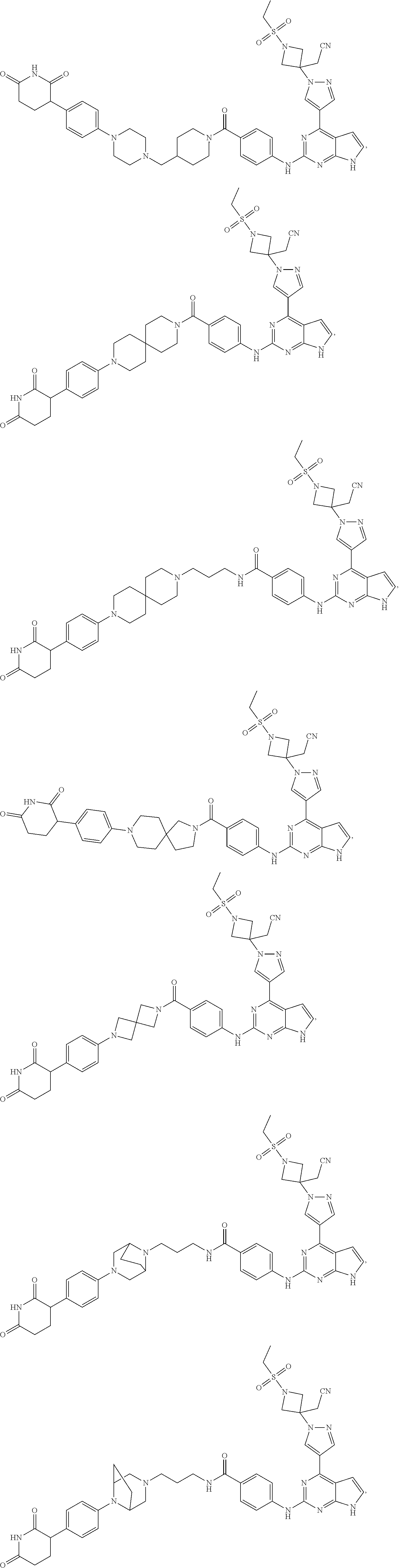

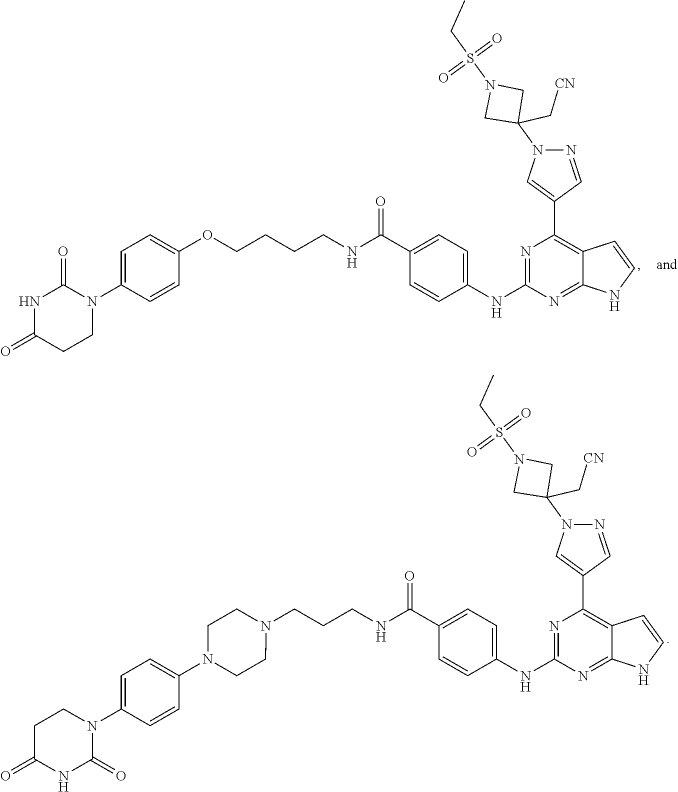 [SMILES: CCS(=O)(=O)N1CC(CC#N)(n2cc(-c3nc(Nc4ccc(C(=O)NCCCCOc5ccc(N6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(N7CCC(=O)NC7=O)cc6)CC5)cc4)nc4[nH]ccc34)cn2)C1]

In a further aspect, the compound is not:

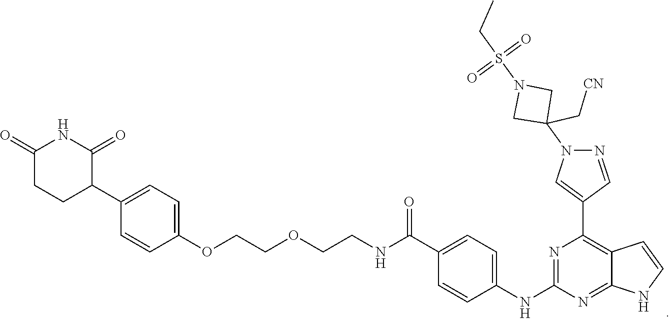 [SMILES: CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1]

In a further aspect, the compound is selected from:

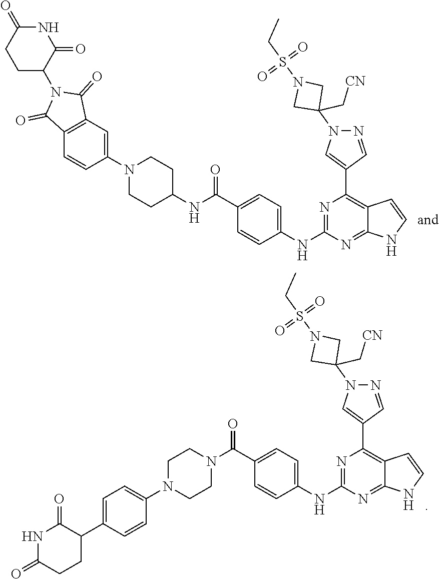

In one aspect, q is selected from 0 and 1. In a further aspect, q is 0. In a still further aspect, q is 1.

In one aspect, n, when present, is selected from 1, 2, 3, 4, 5, 6, 7, and 8. In a further aspect, n, when present, is selected from 1, 2, 3, 4, 5, 6, and 7. In a still further aspect, n, when present, is selected from 1, 2, 3, 4, 5, and 6. In yet a further aspect, n, when present, is selected from 1, 2, 3, 4, and 5. In an even further aspect, n, when present, is selected from 1, 2, 3, and 4. In a still further aspect, n, when present, is selected from 1, 2, and 3. In yet a further aspect, n, when present, is selected from 1 and 2. In an even further aspect, n, when present, is selected from 2, 3, 4, 5, 6, 7, and 8. In a still further aspect, n, when present, is selected from 3, 4, 5, 6, 7, and 8. In yet a further aspect, n, when present, is selected from 4, 5, 6, 7, and 8. In an even further aspect, n, when present, is selected from 5, 6, 7, and 8. In a still further aspect, n, when present, is selected from 6, 7, and 8. In yet a further aspect, n, when present, is selected from 7 and 8. In an even further aspect, n, when present, is selected from 2, 3, 4, 5, 6, and 7. In a still further aspect, n, when present, is selected from 3, 4, 5, and 6. In yet a further aspect, n, when present, is selected from 4 and 5. In an even further aspect, n, when present, is 4.

a. A Groups

In one aspect, A is selected from —O—, —S—, —NH—, —NHCH₂—, —OCH₂C(O)NH—, —CH₂—, and a structure selected from:

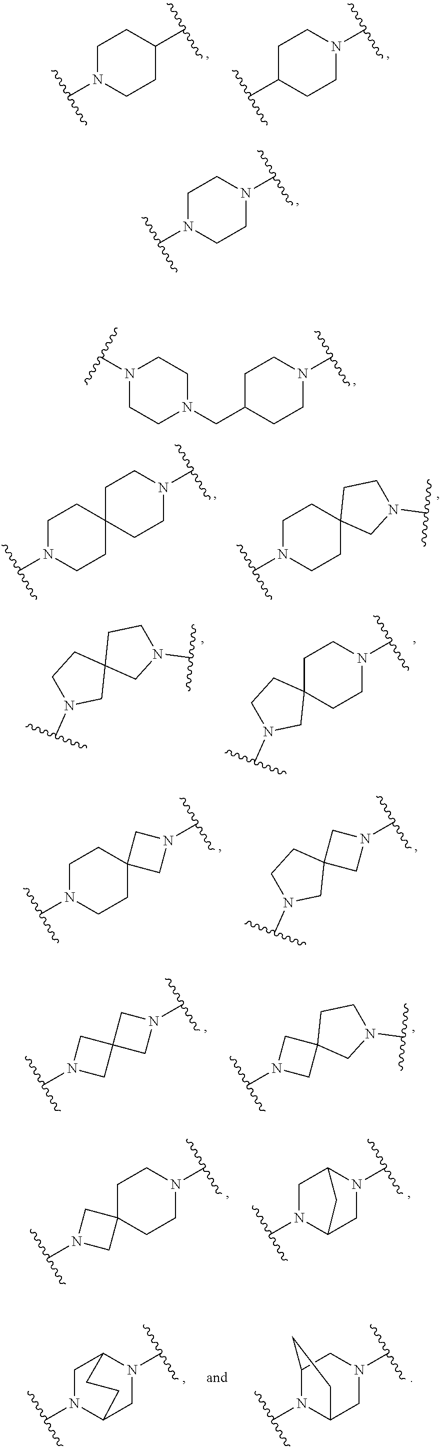

In one aspect, A is selected from —O—, —S—, —NH—, —NHCH₂—, —OCH₂C(O)NH—, —CH₂—, and a structure selected from:

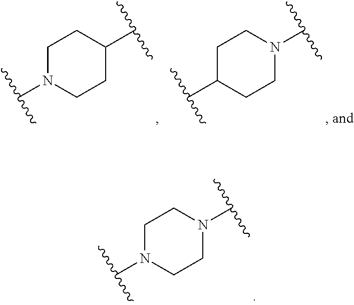 [SMILES: *C1CCN(*)CC1 ; *C1CCN(*)CC1 ; *N1CCN(*)CC1]

In one aspect, A is selected from —O—, —S—, —NH—, —NHCH₂—, —CH₂—, and a structure selected from:

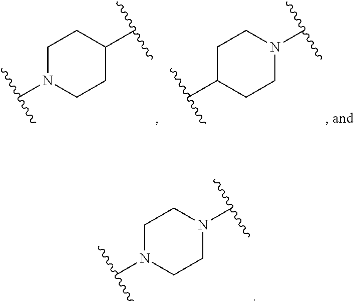 [SMILES: *C1CCN(*)CC1 ; *C1CCN(*)CC1 ; *N1CCN(*)CC1]

In a further aspect, A is selected from —O—, —S—, —NH—, —NHCH₂—, and —CH₂—. In a still further aspect, A is selected from —O—, —S—, and —CH₂—. In yet a further aspect, A is selected from —O— and —CH₂—. In an even further aspect, A is selected from —O— and —S—. In a still further aspect, A is selected from —S— and —CH₂—. In yet a further aspect, A is —O—. In an even further aspect, A is —S—. In a still further aspect, A is —CH₂—.

In a further aspect, A is selected from —O—, —S—, —NH—, and —CH₂—.

In a further aspect, A is selected from —NH— and —NHCH₂—. In a still further aspect, A is —NH—. In yet a further aspect, A is —NHCH₂—.

In a further aspect, A is selected from —NHCH₂— and a structure selected from:

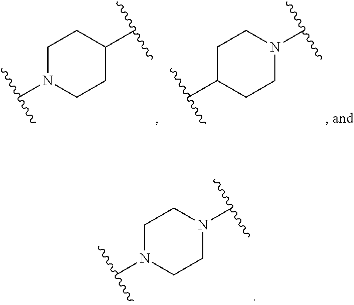 [SMILES: *C1CCN(*)CC1 ; *C1CCN(*)CC1 ; *N1CCN(*)CC1]

In a further aspect, A is a structure selected from:

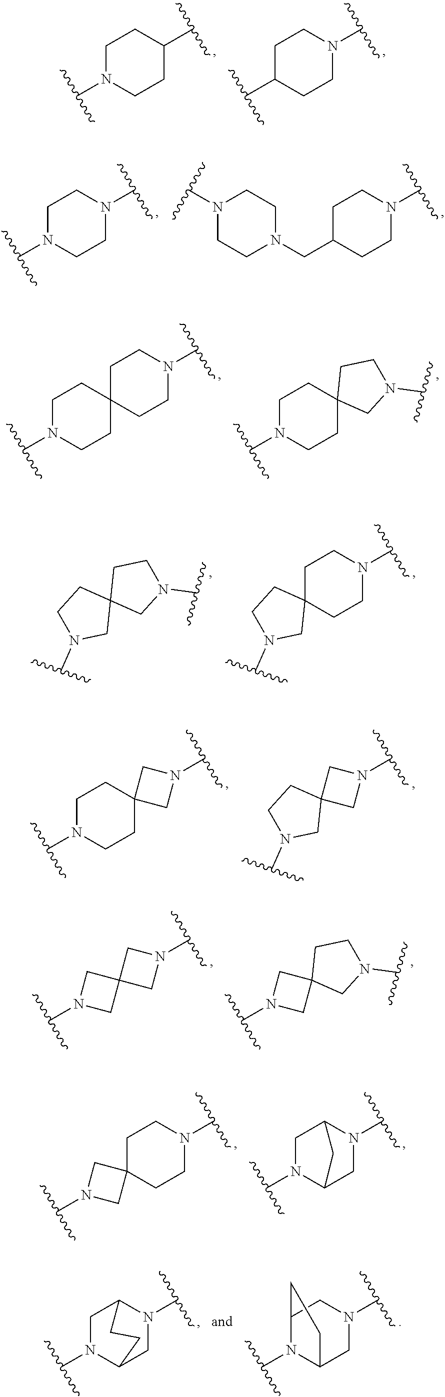

In a further aspect, A is a structure selected from:

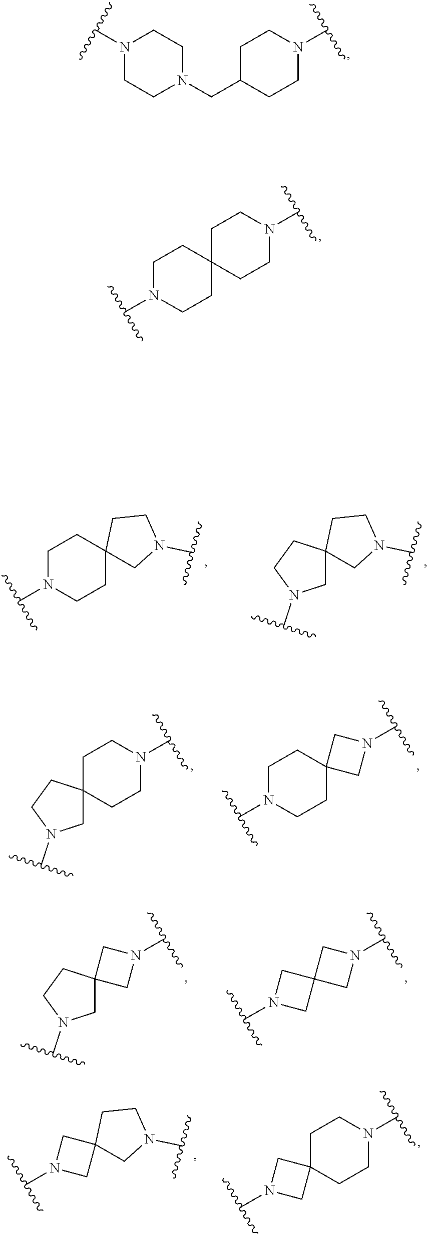

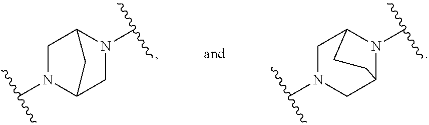 [SMILES: *N1CC2CC1CN2* ; *N1CC2CCC(C1)N2*]

In a further aspect, A is a structure selected from:

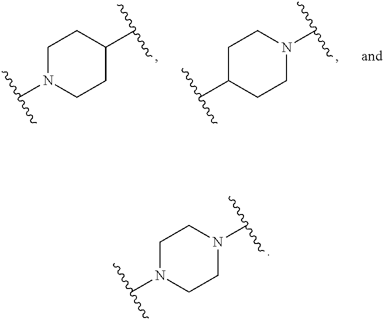 [SMILES: *C1CCN(*)CC1 ; *C1CCN(*)CC1 ; *N1CCN(*)CC1]

In a further aspect, A is a structure:

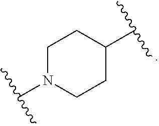 [SMILES: *C1CCN(*)CC1]

In a further aspect, A is a structure:

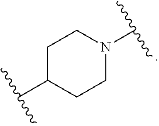 [SMILES: *C1CCN(*)CC1]

In a further aspect, A is a structure:

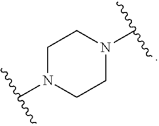 [SMILES: *N1CCN(*)CC1]

b. L Groups

In one aspect, L is selected from C1-C15 alkyl, —(CH₂CH₂O)ₙ—, and —(CH₂CH₂O)ₙ(C1-C4 alkyl)-, wherein n, when present, is selected from 1, 2, 3, 4, 5, 6, 7, and 8.

In one aspect, L is selected from C2-C15 alkyl, —(CH₂CH₂O)ₙ—, and —(CH₂CH₂O)ₙ(C1-C4 alkyl)-, wherein n, when present, is selected from 1, 2, 3, 4, 5, 6, 7, and 8.

In various aspects, L is selected from C2-C15 alkyl and —(CH₂CH₂O)ₙ—. In a further aspect, L is selected from C2-C10 alkyl and —(CH₂CH₂O)ₙ—. In a still further aspect, L is selected from C2-C8 alkyl and —(CH₂CH₂O)ₙ—. In yet a further aspect, L is selected from C2-C4 alkyl and —(CH₂CH₂O)ₙ—. In an even further aspect, L is selected from ethyl, n-propyl, isopropyl, and —(CH₂CH₂O)ₙ—. In a still further aspect, L is selected from ethyl and —(CH₂CH₂O)ₙ—.

In various aspects, L is selected from C2-C15 alkyl and —(CH₂CH₂O)ₙ(C1-C4 alkyl)-. In a further aspect, L is selected from C2-C10 alkyl and —(CH₂CH₂O)ₙ(C1-C4 alkyl)-. In a still further aspect, L is selected from C2-C8 alkyl and —(CH₂CH₂O)ₙ(C1-C4 alkyl)-. In yet a further aspect, L is selected from C2-C4 alkyl and —(CH₂CH₂O)ₙ(C1-C4 alkyl)-. In an even further aspect, L is selected from ethyl, n-propyl, isopropyl, and —(CH₂CH₂O)ₙ(C1-C4 alkyl)-. In a still further aspect, L is selected from ethyl and —(CH₂CH₂O)ₙ(C1-C4 alkyl)-.

In various aspects, L is selected from —(CH₂CH₂O)ₙ— and —(CH₂CH₂O)ₙ(C1-C4 alkyl)-.

In various aspects, L is C2-C15 alkyl. In a further aspect, L is C2-C12 alkyl. In a further aspect, L is C2-C8 alkyl. In a further aspect, L is C2-C6 alkyl. In a further aspect, L is C2-C4 alkyl. In a further aspect, L is C4-C6 alkyl. In a further aspect, L is selected from ethyl, n-propyl, and isopropyl. In a further aspect, L is ethyl.

In a further aspect, L is C5 alkyl. In a still further aspect, L is selected from n-pentyl and neopentyl. In yet a further aspect, L is n-pentyl.

In various aspects, L is —(CH₂CH₂O)ₙ(C1-C4 alkyl)-, wherein n is selected from 1, 2, 3, 4, 5, 6, 7, and 8. In a further aspect, L is —(CH₂CH₂O)ₙ(C1-C4 alkyl)-, wherein n is 1. In a further aspect, L is —(CH₂CH₂O)ₙ(C1-C4 alkyl)-, wherein n is 2. In a further aspect, L is —(CH₂CH₂O)ₙ(C1-C4 alkyl)-, wherein n is 3. In a further aspect, L is —(CH₂CH₂O)ₙ(C1-C4 alkyl)-, wherein n is 4. In a further aspect, L is —(CH₂CH₂O)ₙ(C1-C4 alkyl)-, wherein n is 5. In a further aspect, L is —(CH₂CH₂O)ₙ(C1-C4 alkyl)-, wherein n is 6. In a further aspect, L is —(CH₂CH₂O)ₙ(C1-C4 alkyl)-, wherein n is 7. In a further aspect, L is —(CH₂CH₂O)ₙ(C1-C4 alkyl)-, wherein n is 8.

In various aspects, L is —(CH₂CH₂O)ₙ(C1 alkyl)-, wherein n is selected from 1, 2, 3, 4, 5, 6, 7, and 8. In one aspect, L is —(CH₂CH₂O)ₙ(C2 alkyl)-, wherein n is selected from 1, 2, 3, 4, 5, 6, 7, and 8. In one aspect, L is —(CH₂CH₂O)ₙ(C3 alkyl)-, wherein n is selected from 1, 2, 3, 4, 5, 6, 7, and 8. In one aspect, L is —(CH₂CH₂O)ₙ(C4 alkyl)-, wherein n is selected from 1, 2, 3, 4, 5, 6, 7, and 8. In a further aspect, L is —CH₂CH₂OCH₂CH₂—.

In a further aspect, L is —(CH₂CH₂O)ₙ(C1-C4 alkyl)-. In a still further aspect, L is —(CH₂CH₂O)₁₋₆(C1-C4 alkyl)-. In yet a further aspect, L is —(CH₂CH₂O)₁₋₄(C1-C4 alkyl)-. In an even further aspect, L is —(CH₂CH₂O)₁₋₂(C1-C4 alkyl)-. In a still further aspect, L is —(CH₂CH₂O)(C1-C4 alkyl)-.

In a further aspect, L is selected from —(CH₂CH₂O)ₙ(CH₂)—, —(CH₂CH₂O)ₙ(CH₂CH₂)—, —(CH₂CH₂O)ₙ(CH₂CH₂CH₂)—, and —(CH₂CH₂O)ₙ(CH(CH₃)CH₂)—. In a still further aspect, L is selected from —(CH₂CH₂O)ₙ(CH₂)— and —(CH₂CH₂O)ₙ(CH₂CH₂)—. In yet a further aspect, L is —(CH₂CH₂O)ₙ(CH₂)—. In an even further aspect, L is —(CH₂CH₂O)ₙ(CH₂CH₂)—.

In a further aspect, L is selected from —(CH₂CH₂O)₃(CH₂CH₂)—, —(CH₂CH₂O)₄(CH₂CH₂)—, —(CH₂CH₂O)₅(CH₂CH₂)—, and —(CH₂CH₂O)₆(CH₂CH₂)—. In a still further aspect, L is —CH₂CH₂OCH₂CH₂—.

In various aspects, L is —(CH₂CH₂O)ₙ—, wherein n is selected from 1, 2, 3, 4, 5, 6, 7, and 8. In a further aspect, L is —(CH₂CH₂O)ₙ—, wherein n is 1. In a further aspect, L is —(CH₂CH₂O)ₙ—, wherein n is 2. In a further aspect, L is —(CH₂CH₂O)ₙ—, wherein n is 3. In a further aspect, L is —(CH₂CH₂O)ₙ—, wherein n is 4. In a further aspect, L is —(CH₂CH₂O)ₙ—, wherein n is 5. In a further aspect, L is —(CH₂CH₂O)ₙ—, wherein n is 6. In a further aspect, L is —(CH₂CH₂O)ₙ—, wherein n is 7. In a further aspect, L is —(CH₂CH₂O)ₙ—, wherein n is 8.

In a further aspect, L is —(CH₂CH₂O)ₙ—. In a still further aspect, L is —(CH₂CH₂O)₁₋₆—. In yet a further aspect, L is —(CH₂CH₂O)₁₋₄—. In an even further aspect, L is —(CH₂CH₂O)₁₋₂—. In a still further aspect, L is —(CH₂CH₂O)—.

In a further aspect, L is selected from —(CH₂CH₂O)₃—, —(CH₂CH₂O)₄—, —(CH₂CH₂O)₅—, and —(CH₂CH₂O)₆—. In a still further aspect, L is —CH₂CH₂O—.

c. Q Groups

In one aspect, Q is selected from —C(O)(C₆H₄)—, —NHC(O)(C₆H₄)—, and —C(O)NH(C₆H₄)—. In a further aspect, Q is selected from —C(O)(C₆H₄)— and —NHC(O)(C₆H₄)—. In a still further aspect, Q is selected from —C(O)(C₆H₄)— and —C(O)NH(C₆H₄)—. In yet a further aspect, Q is selected from —NHC(O)(C₆H₄)— and —C(O)NH(C₆H₄)—.

In a further aspect, Q is —NHC(O)(C₆H₄)—. In a further aspect, Q is —C(O)NH(C₆H₄)—. In a further aspect, Q is —C(O)(C₆H₄)—.

In various aspects, Q has a structure selected from:

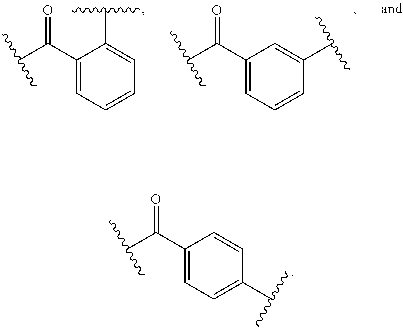 [SMILES: *C(=O)c1ccccc1* ; *C(=O)c1cccc(*)c1 ; *C(=O)c1ccc(*)cc1]

In various aspects, Q has a structure:

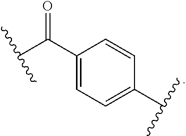 [SMILES: *C(=O)c1ccc(*)cc1]

In various aspects, Q has a structure selected from:

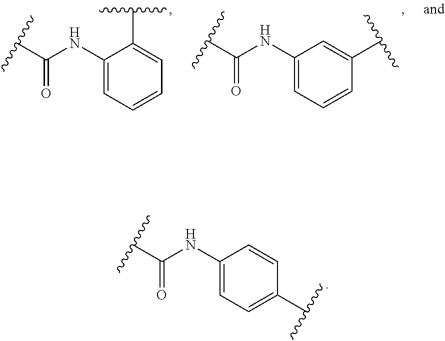 [SMILES: *C(=O)Nc1ccccc1* ; *C(=O)Nc1cccc(*)c1 ; *C(=O)Nc1ccc(*)cc1]

In various aspects, Q has a structure:

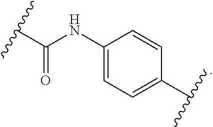 [SMILES: *C(=O)Nc1ccc(*)cc1]

In various aspects, Q has a structure selected from:

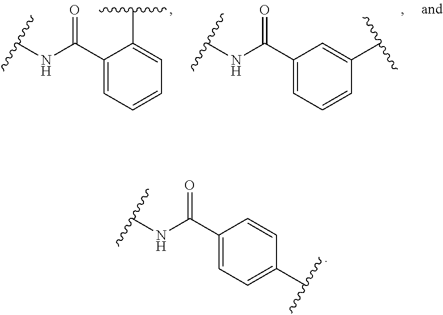 [SMILES: *NC(=O)c1ccccc1* ; *NC(=O)c1cccc(*)c1 ; *NC(=O)c1ccc(*)cc1]

In various aspects, Q has a structure:

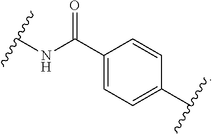 [SMILES: *NC(=O)c1ccc(*)cc1]

d. Z Groups

In one aspect, Z is selected from —CH₂— and —C(O)—. In a further aspect, Z is —CH₂—. In a further aspect, Z is —C(O)—.

e. Z′ Groups

In one aspect, Z′ is selected from —CH— and —N—. In a further aspect, Z′ is —CH—. In a still further aspect, Z′ is —N—.

f. R¹ Groups

In one aspect, R¹ is selected from C1-C8 alkyl, C1-C8 hydroxyalkyl, C1-C8 cyanoalkyl, C1-C8 aminoalkyl, and a structure selected from:

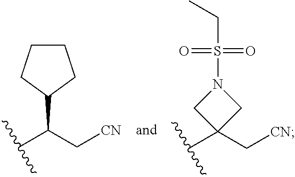 [SMILES: *[C@H](CC#N)C1CCCC1 ; *C1(CC#N)CN(S(=O)(=O)CC)C1]

In a further aspect, R¹ is selected from C1-C8 alkyl, C1-C8 hydroxyalkyl, C1-C8 cyanoalkyl, and C1-C8 aminoalkyl. In a still further aspect, R¹ is selected from C1-C4 alkyl, C1-C4 hydroxyalkyl, C1-C4 cyanoalkyl, and C1-C4 aminoalkyl. In yet a further aspect, R¹ is selected from methyl, ethyl, n-propyl, isopropyl, —CH₂OH, —CH₂CH₂OH, —CH₂CH₂CH₂OH, —CH(CH₃)CH₂OH, —CH₂CN, —CH₂CH₂CN, —CH₂CH₂CH₂CN, —CH(CH₃)CH₂CN, —CH₂NH₂, —CH₂CH₂NH₂, —CH₂CH₂CH₂NH₂, and —CH(CH₃)CH₂NH₂. In a still further aspect, R¹ is selected from methyl, ethyl, —CH₂OH, —CH₂CH₂OH, —CH₂CN, —CH₂CH₂CN, —CH₂NH₂, and —CH₂CH₂NH₂. In yet a further aspect, R¹ is selected from methyl, —CH₂OH, —CH₂CN, and —CH₂NH₂.

In a further aspect, R¹ is selected from n-C1-C8 alkyl, n-C1-C8 hydroxyalkyl, n-C1-C8 cyanoalkyl, n-C1-C8 aminoalkyl, and a structure selected from:

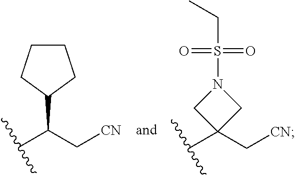 [SMILES: *[C@H](CC#N)C1CCCC1 ; *C1(CC#N)CN(S(=O)(=O)CC)C1]

In a further aspect, R¹ is C1-C8 alkyl. In a further aspect, R¹ is C1-C4 alkyl. In a further aspect, R¹ is n-C3-C8 alkyl. In a further aspect, R¹ is n-C3-C5 alkyl. In a further aspect, R¹ is n-propyl. In a still further aspect, R¹ is selected from methyl, ethyl, n-propyl, and isopropyl. In yet a further aspect, R¹ is selected from methyl and ethyl. In an even further aspect, R¹ is methyl. In an even further aspect, R¹ is n-propyl.

In a further aspect, R¹ is C1-C8 hydroxyalkyl. In a further aspect, R¹ is C1-C4 hydroxyalkyl. In a further aspect, R¹ is n-C3-C8 hydroxyalkyl. In a further aspect, R¹ is n-C3-C5 hydroxyalkyl. In a still further aspect, R¹ is selected from —CH₂OH, —CH₂CH₂OH, —CH₂CH₂CH₂OH, and —CH(CH₃)CH₂OH. In yet a further aspect, R¹ is selected from —CH₂OH, and —CH₂CH₂OH. In an even further aspect, R¹ is —CH₂OH.

In a further aspect, R¹ is C1-C8 cyanoalkyl. In a further aspect, R¹ is C1-C4 cyanoalkyl. In a further aspect, R¹ is n-C3-C8 cyanoalkyl. In a further aspect, R¹ is n-C3-C5 cyanoalkyl. In a still further aspect, R¹ is selected from —CH₂CN, —CH₂CH₂CN, —CH₂CH₂CH₂CN, and —CH(CH₃)CH₂CN. In yet a further aspect, R¹ is selected from —CH₂CN, and —CH₂CH₂CN. In an even further aspect, R¹ is —CH₂CN.

In a further aspect, R¹ is C1-C8 aminoalkyl. In a further aspect, R¹ is C1-C4 aminoalkyl. In a further aspect, R¹ is n-C3-C8 aminoalkyl. In a further aspect, R¹ is n-C3-C5 aminoalkyl. In a still further aspect, R¹ is selected from —CH₂NH₂, —CH₂CH₂NH₂, —CH₂CH₂CH₂NH₂, and —CH(CH₃)CH₂NH₂. In yet a further aspect, R¹ is selected from —CH₂NH₂, and —CH₂CH₂NH₂. In an even further aspect, R¹ is —CH₂NH₂.

In a further aspect, R¹ is a structure selected from:

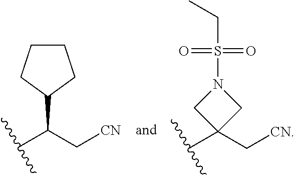 [SMILES: *[C@H](CC#N)C1CCCC1 ; *C1(CC#N)CN(S(=O)(=O)CC)C1]

In a further aspect, R¹ is a structure:

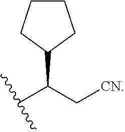 [SMILES: *[C@H](CC#N)C1CCCC1]

In a further aspect, R¹ is a structure:

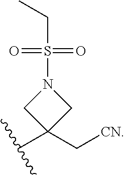 [SMILES: *C1(CC#N)CN(S(=O)(=O)CC)C1]

g. R^{7A}, R^{7B}, R^{7c}, and R^{7D} Groups

In one aspect, each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{7a}, R^{7b}, R^{7c}, and R^{7d} is independently selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃), —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{7a}, R^{7b}, R^{7c}, and R^{7d} is independently selected from hydrogen, —F, —Cl, —NH₂, —OH, —NO₂, —CN, methyl, ethyl, ethenyl, —CH₂F, —CHF₂, —CF₃, —CH₂CH₂F, —CH₂CN, —CH₂CH₂CN, —CH₂OH, —CH₂CH₂OH, —OCH₂F, —OCHF₂, —OCF₃, —OCH₂CH₂F, —OCH₃, —OCH₂CH₃, —NHCH₃, —NHCH₂CH₃, —N(CH₃)₂, —N(CH₃)CH₂CH₃, —N(CH₂CH₃), —CH₂NH₂, and —CH₂CH₂NH₂. In yet a further aspect, each of R^{7a}, R^{7b}, R^{7c}, and R^{7d} is independently selected from hydrogen, —F, —Cl, —NH₂, —OH, —NO₂, —CN, methyl, —CH₂F, —CHF₂, —CF₃, —CH₂CN, —CH₂OH, —OCH₂F, —OCHF₂, —OCF₃, —OCH₃, —NHCH₃, —N(CH₃)₂, and —CH₂NH₂.

In a further aspect, each of R^{7a}, R^{7b}, R^{7c}, and R^{7d} is hydrogen.

In a further aspect, each of R^{7a}, R^{7b}, R^{7c}, and R^{7d} is independently hydrogen. In a further aspect, each of R^{7a}, R^{7b}, R^{7c}, and R^{7d} is independently selected from hydrogen and C1-C4 alkyl. In a further aspect, each of R^{7a}, R^{7b}, R^{7c}, and R^{7d} is independently selected from hydrogen, halogen, —NH₂, —OH, —NO₂, and —CN. In a further aspect, each of R^{7a}, R^{7b}, R^{7c}, and R^{7d} is independently selected from C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl.

In a further aspect, three of R^{7a}, R^{7b}, R^{7c}, and R^{7d} is hydrogen and one of R^{7a}, R^{7b}, R^{7c}, and R^{7d} is selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. For example, R^{7b}, R^{7c}, and R^{7d} can be hydrogen and R⁷a is selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In another example, R^{7a}, R^{7b}, and R^{7d} can be hydrogen and R^{7c} is selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl.

In a further aspect, two of R^{7a}, R^{7b}, R^{7c}, and R^{7d} is hydrogen and two of R^{7a}, R^{7b}, R^{7c}, and R^{7d} is independently selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. For example, R^{7a} and R^{7b} can be hydrogen and each of R^{7b} and R^{7d} is independently selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl.

In various aspects, each of R^{7a}, R^{7b}, R^{7c}, and R^{7d} is independently selected from hydrogen, halogen, —NH₂, —OH, —NO₂, —CN, C1-C4 alkyl, and C2-C4 alkenyl. In a further aspect, each of R^{7a}, R^{7b}, R^{7c}, and R^{7d} is independently selected from hydrogen, —F, —Cl, —NH₂, —OH, —NO₂, —CN, methyl, ethyl, n-propyl, isopropyl, ethenyl, n-propenyl, and isopropenyl. In a still further aspect, each of R^{7a}, R^{7b}, R^{7c}, and R^{7d} is independently selected from hydrogen, —F, —Cl, —NH₂, —OH, —NO₂, —CN, methyl, ethyl, and ethenyl. In yet a further aspect, each of R^{7a}, R^{7b}, R^{7c}, and R^{7d} is independently selected from hydrogen, —F, —Cl, —NH₂, —OH, —NO₂, —CN, and methyl.

In various aspects, each of R^{7a}, R^{7b}, R^{7c}, and R^{7d} is independently selected from hydrogen, halogen, —NH₂, —OH, —NO₂, —CN, C1-C4 haloalkyl, and C1-C4 cyanoalkyl. In a further aspect, each of R^{7a}, R^{7b}, R^{7c}, and R^{7d} is independently selected from hydrogen, —F, —Cl, —NH₂, —OH, —NO₂, —CN, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, and —CH(CH₃)CH₂CN. In a still further aspect, each of R^{7a}, R^{7b}, R^{7c}, and R^{7d} is independently selected from hydrogen, —F, —Cl, —NH₂, —OH, —NO₂, —CN, —CH₂F, —CHF₂, —CF₃, —CH₂CH₂F, —CH₂CN, and —CH₂CH₂CN. In yet a further aspect, each of R^{7a}, R^{7b}, R^{7c}, and R^{7d} is independently selected from hydrogen, —F, —Cl, —NH₂, —OH, —NO₂, —CN, —CH₂F, —CHF₂, —CF₃, and —CH₂CN.

In various aspects, each of R^{7a}, R^{7b}, R^{7c}, and R^{7d} is independently selected from hydrogen, halogen, —NH₂, —OH, —NO₂, —CN, C1-C4 hydroxyalkyl, C1-C4 haloalkoxy, and C1-C4 alkoxy. In a further aspect, each of R^{7a}, R^{7b}, R^{7c}, and R^{7d} is independently selected from hydrogen, —F, —Cl, —NH₂, —OH, —NO₂, —CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, and —OCH(CH₃)₂. In a still further aspect, each of R^{7a}, R^{7b}, R^{7c}, and R^{7d} is independently selected from hydrogen, —F, —Cl, —NH₂, —OH, —NO₂, —CN, —CH₂OH, —CH₂CH₂OH, —OCH₂F, —OCHF₂, —OCF₃, —OCH₂CH₂F, —OCH₃, and —OCH₂CH₃. In yet a further aspect, each of R^{7a}, R^{7b}, R^{7c}, and R^{7d} is independently selected from hydrogen, —F, —Cl, —NH₂, —OH, —NO₂, —CN, —CH₂OH, —OCH₂F, —OCHF₂, —OCF₃, and —OCH₃.

In various aspects, each of R^{7a}, R^{7b}, R^{7c}, and R^{7d} is independently selected from hydrogen, halogen, —NH₂, —OH, —NO₂, —CN, C1-C4 alkylamino, (C1-C4)(C1-C4) dialkylamino, and C1-C4 aminoalkyl. In a further aspect, each of R^{7a}, R^{7b}, R^{7c}, and R^{7d} is independently selected from hydrogen, —F, —Cl, —NH₂, —OH, —NO₂, —CN, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃), —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{7a}, R^{7b}, R^{7c}, and R^{7d} is independently selected from hydrogen, —F, —Cl, —NH₂, —OH, —NO₂, —CN, —NHCH₃, —NHCH₂CH₃, —N(CH₃)₂, —N(CH₃)CH₂CH₃, —N(CH₂CH₃), —CH₂NH₂, and —CH₂CH₂NH₂. In yet a further aspect, each of R^{7a}, R^{7b}, R^{7c}, and R^{7d} is independently selected from hydrogen, —F, —Cl, —NH₂, —OH, —NO₂, —CN, —NHCH₃, —N(CH₃)₂, and —CH₂NH₂.

In various aspects, each of R^{7a}, R^{7b}, R^{7c}, and R^{7d} is independently selected from hydrogen, C1-C4 alkyl, and C2-C4 alkenyl. In a further aspect, each of R^{7a}, R^{7b}, R^{7c}, and R^{7d} is independently selected from hydrogen, methyl, ethyl, n-propyl, isopropyl, ethenyl, n-propenyl, and isopropenyl. In a still further aspect, each of R^{7a}, R^{7b}, R^{7c}, and R^{7d} is independently selected from hydrogen, methyl, ethyl, and ethenyl. In yet a further aspect, each of R^{7a}, R^{7b}, R^{7c}, and R^{7d} is independently selected from hydrogen and methyl.

h. R^{8A}, R^{8B}, R^{8C}, and R^{8D} Groups

In one aspect, each of R^{8a}, R^{8b}, R^{8c}, and R^{8d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{8a}, R^{8b}, R^{8c}, and R^{8d} is independently selected from hydrogen, —F, —Cl, —NH₂, —OH, —NO₂, —CN, methyl, ethyl, n-propyl, isopropyl, ethenyl, n-propenyl, isopropenyl, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃), —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{8a}, R^{8b}, R^{8c}, and R^{8d} is independently selected from hydrogen, —F, —Cl, —NH₂, —OH, —NO₂, —CN, methyl, ethyl, ethenyl, —CH₂F, —CHF₂, —CF₃, —CH₂CH₂F, —CH₂CN, —CH₂CH₂CN, —CH₂OH, —CH₂CH₂OH, —OCH₂F, —OCHF₂, —OCF₃, —OCH₂CH₂F, —OCH₃, —OCH₂CH₃, —NHCH₃, —NHCH₂CH₃, —N(CH₃)₂, —N(CH₃)CH₂CH₃, —N(CH₂CH₃), —CH₂NH₂, and —CH₂CH₂NH₂. In yet a further aspect, each of R^{8a}, R^{8b}, R^{8c}, and R^{8d} is independently selected from hydrogen, —F, —Cl, —NH₂, —OH, —NO₂, —CN, methyl, —CH₂F, —CHF₂, —CF₃, —CH₂CN, —CH₂OH, —OCH₂F, —OCHF₂, —OCF₃, —OCH₃, —NHCH₃, —N(CH₃)₂, and —CH₂NH₂.

In a further aspect, each of R^{8a}, R^{8b}, R^{8c}, and R^{8d} is hydrogen. It is understood that in the structure Ar¹ only three of R^{8a}, R^{8b}, R^{8c}, and R^{8d} are present at the same time. For example, R^{8a}, R^{8b}, and R^{8c} can be present at the same time. In another example, R^{8a}, R^{8b}, and R^{8d} can be present at the same time. In yet another example, R^{8a}, R^{8c}, and R^{8d} can be present at the same time. In yet another example, R^{8b}, R^{8c}, and R^{8d} can be present at the same time.

In a further aspect, each of R^{8a}, R^{8b}, and R^{8c} is independently hydrogen. In a further aspect, each of R^{8a}, R^{8b}, and R^{8d} is independently hydrogen. In a further aspect, each of R^{8a}, R^{8c}, and R^{8d} is independently hydrogen. In a further aspect, each of R^{8b}, R^{8c}, and R^{8d} is independently hydrogen. In a further aspect, each of R^{8a}, R^{8b}, R^{8c}, and R^{8d} is independently selected from hydrogen and C1-C4 alkyl. In a further aspect, each of R^{8a}, R^{8b}, R^{8c}, and R^{8d} is independently selected from hydrogen, halogen, —NH₂, —OH, —NO₂, and —CN. In a further aspect, each of R^{8a}, R^{8b}, R^{8c}, and R^{8d} is independently selected from C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl.

In a further aspect, two of R^{8a}, R^{8b}, R^{8c}, and R^{8d} is hydrogen and one of R^{8a}, R^{8b}, R^{8c}, and R^{8d} is selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. For example, R^{8a} and R^{8b} can be hydrogen and R^{8c} is selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In another example, R^{8b} and R^{8c} can be hydrogen and R^{8a} is selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In another example, R^{8a} and R^{8c} can be hydrogen and R^{8b} is selected from halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl.

In various aspects, each of R^{8a}, R^{8b}, R^{8c}, and R^{8d} is independently selected from hydrogen, halogen, —NH₂, —OH, —NO₂, —CN, C1-C4 alkyl, and C2-C4 alkenyl. In a further aspect, each of R^{8a}, R^{8b}, R^{8c}, and R^{8d} is independently selected from hydrogen, —F, —Cl, —NH₂, —OH, —NO₂, —CN, methyl, ethyl, n-propyl, isopropyl, ethenyl, n-propenyl, and isopropenyl. In a still further aspect, each of R^{8a}, R^{8b}, R^{8c}, and R^{8d} is independently selected from hydrogen, —F, —Cl, —NH₂, —OH, —NO₂, —CN, methyl, ethyl, and ethenyl. In yet a further aspect, each of R^{8a}, R^{8b}, R^{8c}, and R^{8d} is independently selected from hydrogen, —F, —Cl, —NH₂, —OH, —NO₂, —CN, and methyl.

In various aspects, each of R^{8a}, R^{8b}, R^{8c}, and R^{8d} is independently selected from hydrogen, halogen, —NH₂, —OH, —NO₂, —CN, C1-C4 haloalkyl, and C1-C4 cyanoalkyl. In a further aspect, each of R^{8a}, R^{8b}, R^{8c}, and R^{8d} is independently selected from hydrogen, —F, —Cl, —NH₂, —OH, —NO₂, —CN, —CH₂F, —CHF₂, —CF₃, —CH₂CH₂F, —CH₂CH₂CH₂F, —CH(CH₃)CH₂F, —CH₂CN, —CH₂CH₂CN, —CH₂CH₂CH₂CN, and —CH(CH₃)CH₂CN. In a still further aspect, each of R^{8a}, R^{8b}, R^{8c}, and R^{8d} is independently selected from hydrogen, —F, —Cl, —NH₂, —OH, —NO₂, —CN, —CH₂F, —CHF₂, —CF₃, —CH₂CH₂F, —CH₂CN, and —CH₂CH₂CN. In yet a further aspect, each of R^{8a}, R^{8b}, R^{8c}, and R^{8d} is independently selected from hydrogen, —F, —Cl, —NH₂, —OH, —NO₂, —CN, —CH₂F, —CHF₂, —CF₃, and —CH₂CN.

In various aspects, each of R^{8a}, R^{8b}, R^{8c}, and R^{8d} is independently selected from hydrogen, halogen, —NH₂, —OH, —NO₂, —CN, C1-C4 hydroxyalkyl, C1-C4 haloalkoxy, and C1-C4 alkoxy. In a further aspect, each of R^{8a}, R^{8b}, R^{8c}, and R^{8d} is independently selected from hydrogen, —F, —Cl, —NH₂, —OH, —NO₂, —CN, —CH₂OH, —CH₂CH₂OH, —CH₂CH₂CH₂OH, —CH(CH₃)CH₂OH, —OCH₂F, —OCHF₂, —OCF₃, —OCH₂CH₂F, —OCH₂CH₂CH₂F, —OCH(CH₃)CH₂F, —OCH₃, —OCH₂CH₃, —OCH₂CH₂CH₃, and —OCH(CH₃)₂. In a still further aspect, each of R^{8a}, R^{8b}, R^{8c}, and R^{8d} is independently selected from hydrogen, —F, —Cl, —NH₂, —OH, —NO₂, —CN, —CH₂OH, —CH₂CH₂OH, —OCH₂F, —OCHF₂, —OCF₃, —OCH₂CH₂F, —OCH₃, and —OCH₂CH₃. In yet a further aspect, each of R^{8a}, R^{8b}, R^{8c}, and R^{8d} is independently selected from hydrogen, —F, —Cl, —NH₂, —OH, —NO₂, —CN, —CH₂OH, —OCH₂F, —OCHF₂, —OCF₃, and —OCH₃.

In various aspects, each of R^{8a}, R^{8b}, R^{8c}, and R^{8d} is independently selected from hydrogen, halogen, —NH₂, —OH, —NO₂, —CN, C1-C4 alkylamino, (C1-C4)(C1-C4) dialkylamino, and C1-C4 aminoalkyl. In a further aspect, each of R^{8a}, R^{8b}, R^{8c}, and R^{8d} is independently selected from hydrogen, —F, —Cl, —NH₂, —OH, —NO₂, —CN, —NHCH₃, —NHCH₂CH₃, —NHCH₂CH₂CH₃, —NHCH(CH₃)₂, —N(CH₃)₂, —N(CH₃)CH₂CH₃, —N(CH₂CH₃), —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{8a}, R^{8b}, R^{8c}, and R^{8d} is independently selected from hydrogen, —F, —Cl, —NH₂, —OH, —NO₂, —CN, —NHCH₃, —NHCH₂CH₃, —N(CH₃)₂, —N(CH₃)CH₂CH₃, —N(CH₂CH₃), —CH₂NH₂, and —CH₂CH₂NH₂. In yet a further aspect, each of R^{8a}, R^{8b}, R^{8c}, and R^{8d} is independently selected from hydrogen, —F, —Cl, —NH₂, —OH, —NO₂, —CN, —NHCH₃, —N(CH₃)₂, and —CH₂NH₂.

In various aspects, each of R^{8a}, R^{8b}, R^{8c}, and R^{8d} is independently selected from hydrogen, C1-C4 alkyl, and C2-C4 alkenyl. In a further aspect, each of R^{8a}, R^{8b}, R^{8c}, and R^{8d} is independently selected from hydrogen, methyl, ethyl, n-propyl, isopropyl, ethenyl, n-propenyl, and isopropenyl. In a still further aspect, each of R^{8a}, R^{8b}, R^{8c}, and R^{8d} is independently selected from hydrogen, methyl, ethyl, and ethenyl. In yet a further aspect, each of R^{8a}, R^{8b}, R^{8c}, and R^{8d} is independently selected from hydrogen and methyl.

i. AR¹ Groups

In one aspect, Ar¹ is a structure represented by a formula selected from:

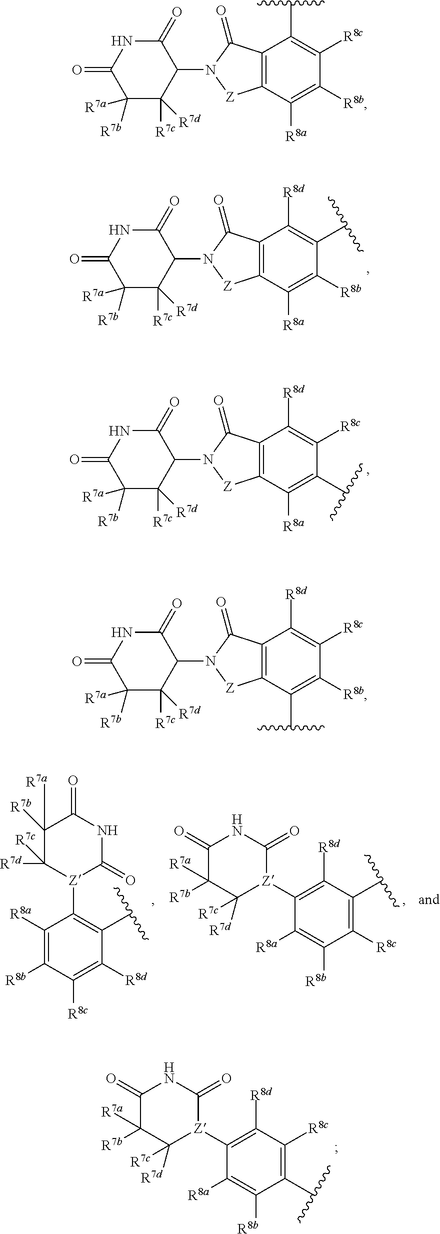

In one aspect, Ar¹ is a structure represented by a formula selected from:

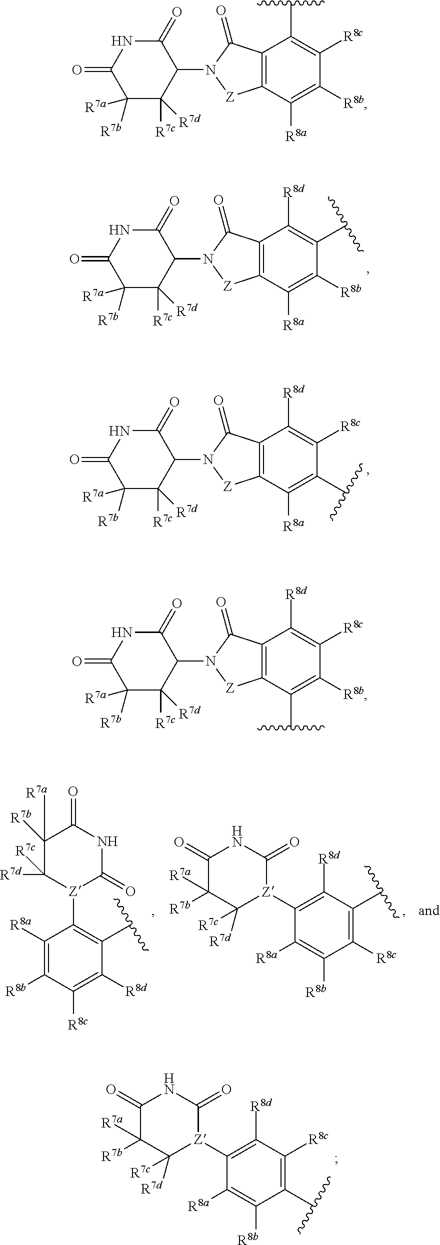

In one aspect, Ar¹ is a structure represented by a formula selected from:

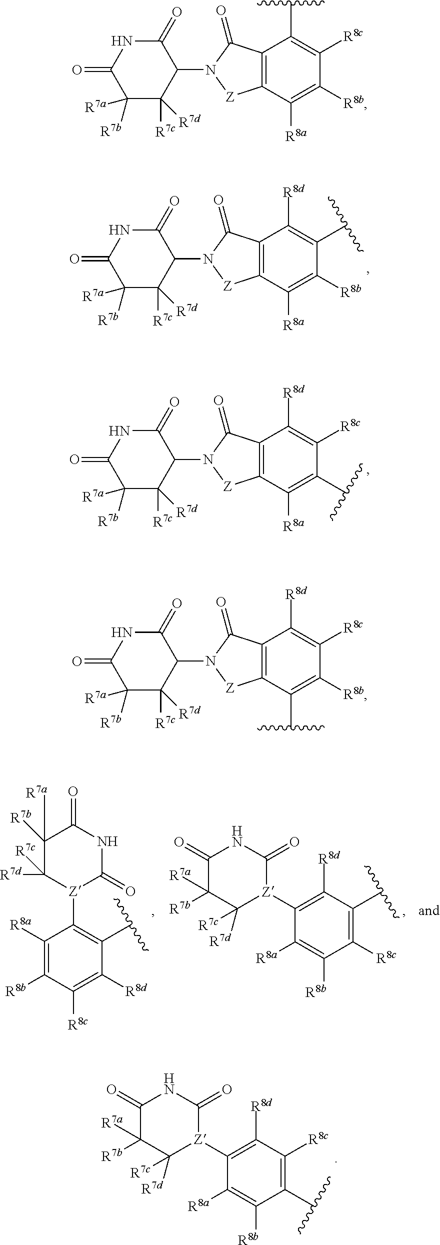

In a further aspect, Ar¹ is a structure selected from:

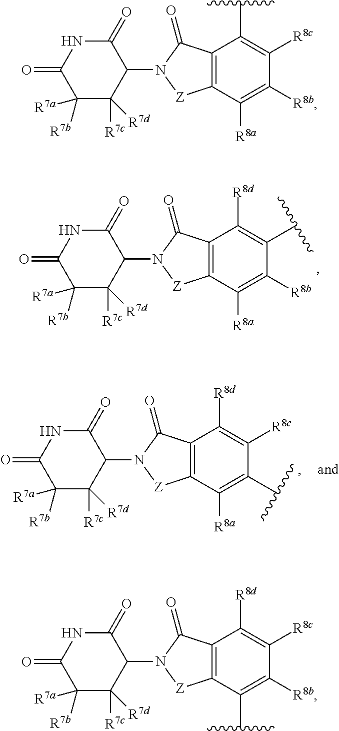

In a further aspect, Ar¹ is a structure represented by a formula:

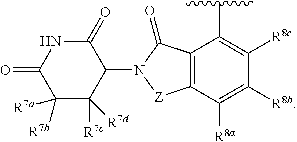

In a further aspect, Ar¹ is a structure represented by a formula:

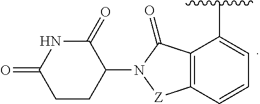

In a further aspect, Ar¹ is a structure:

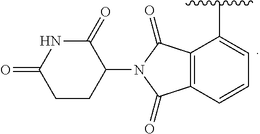 [SMILES: *c1cccc2c1C(=O)N(C1CCC(=O)NC1=O)C2=O]

In a further aspect, Ar¹ is a structure represented by a formula:

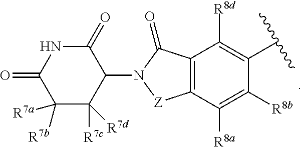

In a further aspect, Ar¹ is a structure represented by a formula:

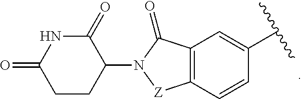

In a further aspect, Ar¹ is a structure represented by a formula:

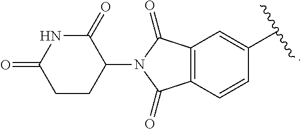 [SMILES: *c1ccc2c(c1)C(=O)N(C1CCC(=O)NC1=O)C2=O ; "."]

In a further aspect, Ar¹ is a structure represented by a formula:

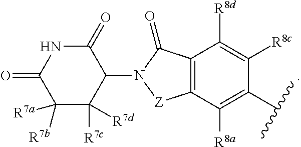

In a further aspect, Ar¹ is a structure represented by a formula:

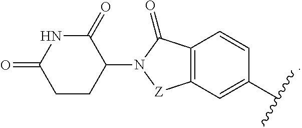

In a further aspect, Ar¹ is a structure represented by a formula:

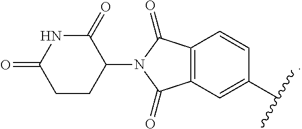 [SMILES: *c1ccc2c(c1)C(=O)N(C1CCC(=O)NC1=O)C2=O ; "."]

In a further aspect, Ar¹ is a structure represented by a formula:

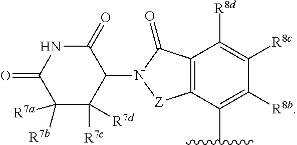

In a further aspect, Ar¹ is a structure represented by a formula:

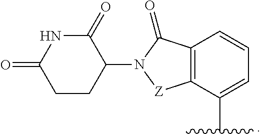

In a further aspect, Ar¹ is a structure represented by a formula:

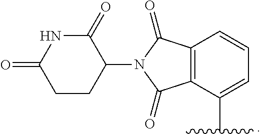 [SMILES: *c1cccc2c1C(=O)N(C1CCC(=O)NC1=O)C2=O ; "."]

In a further aspect, Ar¹ is a structure selected from:

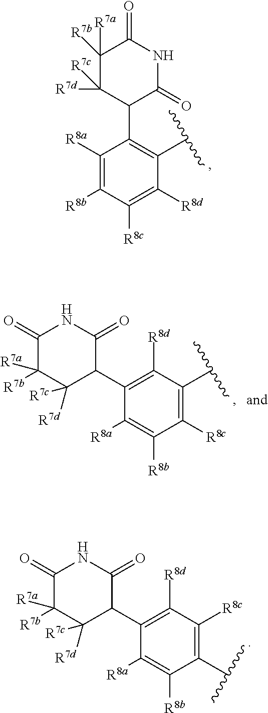

In a further aspect, Ar¹ is a structure:

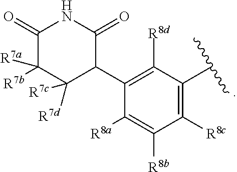

In a further aspect, Ar¹ is a structure:

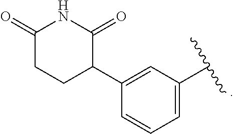 [SMILES: *c1cccc(C2CCC(=O)NC2=O)c1 ; "."]

In a further aspect, Ar¹ is a structure:

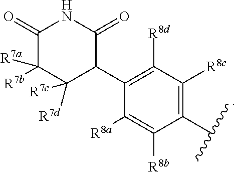

In a further aspect, Ar¹ is a structure:

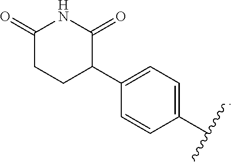 [SMILES: *c1ccc(C2CCC(=O)NC2=O)cc1 ; "."]

In a further aspect, Ar¹ is a structure:

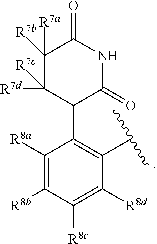

In a further aspect, Ar¹ is a structure:

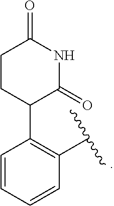 [SMILES: *c1ccccc1C1CCC(=O)NC1=O ; "."]

In a further aspect, Ar¹ is a structure represented by a formula selected from:

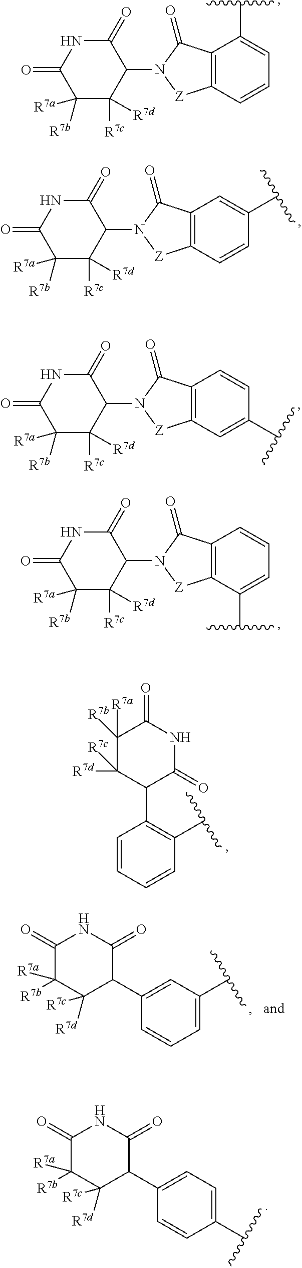

In a further aspect, Ar¹ is a structure represented by a formula selected from:

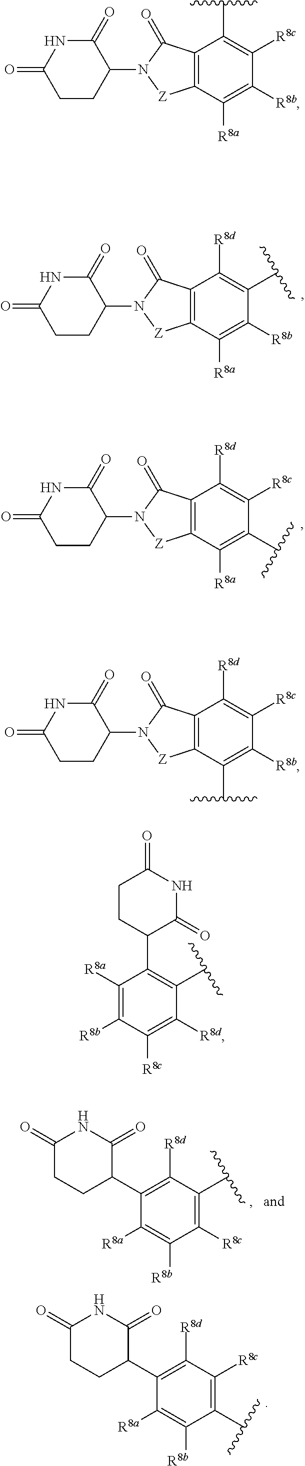

In a further aspect, Ar¹ is a structure represented by a formula selected from:

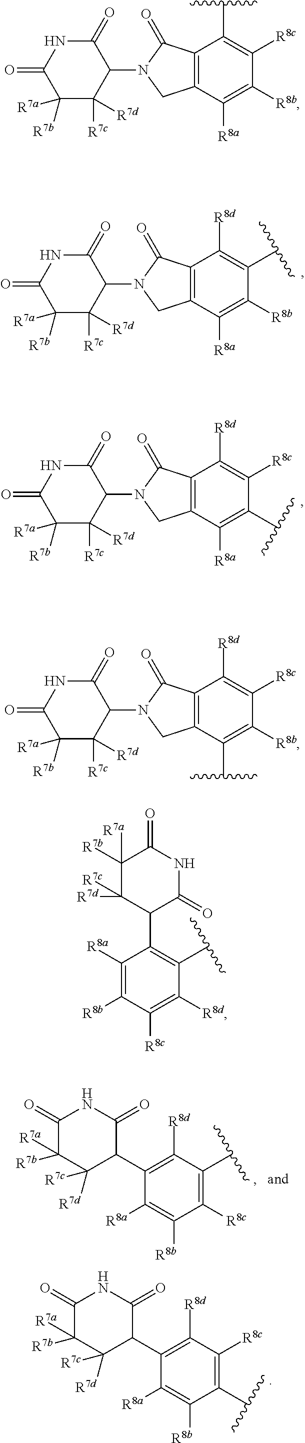

In a further aspect, Ar¹ is a structure represented by a formula selected from:

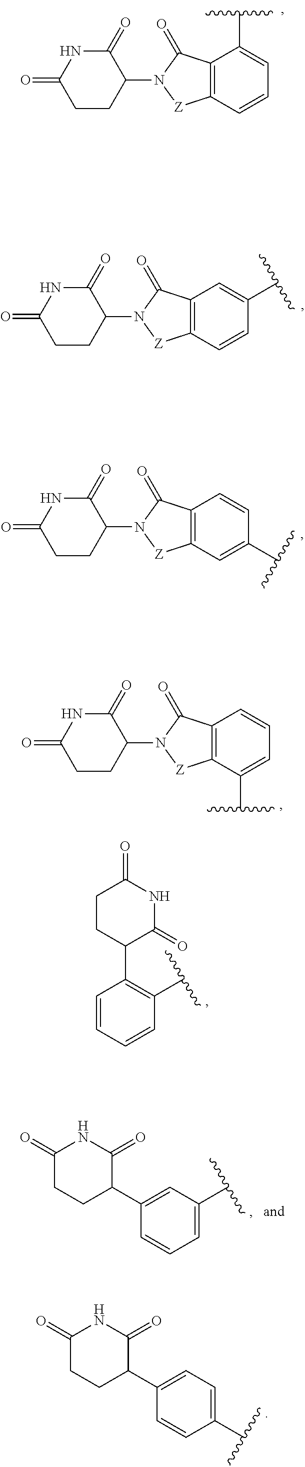

In a further aspect, Ar¹ is a structure represented by a formula selected from:

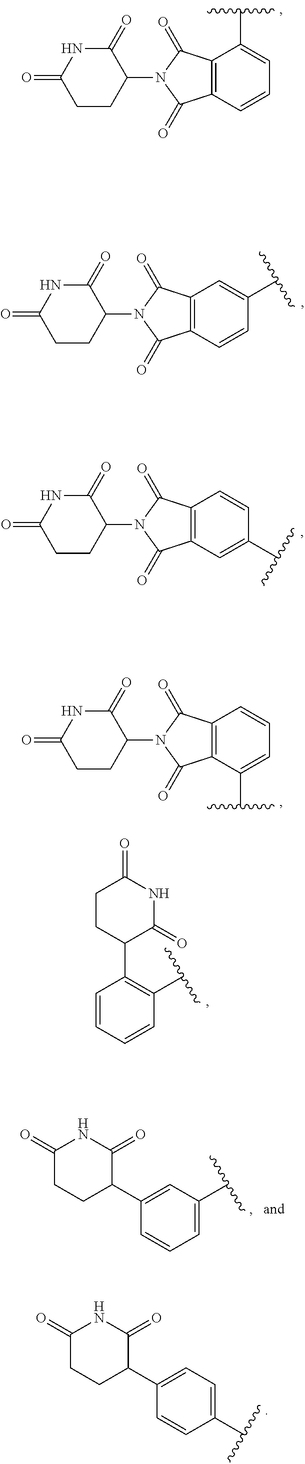

In a further aspect, Ar¹ is a structure represented by a formula selected from:

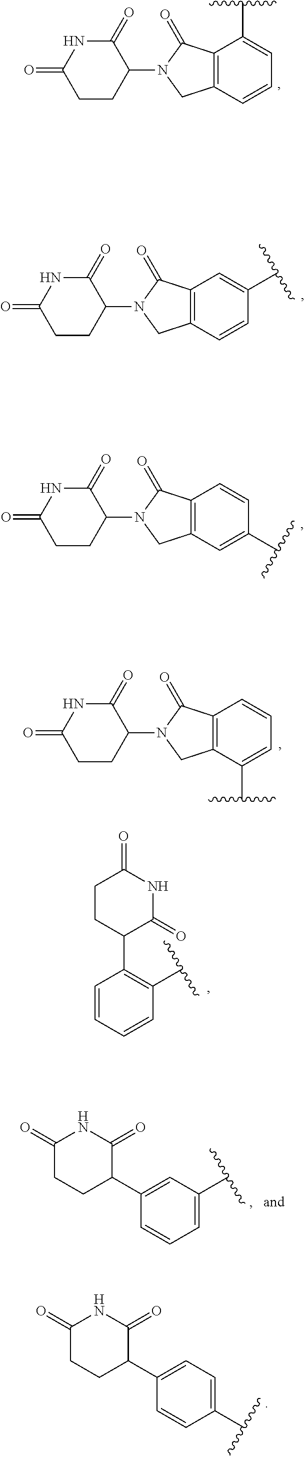

2. Example Compounds

In one aspect, a compound can be present as one or more of the following structures:

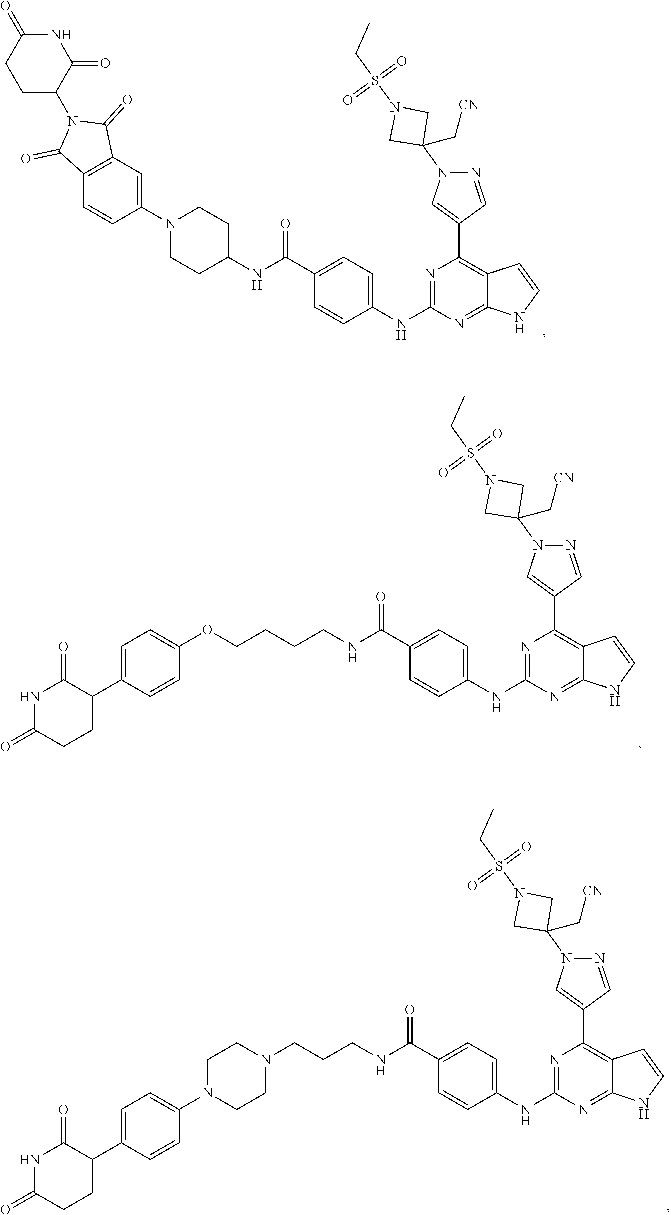 [SMILES: CCS(=O)(=O)N1CC(CC#N)(n2cc(-c3nc(Nc4ccc(C(=O)NC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCOc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(C7CCC(=O)NC7=O)cc6)CC5)cc4)nc4[nH]ccc34)cn2)C1]

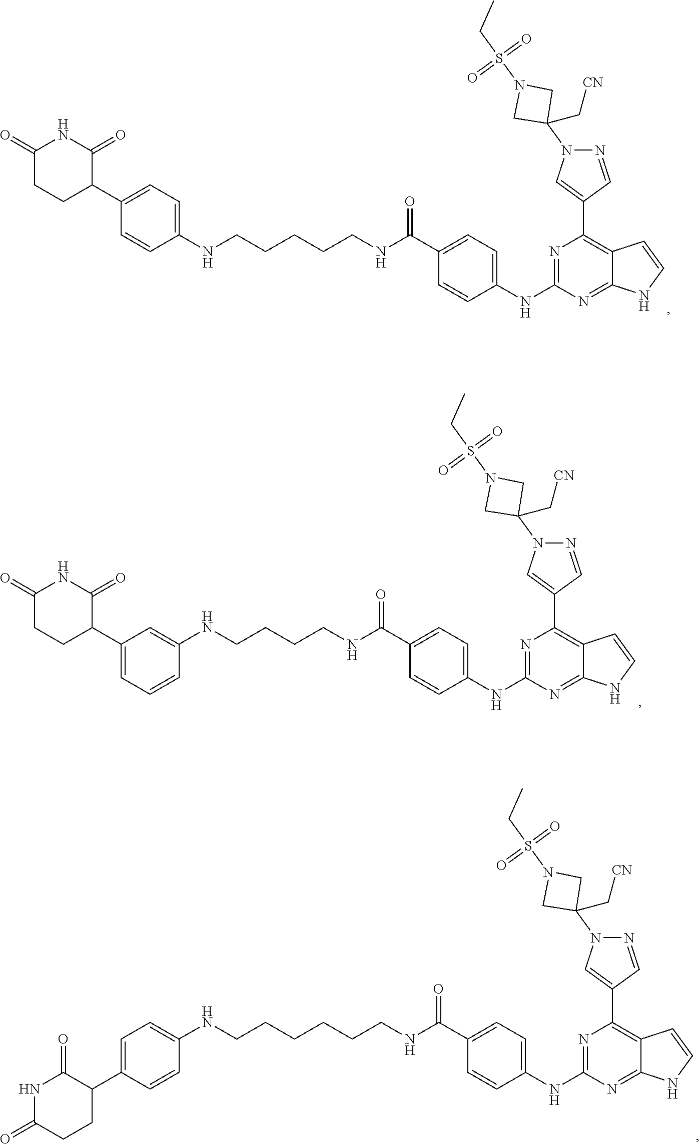 [SMILES: CCS(=O)(=O)N1CC(CC#N)(n2cc(-c3nc(Nc4ccc(C(=O)NCCCCCNc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCNc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCCCCNc5ccc(C6CCC(=O)NC6=O)cc5)cc4)nc4[nH]ccc34)cn2)C1]

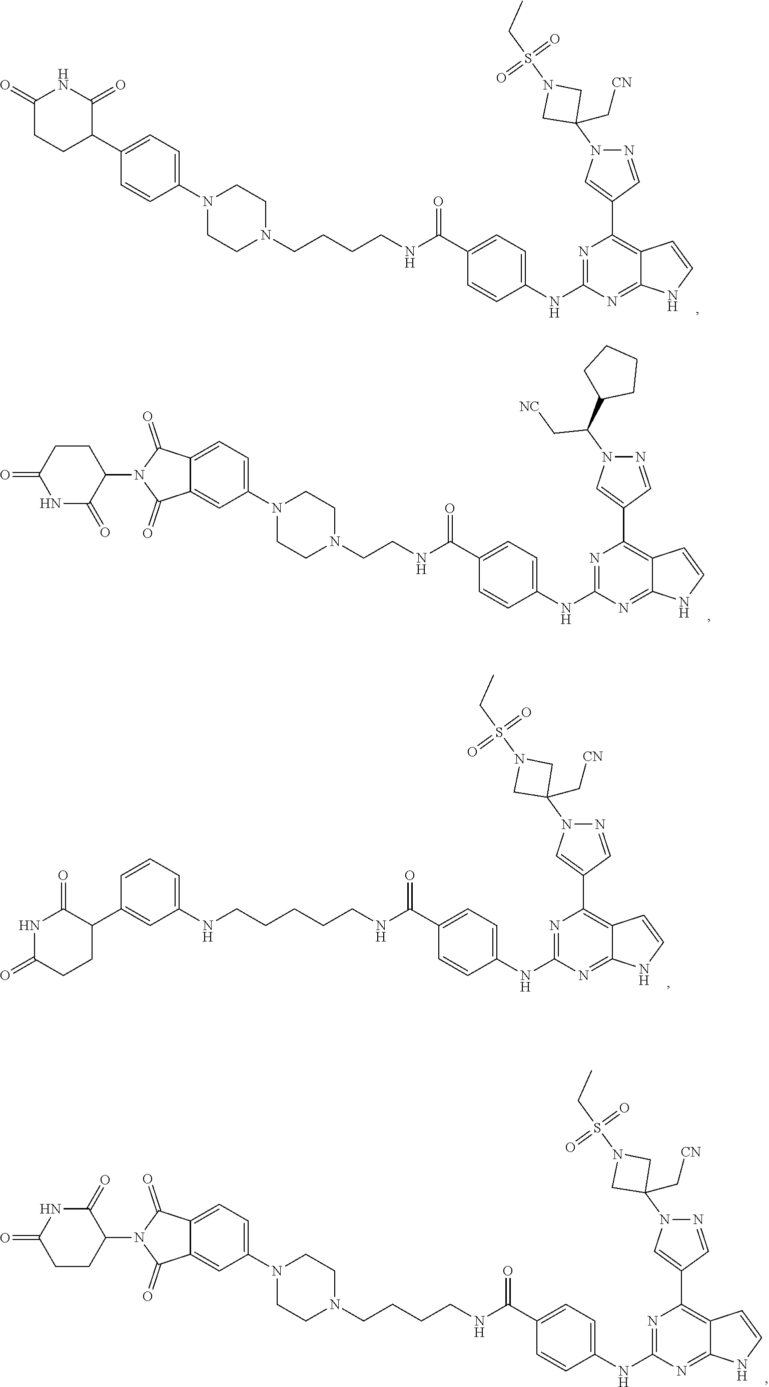

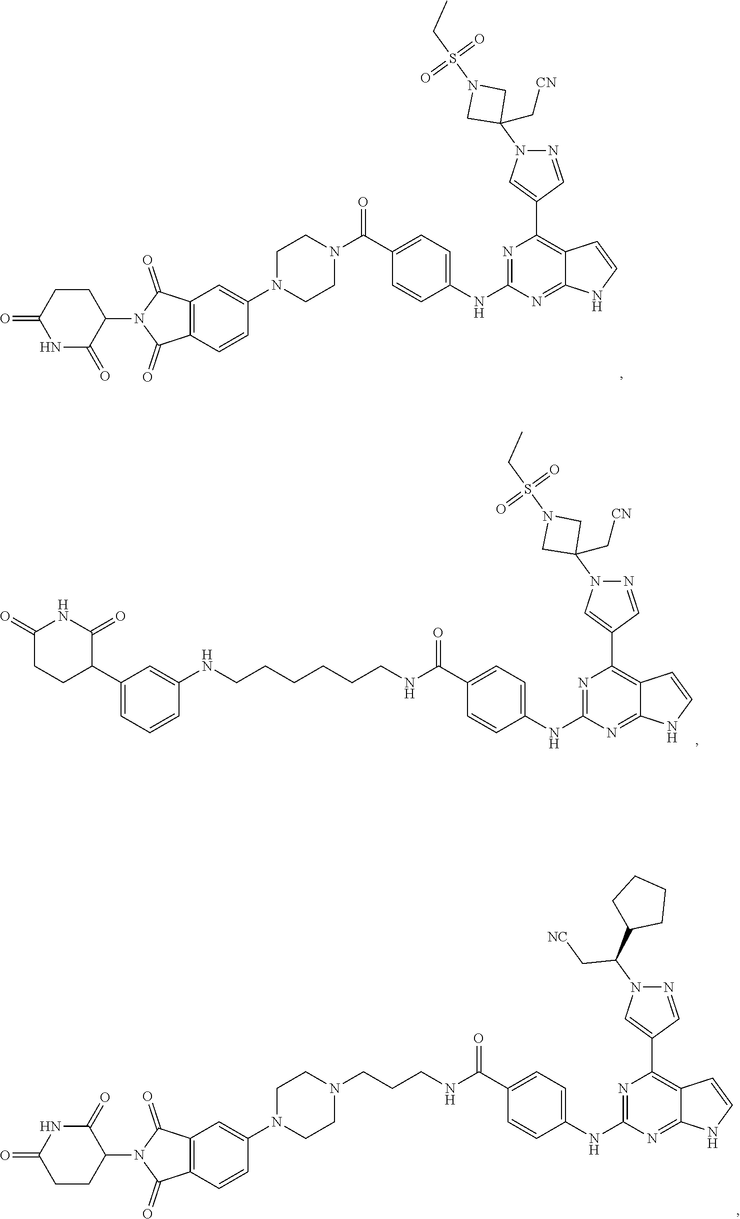 [SMILES: CCS(=O)(=O)N1CC(CC#N)(n2cc(-c3nc(Nc4ccc(C(=O)N5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCCCNc5cccc(C6CCC(=O)NC6=O)c5)cc4)nc4[nH]ccc34)cn2)C1 ; N#CC[C@@H](C1CCCC1)n1cc(-c2nc(Nc3ccc(C(=O)NCCCN4CCN(c5ccc6c(c5)C(=O)N(C5CCC(=O)NC5=O)C6=O)CC4)cc3)nc3[nH]ccc23)cn1]

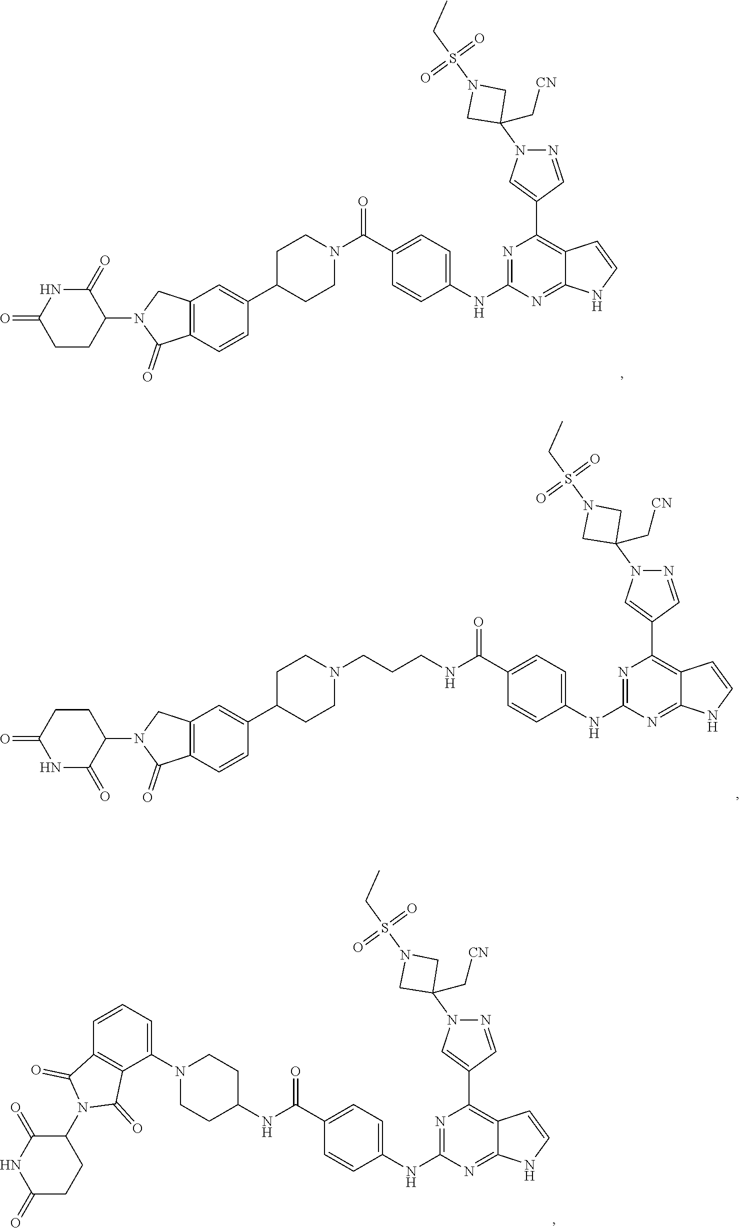 [SMILES: CCS(=O)(=O)N1CC(CC#N)(n2cc(-c3nc(Nc4ccc(C(=O)N5CCC(c6ccc7c(c6)CN(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCN5CCC(c6ccc7c(c6)CN(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NC5CCN(c6cccc7c6C(=O)N(C6CCC(=O)NC6=O)C7=O)CC5)cc4)nc4[nH]ccc34)cn2)C1]

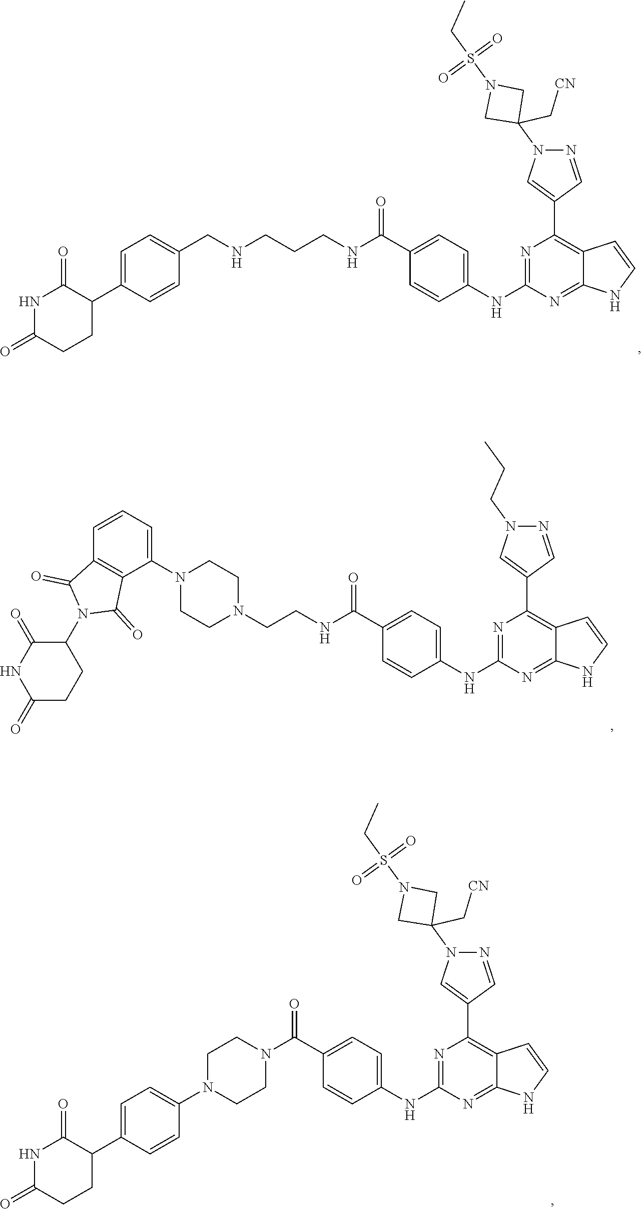 [SMILES: CCS(=O)(=O)N1CC(CC#N)(n2cc(-c3nc(Nc4ccc(C(=O)NCCCNCc5ccc(C6CCC(=O)NC6=O)cc5)cc4)nc4[nH]ccc34)cn2)C1 ; CCCn1cc(-c2nc(Nc3ccc(C(=O)NCCN4CCN(c5cccc6c5C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1]

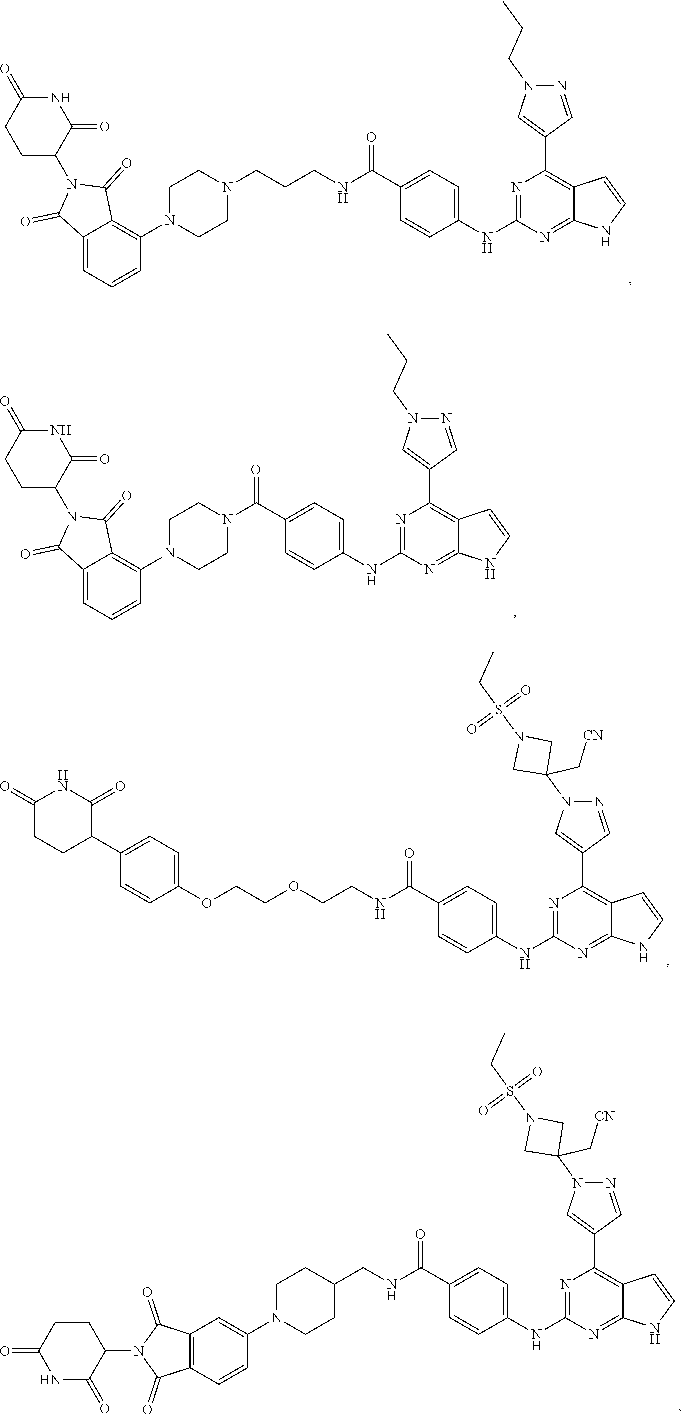 [SMILES: CCCn1cc(-c2nc(Nc3ccc(C(=O)NCCCN4CCN(c5cccc6c5C(=O)N(C5CCC(=O)NC5=O)C6=O)CC4)cc3)nc3[nH]ccc23)cn1 ; CCCn1cc(-c2nc(Nc3ccc(C(=O)N4CCN(c5cccc6c5C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCC5CCN(c6ccc7c(c6)C(=O)N(C6CCC(=O)NC6=O)C7=O)CC5)cc4)nc4[nH]ccc34)cn2)C1]

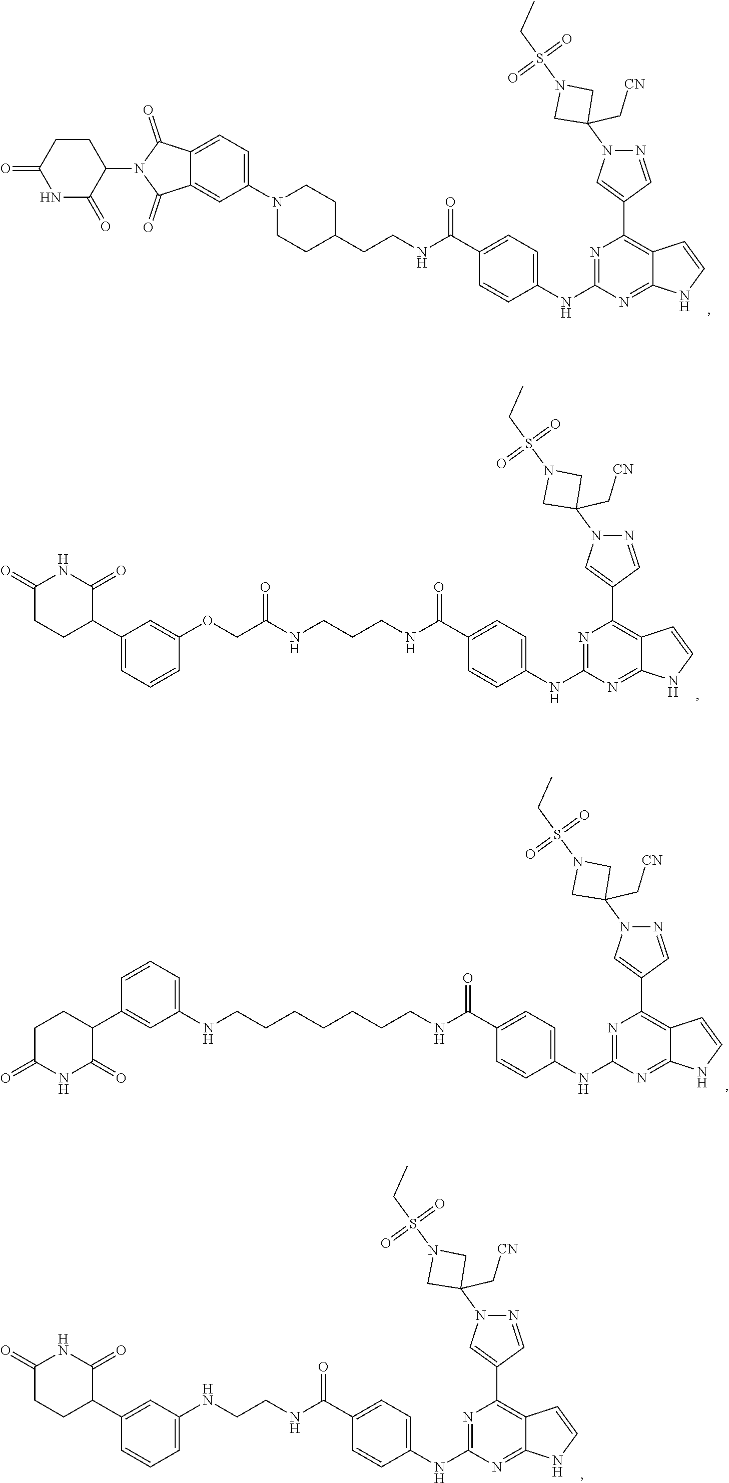 [SMILES: CCS(=O)(=O)N1CC(CC#N)(n2cc(-c3nc(Nc4ccc(C(=O)NCCC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCNC(=O)COc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCCCCCNc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCNc5cccc(C6CCC(=O)NC6=O)c5)cc4)nc4[nH]ccc34)cn2)C1]

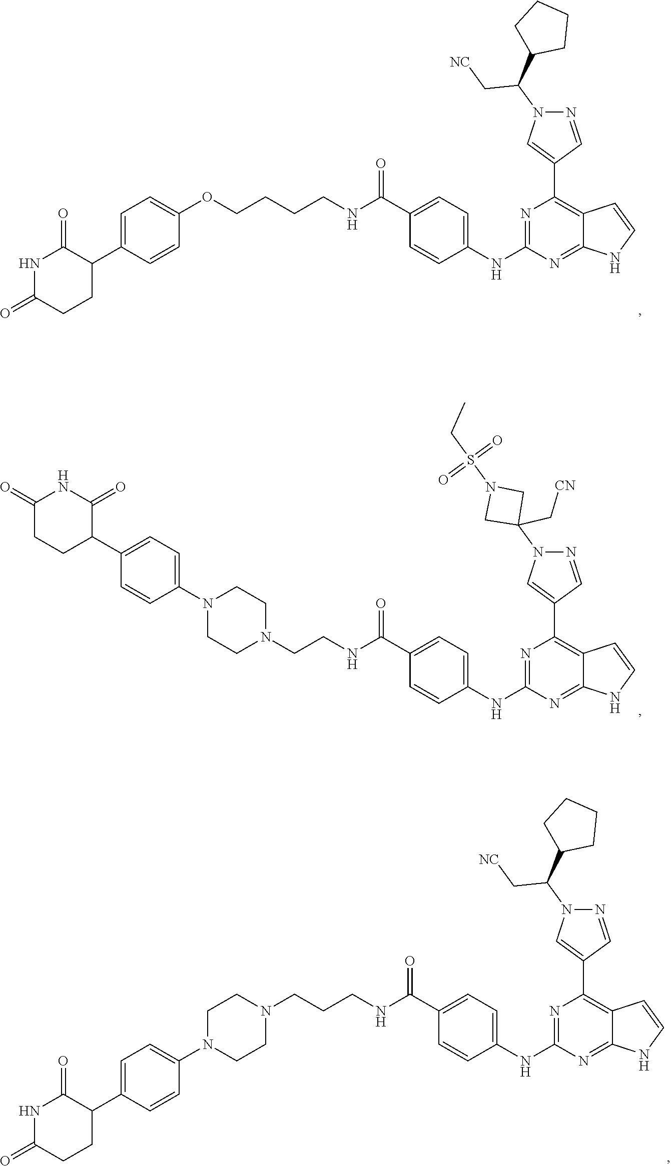 [SMILES: N#CC[C@@H](C1CCCC1)n1cc(-c2nc(Nc3ccc(C(=O)NCCCCOc4ccc(C5CCC(=O)NC5=O)cc4)cc3)nc3[nH]ccc23)cn1 ; CCS(=O)(=O)N1CC(CC#N)(n2cc(-c3nc(Nc4ccc(C(=O)NCCN5CCN(c6ccc(C7CCC(=O)NC7=O)cc6)CC5)cc4)nc4[nH]ccc34)cn2)C1 ; N#CC[C@@H](C1CCCC1)n1cc(-c2nc(Nc3ccc(C(=O)NCCCN4CCN(c5ccc(C6CCC(=O)NC6=O)cc5)CC4)cc3)nc3[nH]ccc23)cn1]

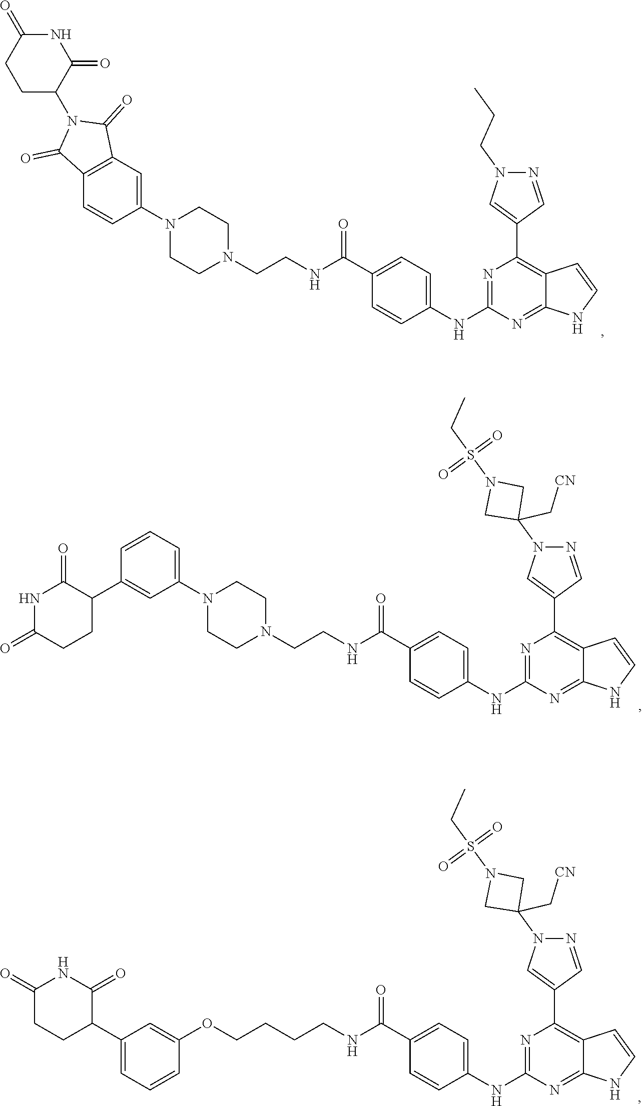 [SMILES: CCCn1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCN5CCN(c6cccc(C7CCC(=O)NC7=O)c6)CC5)cc4)nc4[nH]ccc34)cn2)C1 ; CCS(=O)(=O)N1CC(CC#N)(n2cc(-c3nc(Nc4ccc(C(=O)NCCCCOc5cccc(C6CCC(=O)NC6=O)c5)cc4)nc4[nH]ccc34)cn2)C1]

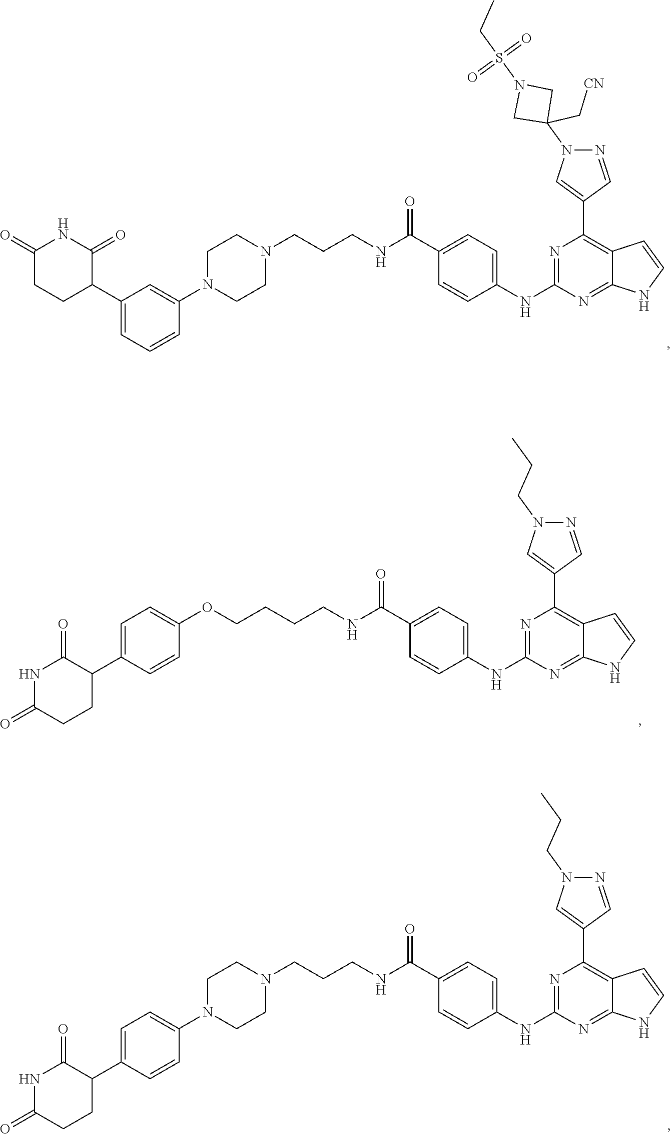 [SMILES: CCS(=O)(=O)N1CC(CC#N)(n2cc(-c3nc(Nc4ccc(C(=O)NCCCN5CCN(c6cccc(C7CCC(=O)NC7=O)c6)CC5)cc4)nc4[nH]ccc34)cn2)C1 ; CCCn1cc(-c2nc(Nc3ccc(C(=O)NCCCCOc4ccc(C5CCC(=O)NC5=O)cc4)cc3)nc3[nH]ccc23)cn1 ; CCCn1cc(-c2nc(Nc3ccc(C(=O)NCCCN4CCN(c5ccc(C6CCC(=O)NC6=O)cc5)CC4)cc3)nc3[nH]ccc23)cn1]

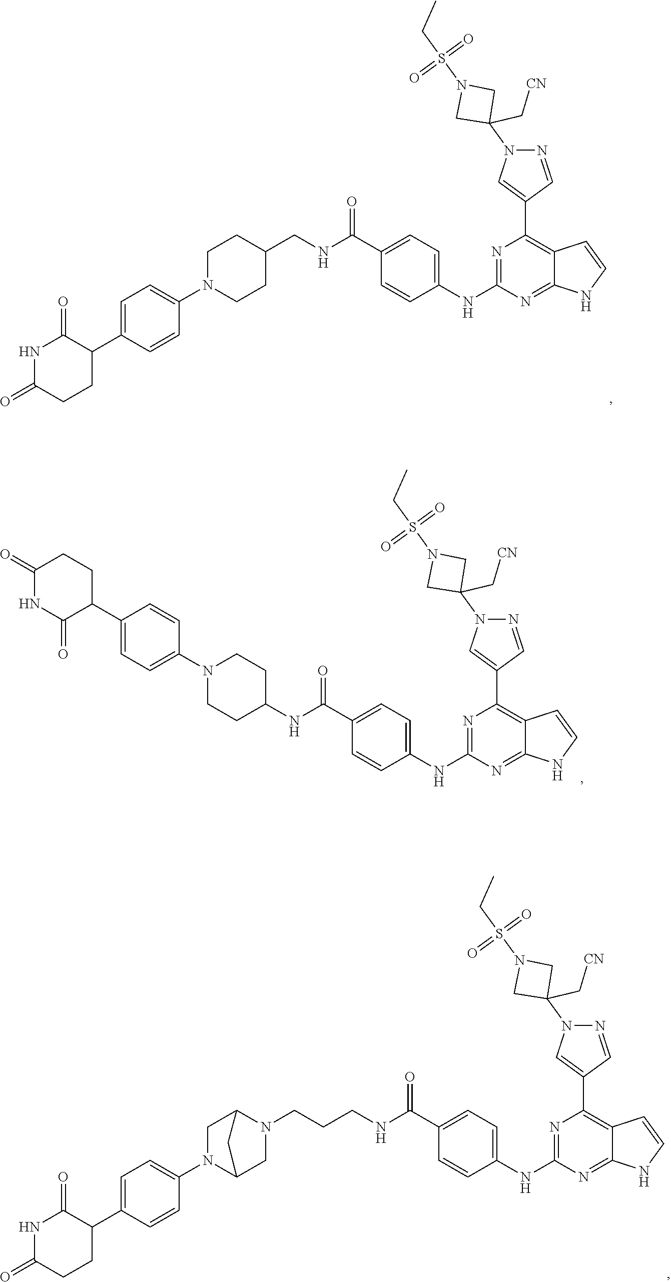 [SMILES: CCS(=O)(=O)N1CC(CC#N)(n2cc(-c3nc(Nc4ccc(C(=O)NCC5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NC5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NCCCN5CC6CC5CN6c5ccc(C6CCC(=O)NC6=O)cc5)cc4)nc4[nH]ccc34)cn2)C1]

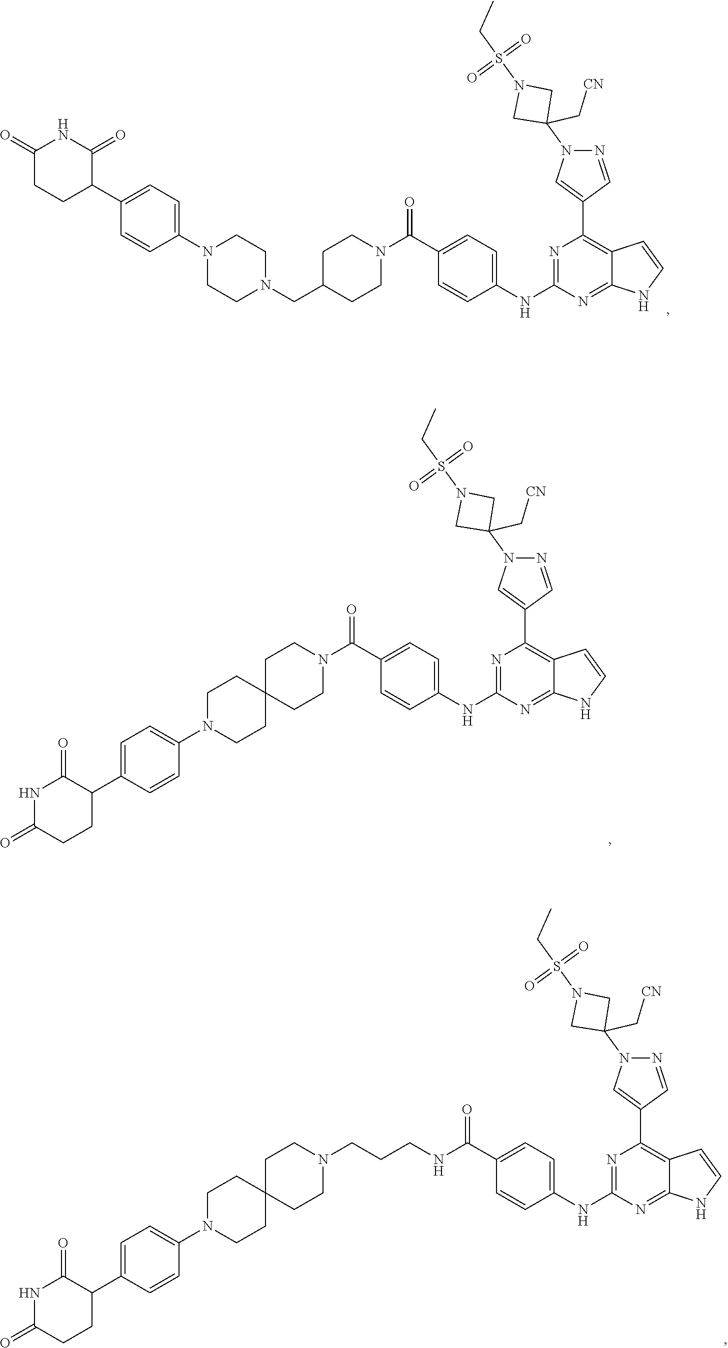 [SMILES: CCS(=O)(=O)N1CC(CC#N)(n2cc(-c3nc(Nc4ccc(C(=O)N5CCC(CN6CCN(c7ccc(C8CCC(=O)NC8=O)cc7)CC6)CC5)cc4)nc4[nH]ccc34)cn2)C1 ; CCS(=O)(=O)N1CC(CC#N)(n2cc(-c3nc(Nc4ccc(C(=O)N5CCC6(CC5)CCN(c5ccc(C7CCC(=O)NC7=O)cc5)CC6)cc4)nc4[nH]ccc34)cn2)C1 ; CCS(=O)(=O)N1CC(CC#N)(n2cc(-c3nc(Nc4ccc(C(=O)NCCCN5CCC6(CC5)CCN(c5ccc(C7CCC(=O)NC7=O)cc5)CC6)cc4)nc4[nH]ccc34)cn2)C1]

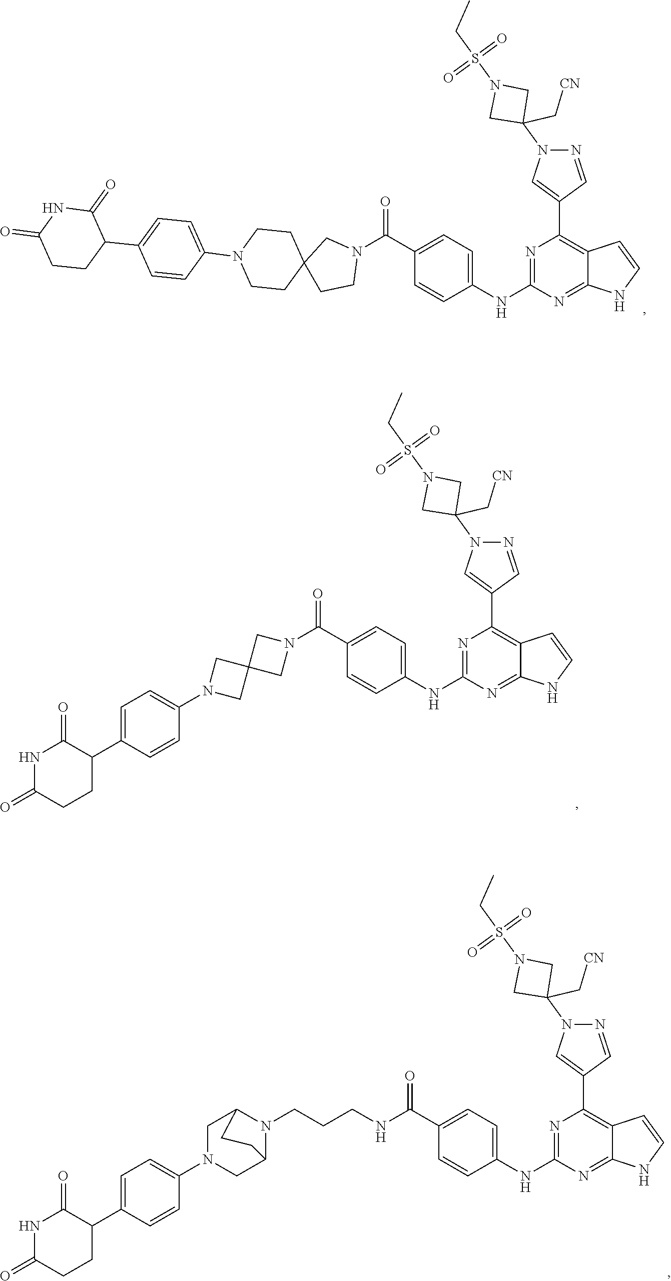 [SMILES: CCS(=O)(=O)N1CC(CC#N)(n2cc(-c3nc(Nc4ccc(C(=O)N5CCC6(CCN(c7ccc(C8CCC(=O)NC8=O)cc7)CC6)C5)cc4)nc4[nH]ccc34)cn2)C1 ; CCS(=O)(=O)N1CC(CC#N)(n2cc(-c3nc(Nc4ccc(C(=O)N5CC6(C5)CN(c5ccc(C7CCC(=O)NC7=O)cc5)C6)cc4)nc4[nH]ccc34)cn2)C1 ; CCS(=O)(=O)N1CC(CC#N)(n2cc(-c3nc(Nc4ccc(C(=O)NCCCN5C6CCC5CN(c5ccc(C7CCC(=O)NC7=O)cc5)C6)cc4)nc4[nH]ccc34)cn2)C1]

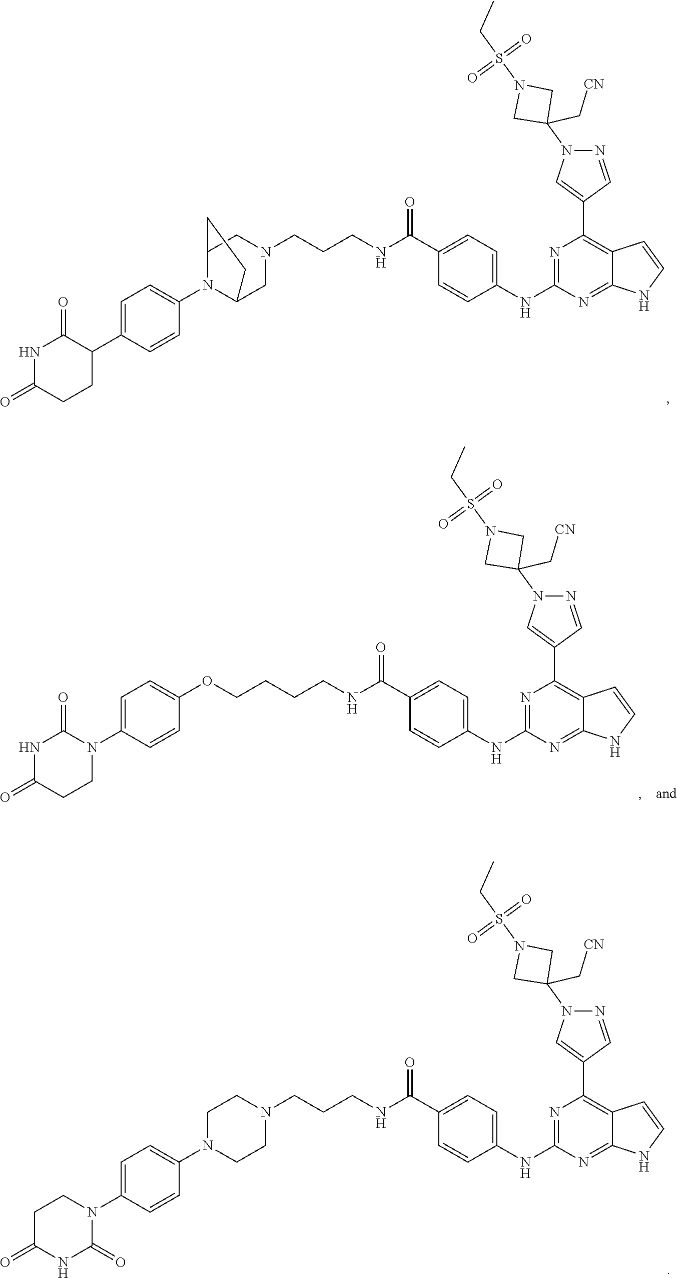 [SMILES: CCS(=O)(=O)N1CC(CC#N)(n2cc(-c3nc(Nc4ccc(C(=O)NCCCN5CC6CCC(C5)N6c5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCOc5ccc(N6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(N7CCC(=O)NC7=O)cc6)CC5)cc4)nc4[nH]ccc34)cn2)C1]

or a pharmaceutically acceptable salt thereof.

In one aspect, the compound is not:

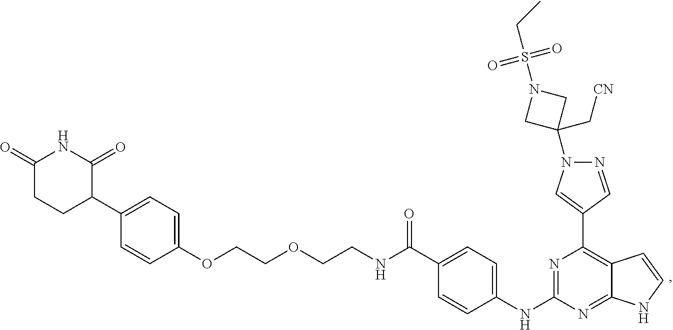 [SMILES: CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1]

or a pharmaceutically acceptable salt thereof.

In one aspect, a compound can be present as one or more of the following structures:

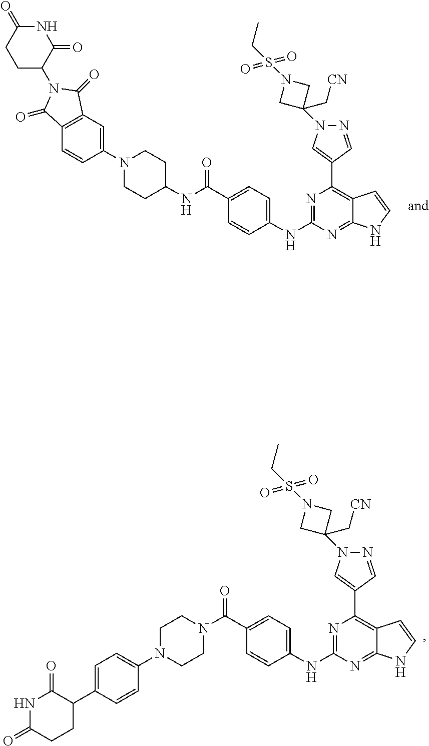 [SMILES: CCS(=O)(=O)N1CC(CC#N)(n2cc(-c3nc(Nc4ccc(C(=O)NC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1]

or a pharmaceutically acceptable salt thereof.

### C. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-IV, as described and exemplified below. The following examples are provided so that the invention might be more fully understood, are illustrative only, and should not be construed as limiting.

1. Route I

In one aspect, the compounds disclosed herein can be prepared as shown below.

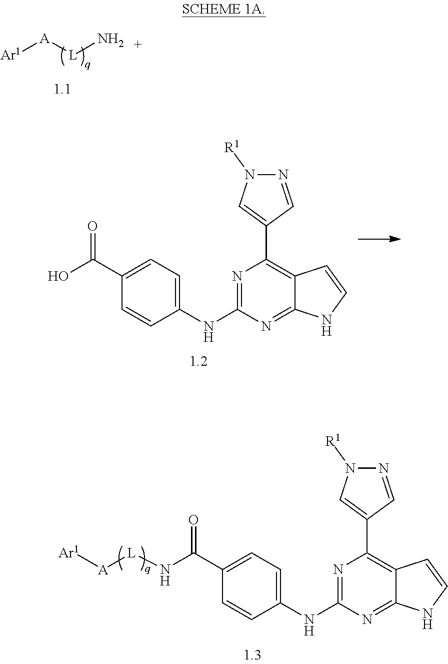

Compounds are represented in generic form, with substituents as noted in compound descriptions elsewhere herein. A more specific example is set forth below.

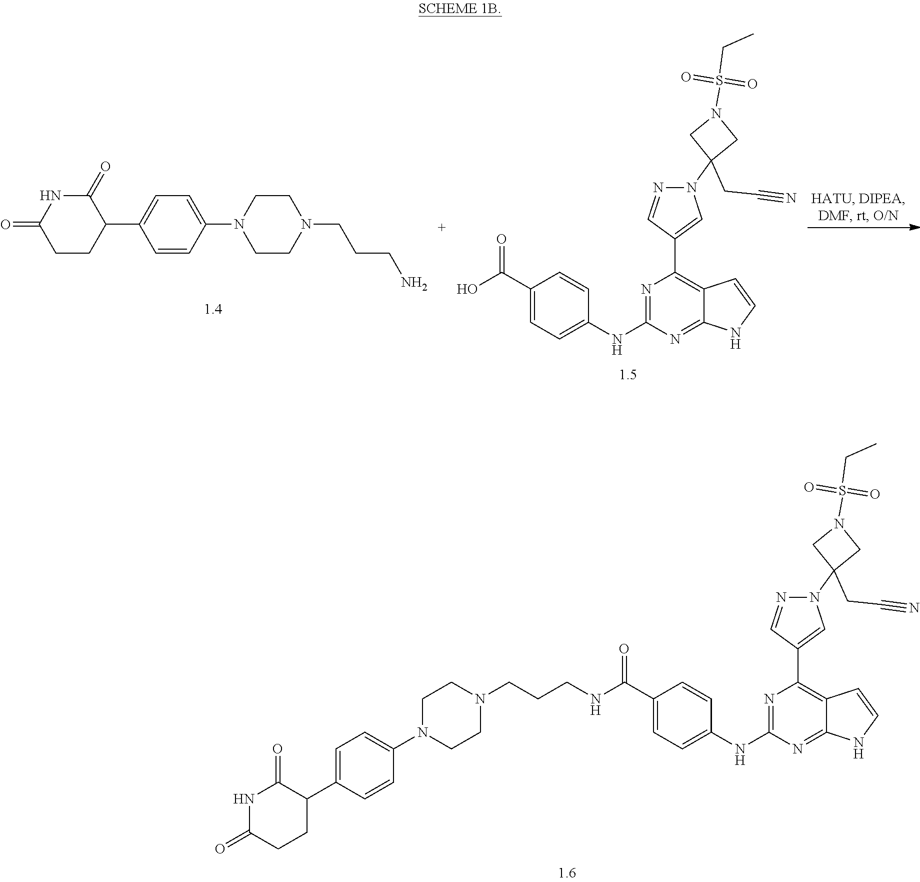 [SMILES: NCCCN1CCN(c2ccc(C3CCC(=O)NC3=O)cc2)CC1 ; CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)NCCCN5CCN(c6ccc(C7CCC(=O)NC7=O)cc6)CC5)cc4)nc4[nH]ccc34)cn2)C1]

In one aspect, compounds of type 1.6, and similar compounds, can be prepared according to reaction Scheme 1B above. Thus, compounds of type 1.6 can be prepared by a coupling reaction between an appropriate amine, e.g., 1.4 as shown above, and an appropriate carboxylic acid, e.g., 1.5 as shown above. Appropriate amines and appropriate carboxylic acids are commercially available or prepared by methods known to one skilled in the art. The coupling reaction is carried out in the presence of an appropriate coupling agent, e.g., 1-[bis(dimethylamino)methylene]-1H-1,2,3-triazolo[4,5-b]pyridinium 3-oxid hexafluorophosphate (HATU), and an appropriate base, e.g., diisopropylethylamine (DIPEA), in an appropriate solvent, e.g., dimethylformamide. As can be appreciated by one skilled in the art, the above reaction provides an example of a generalized approach wherein compounds similar in structure to the specific reactants above (compounds similar to compounds of type 1.1 and 1.2), can be substituted in the reaction to provide amide derivatives similar to Formula 1.3.

2. Route II

In one aspect, the compounds disclosed herein can be prepared as shown below.

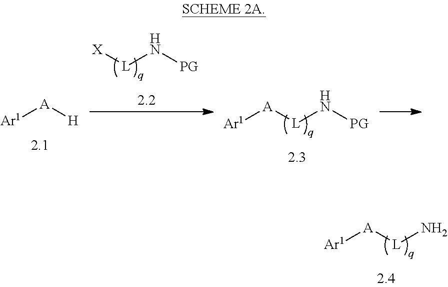

Compounds are represented in generic form, where X is a halide, PG is an amine protecting group, and with other substituents as noted in compound descriptions elsewhere herein. A more specific example is set forth below.

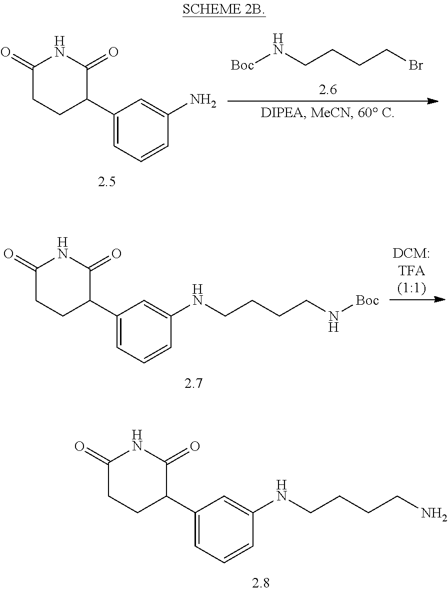 [SMILES: Nc1cccc(C2CCC(=O)NC2=O)c1 ; CC(C)(C)OC(=O)NCCCCBr ; CC(C)(C)OC(=O)NCCCCNc1cccc(C2CCC(=O)NC2=O)c1 ; NCCCCNc1cccc(C2CCC(=O)NC2=O)c1]

In one aspect, compounds of type 2.8, and similar compounds, can be prepared according to reaction Scheme 2B above. Thus, compounds of type 2.7 can be prepared by a coupling reaction between an appropriate amine, e.g., 2.5 as shown above, and an appropriate halide, e.g., 2.6 as shown above. Appropriate halides are commercially available or prepared by methods known to one skilled in the art. The coupling reaction is carried out in the presence of an appropriate base, e.g., diisopropylethylamine (DIPEA), in an appropriate solvent, e.g., acetonitrile, at an appropriate temperature, e.g., 60° C. Compounds of type 2.8 can be prepared by deprotection of an appropriate protected amine, e.g., 2.7 as shown above. The deprotection is carried out in the presence of an appropriate deprotecting agent, e.g., an acid such as trifluoroacetic acid, in an appropriate solvent, e.g., dichloromethane. As can be appreciated by one skilled in the art, the above reaction provides an example of a generalized approach wherein compounds similar in structure to the specific reactants above (compounds similar to compounds of type 2.1, 2.2, and 2.3), can be substituted in the reaction to provide substituted derivatives similar to Formula 2.4.

3. Route III

In one aspect, the compounds disclosed herein can be prepared as shown below.

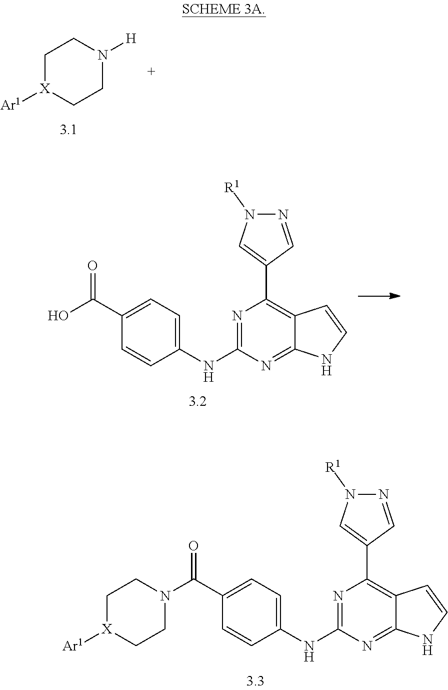

Compounds are represented in generic form, with substituents as noted in compound descriptions elsewhere herein. A more specific example is set forth below.

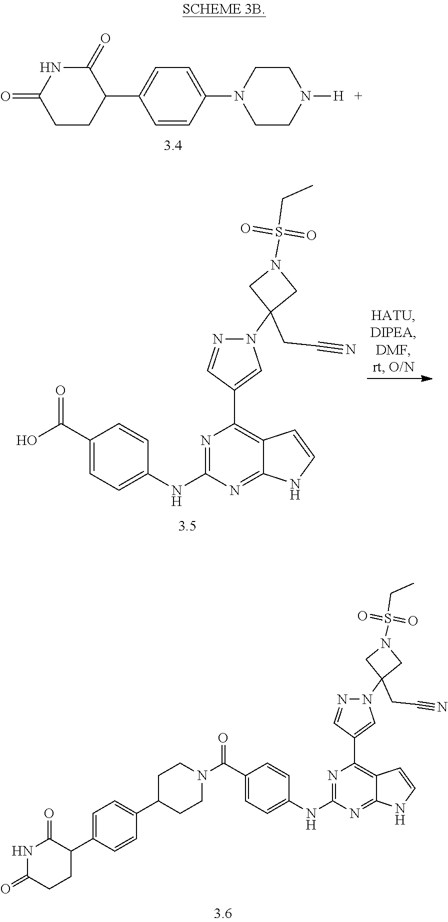 [SMILES: O=C1CCC(c2ccc(N3CCNCC3)cc2)C(=O)N1 ; CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)N5CCC(c6ccc(C7CCC(=O)NC7=O)cc6)CC5)cc4)nc4[nH]ccc34)cn2)C1]

In one aspect, compounds of type 3.6, and similar compounds, can be prepared according to reaction Scheme 3B above. Thus, compounds of type 3.6 can be prepared by a coupling reaction between an appropriate amine, e.g., 3.4 as shown above, and an appropriate carboxylic acid, e.g., 3.5 as shown above. Appropriate amines and appropriate carboxylic acids are commercially available or prepared by methods known to one skilled in the art. The coupling reaction is carried out in the presence of an appropriate coupling agent, e.g., 1-[bis(dimethylamino)methylene]-1H-1,2,3-triazolo[4,5-b]pyridinium 3-oxid hexafluorophosphate (HATU), and an appropriate base, e.g., diisopropylethylamine (DIPEA), in an appropriate solvent, e.g., dimethylformamide. As can be appreciated by one skilled in the art, the above reaction provides an example of a generalized approach wherein compounds similar in structure to the specific reactants above (compounds similar to compounds of type 3.1 and 3.2), can be substituted in the reaction to provide amide derivatives similar to Formula 3.3.

4. Route IV

In one aspect, the compounds disclosed herein can be prepared as shown below.

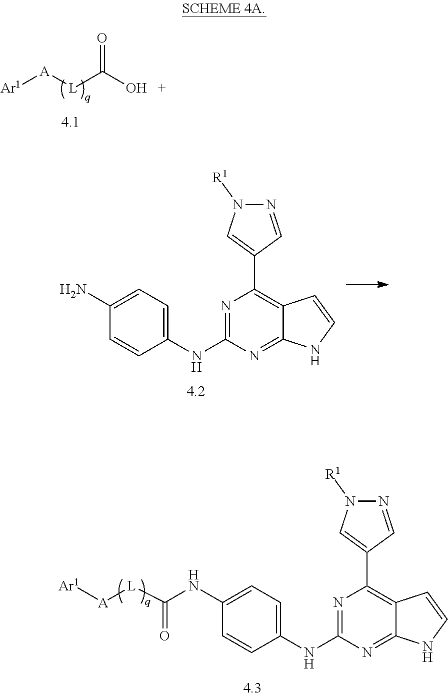

Compounds are represented in generic form, with substituents as noted in compound descriptions elsewhere herein. A more specific example is set forth below.

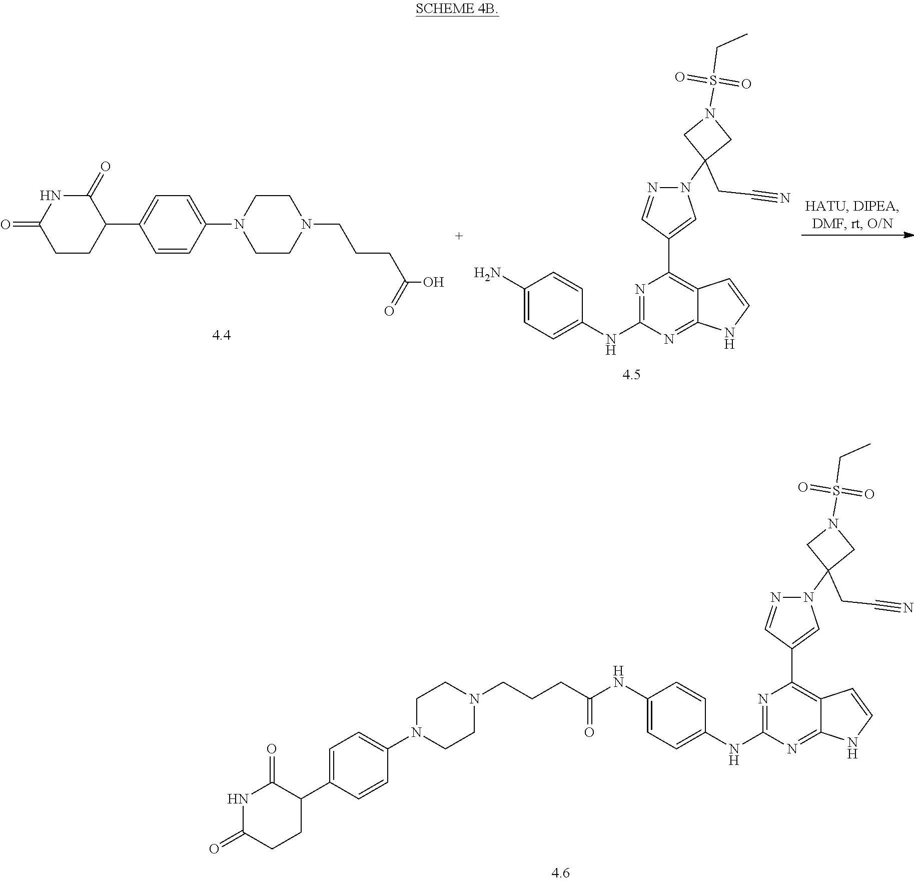 [SMILES: O=C(O)CCCN1CCN(c2ccc(C3CCC(=O)NC3=O)cc2)CC1 ; CCS(=O)(=O)N1CC(CC#N)(n2cc(-c3nc(Nc4ccc(N)cc4)nc4[nH]ccc34)cn2)C1 ; CCS(=O)(=O)N1CC(CC#N)(n2cc(-c3nc(Nc4ccc(NC(=O)CCCN5CCN(c6ccc(C7CCC(=O)NC7=O)cc6)CC5)cc4)nc4[nH]ccc34)cn2)C1]

In one aspect, compounds of type 4.6, and similar compounds, can be prepared according to reaction Scheme 4B above. Thus, compounds of type 4.6 can be prepared by a coupling reaction between an appropriate amine, e.g., 4.4 as shown above, and an appropriate carboxylic acid, e.g., 4.5 as shown above. Appropriate amines and appropriate carboxylic acids are commercially available or prepared by methods known to one skilled in the art. The coupling reaction is carried out in the presence of an appropriate coupling agent, e.g., 1-[bis(dimethylamino)methylene]-1H-1,2,3-triazolo[4,5-b]pyridinium 3-oxid hexafluorophosphate (HATU), and an appropriate base, e.g., diisopropylethylamine (DIPEA), in an appropriate solvent, e.g., dimethylformamide. As can be appreciated by one skilled in the art, the above reaction provides an example of a generalized approach wherein compounds similar in structure to the specific reactants above (compounds similar to compounds of type 4.1 and 4.2), can be substituted in the reaction to provide amide derivatives similar to Formula 4.3.

### D. Pharmaceutical Compositions

In one aspect, disclosed are pharmaceutical compositions comprising a disclosed compound, or a pharmaceutically acceptable salt thereof and a pharmaceutically acceptable carrier.

Thus, in one aspect, disclosed are pharmaceutical compositions comprising a therapeutically effective amount of a compound having a structure represented by a formula:

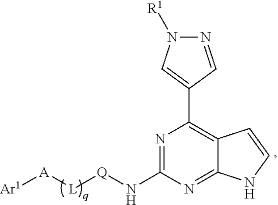

wherein q is selected from 0 and 1; wherein A is selected from —O—, —S—, —NH—, —NHCH₂—, —OCH₂C(O)NH—, —CH₂—, and a structure selected from:

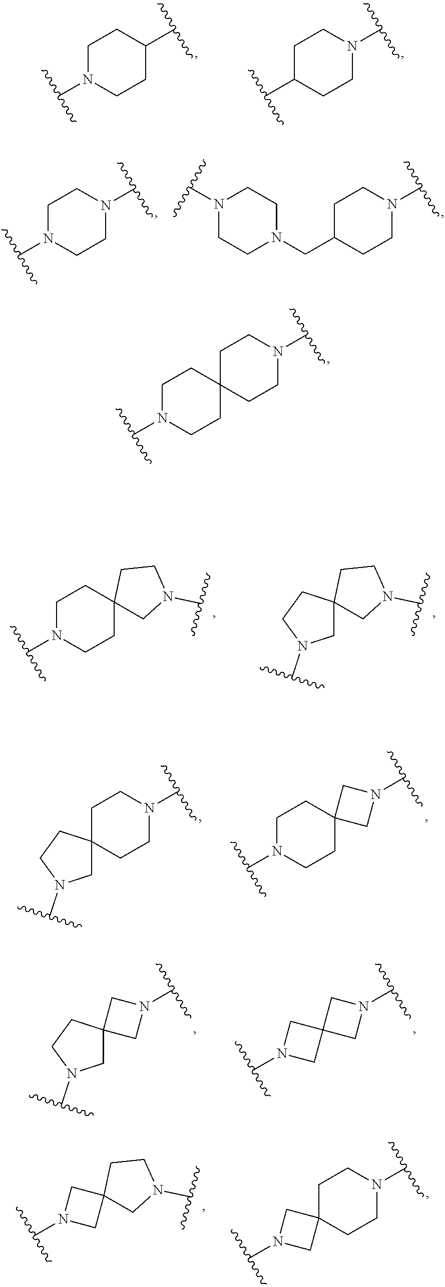

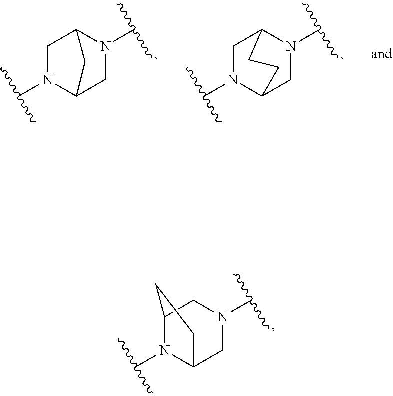

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

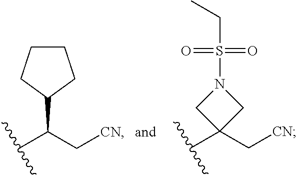 [SMILES: *[C@H](CC#N)C1CCCC1 ; *C1(CC#N)CN(S(=O)(=O)CC)C1]

and
wherein Ar¹ is a structure selected from:

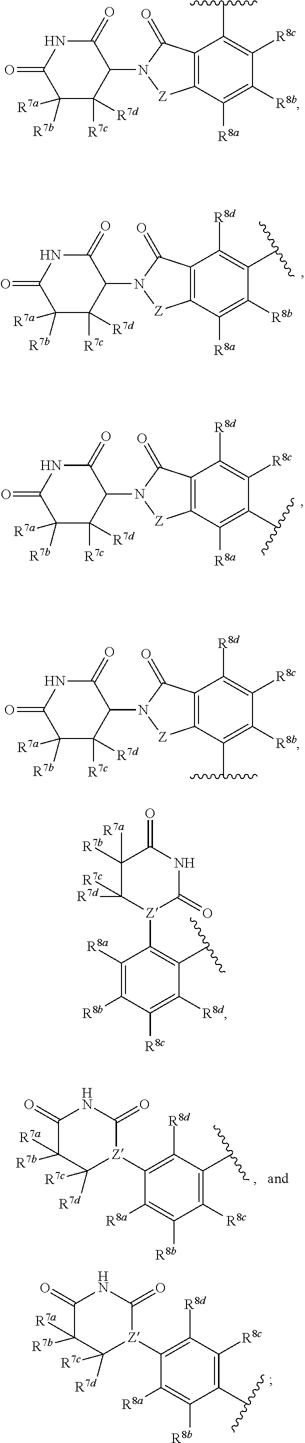

wherein Z is selected from —CH₂— and C(O); wherein Z′ is selected from —CH— and —N—; wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

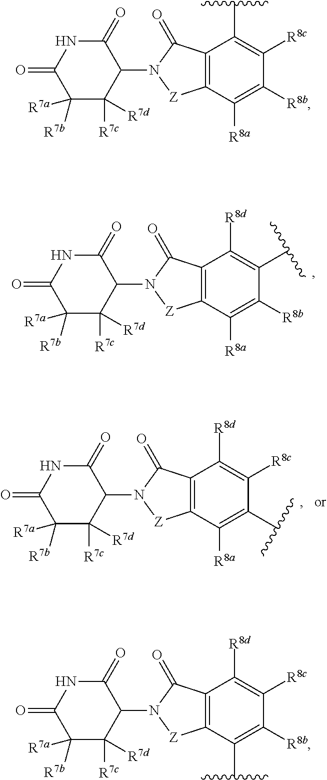

and q is 1, then A is a structure selected from:

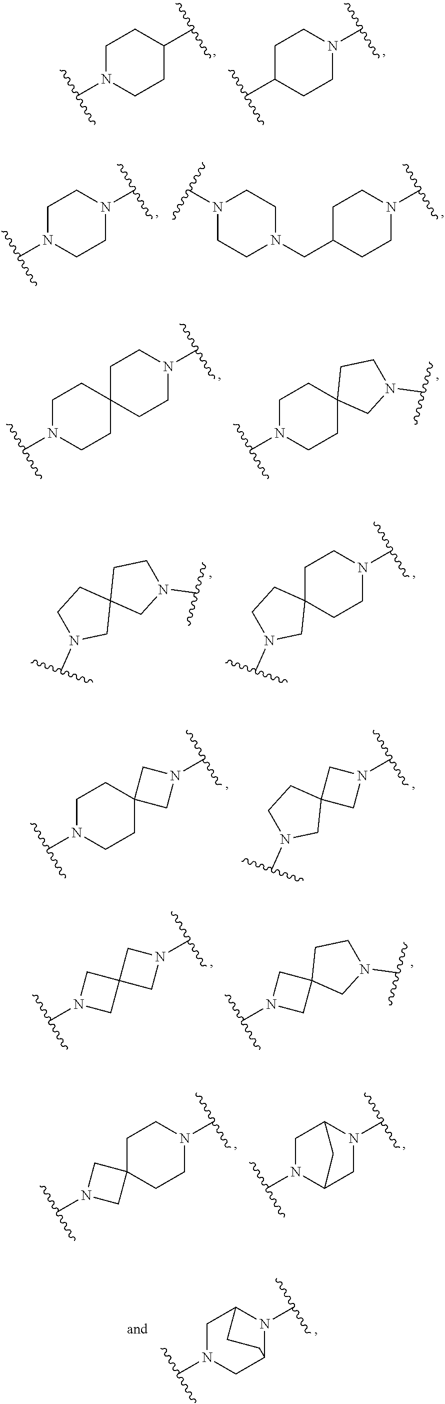

or a pharmaceutically acceptable salt thereof, and a pharmaceutically acceptable carrier.

In one aspect, disclosed are pharmaceutical compositions comprising a therapeutically effective amount of a compound having a structure compounds having a structure represented by a formula:

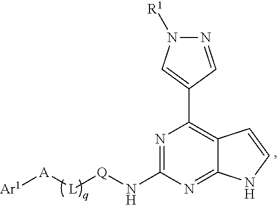

wherein q is selected from 0 and 1; wherein A is selected from —O—, —S—, —NH—, —NHCH₂—, —OCH₂C(O)NH—, —CH₂—, and a structure selected from:

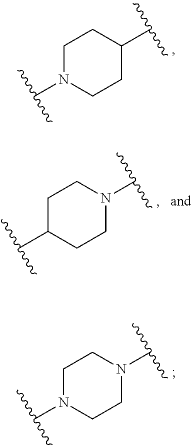

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

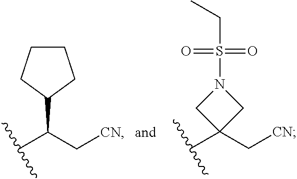 [SMILES: *[C@H](CC#N)C1CCCC1 ; *C1(CC#N)CN(S(=O)(=O)CC)C1]

and
wherein Ar¹ is a structure selected from:

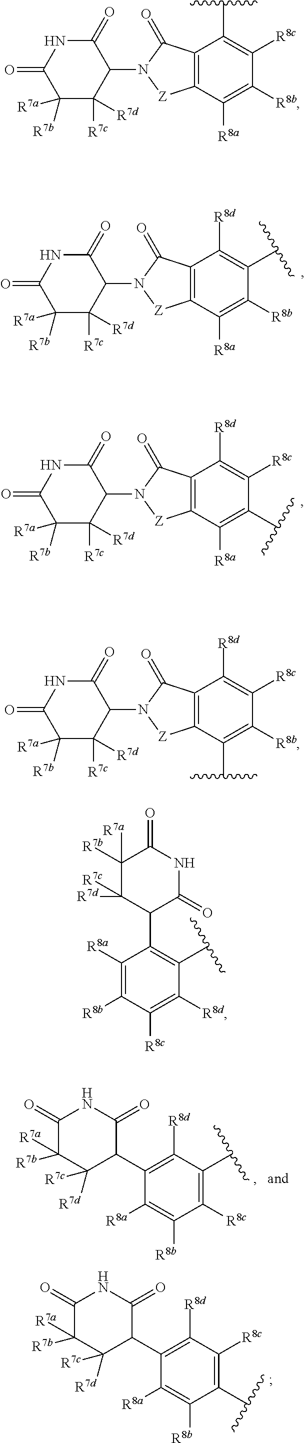

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

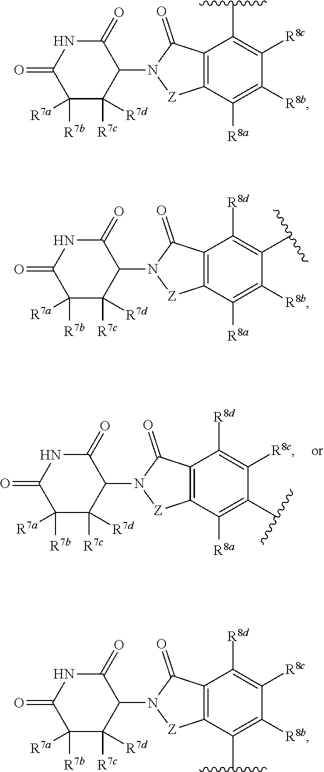

and q is 1, then A is a structure selected from:

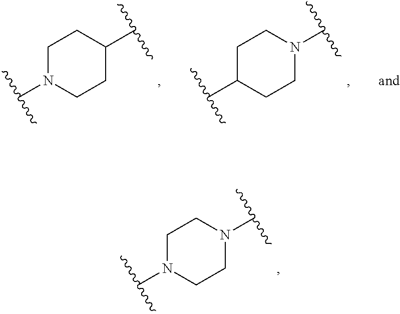 [SMILES: *C1CCN(*)CC1 ; *C1CCN(*)CC1 ; *N1CCN(*)CC1]

or a pharmaceutically acceptable salt thereof, and a pharmaceutically acceptable carrier.

In one aspect, disclosed are pharmaceutical compositions comprising a therapeutically effective amount of at least one compound having a structure selected from:

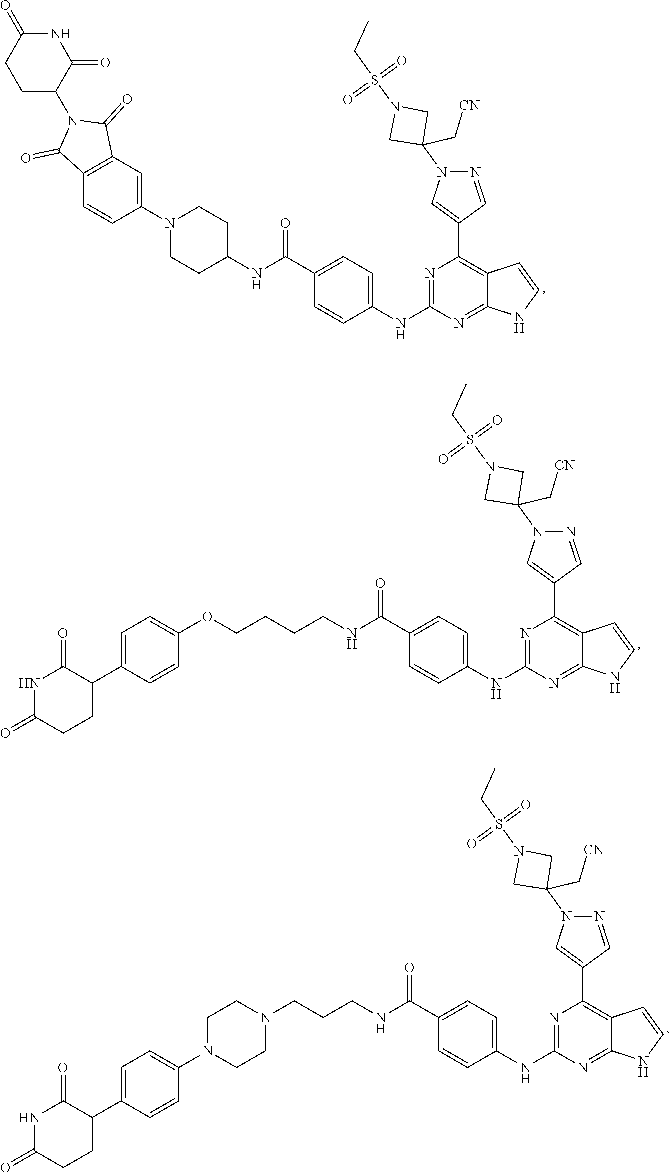 [SMILES: CCS(=O)(=O)N1CC(CC#N)(n2cc(-c3nc(Nc4ccc(C(=O)NC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCOc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(C7CCC(=O)NC7=O)cc6)CC5)cc4)nc4[nH]ccc34)cn2)C1]

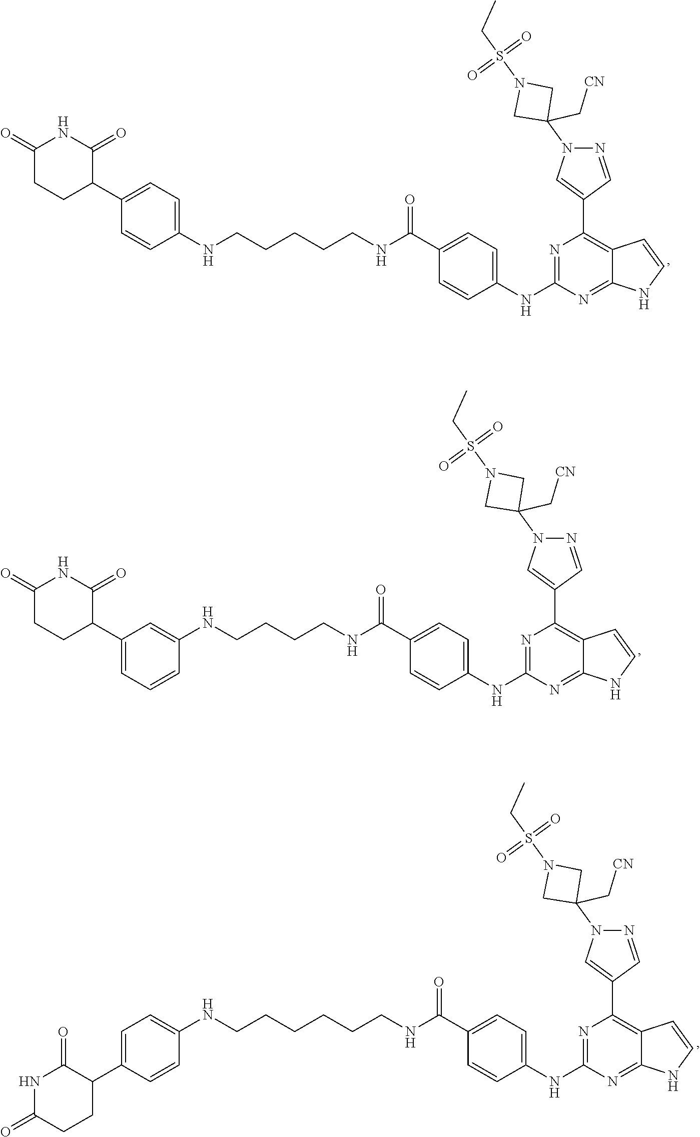 [SMILES: CCS(=O)(=O)N1CC(CC#N)(n2cc(-c3nc(Nc4ccc(C(=O)NCCCCCNc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCNc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCCCCNc5ccc(C6CCC(=O)NC6=O)cc5)cc4)nc4[nH]ccc34)cn2)C1]

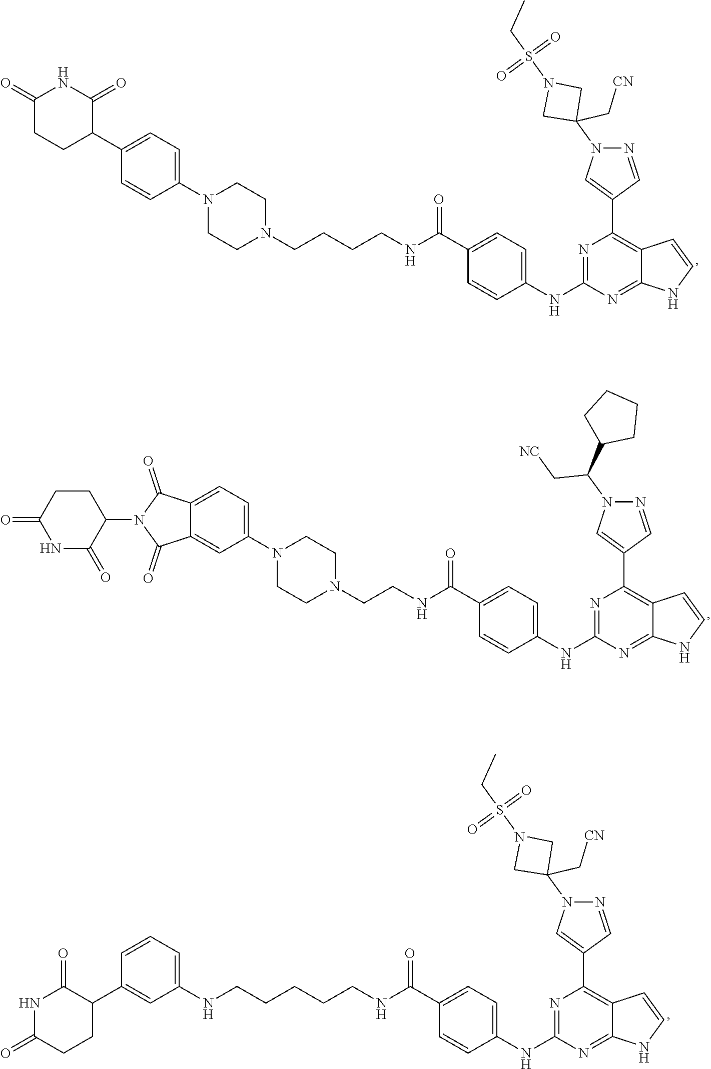 [SMILES: CCS(=O)(=O)N1CC(CC#N)(n2cc(-c3nc(Nc4ccc(C(=O)NCCCCN5CCN(c6ccc(C7CCC(=O)NC7=O)cc6)CC5)cc4)nc4[nH]ccc34)cn2)C1 ; N#CC[C@@H](C1CCCC1)n1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCCCCNc5cccc(C6CCC(=O)NC6=O)c5)cc4)nc4[nH]ccc34)cn2)C1]

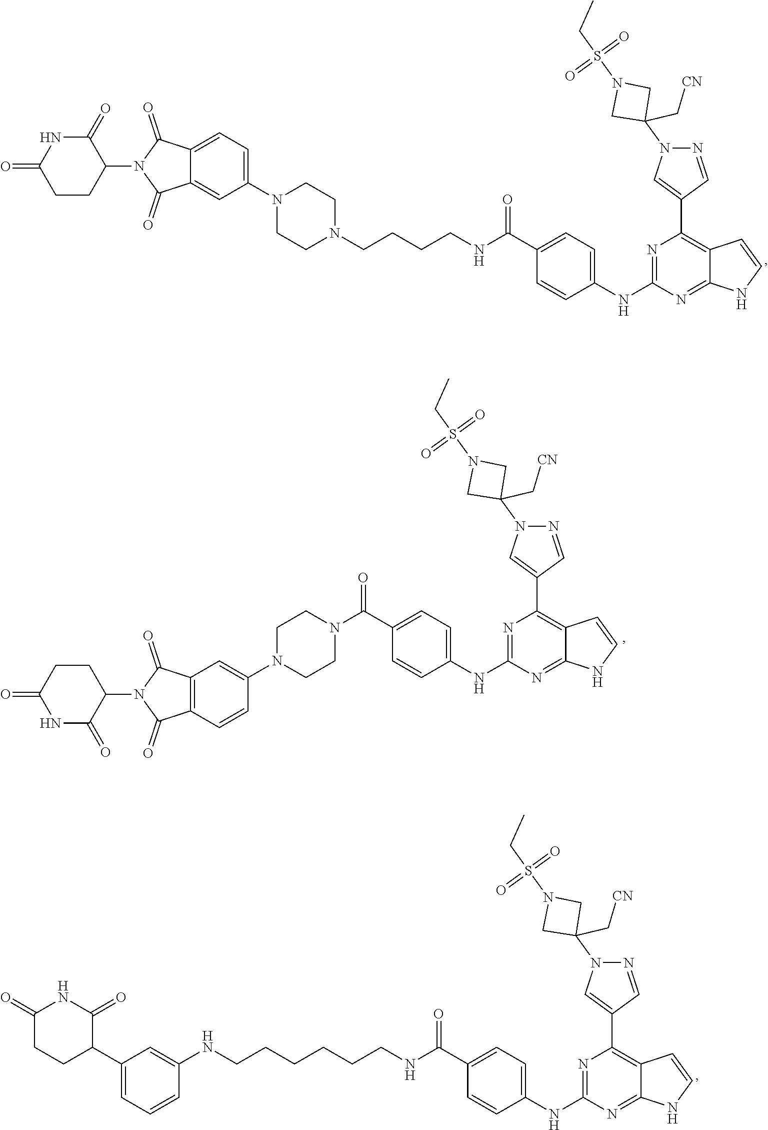 [SMILES: CCS(=O)(=O)N1CC(CC#N)(n2cc(-c3nc(Nc4ccc(C(=O)NCCCCN5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)N5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCCCNc5cccc(C6CCC(=O)NC6=O)c5)cc4)nc4[nH]ccc34)cn2)C1]

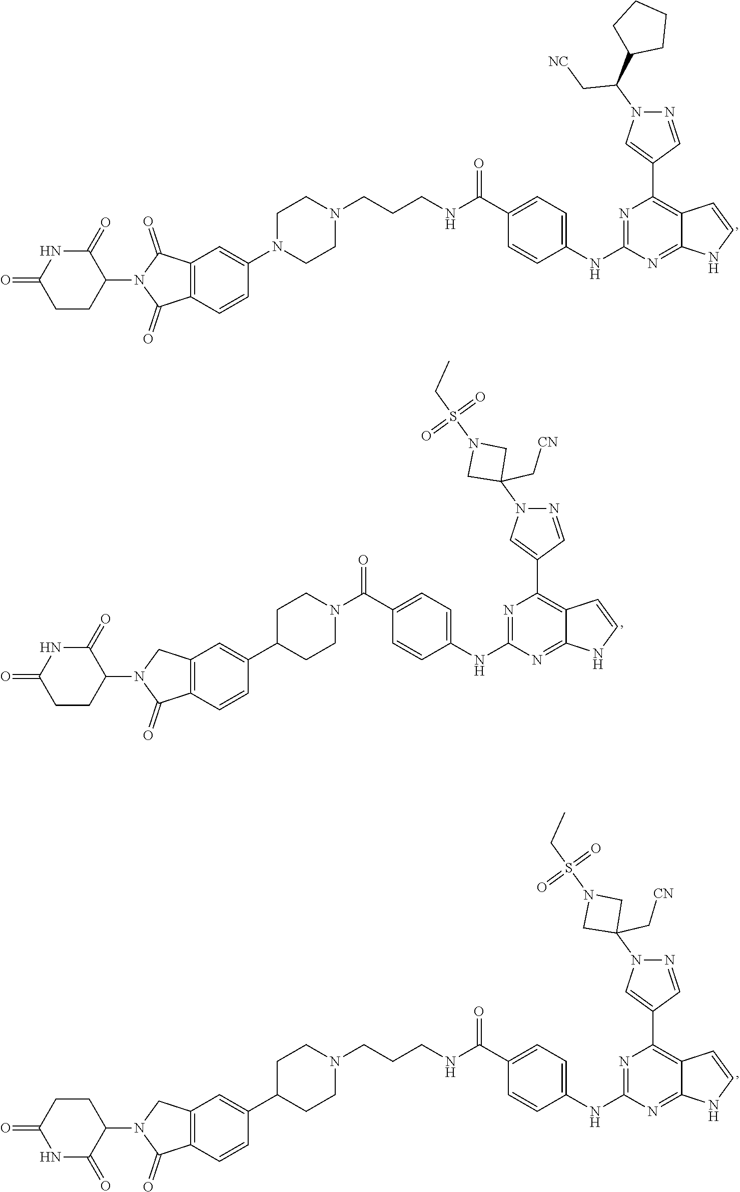 [SMILES: N#CC[C@@H](C1CCCC1)n1cc(-c2nc(Nc3ccc(C(=O)NCCCN4CCN(c5ccc6c(c5)C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)N5CCC(c6ccc7c(c6)CN(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCN5CCC(c6ccc7c(c6)CN(C6CCC(=O)NC6=O)C7=O)CC5)cc4)nc4[nH]ccc34)cn2)C1]

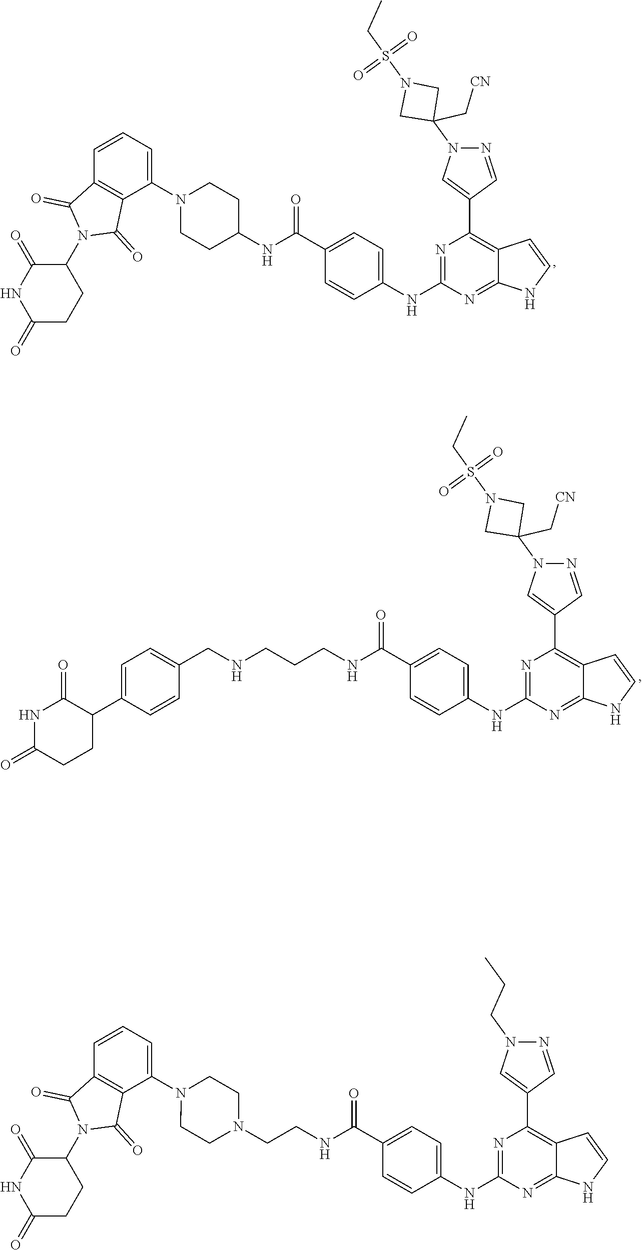 [SMILES: CCS(=O)(=O)N1CC(CC#N)(n2cc(-c3nc(Nc4ccc(C(=O)NC5CCN(c6cccc7c6C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCNCc5ccc(C6CCC(=O)NC6=O)cc5)cc4)nc4[nH]ccc34)cn2)C1 ; CCCn1cc(-c2nc(Nc3ccc(C(=O)NCCN4CCN(c5cccc6c5C(=O)N(C5CCC(=O)NC5=O)C6=O)CC4)cc3)nc3[nH]ccc23)cn1]

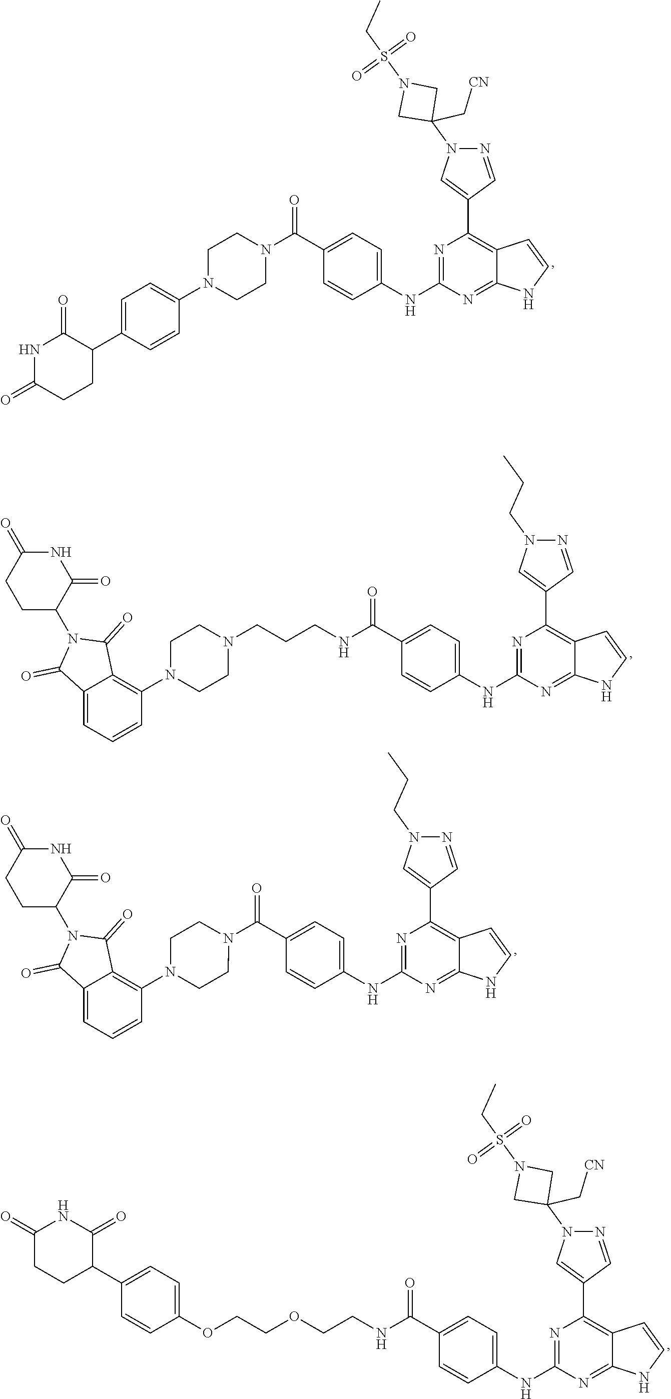 [SMILES: CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1 ; CCCn1cc(-c2nc(Nc3ccc(C(=O)NCCCN4CCN(c5cccc6c5C(=O)N(C5CCC(=O)NC5=O)C6=O)CC4)cc3)nc3[nH]ccc23)cn1 ; CCCn1cc(-c2nc(Nc3ccc(C(=O)N4CCN(c5cccc6c5C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1]

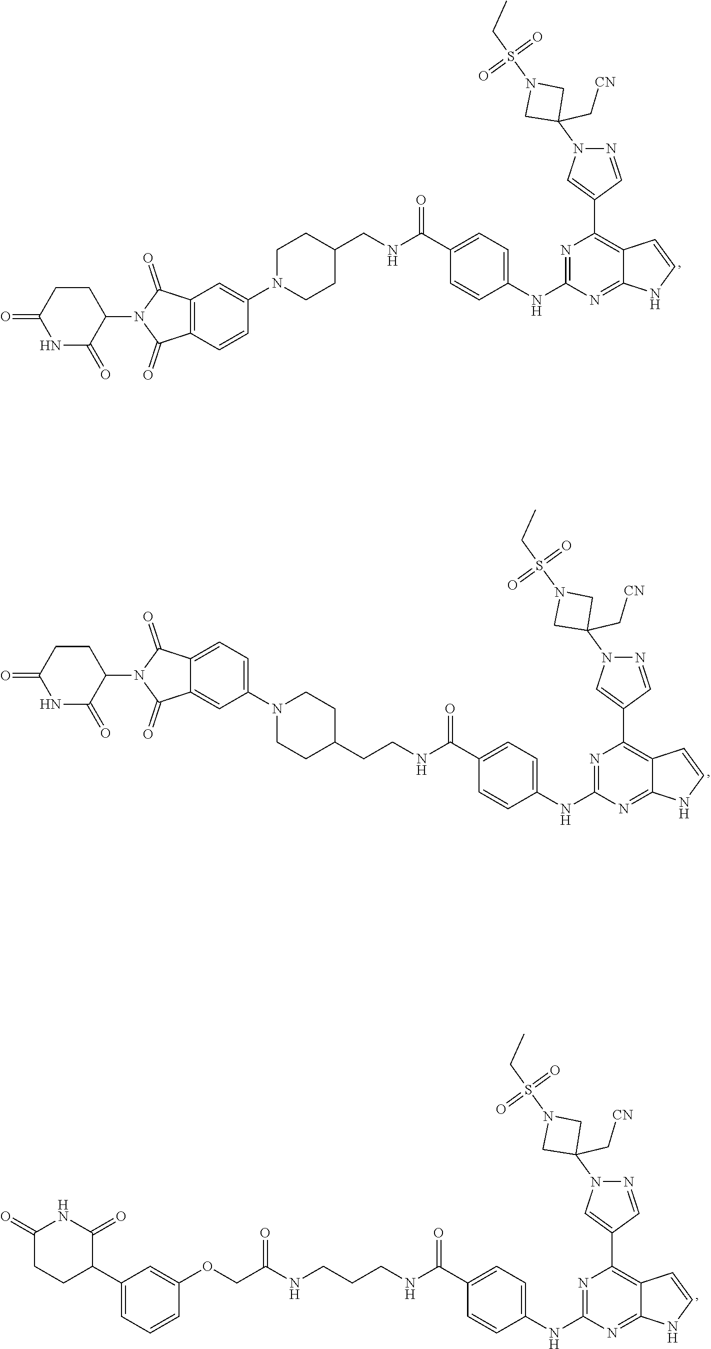 [SMILES: CCS(=O)(=O)N1CC(CC#N)(n2cc(-c3nc(Nc4ccc(C(=O)NCC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCNC(=O)COc5cccc(C6CCC(=O)NC6=O)c5)cc4)nc4[nH]ccc34)cn2)C1]

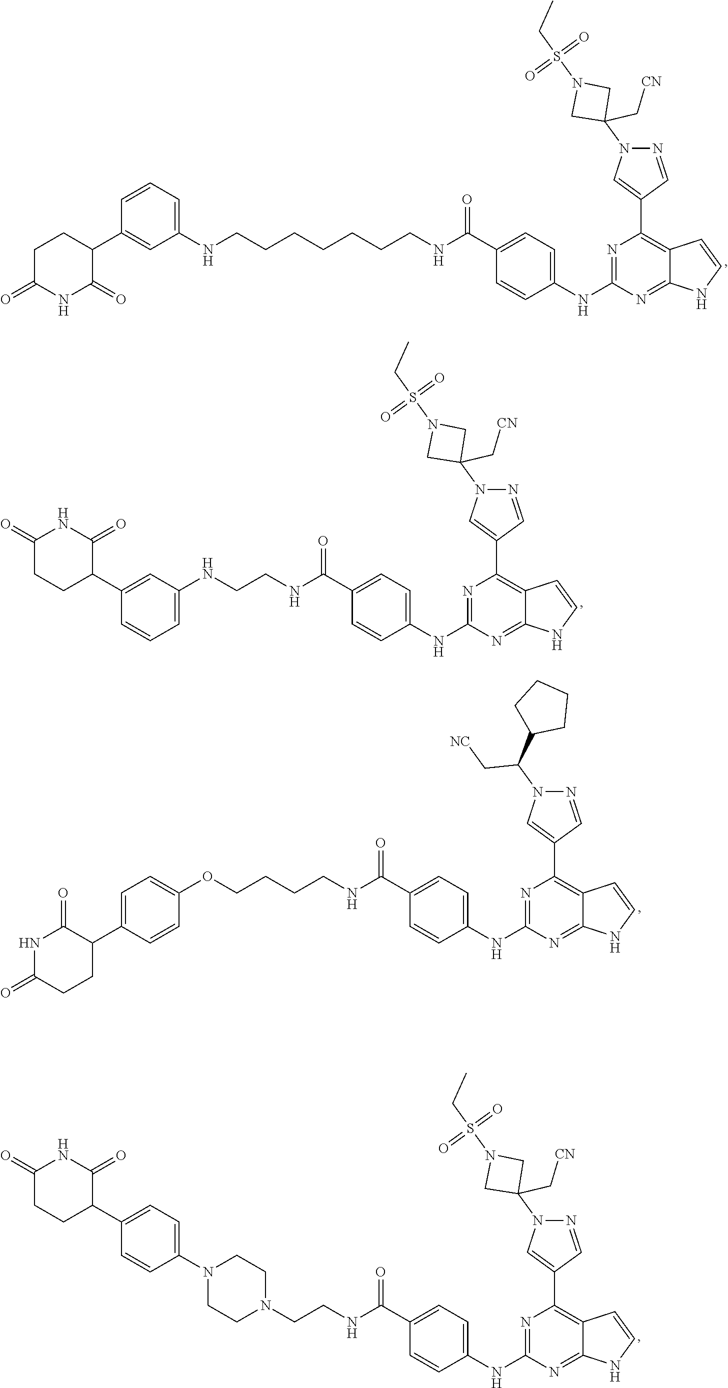 [SMILES: CCS(=O)(=O)N1CC(CC#N)(n2cc(-c3nc(Nc4ccc(C(=O)NCCCCCCCNc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCNc5cccc(C6CCC(=O)NC6=O)c5)cc4)nc4[nH]ccc34)cn2)C1 ; N#CC[C@@H](C1CCCC1)n1cc(-c2nc(Nc3ccc(C(=O)NCCCCOc4ccc(C5CCC(=O)NC5=O)cc4)cc3)nc3[nH]ccc23)cn1 ; CCS(=O)(=O)N1CC(CC#N)(n2cc(-c3nc(Nc4ccc(C(=O)NCCN5CCN(c6ccc(C7CCC(=O)NC7=O)cc6)CC5)cc4)nc4[nH]ccc34)cn2)C1]

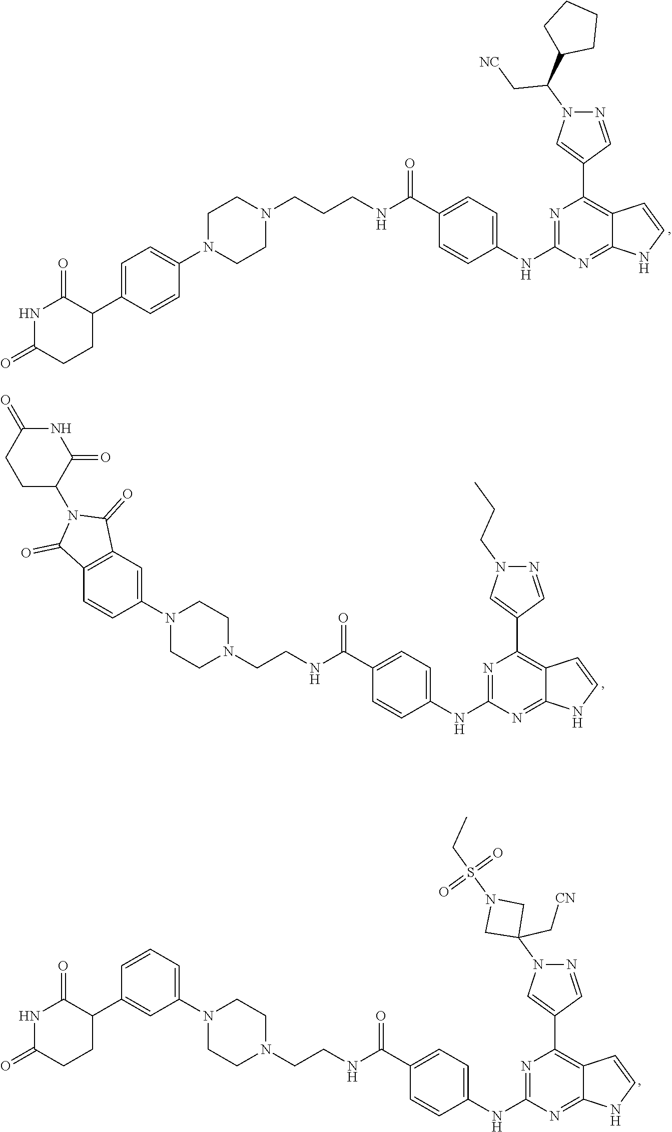

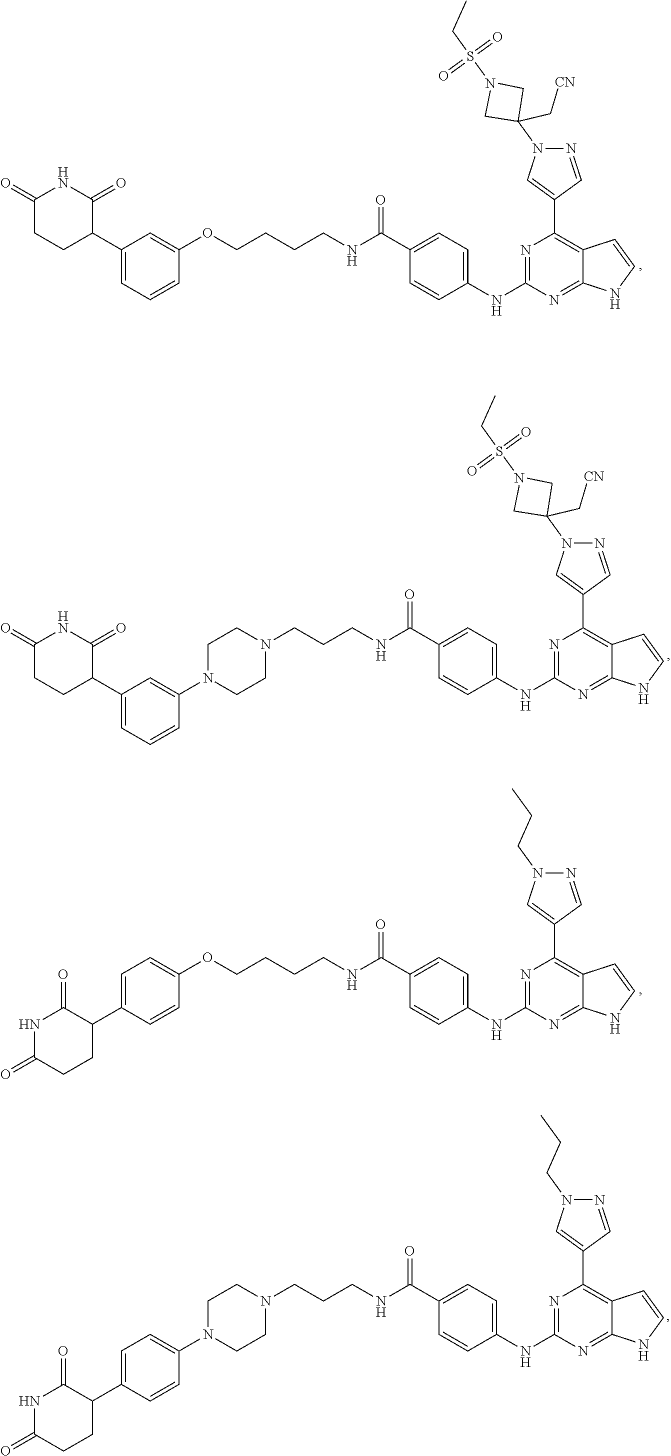 [SMILES: CCS(=O)(=O)N1CC(CC#N)(n2cc(-c3nc(Nc4ccc(C(=O)NCCCCOc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCN5CCN(c6cccc(C7CCC(=O)NC7=O)c6)CC5)cc4)nc4[nH]ccc34)cn2)C1 ; CCCn1cc(-c2nc(Nc3ccc(C(=O)NCCCCOc4ccc(C5CCC(=O)NC5=O)cc4)cc3)nc3[nH]ccc23)cn1 ; CCCn1cc(-c2nc(Nc3ccc(C(=O)NCCCN4CCN(c5ccc(C6CCC(=O)NC6=O)cc5)CC4)cc3)nc3[nH]ccc23)cn1]

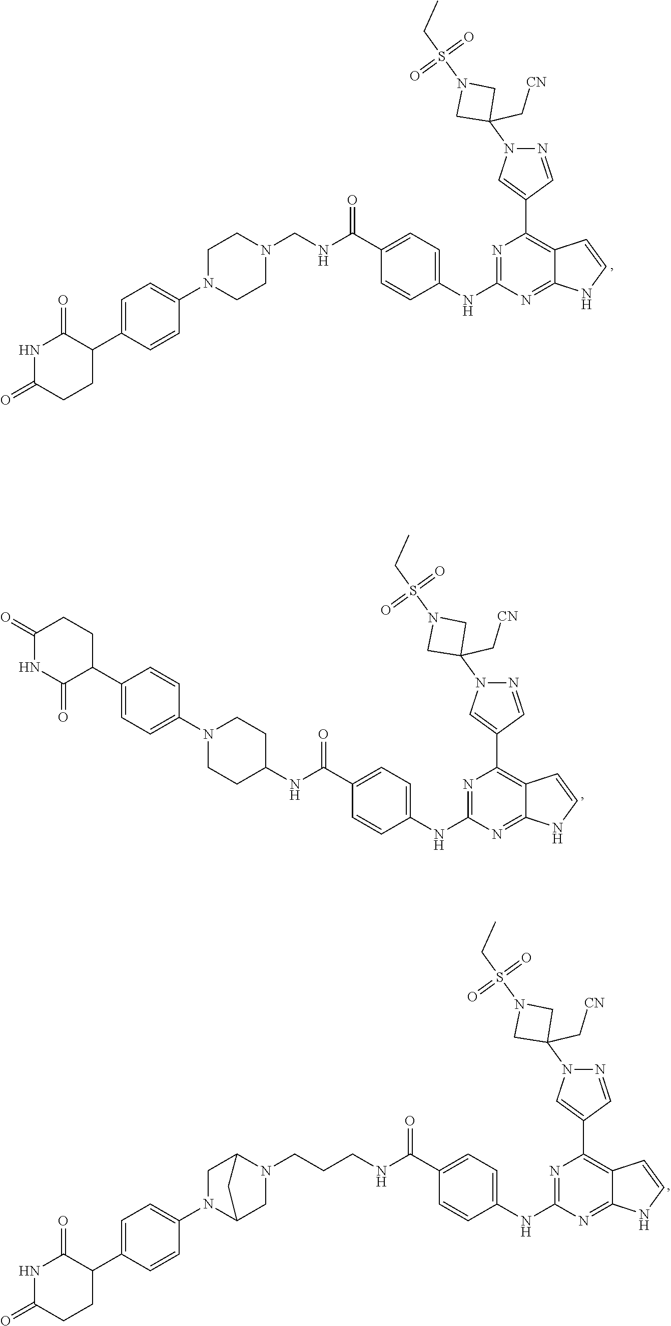 [SMILES: CCS(=O)(=O)N1CC(CC#N)(n2cc(-c3nc(Nc4ccc(C(=O)NCN5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NC5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NCCCN5CC6CC5CN6c5ccc(C6CCC(=O)NC6=O)cc5)cc4)nc4[nH]ccc34)cn2)C1]

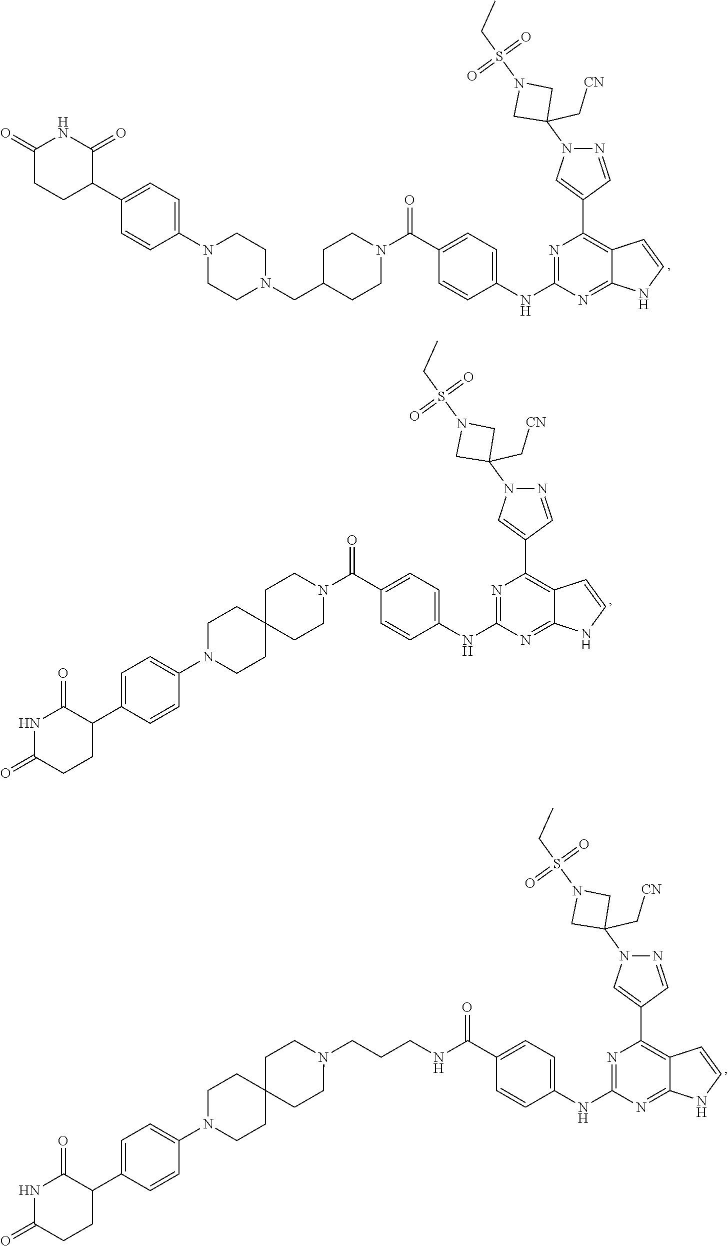 [SMILES: CCS(=O)(=O)N1CC(CC#N)(n2cc(-c3nc(Nc4ccc(C(=O)N5CCC(CN6CCN(c7ccc(C8CCC(=O)NC8=O)cc7)CC6)CC5)cc4)nc4[nH]ccc34)cn2)C1 ; CCS(=O)(=O)N1CC(CC#N)(n2cc(-c3nc(Nc4ccc(C(=O)N5CCC6(CC5)CCN(c5ccc(C7CCC(=O)NC7=O)cc5)CC6)cc4)nc4[nH]ccc34)cn2)C1 ; CCS(=O)(=O)N1CC(CC#N)(n2cc(-c3nc(Nc4ccc(C(=O)NCCCN5CCC6(CC5)CCN(c5ccc(C7CCC(=O)NC7=O)cc5)CC6)cc4)nc4[nH]ccc34)cn2)C1]

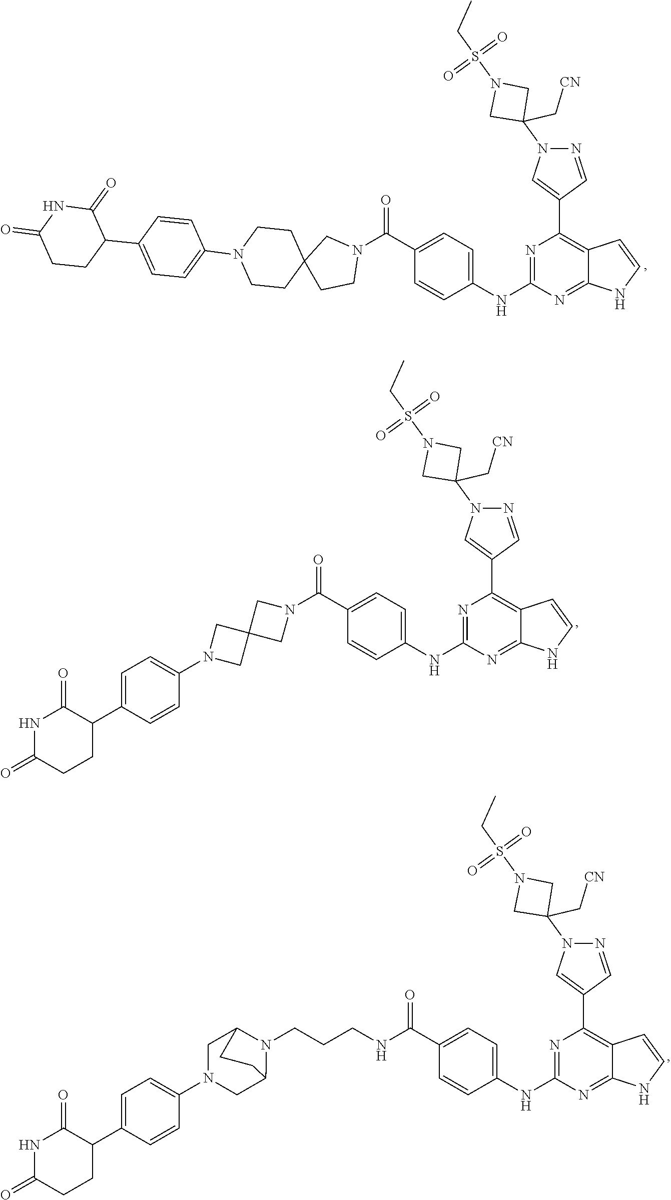 [SMILES: CCS(=O)(=O)N1CC(CC#N)(n2cc(-c3nc(Nc4ccc(C(=O)N5CCC6(CCN(c7ccc(C8CCC(=O)NC8=O)cc7)CC6)C5)cc4)nc4[nH]ccc34)cn2)C1 ; CCS(=O)(=O)N1CC(CC#N)(n2cc(-c3nc(Nc4ccc(C(=O)N5CC6(C5)CN(c5ccc(C7CCC(=O)NC7=O)cc5)C6)cc4)nc4[nH]ccc34)cn2)C1 ; CCS(=O)(=O)N1CC(CC#N)(n2cc(-c3nc(Nc4ccc(C(=O)NCCCN5C6CCC5CN(c5ccc(C7CCC(=O)NC7=O)cc5)C6)cc4)nc4[nH]ccc34)cn2)C1]

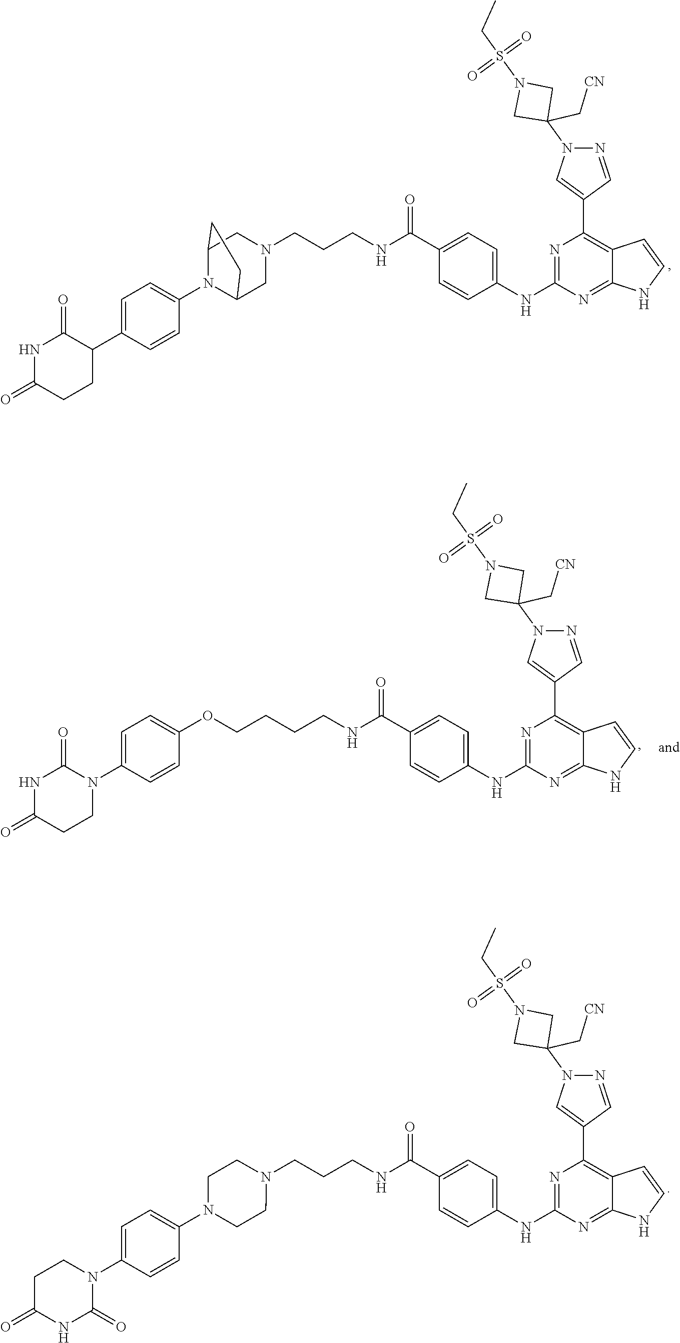 [SMILES: CCS(=O)(=O)N1CC(CC#N)(n2cc(-c3nc(Nc4ccc(C(=O)NCCCN5CC6CCC(C5)N6c5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCOc5ccc(N6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(N7CCC(=O)NC7=O)cc6)CC5)cc4)nc4[nH]ccc34)cn2)C1]

or a pharmaceutically acceptable salt thereof, and a pharmaceutically acceptable carrier.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, P A 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouthwashes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a disorder associated with JAK-2 signaling dysfunction such as, for example, a disorder associated with uncontrolled cellular proliferation, (e.g., cancer), and an autoimmune and inflammatory disorder (e.g., psoriasis, rheumatoid arthritis, pruritus, atopic dermatitis, Crohn's disease, inflammatory bowel disease (IBD), human immunodeficiency virus type-I (HIV)).

In a further aspect, the pharmaceutical composition is used to treat an autoimmune and inflammatory disorder such as, for example, psoriasis, rheumatoid arthritis, systemic lupus erythematosus, multiple sclerosis (MS), type I diabetes mellitus, Guillain-Barre syndrome, chronic inflammatory demyelinating polyneuropathy, Graves' disease, Hashimoto's thyroiditis, myasthenia gravis, vasculitis, pruritus, atopic dermatitis, Crohn's disease, inflammatory bowel disease (IBD), and human immunodeficiency virus type-I (HIV).

In a further aspect, the pharmaceutical composition is used to treat a disorder associated with uncontrolled cellular proliferation such as, for example, cancer, such as for example, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma). In one aspect, the cancer is acute lymphoblastic leukemia.

It is understood that the disclosed compositions can be prepared from the disclosed compounds. It is also understood that the disclosed compositions can be employed in the disclosed methods of using.

### E. Methods of Treating a Disorder Associated with JAK-2 Signaling Dysfunction

In various aspects, the compounds and compositions disclosed herein are useful for treating, preventing, ameliorating, controlling or reducing the risk of a variety of disorders associated with JAK-2 signaling dysfunction, such as, for example, a disorder associated with uncontrolled cellular proliferation, (e.g., cancer) and an autoimmune and inflammatory disorder (e.g., psoriasis, rheumatoid arthritis, pruritus, atopic dermatitis, Crohn's disease). Thus, in one aspect, disclosed are methods of treating a disorder associated with JAK-2 signaling dysfunction in a subject, the method comprising administering to the subject an effective amount of at least one disclosed compound or a pharmaceutically acceptable salt thereof.

In one aspect, disclosed are methods of treating a disorder associated with uncontrolled cellular proliferation such as, for example, cancer, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, myelofibrosis, polycythemia vera, and plasma cell neoplasm (myeloma), in another example acute lymphoblastic leukemia activity in a subject, the method comprising administering to the subject an effective amount of a disclosed compound.

In one aspect, disclosed are methods of treating an autoimmune and inflammatory disorder such as, for example, psoriasis, rheumatoid arthritis, systemic lupus erythematosus, multiple sclerosis (MS), type I diabetes mellitus, Guillain-Barre syndrome, chronic inflammatory demyelinating polyneuropathy, Graves' disease, Hashimoto's thyroiditis, myasthenia gravis, vasculitis, pruritus, atopic dermatitis, Crohn's disease, inflammatory bowel disease (IBD), and human immunodeficiency virus type-I (HIV) in a subject, the method comprising administering to the subject an effective amount of a disclosed compound.

In a further aspect, the compound has a structure represented by a formula:

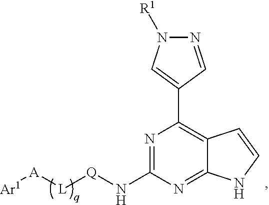

wherein q is selected from 0 and 1; wherein A is selected from —O—, —S—, —NH—, —NHCH₂—, —OCH₂C(O)NH—, —CH₂—, and a structure selected from:

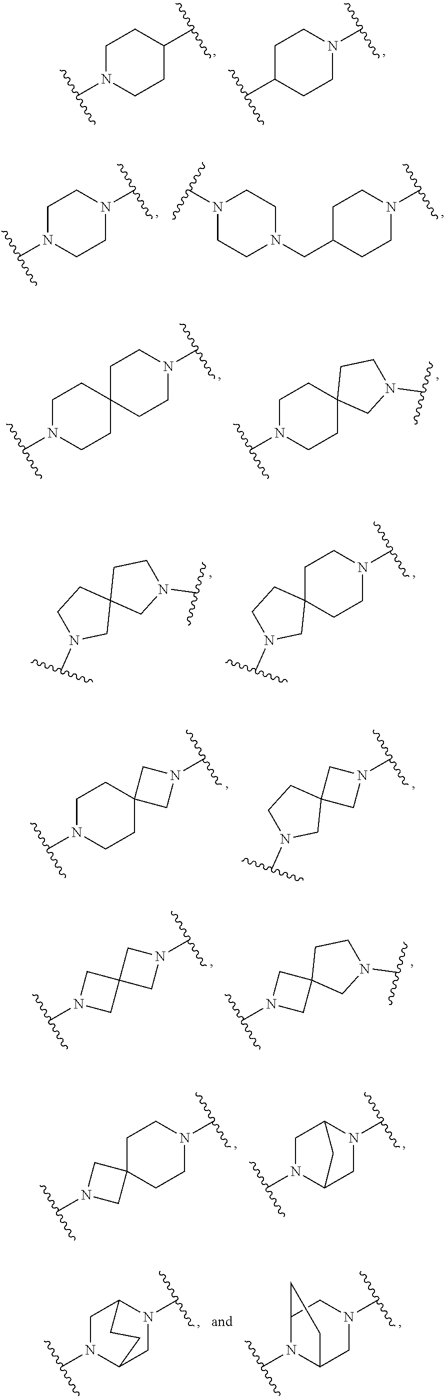

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

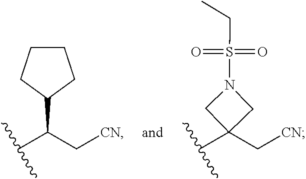 [SMILES: *[C@H](CC#N)C1CCCC1 ; *C1(CC#N)CN(S(=O)(=O)CC)C1]

and
wherein Ar¹ is a structure selected from:

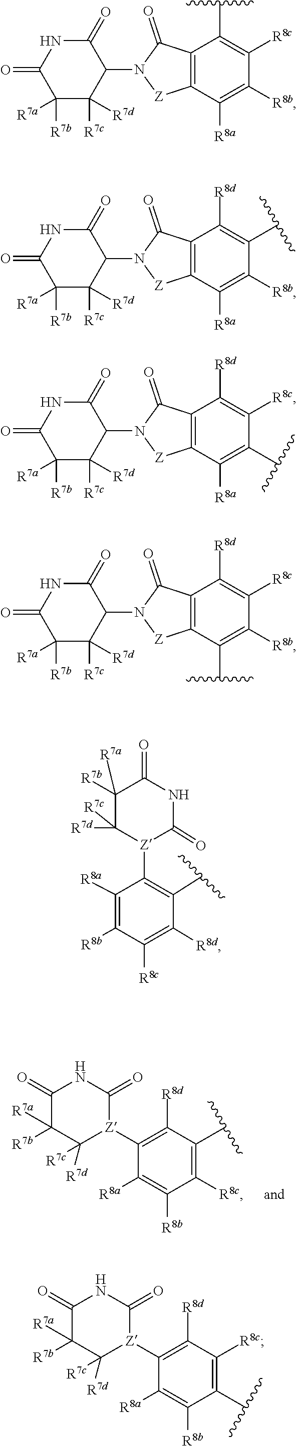

wherein Z is selected from —CH₂— and C(O); wherein Z′ is selected from —CH— and —N—; wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

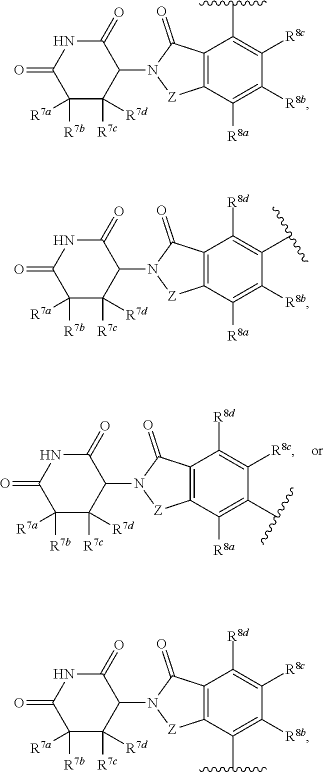

and q is 1, then A is a structure selected from:

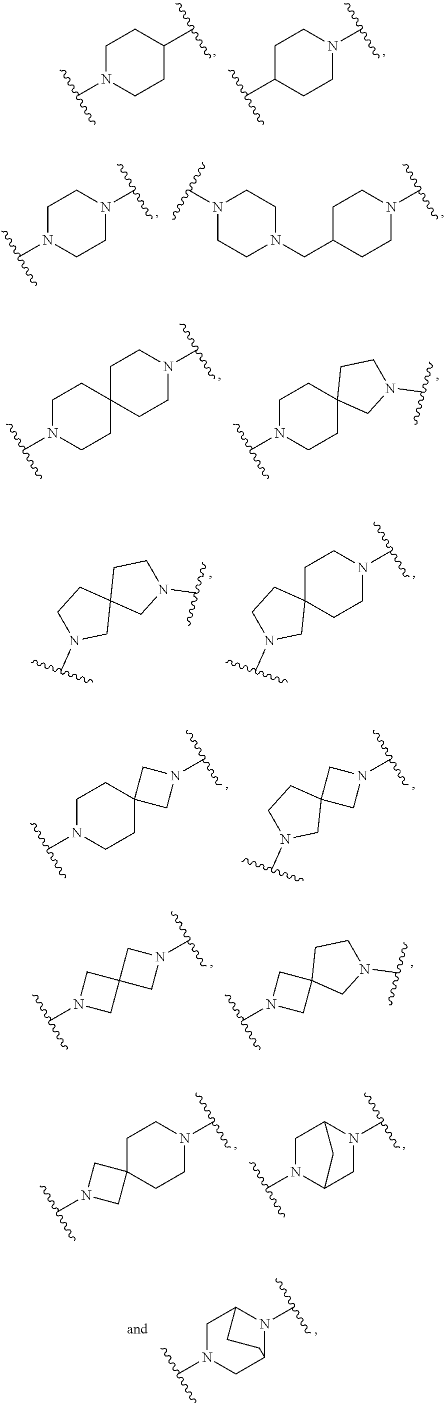

or a pharmaceutically acceptable salt thereof.

In a further aspect, the compound has a structure represented by a formula:

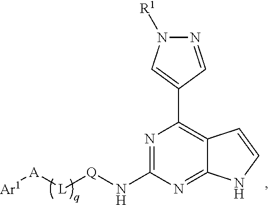

wherein q is selected from 0 and 1; wherein A is selected from —O—, —S—, —NH—, —NHCH₂—, —OCH₂C(O)NH—, —CH₂—, and a structure selected from:

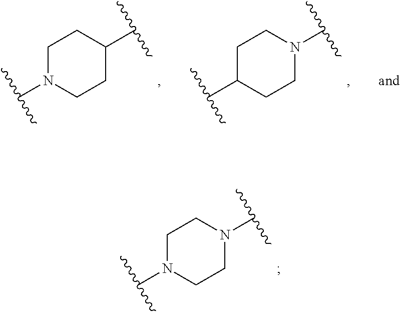

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

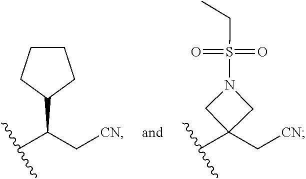 [SMILES: *[C@H](CC#N)C1CCCC1 ; *C1(CC#N)CN(S(=O)(=O)CC)C1]

and
wherein Ar¹ is a structure selected from:

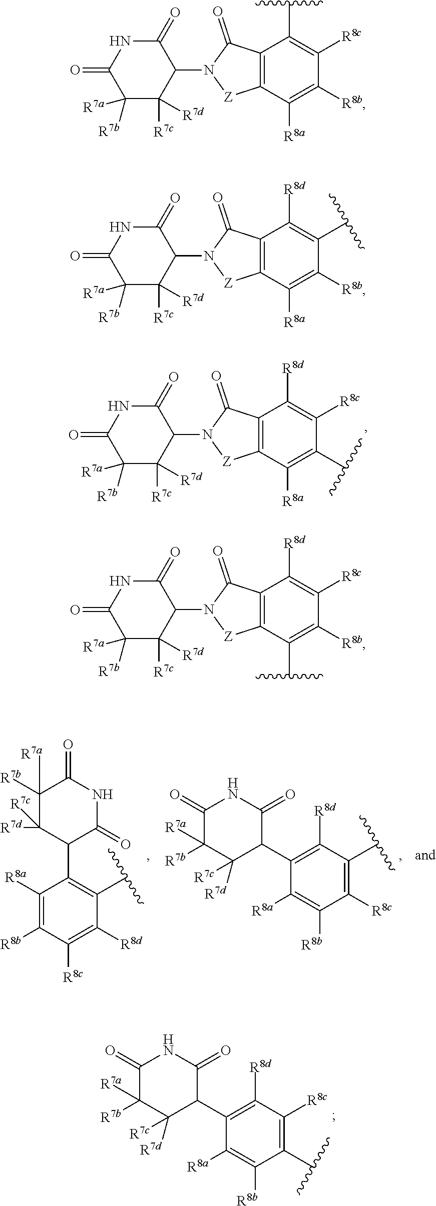

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

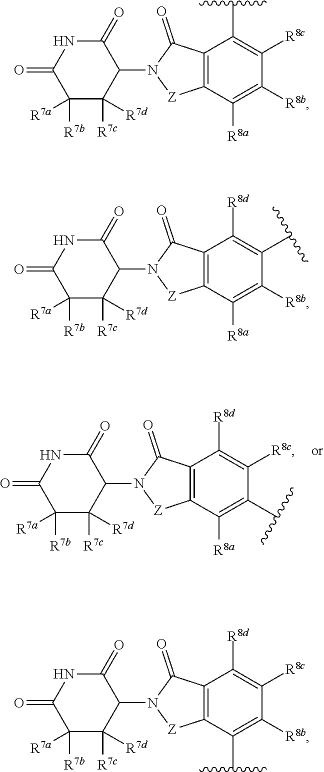

and q is 1, then A is a structure selected from:

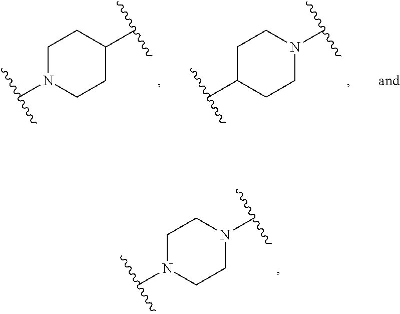 [SMILES: *C1CCN(*)CC1 ; *C1CCN(*)CC1 ; *N1CCN(*)CC1]

or a pharmaceutically acceptable salt thereof.

In a further aspect, the compound has a structure selected from:

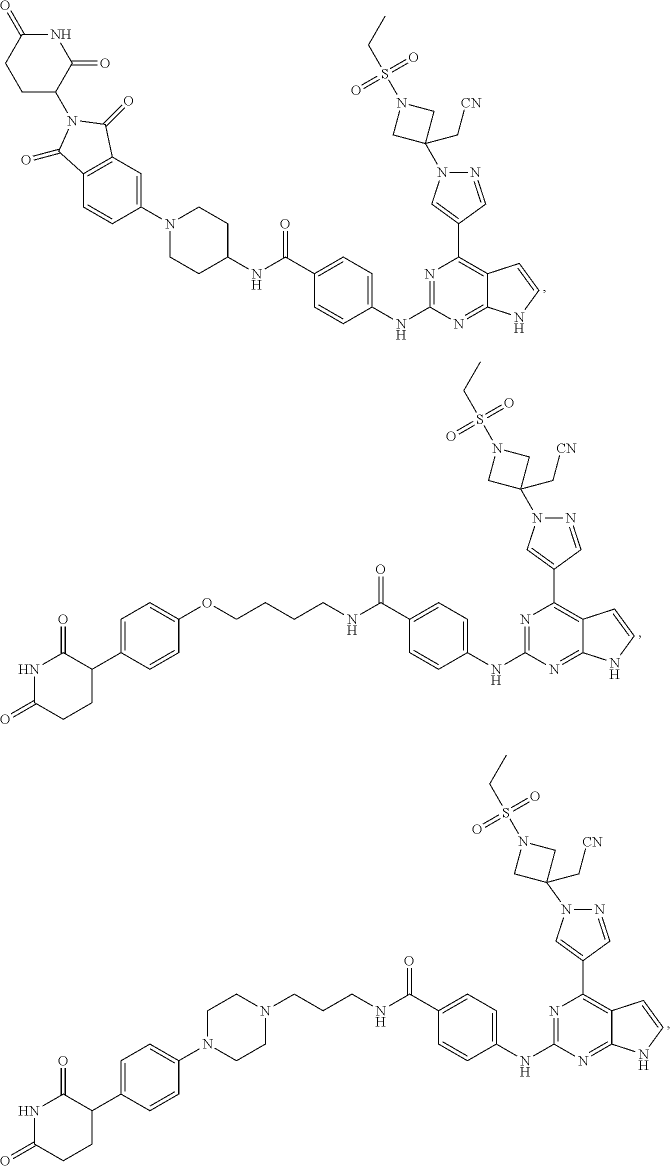

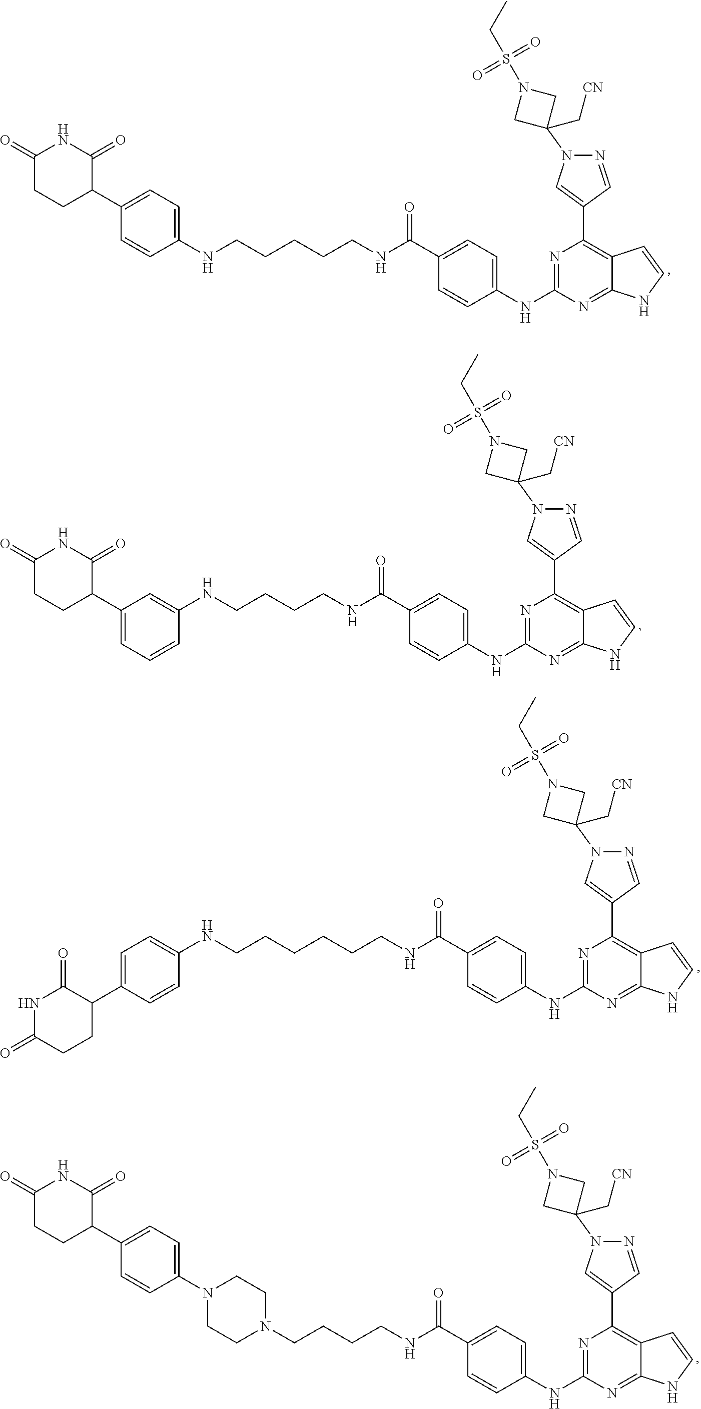

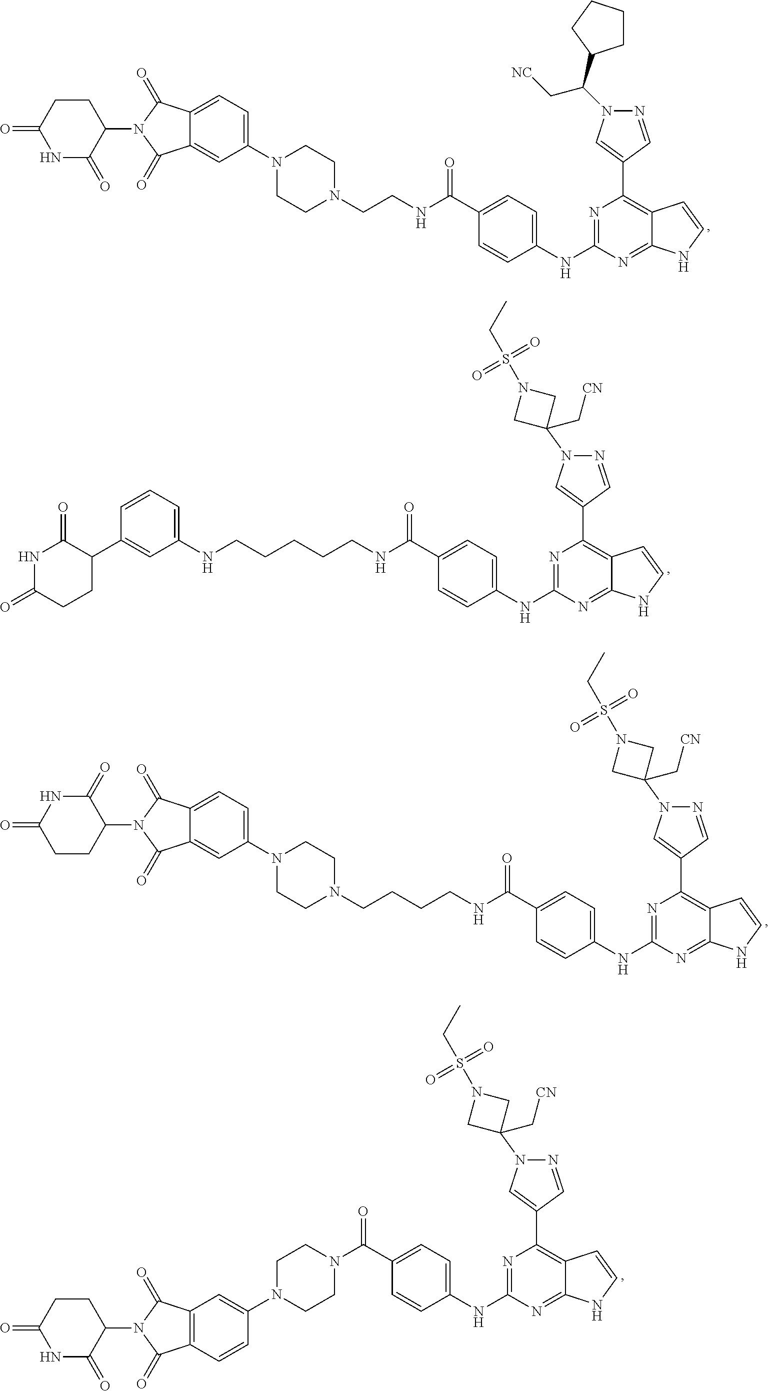

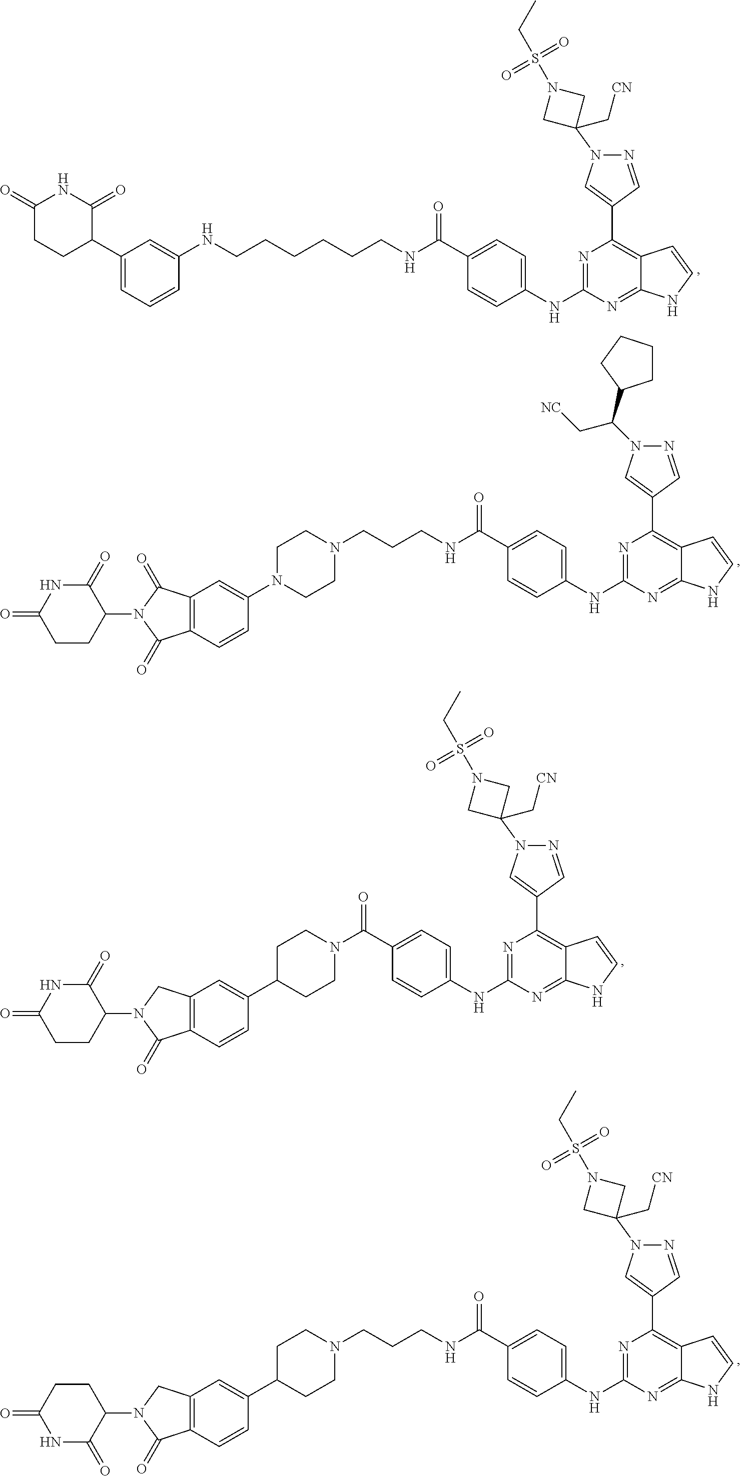

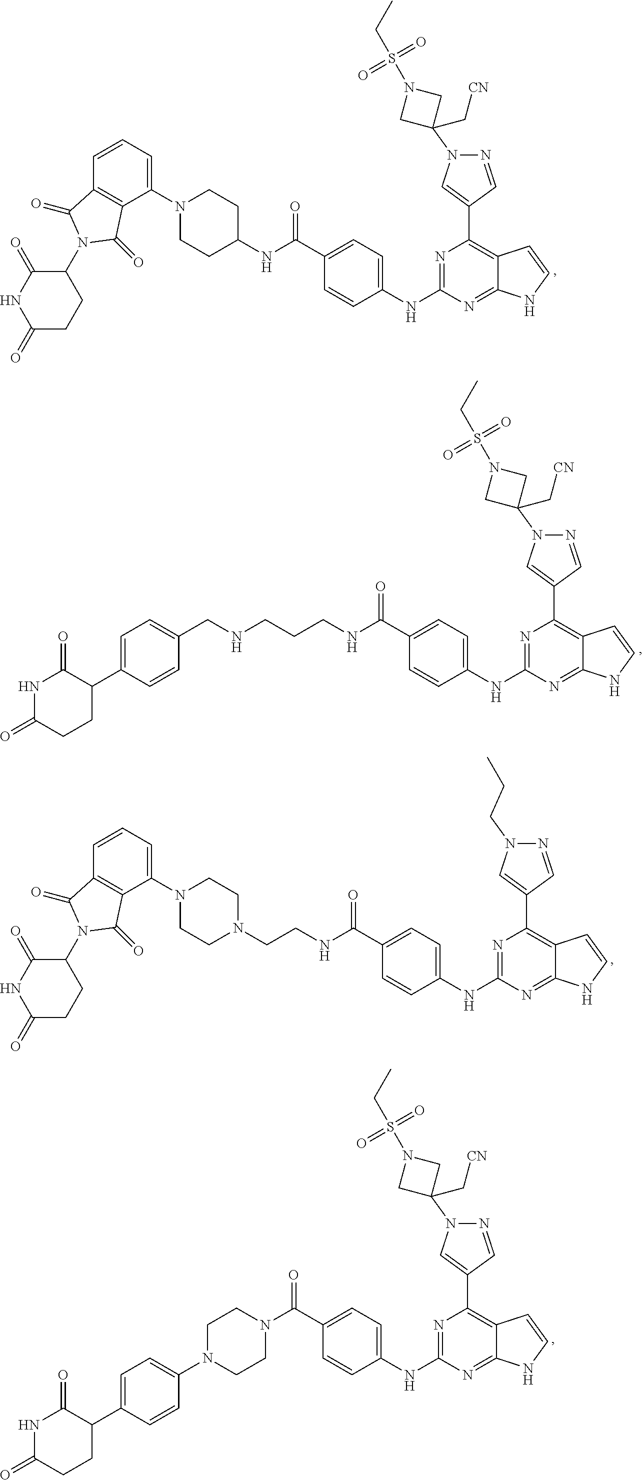 [SMILES: CCS(=O)(=O)N1CC(CC#N)(n2cc(-c3nc(Nc4ccc(C(=O)NC5CCN(c6cccc7c6C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCNCc5ccc(C6CCC(=O)NC6=O)cc5)cc4)nc4[nH]ccc34)cn2)C1 ; CCCn1cc(-c2nc(Nc3ccc(C(=O)NCCN4CCN(c5cccc6c5C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1]

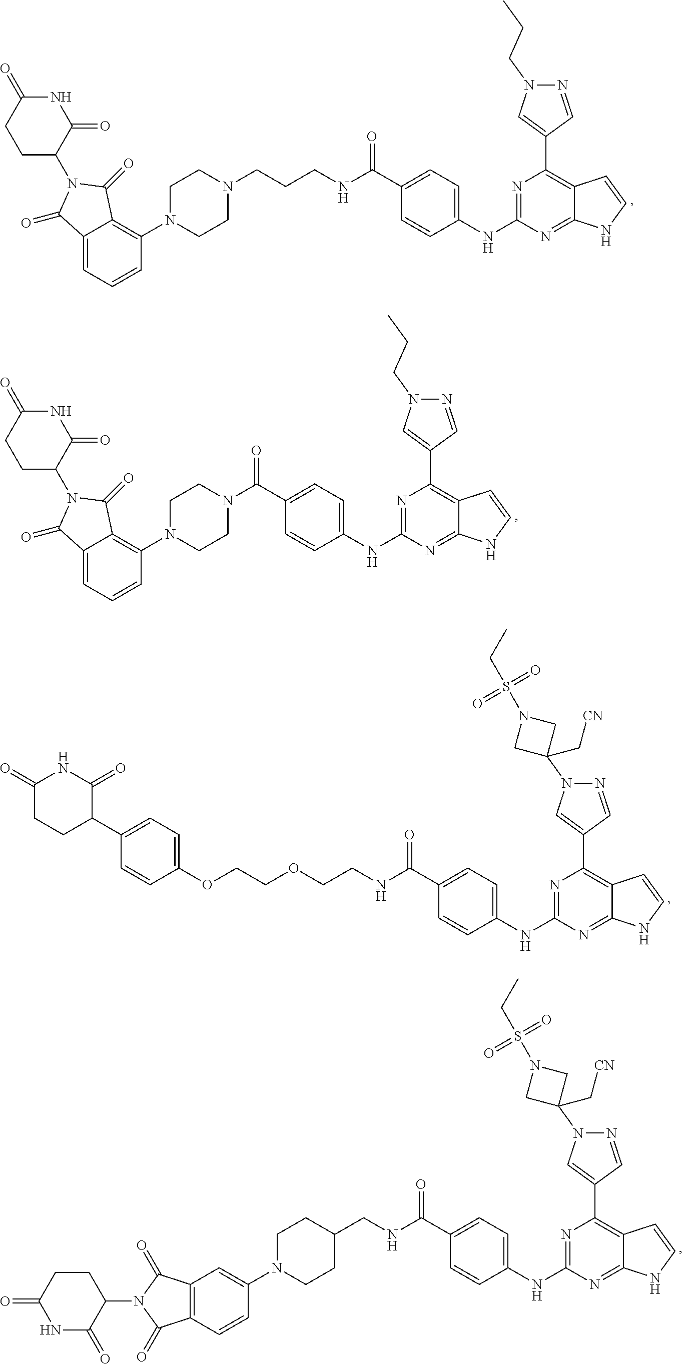

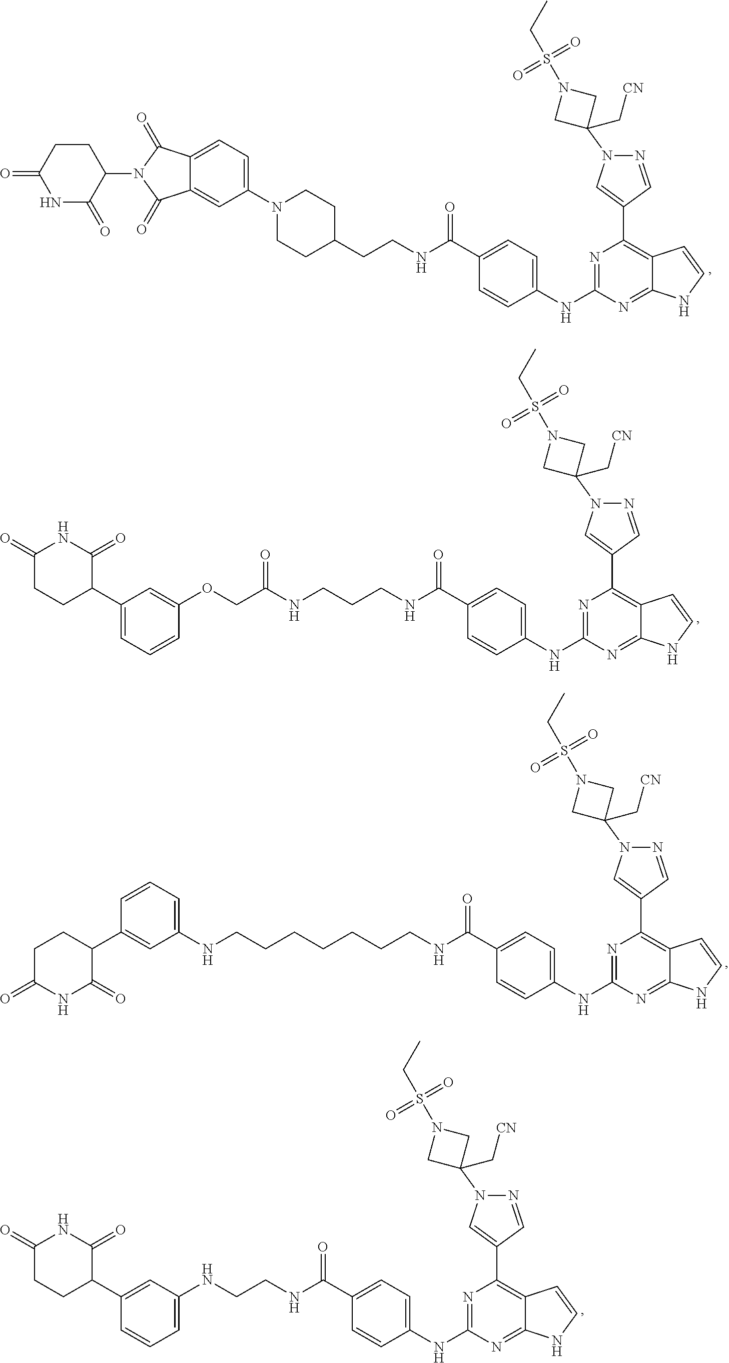

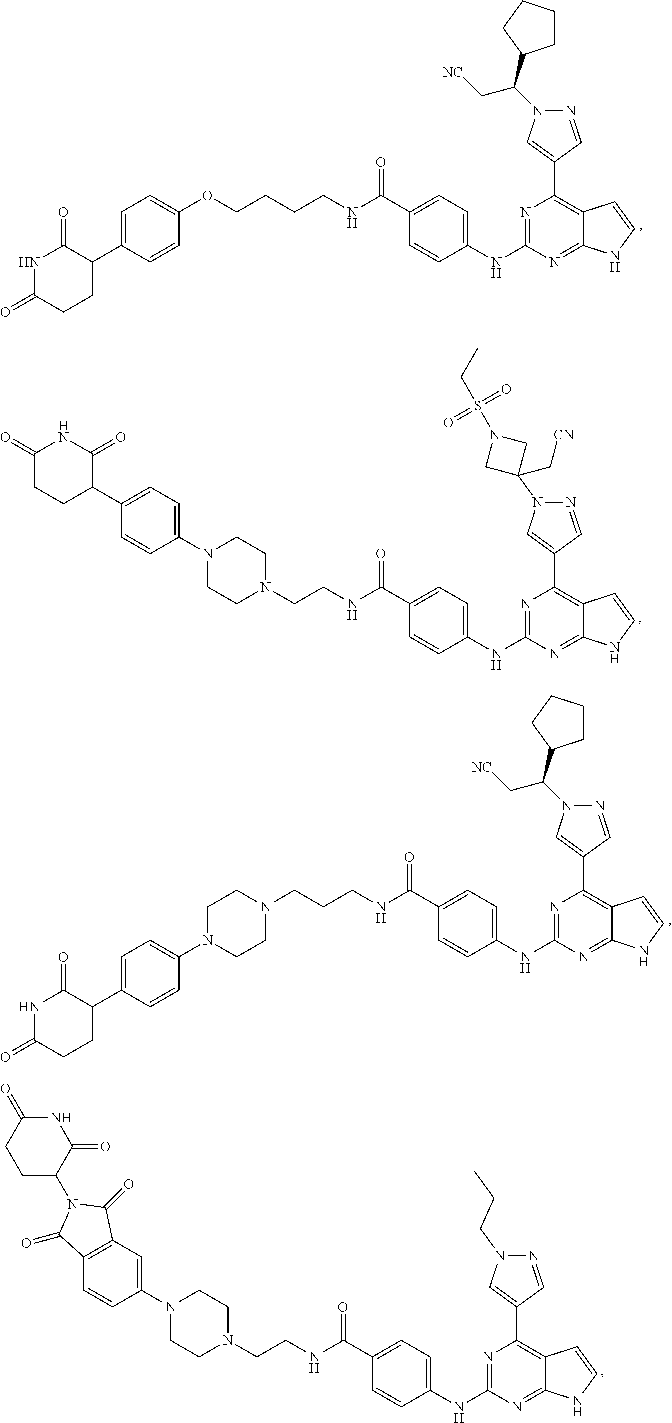

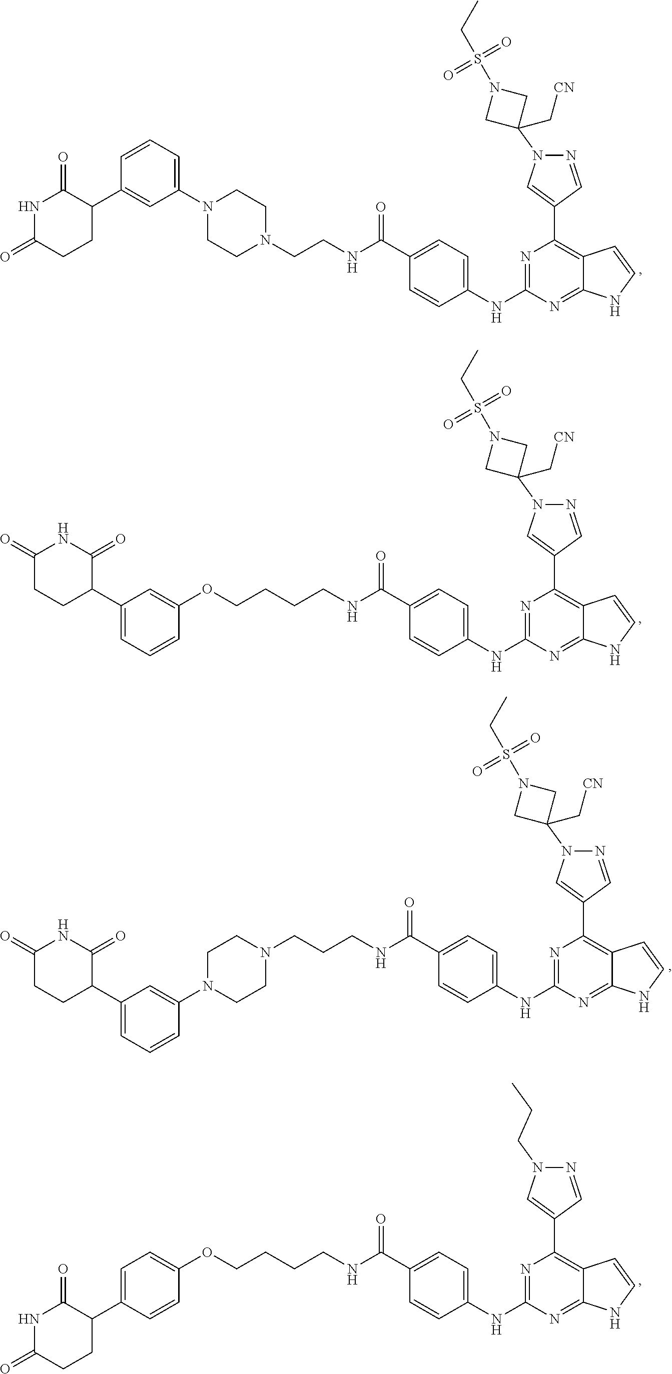

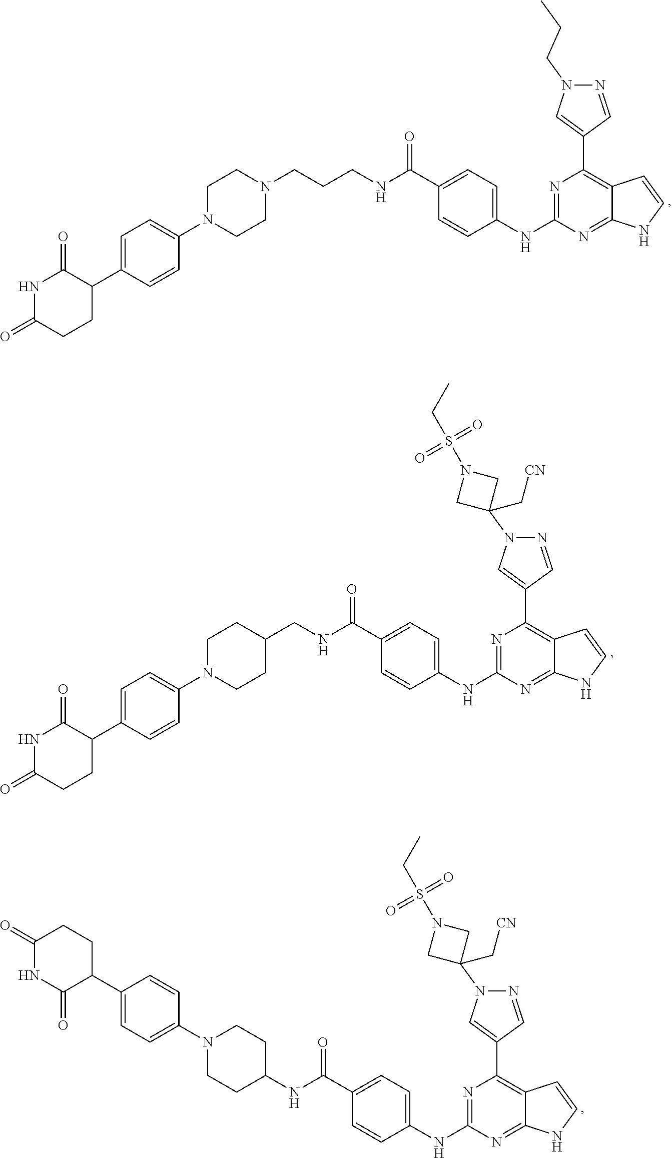 [SMILES: CCCn1cc(-c2nc(Nc3ccc(C(=O)NCCCN4CCN(c5ccc(C6CCC(=O)NC6=O)cc5)CC4)cc3)nc3[nH]ccc23)cn1 ; CCS(=O)(=O)N1CC(CC#N)(n2cc(-c3nc(Nc4ccc(C(=O)NCC5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NC5CCN(c6ccc(C7CCC(=O)NC7=O)cc6)CC5)cc4)nc4[nH]ccc34)cn2)C1]

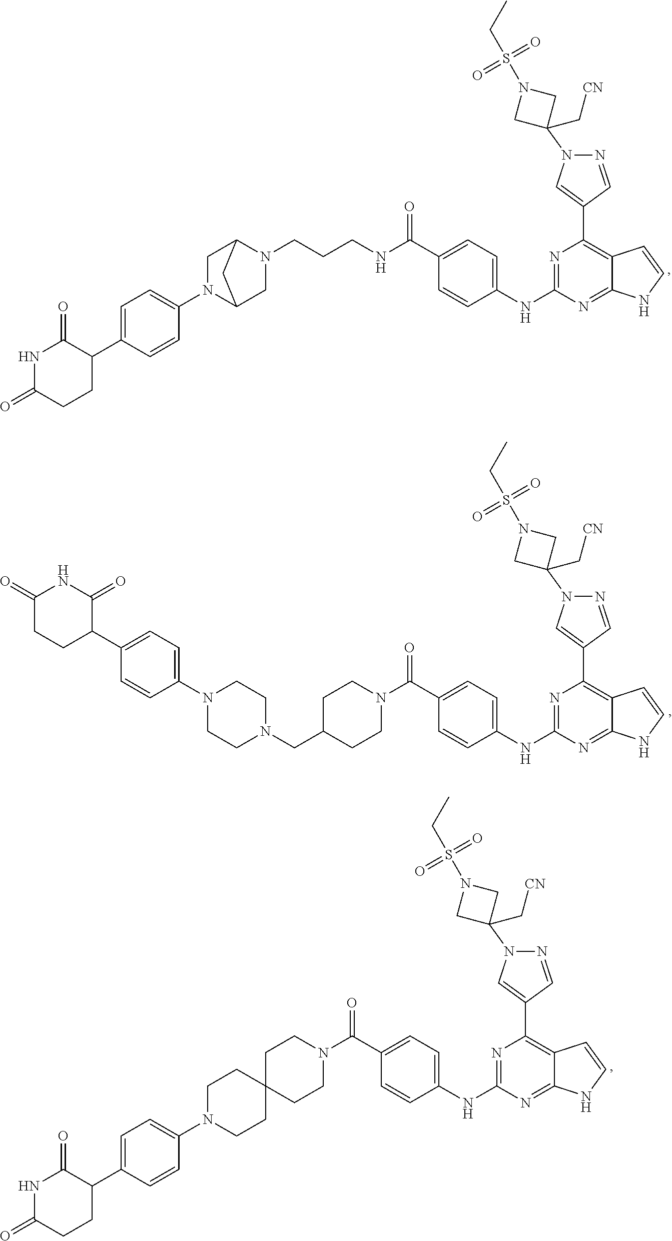 [SMILES: CCS(=O)(=O)N1CC(CC#N)(n2cc(-c3nc(Nc4ccc(C(=O)NCCCN5CC6CC5CN6c5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)N5CCC(CN6CCN(c7ccc(C8CCC(=O)NC8=O)cc7)CC6)CC5)cc4)nc4[nH]ccc34)cn2)C1 ; CCS(=O)(=O)N1CC(CC#N)(n2cc(-c3nc(Nc4ccc(C(=O)N5CCC6(CC5)CCN(c5ccc(C7CCC(=O)NC7=O)cc5)CC6)cc4)nc4[nH]ccc34)cn2)C1]

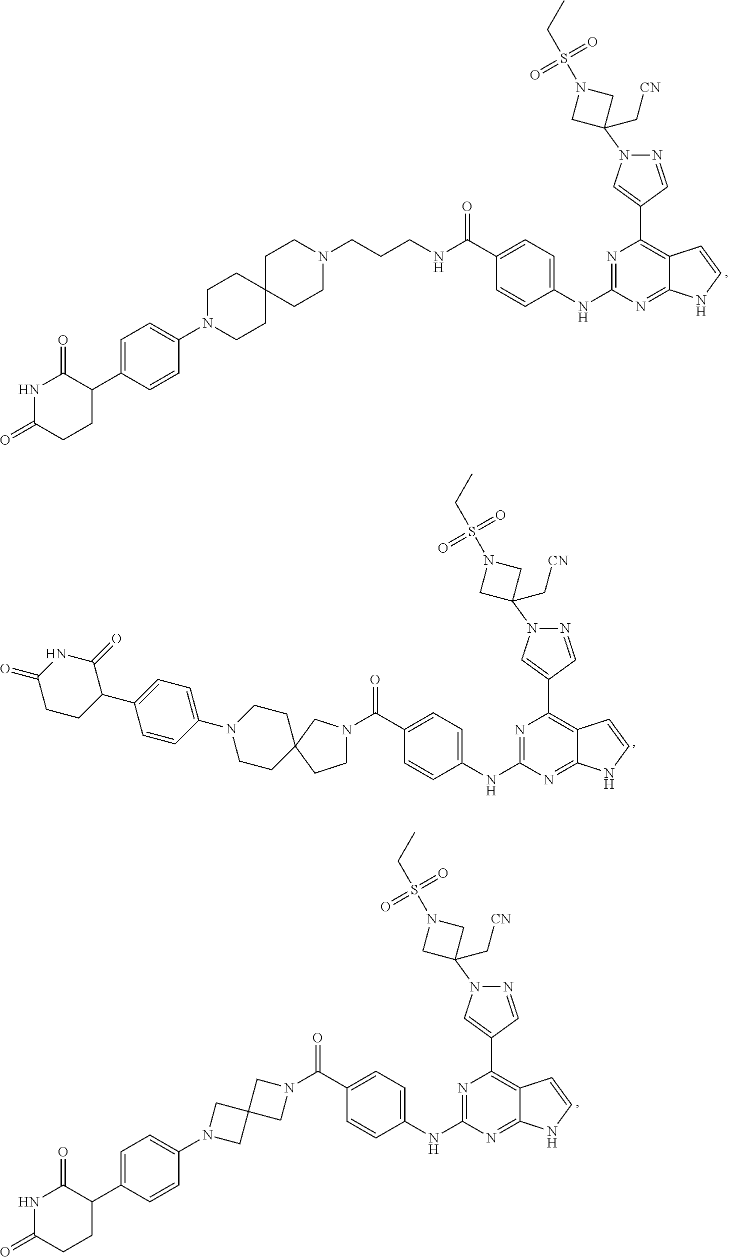 [SMILES: CCS(=O)(=O)N1CC(CC#N)(n2cc(-c3nc(Nc4ccc(C(=O)NCCCN5CCC6(CC5)CCN(c5ccc(C7CCC(=O)NC7=O)cc5)CC6)cc4)nc4[nH]ccc34)cn2)C1 ; CCS(=O)(=O)N1CC(CC#N)(n2cc(-c3nc(Nc4ccc(C(=O)N5CCC6(CCN(c7ccc(C8CCC(=O)NC8=O)cc7)CC6)C5)cc4)nc4[nH]ccc34)cn2)C1 ; CCS(=O)(=O)N1CC(CC#N)(n2cc(-c3nc(Nc4ccc(C(=O)N5CC6(C5)CN(c5ccc(C7CCC(=O)NC7=O)cc5)C6)cc4)nc4[nH]ccc34)cn2)C1]

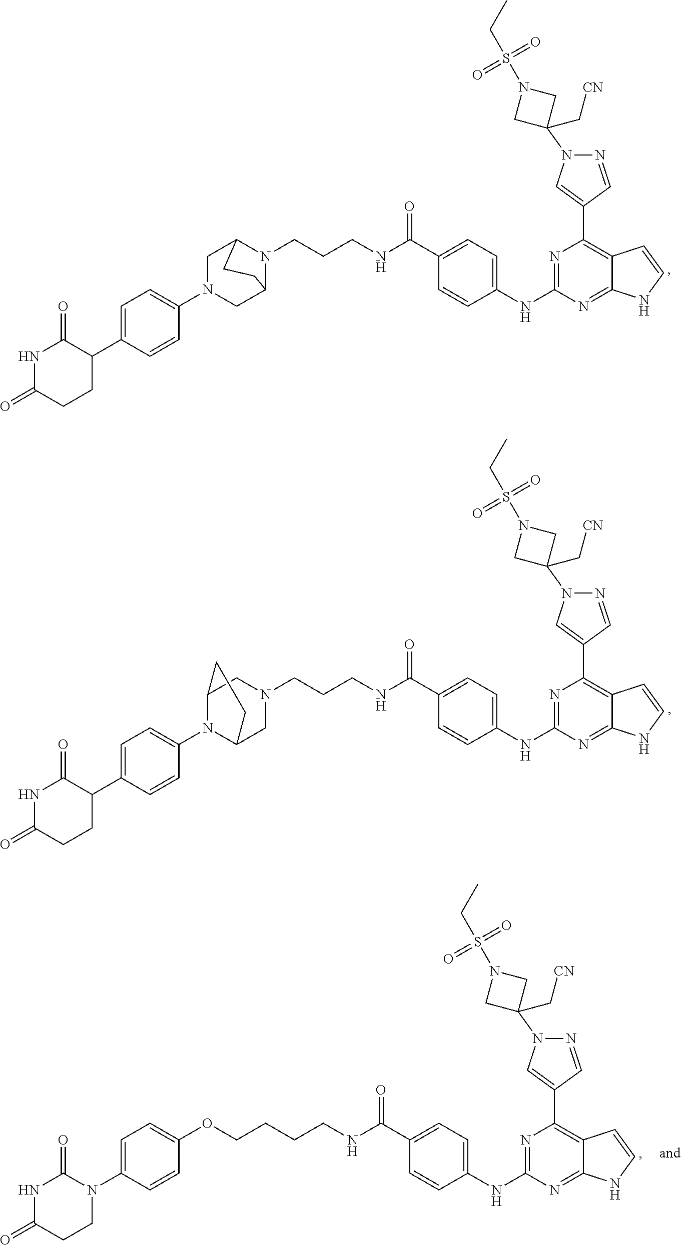 [SMILES: CCS(=O)(=O)N1CC(CC#N)(n2cc(-c3nc(Nc4ccc(C(=O)NCCCN5C6CCC5CN(c5ccc(C7CCC(=O)NC7=O)cc5)C6)cc4)nc4[nH]ccc34)cn2)C1 ; CCS(=O)(=O)N1CC(CC#N)(n2cc(-c3nc(Nc4ccc(C(=O)NCCCN5CC6CCC(C5)N6c5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCOc5ccc(N6CCC(=O)NC6=O)cc5)cc4)nc4[nH]ccc34)cn2)C1]

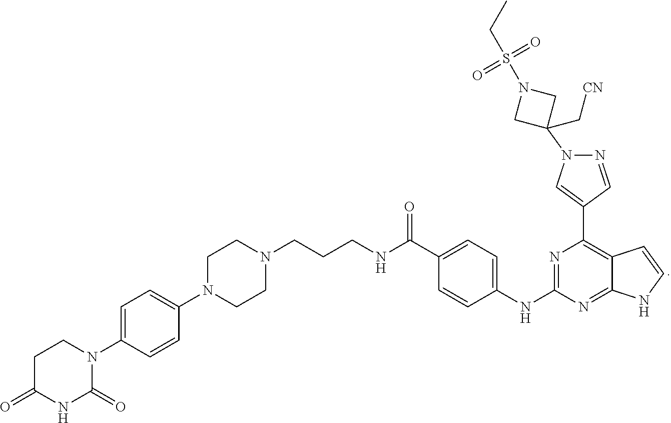 [SMILES: CCS(=O)(=O)N1CC(CC#N)(n2cc(-c3nc(Nc4ccc(C(=O)NCCCN5CCN(c6ccc(N7CCC(=O)NC7=O)cc6)CC5)cc4)nc4[nH]ccc34)cn2)C1]

or a pharmaceutical acceptable salt thereof.

In various aspects, the disclosed compounds can be used in combination with one or more other drugs in the treatment, prevention, control, amelioration, or reduction of risk of a disorder associated with JAK-2 signaling dysfunction, wherein the combination of the drugs together are safer or more effective than either drug alone.

In various aspects, the disclosed compounds can be used in combination with one or more other drugs in the treatment, prevention, control, amelioration, or reduction of risk of disorders associated with uncontrolled cellular proliferation, such as for example cancer, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), in another example acute lymphoblastic leukemia, activity for which disclosed compounds or the other drugs can have utility, where the combination of the drugs together are safer or more effective than either drug alone.

Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly. Accordingly, the pharmaceutical compositions include those that contain one or more other active ingredients, in addition to a compound of the present invention.

In one aspect, the one or more other active ingredients is a chemotherapeutic agent. The chemotherapeutic agent can be selected from an alkylating agent, an antimetabolite agent, an antineoplastic antibiotic agent, a mitotic inhibitor agent, and a mTor inhibitor agent. In one aspect, the one or more other active ingredients is an antineoplastic antibiotic agent selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof. In a further aspect, the one or more other active ingredients is an antimetabolite agent selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof. In a further aspect, the one or more other active ingredients is an alkylating agent selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof. In a further aspect, the one or more other active ingredients is a mitotic inhibitor agent selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof. In a further aspect, the one or more other active ingredients is an mTor inhibitor agent selected from everolimus, siroliumus, and temsirolimus, or a pharmaceutically acceptable salt, hydrate, solvate, or polymorph thereof.

In a further aspect, the compound exhibits inhibition of uncontrolled cellular proliferation, such as for example cancer, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), in another example acute lymphoblastic leukemia, activity. In a still further aspect, the compound exhibits a decrease in uncontrolled cellular proliferation, such as for example cancer, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), in another example acute lymphoblastic leukemia, activity.

In another aspect, disclosed herein is a method of treating a JAK-associated disease or disorder in a subject comprising administering to the subject an effective amount of at least one compound disclosed herein. The JAK-associated disease can include any disease, disorder or condition that is directly or indirectly linked to expression or activity of the JAK, including overexpression and/or abnormal activity levels. A JAK-associated disease can also include any disease, disorder or condition that can be prevented, ameliorated, or cured by modulating JAK activity.

In another aspect, JAK-associated diseases include diseases involving the immune system including, for example, organ transplant rejection (e.g., allograft rejection and graft versus host disease).

In another aspect, JAK-associated diseases include autoimmune diseases such as multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, autoimmune thyroid disorders, and the like. In some embodiments, the autoimmune disease is an autoimmune bullous skin disorder such as pemphigus vulgaris (PV) or bullous pemphigoid (BP).

In another example, JAK-associated diseases include allergic conditions such as asthma, food allergies, atopic dermatitis and rhinitis. Further examples of JAK-associated diseases include viral diseases such as Epstein Barr Virus (EBV), Hepatitis B, Hepatitis C, HIV, HTLV 1, Varicella-Zoster Virus (VZV) and Human Papilloma Virus (HPV). Further examples of JAK-associated diseases or conditions include skin disorders such as psoriasis (for example, psoriasis vulgaris), atopic dermatitis, skin rash, skin irritation, skin sensitization (e.g., contact dermatitis or allergic contact dermatitis).

In a further aspect, the compound exhibits inhibition of uncontrolled cellular proliferation activity with an IC₅₀ of from about 0.001 μM to about 25 μM. In a still further aspect, the compound exhibits inhibition of uncontrolled cellular proliferation activity with an IC₅₀ of from about 0.001 μM to about 15 μM. In yet a further aspect, the compound exhibits inhibition of uncontrolled cellular proliferation activity with an IC₅₀ of from about 0.001 μM to about 10 μM. In an even further aspect, the compound exhibits inhibition of uncontrolled cellular proliferation activity with an IC₅₀ of from about 0.001 μM to about 5 μM. In a still further aspect, the compound exhibits inhibition of uncontrolled cellular proliferation activity with an IC₅₀ of from about 0.001 μM to about 1 μM. In yet a further aspect, the compound exhibits inhibition of uncontrolled cellular proliferation activity with an IC₅₀ of from about 0.001 μM to about 0.5 μM. In an even further aspect, the compound exhibits inhibition of uncontrolled cellular proliferation activity with an IC₅₀ of from about 0.001 μM to about 0.1 μM. In a still further aspect, the compound exhibits inhibition of uncontrolled cellular proliferation activity with an IC₅₀ of from about 0.001 μM to about 0.05 μM. In yet a further aspect, the compound exhibits inhibition of uncontrolled cellular proliferation activity with an IC₅₀ of from about 0.001 μM to about 0.01 μM. In an even further aspect, the compound exhibits inhibition of uncontrolled cellular proliferation activity with an IC₅₀ of from about 0.001 μM to about 0.005 μM. In a still further aspect, the compound exhibits inhibition of uncontrolled cellular proliferation activity with an IC₅₀ of from about 0.005 μM to about 25 μM. In yet a further aspect, the compound exhibits inhibition of uncontrolled cellular proliferation activity with an IC₅₀ of from about 0.01 μM to about 25 μM. In an even further aspect, the compound exhibits inhibition of uncontrolled cellular proliferation activity with an IC₅₀ of from about 0.05 μM to about 25 μM. In a still further aspect, the compound exhibits inhibition of uncontrolled cellular proliferation activity with an IC₅₀ of from about 0.1 μM to about 25 μM. In yet a further aspect, the compound exhibits inhibition of uncontrolled cellular proliferation activity with an IC₅₀ of from about 0.5 μM to about 25 μM. In an even further aspect, the compound exhibits inhibition of uncontrolled cellular proliferation activity with an IC₅₀ of from about 1 μM to about 25 μM. In a still further aspect, the compound exhibits inhibition of uncontrolled cellular proliferation activity with an IC₅₀ of from about 5 μM to about 25 μM. In yet a further aspect, the compound exhibits inhibition of uncontrolled cellular proliferation activity with an IC₅₀ of from about 10 μM to about 25 μM. In an even further aspect, the compound exhibits inhibition of uncontrolled cellular proliferation activity with an IC₅₀ of from about 15 μM to about 25 μM.

In a further aspect, the subject is a mammal. In a still further aspect, the mammal is human.

In a further aspect, the subject has been diagnosed with a need for treatment of the disorder prior to the administering step. In a still further aspect, the subject is at risk for developing the disorder prior to the administering step.

In a further aspect, the method further comprises identifying a subject at risk for developing the disorder prior to the administering step.

In a further aspect, the disorder associated with uncontrolled cellular proliferation is cancer, for example a cancer selected from sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), in another example acute lymphoblastic leukemia (ALL).

### F. Methods of Modulating Cellular Proliferation in a Subject

In one aspect, disclosed are methods of modulating cellular proliferation in a subject, the method comprising administering to the subject an effective amount of at least one disclosed compound, or a pharmaceutically acceptable salt thereof. In a further aspect, modulating is inhibiting.

Thus, in one aspect, disclosed are methods of modulating cellular proliferation in a subject in need thereof, the method comprising administering to the subject an effective amount of a compound having a structure represented by a formula:

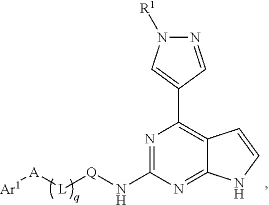

wherein q is selected from 0 and 1; wherein A is selected from —O—, —S—, —NH—, —NHCH₂—, —OCH₂C(O)NH—, —CH₂—, and a structure selected from:

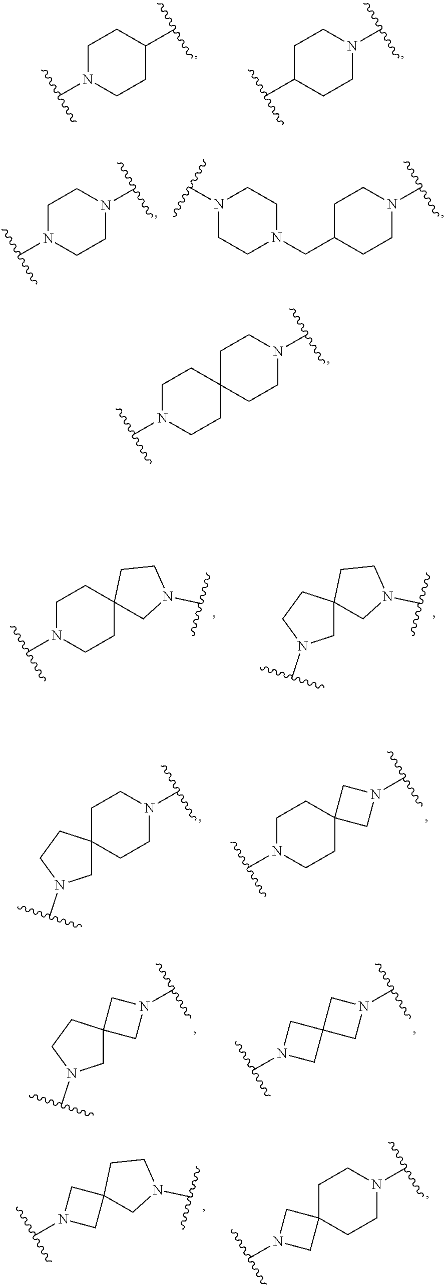

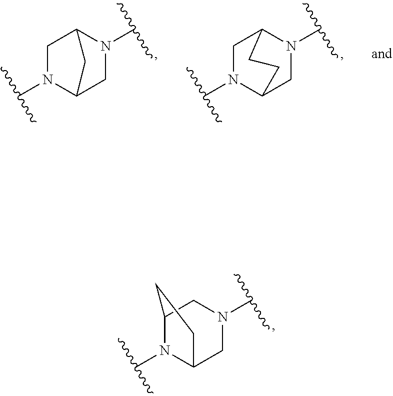

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

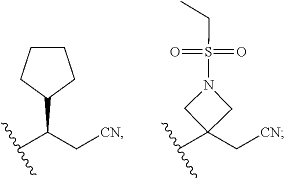 [SMILES: *[C@H](CC#N)C1CCCC1 ; *C1(CC#N)CN(S(=O)(=O)CC)C1]

and
wherein Ar¹ is a structure selected from:

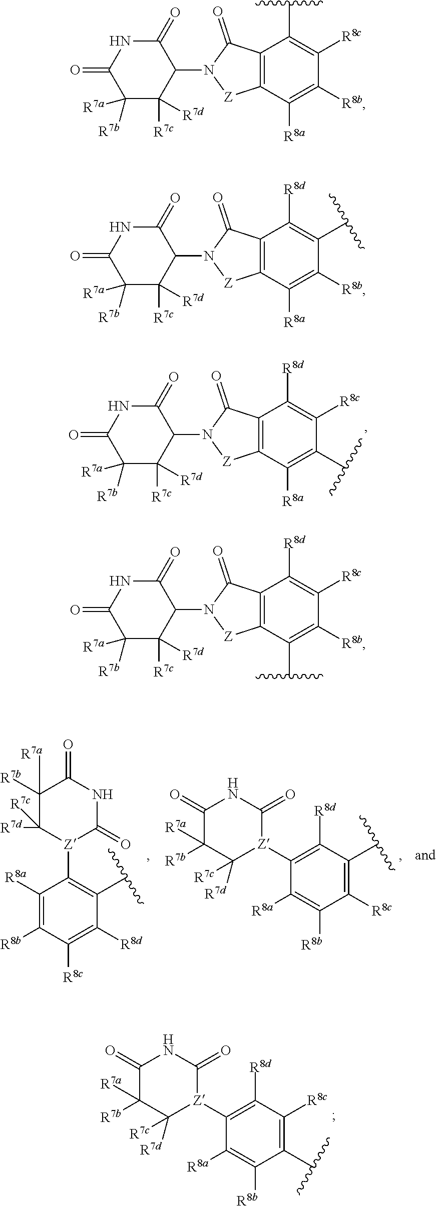

wherein Z is selected from —CH₂— and C(O); wherein Z′ is selected from —CH— and —N—; wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

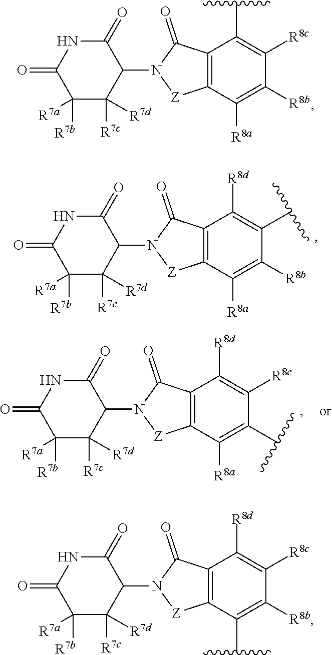

and q is 1, then A is a structure selected from:

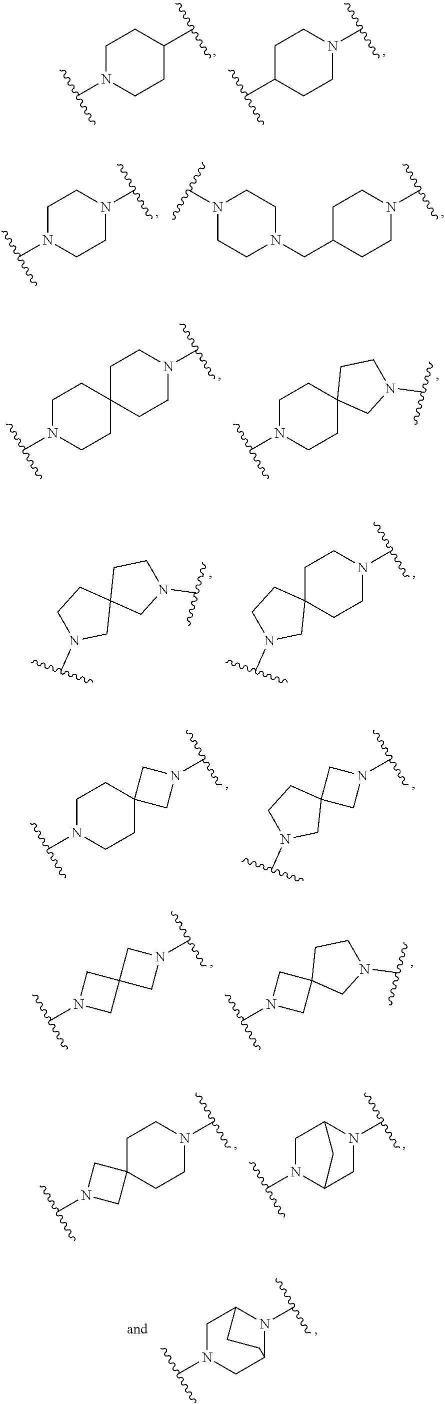

or a pharmaceutically acceptable salt thereof.

In one aspect, disclosed are methods of modulating cellular proliferation in a subject, the method comprising administering to the subject an effective amount of a compound having a structure represented by a formula:

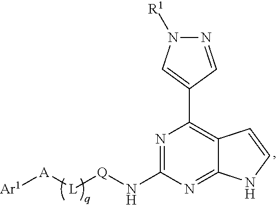

wherein q is selected from 0 and 1; wherein A is selected from —O—, —S—, —NH—, —NHCH₂—, —OCH₂C(O)NH—, —CH₂—, and a structure selected from:

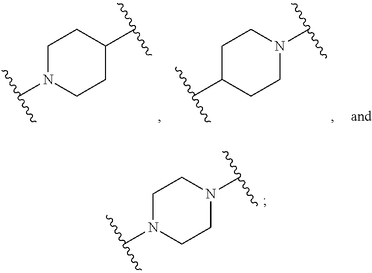

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

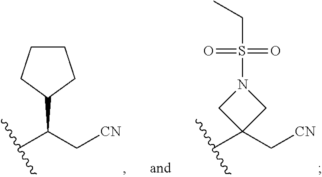 [SMILES: *[C@H](CC#N)C1CCCC1 ; *C1(CC#N)CN(S(=O)(=O)CC)C1]

and
wherein Ar¹ is a structure selected from:

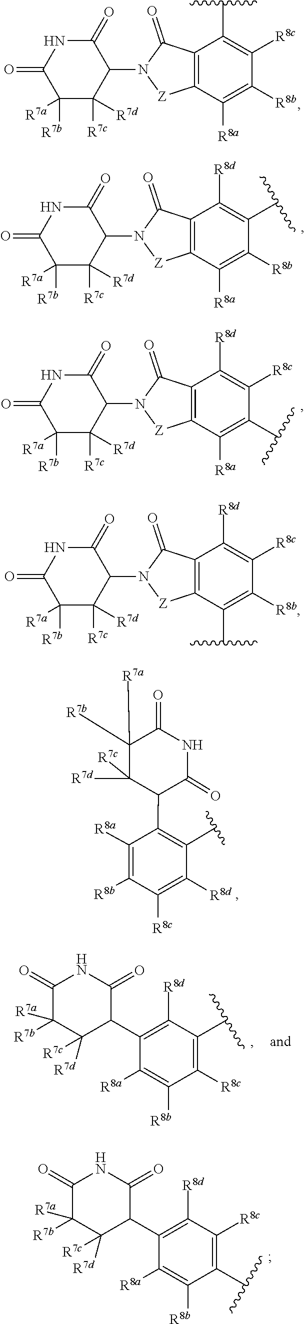

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

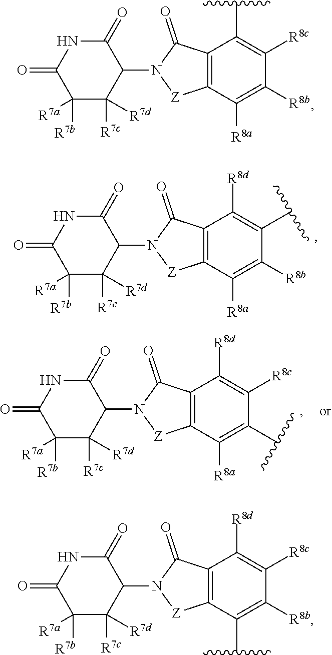

and q is 1, then A is a structure selected from:

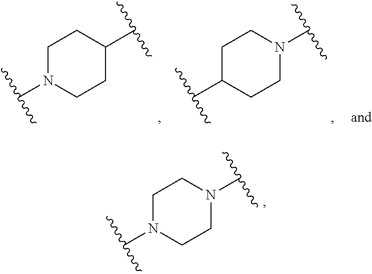 [SMILES: *C1CCN(*)CC1 ; *C1CCN(*)CC1 ; *N1CCN(*)CC1]

or a pharmaceutically acceptable salt thereof.

Also disclosed are disclosed are methods of treating a disorder associated with uncontrolled cellular proliferation, such as for example cancer, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), in another example acute lymphoblastic leukemia activity in a subject, the method comprising administering to the subject an effective amount of a compound having a structure represented by a formula:

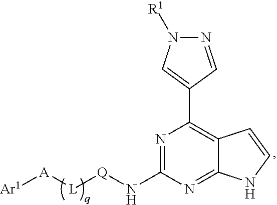

wherein q is selected from 0 and 1; wherein A is selected from —O—, —S—, —NH—, —NHCH₂—, —CH₂—, and a structure selected from:

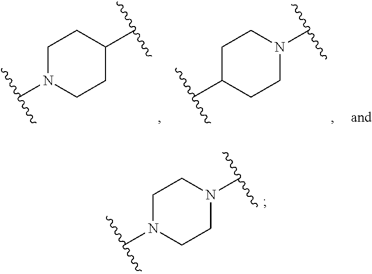

wherein L is selected from C2-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

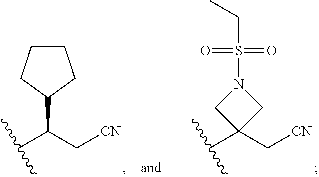 [SMILES: *[C@H](CC#N)C1CCCC1 ; *C1(CC#N)CN(S(=O)(=O)CC)C1]

and
wherein Ar¹ is a structure selected from:

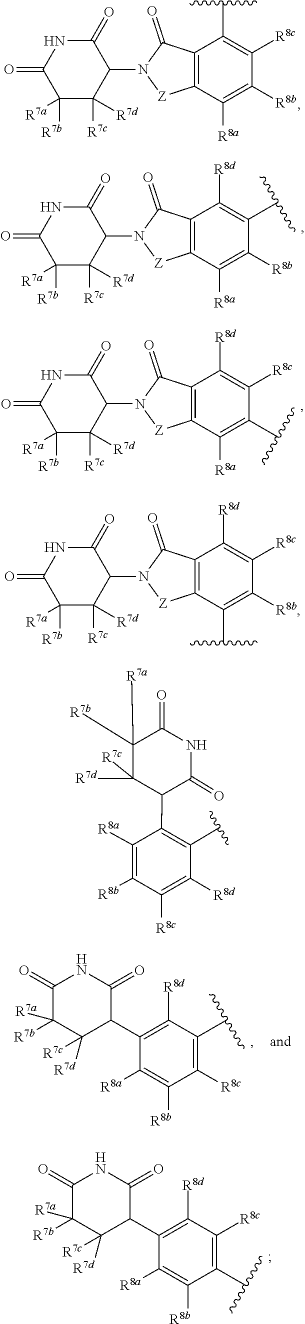

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

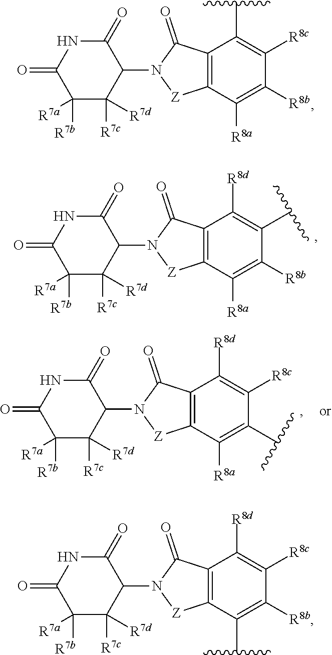

and q is 1, then A is a structure selected from:

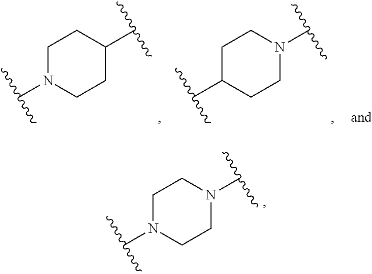 [SMILES: *C1CCN(*)CC1 ; *C1CCN(*)CC1 ; *N1CCN(*)CC1]

or a pharmaceutically acceptable salt thereof.

In one aspect, disclosed are methods of modulating cellular proliferation in a subject, the method comprising administering to the subject an effective amount of at least one compound having a structure selected from:

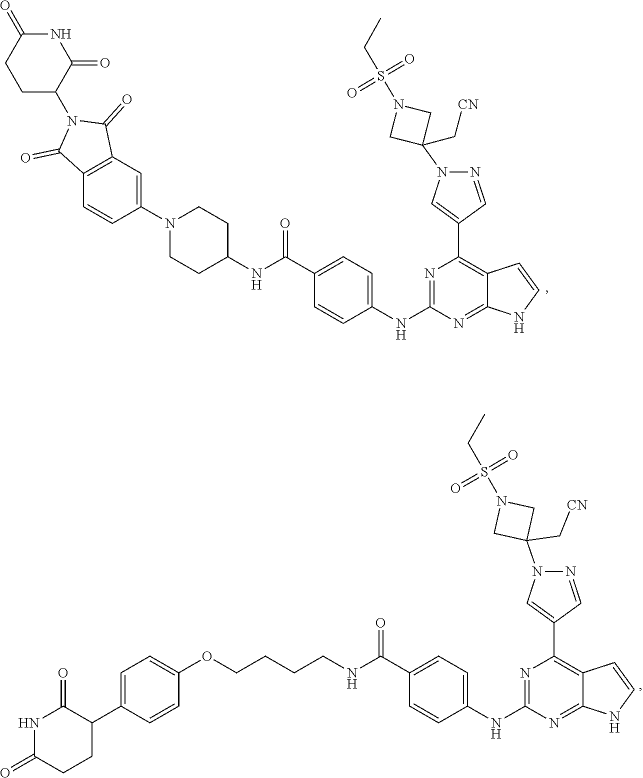 [SMILES: CCS(=O)(=O)N1CC(CC#N)(n2cc(-c3nc(Nc4ccc(C(=O)NC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCOc5ccc(C6CCC(=O)NC6=O)cc5)cc4)nc4[nH]ccc34)cn2)C1]

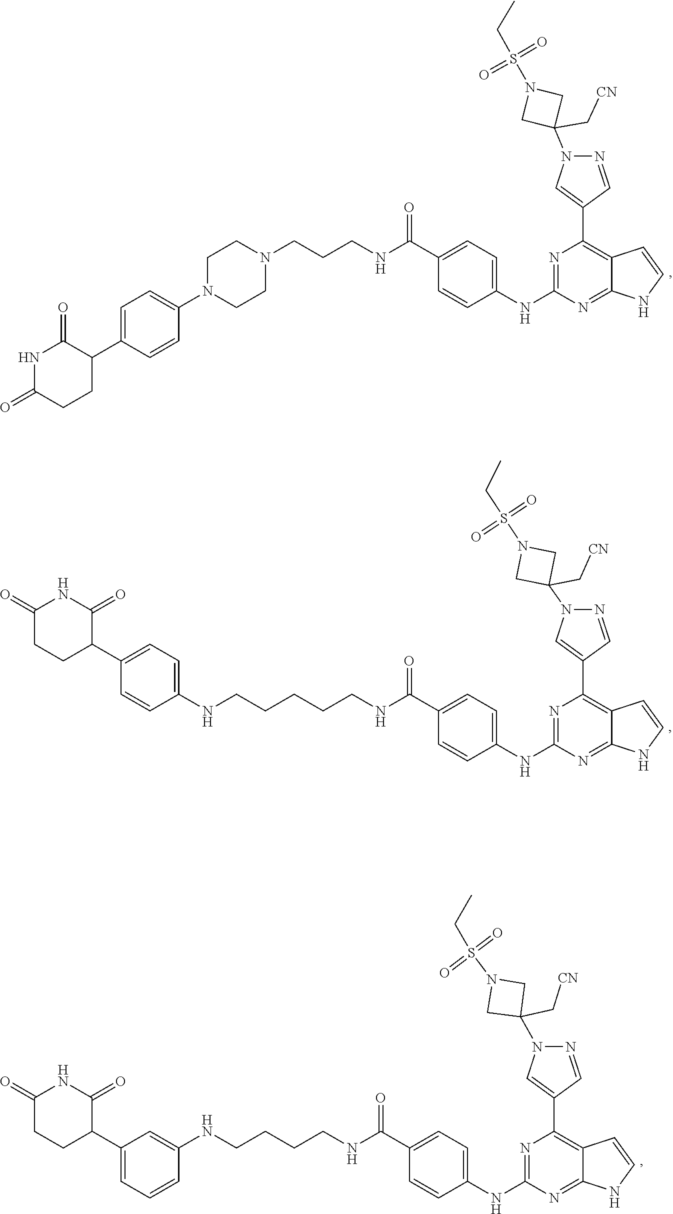 [SMILES: CCS(=O)(=O)N1CC(CC#N)(n2cc(-c3nc(Nc4ccc(C(=O)NCCCN5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NCCCCCNc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCNc5cccc(C6CCC(=O)NC6=O)c5)cc4)nc4[nH]ccc34)cn2)C1]

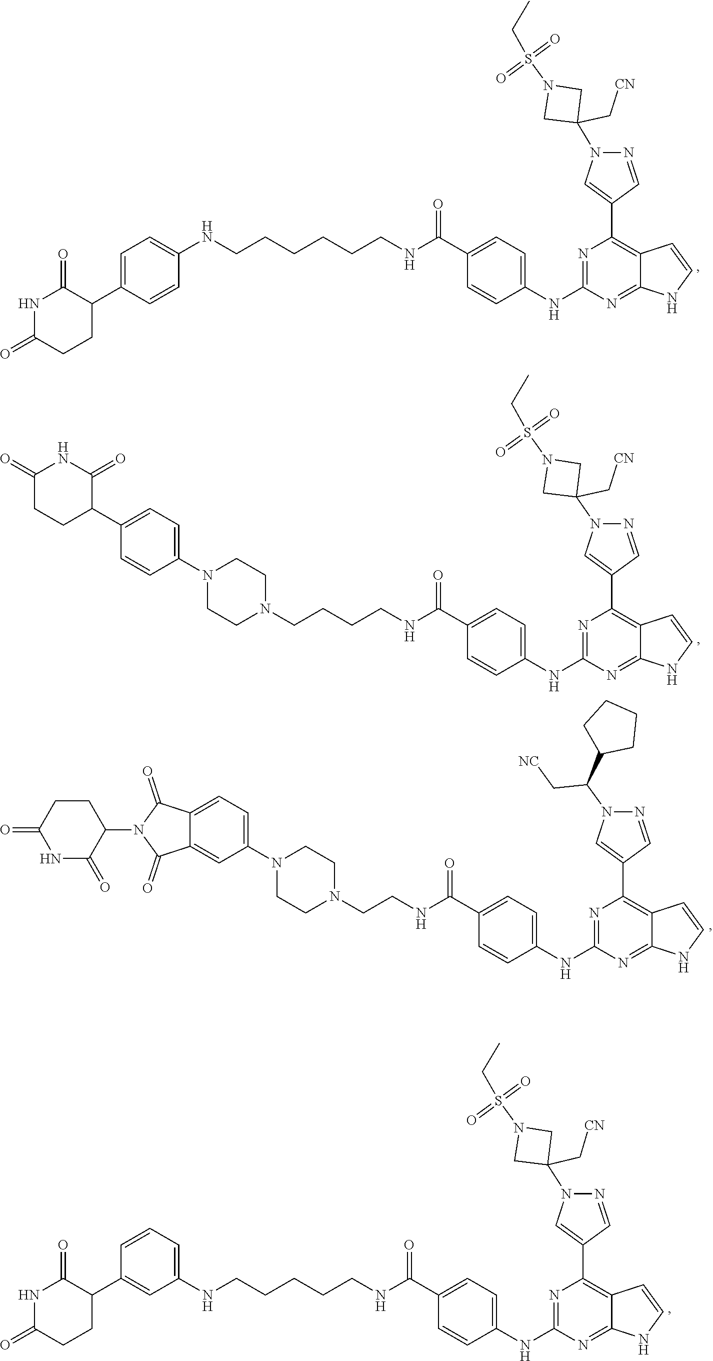

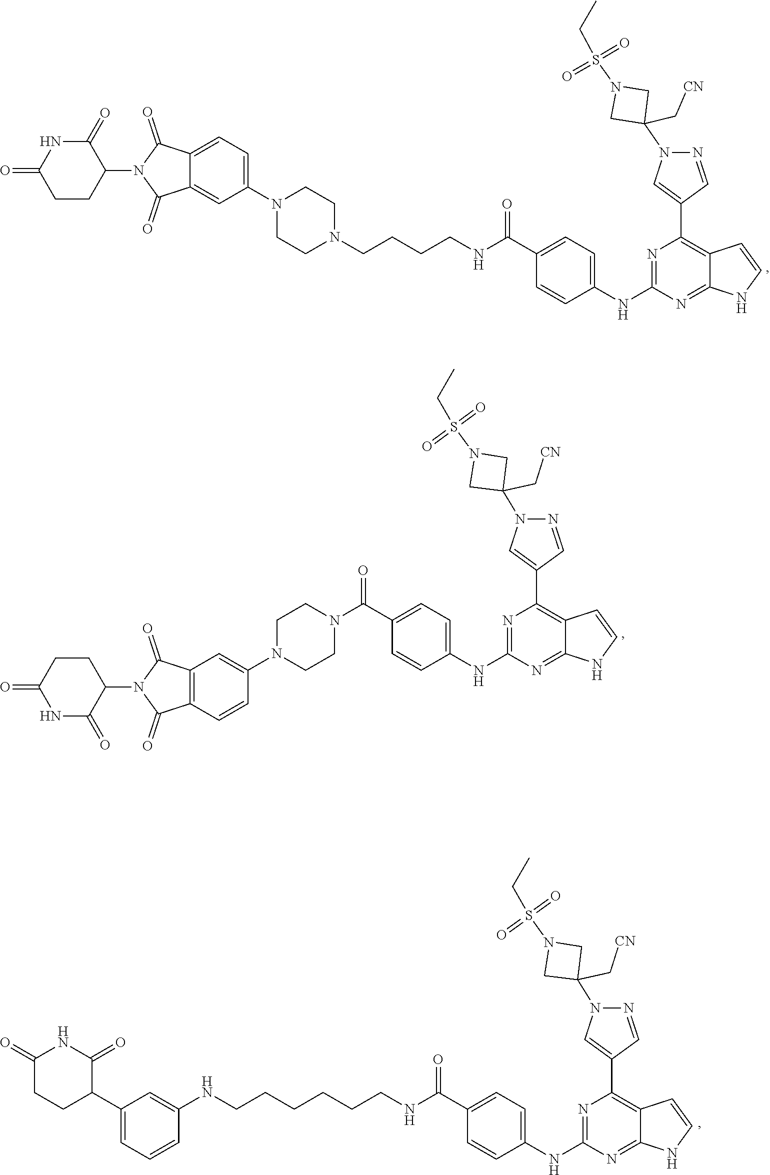 [SMILES: CCS(=O)(=O)N1CC(CC#N)(n2cc(-c3nc(Nc4ccc(C(=O)NCCCCN5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)N5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCCCNc5cccc(C6CCC(=O)NC6=O)c5)cc4)nc4[nH]ccc34)cn2)C1]

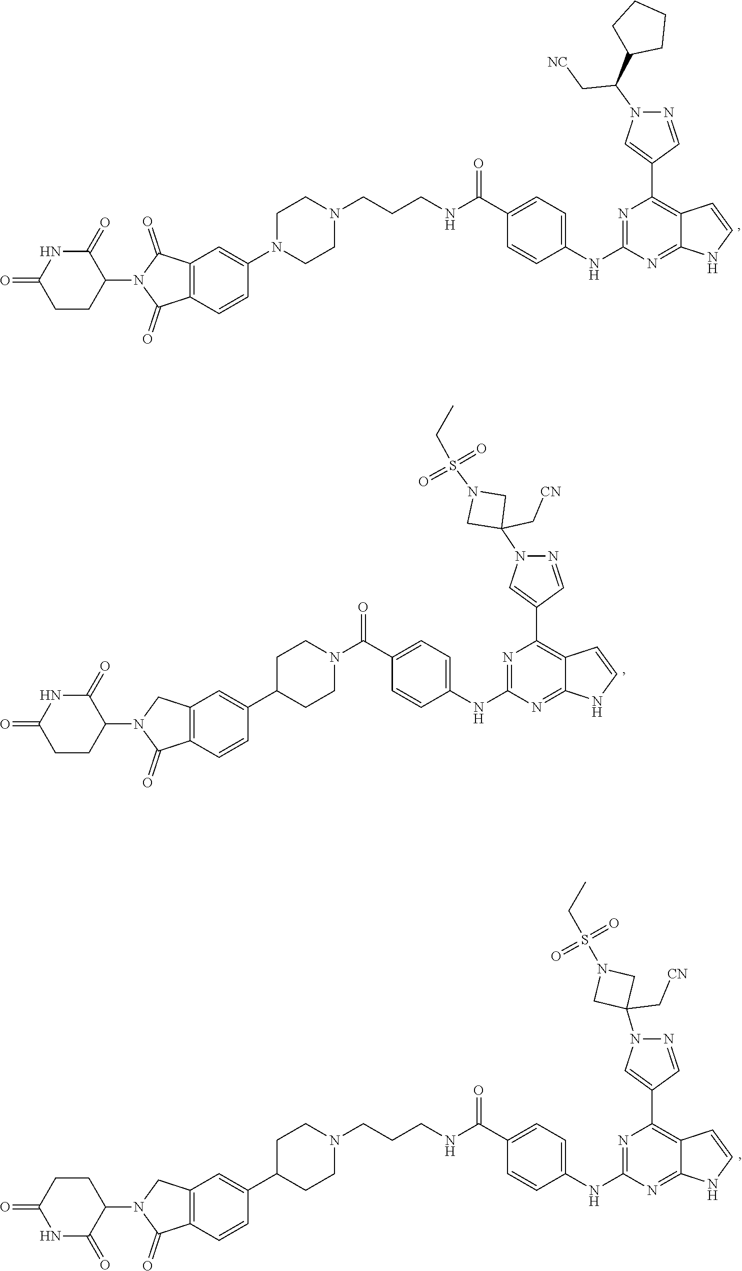 [SMILES: N#CC[C@@H](C1CCCC1)n1cc(-c2nc(Nc3ccc(C(=O)NCCCN4CCN(c5ccc6c(c5)C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)N5CCC(c6ccc7c(c6)CN(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCN5CCC(c6ccc7c(c6)CN(C6CCC(=O)NC6=O)C7=O)CC5)cc4)nc4[nH]ccc34)cn2)C1]

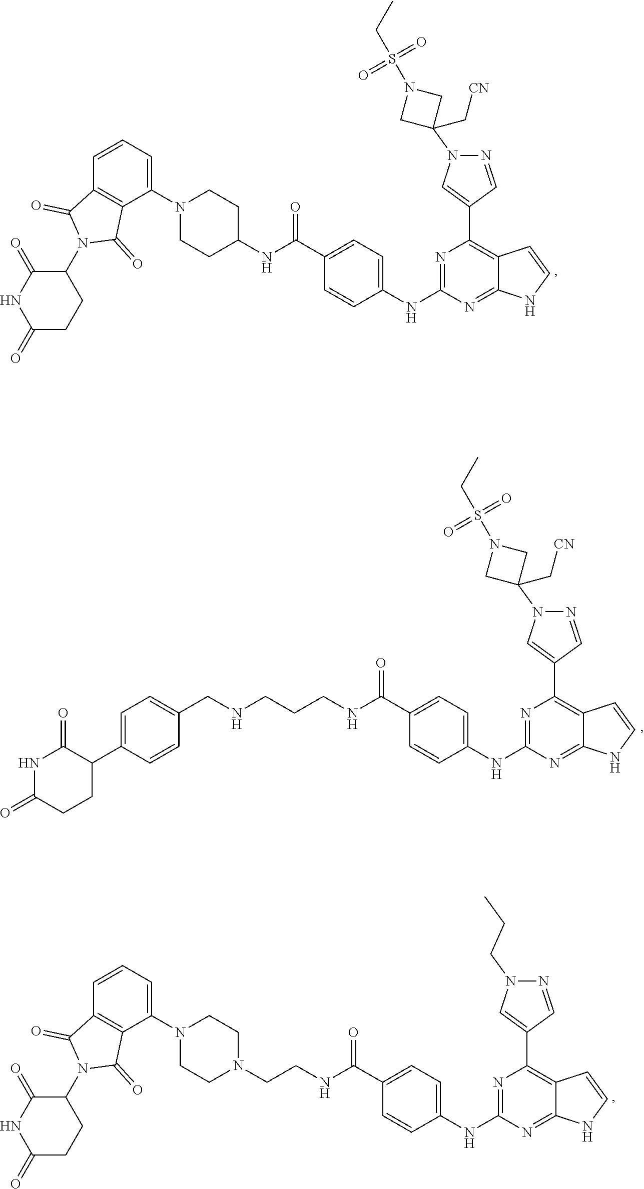 [SMILES: CCS(=O)(=O)N1CC(CC#N)(n2cc(-c3nc(Nc4ccc(C(=O)NC5CCN(c6cccc7c6C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCNCc5ccc(C6CCC(=O)NC6=O)cc5)cc4)nc4[nH]ccc34)cn2)C1 ; CCCn1cc(-c2nc(Nc3ccc(C(=O)NCCN4CCN(c5cccc6c5C(=O)N(C5CCC(=O)NC5=O)C6=O)CC4)cc3)nc3[nH]ccc23)cn1]

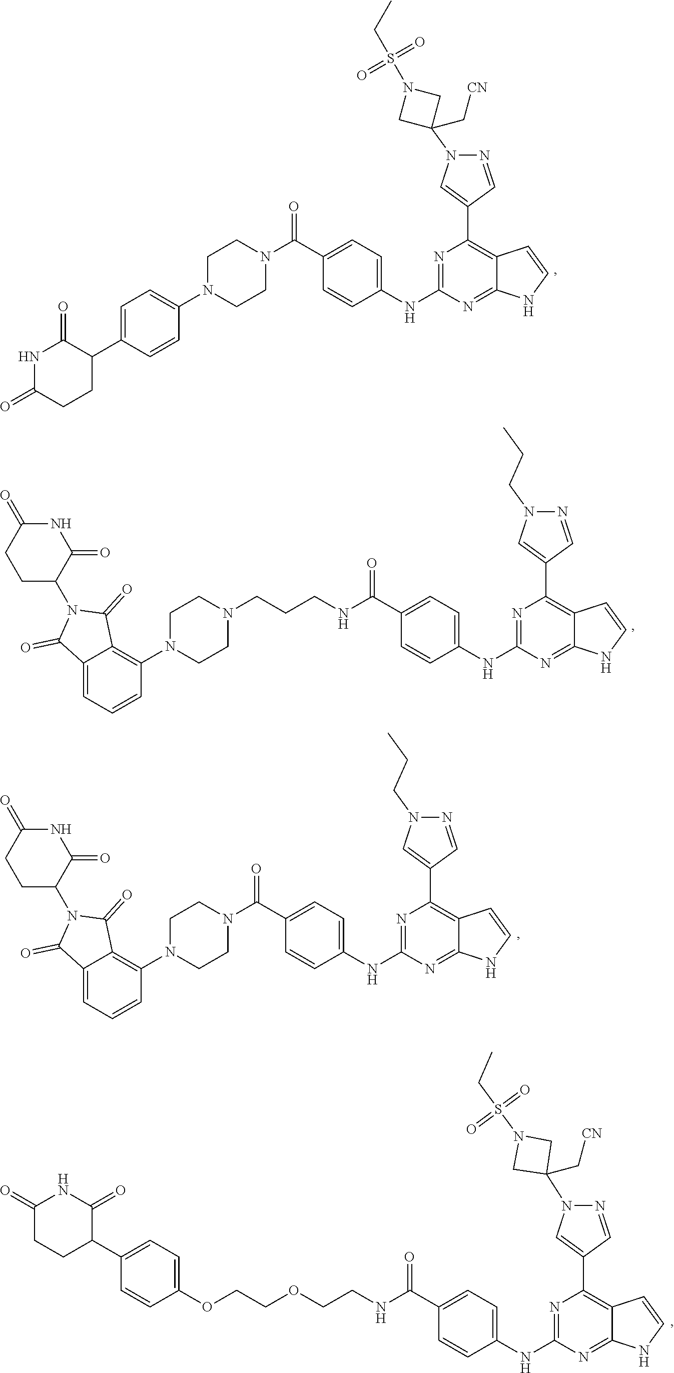 [SMILES: CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1 ; CCCn1cc(-c2nc(Nc3ccc(C(=O)NCCCN4CCN(c5cccc6c5C(=O)N(C5CCC(=O)NC5=O)C6=O)CC4)cc3)nc3[nH]ccc23)cn1 ; CCCn1cc(-c2nc(Nc3ccc(C(=O)N4CCN(c5cccc6c5C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1]

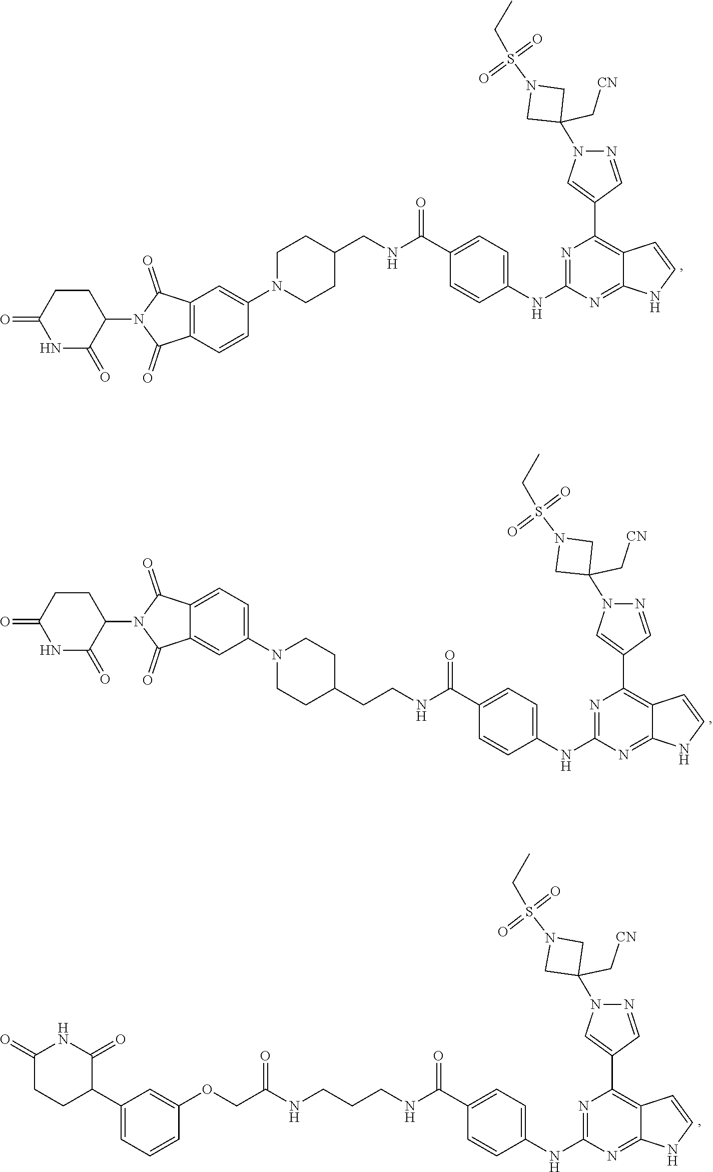 [SMILES: CCS(=O)(=O)N1CC(CC#N)(n2cc(-c3nc(Nc4ccc(C(=O)NCC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCNC(=O)COc5cccc(C6CCC(=O)NC6=O)c5)cc4)nc4[nH]ccc34)cn2)C1]

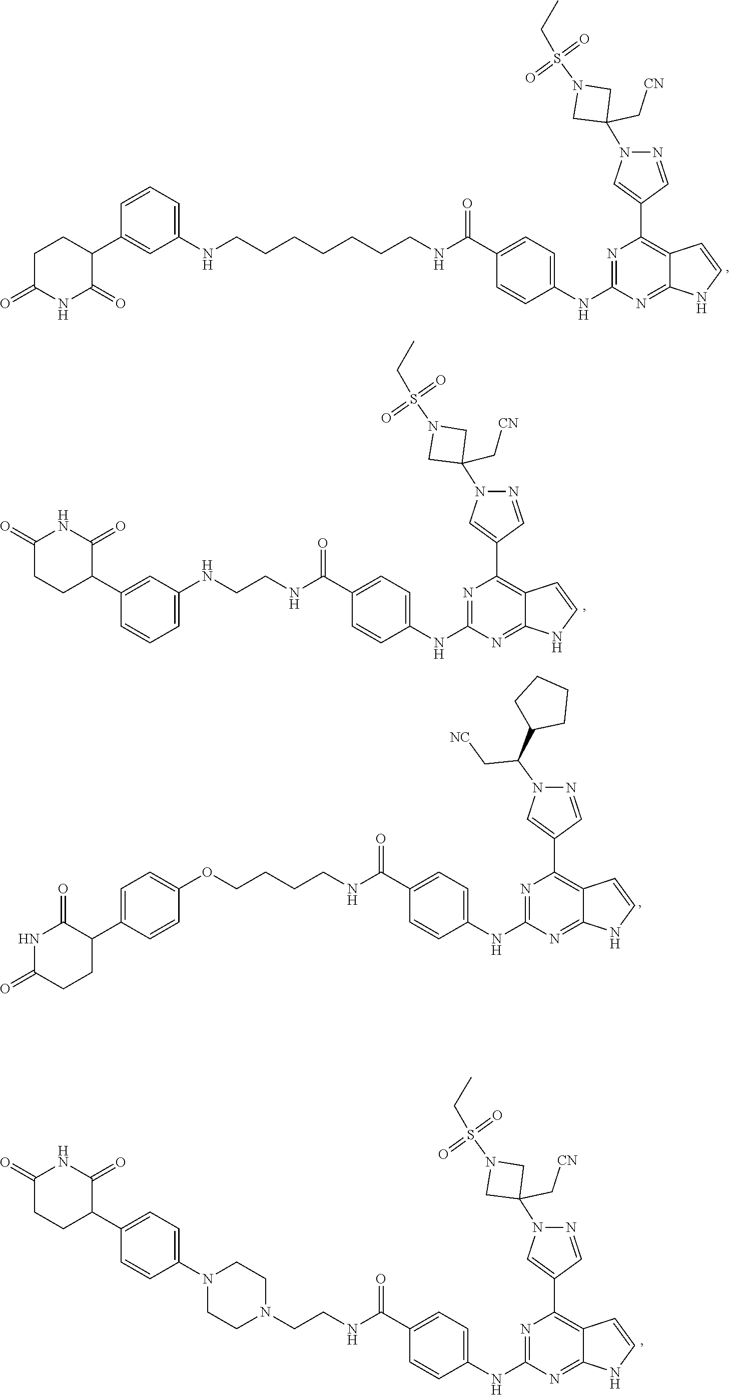

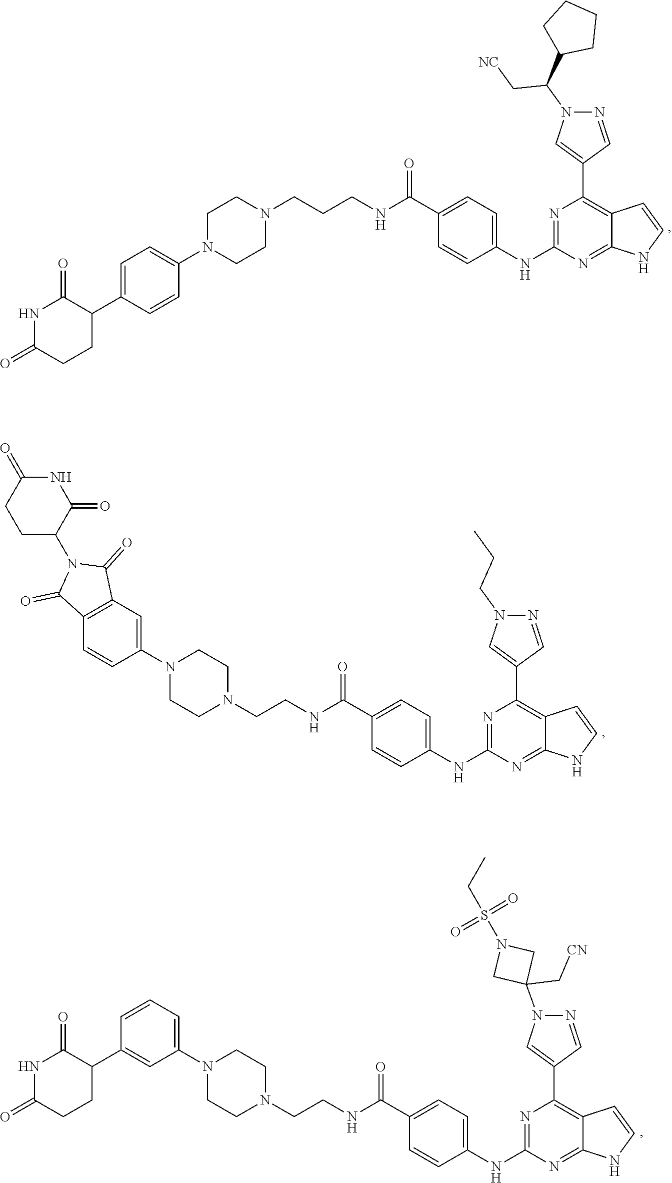 [SMILES: N#CC[C@@H](C1CCCC1)n1cc(-c2nc(Nc3ccc(C(=O)NCCCN4CCN(c5ccc(C6CCC(=O)NC6=O)cc5)CC4)cc3)nc3[nH]ccc23)cn1 ; CCCn1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCN5CCN(c6cccc(C7CCC(=O)NC7=O)c6)CC5)cc4)nc4[nH]ccc34)cn2)C1]

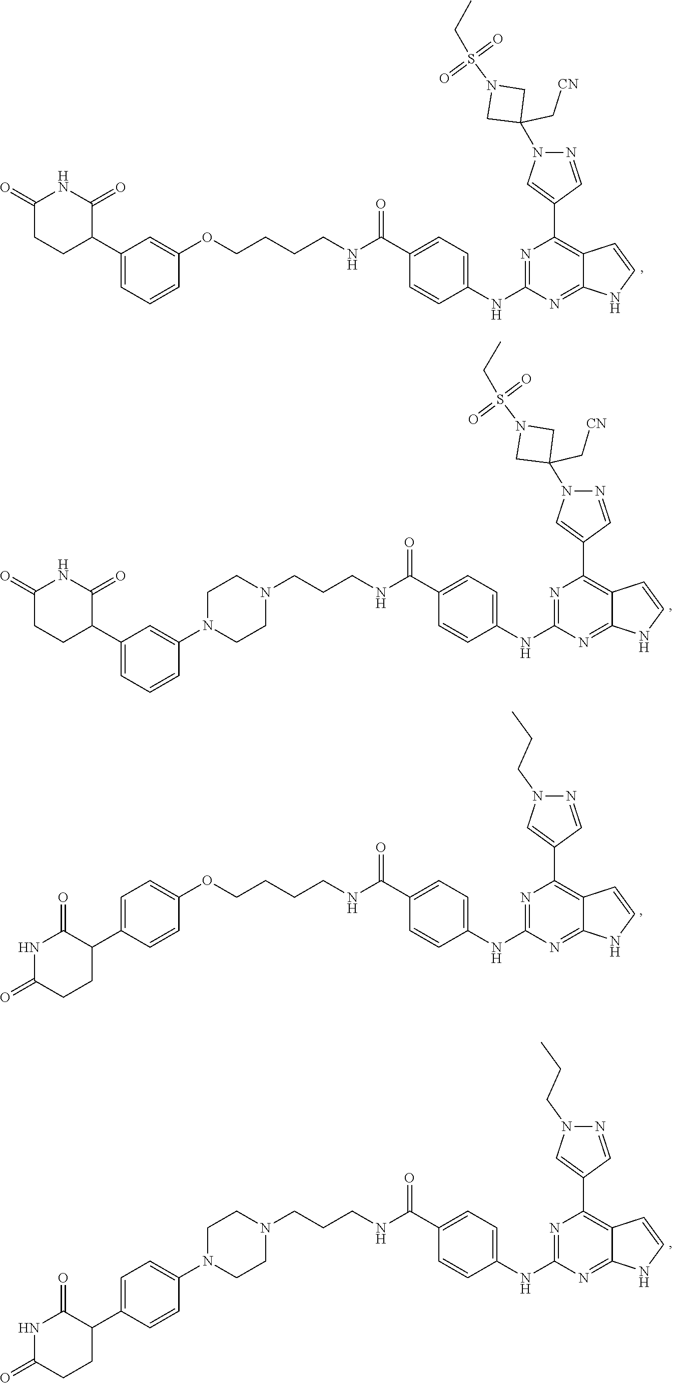

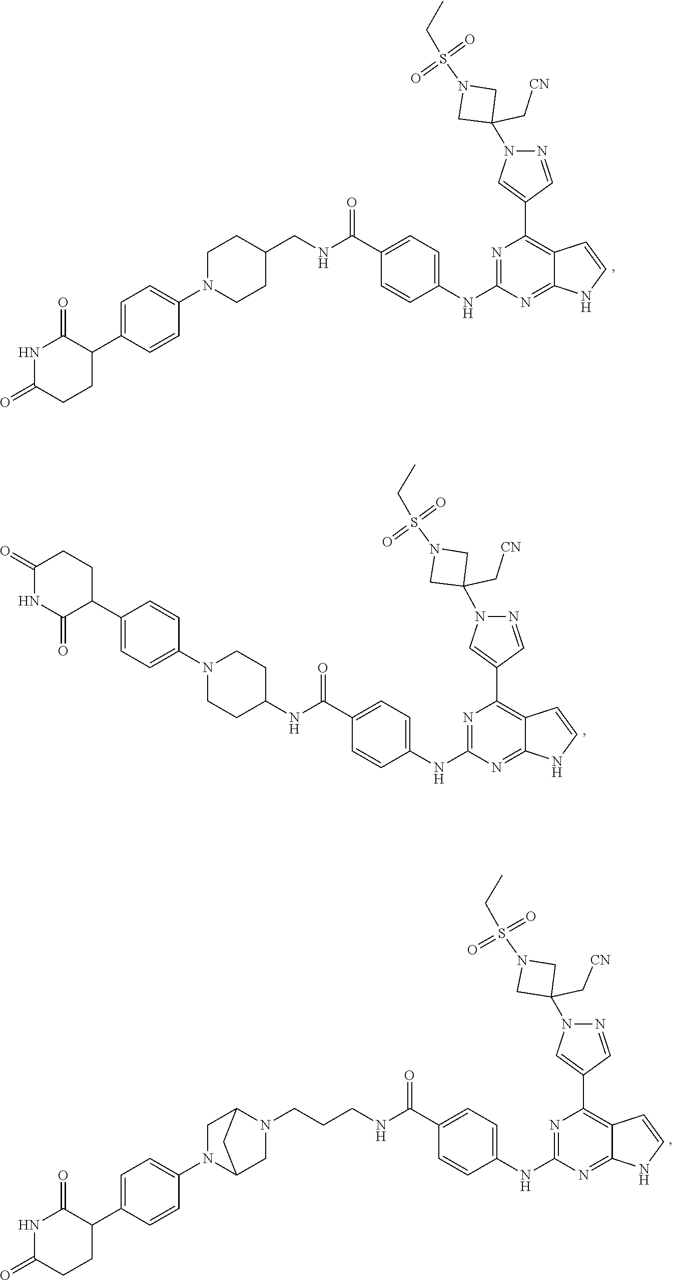 [SMILES: CCS(=O)(=O)N1CC(CC#N)(n2cc(-c3nc(Nc4ccc(C(=O)NCC5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NC5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NCCCN5CC6CC5CN6c5ccc(C6CCC(=O)NC6=O)cc5)cc4)nc4[nH]ccc34)cn2)C1]

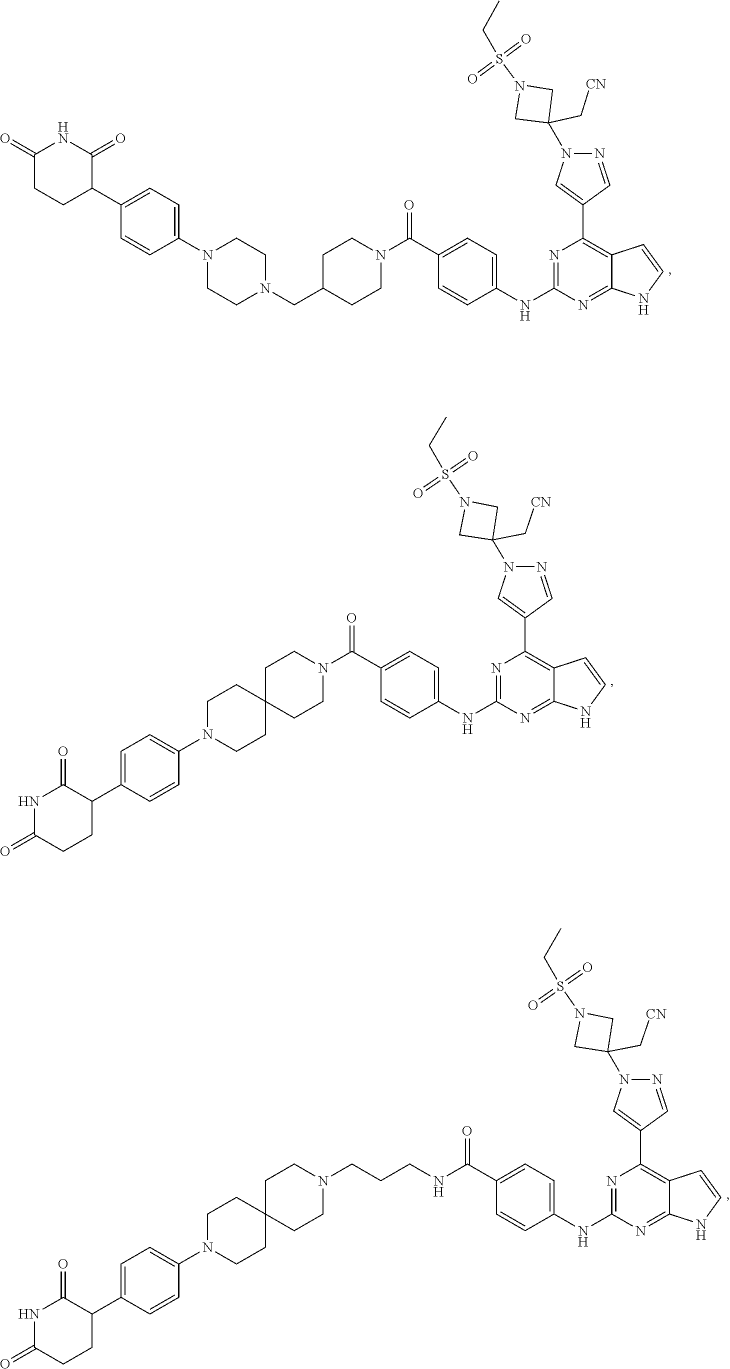 [SMILES: CCS(=O)(=O)N1CC(CC#N)(n2cc(-c3nc(Nc4ccc(C(=O)N5CCC(CN6CCN(c7ccc(C8CCC(=O)NC8=O)cc7)CC6)CC5)cc4)nc4[nH]ccc34)cn2)C1 ; CCS(=O)(=O)N1CC(CC#N)(n2cc(-c3nc(Nc4ccc(C(=O)N5CCC6(CC5)CCN(c5ccc(C7CCC(=O)NC7=O)cc5)CC6)cc4)nc4[nH]ccc34)cn2)C1 ; CCS(=O)(=O)N1CC(CC#N)(n2cc(-c3nc(Nc4ccc(C(=O)NCCCN5CCC6(CC5)CCN(c5ccc(C7CCC(=O)NC7=O)cc5)CC6)cc4)nc4[nH]ccc34)cn2)C1]

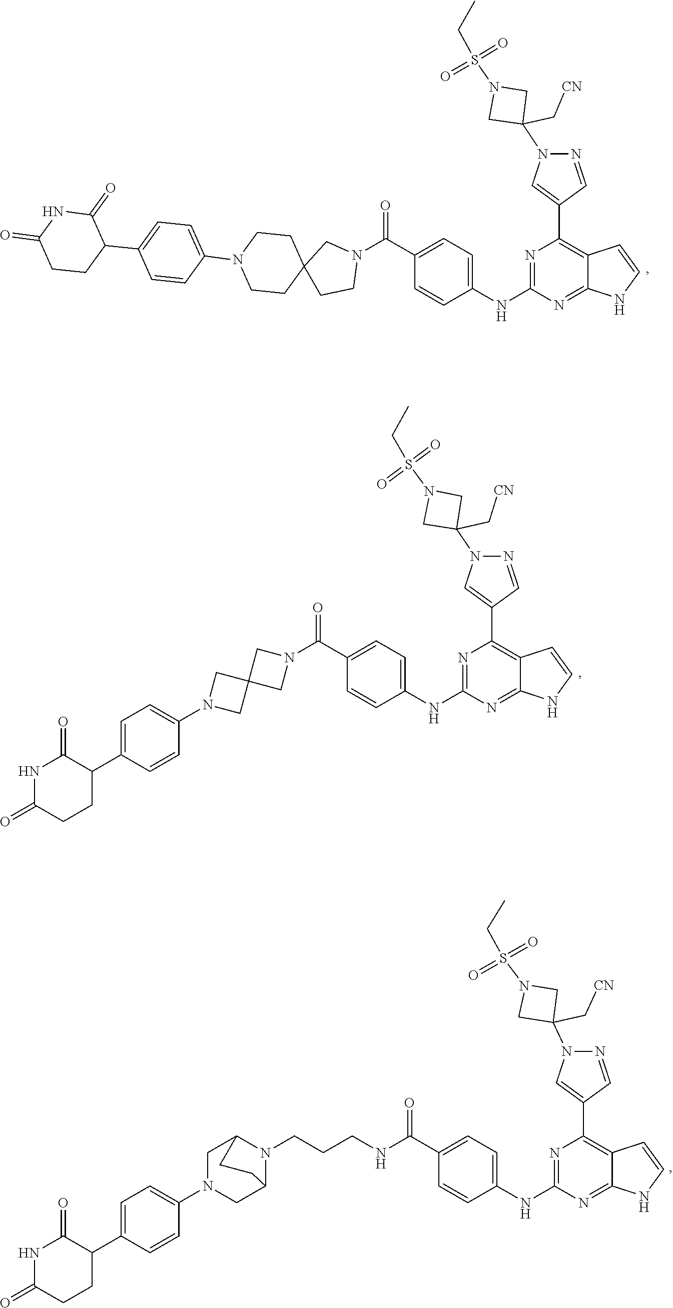 [SMILES: CCS(=O)(=O)N1CC(CC#N)(n2cc(-c3nc(Nc4ccc(C(=O)N5CCC6(CCN(c7ccc(C8CCC(=O)NC8=O)cc7)CC6)C5)cc4)nc4[nH]ccc34)cn2)C1 ; CCS(=O)(=O)N1CC(CC#N)(n2cc(-c3nc(Nc4ccc(C(=O)N5CC6(C5)CN(c5ccc(C7CCC(=O)NC7=O)cc5)C6)cc4)nc4[nH]ccc34)cn2)C1 ; CCS(=O)(=O)N1CC(CC#N)(n2cc(-c3nc(Nc4ccc(C(=O)NCCCN5C6CCC5CN(c5ccc(C7CCC(=O)NC7=O)cc5)C6)cc4)nc4[nH]ccc34)cn2)C1]

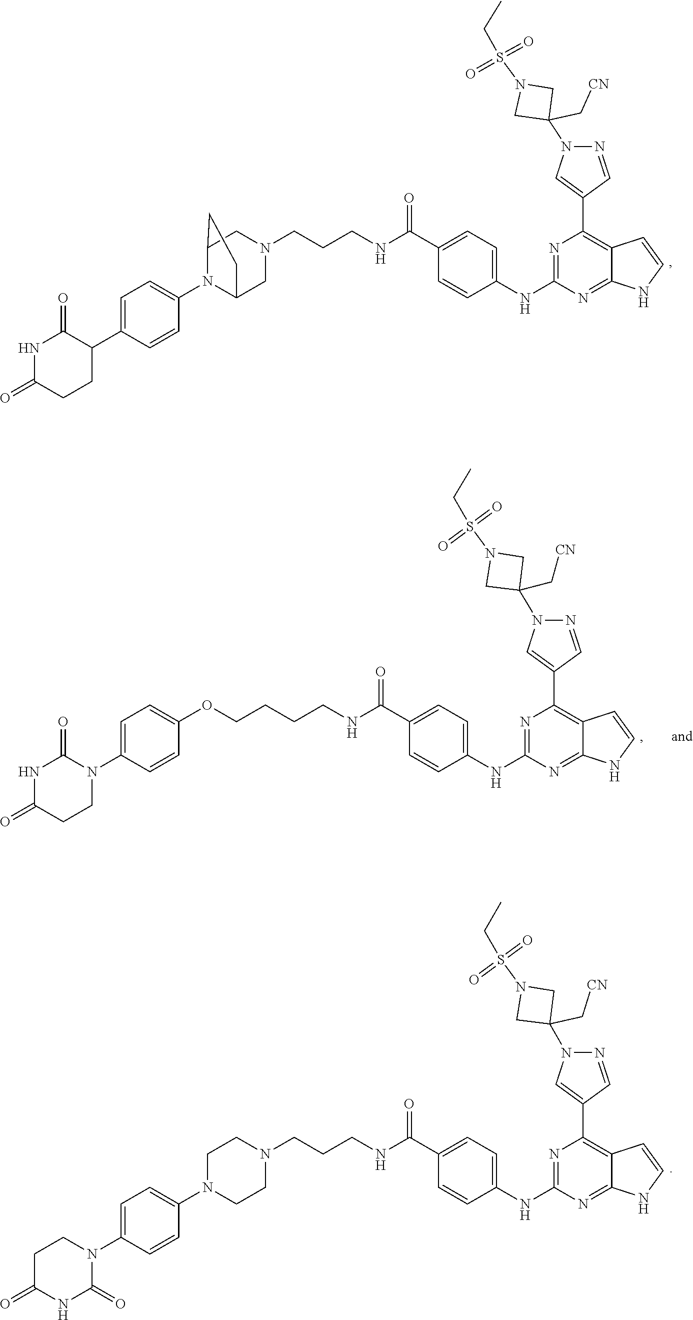 [SMILES: CCS(=O)(=O)N1CC(CC#N)(n2cc(-c3nc(Nc4ccc(C(=O)NCCCN5CC6CCC(C5)N6c5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCOc5ccc(N6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(N7CCC(=O)NC7=O)cc6)CC5)cc4)nc4[nH]ccc34)cn2)C1]

or a pharmaceutically acceptable salt thereof.

In a further aspect, modifying is decreasing. In a still further aspect, modifying is inhibiting.

In a further aspect, the subject has been diagnosed with a disorder of uncontrolled cellular proliferation prior to the administering step.

In a further aspect, the subject has been diagnosed with a need for modifying cellular proliferation prior to the administering step.

In a further aspect, the method further comprises the step of identifying a subject in need of treatment of a disorder associated with cellular proliferation dysfunction.

In a further aspect, the subject has been diagnosed with a need for treatment of a disorder associated with cellular activity prior to the administering step. In a still further aspect, the disorder associated with cellular proliferation activity is cancer, for example a cancer selected from for example a cancer selected from sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and in another example acute lymphoblastic leukemia.

In a further aspect, the subject has been diagnosed with a need for modulating cellular proliferation activity prior to the administering step.

### G. Methods of Modulating Cellular Proliferation Activity in a Cell

In one aspect, disclosed are methods of cellular proliferation activity in a cell, the method comprising the step of contacting the cell with an effective amount of at least one disclosed compound, or a pharmaceutically acceptable salt thereof. In a further aspect, modulating is inhibiting.

Thus, in one aspect, disclosed are methods of modulating cellular proliferation activity in a cell, the method comprising the step of contacting the cell with an effective amount of a compound having a structure represented by a formula:

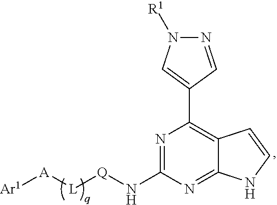

wherein q is selected from 0 and 1; wherein A is selected from —O—, —S—, —NH—, —NHCH₂—, —OCH₂C(O)NH—, —CH₂—, and a structure selected from:

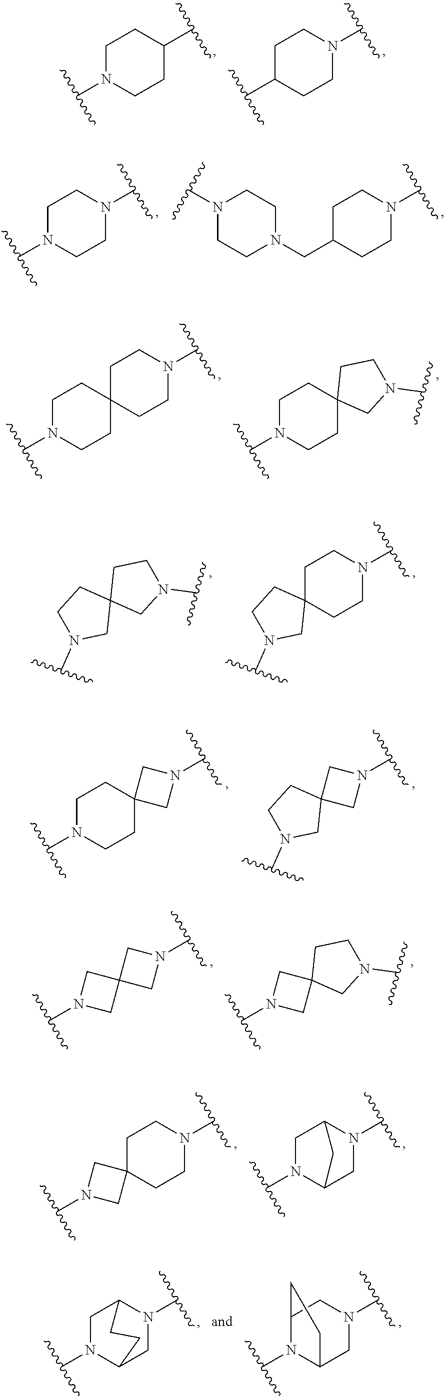

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

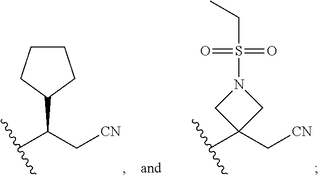 [SMILES: *[C@H](CC#N)C1CCCC1 ; *C1(CC#N)CN(S(=O)(=O)CC)C1]

and
wherein Ar¹ is a structure selected from:

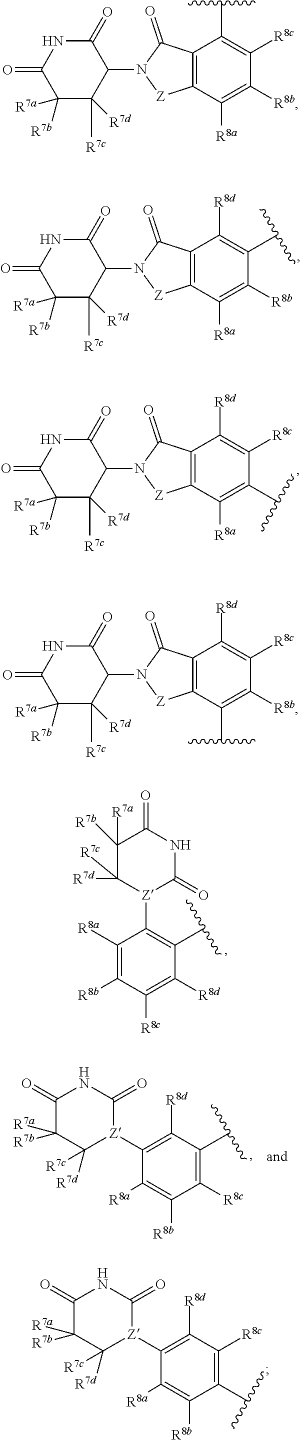

wherein Z is selected from —CH₂— and C(O); wherein Z′ is selected from —CH— and —N—; wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

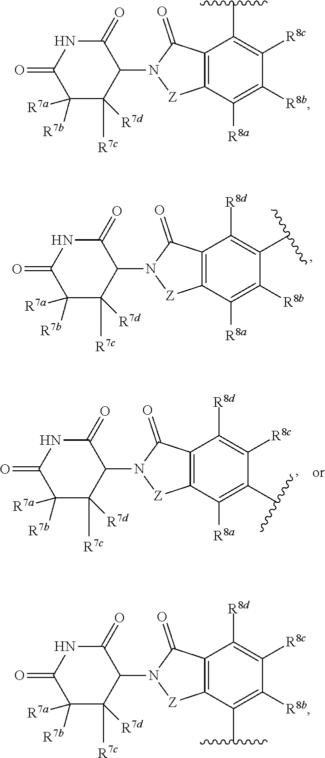

and q is 1, then A is a structure selected from:

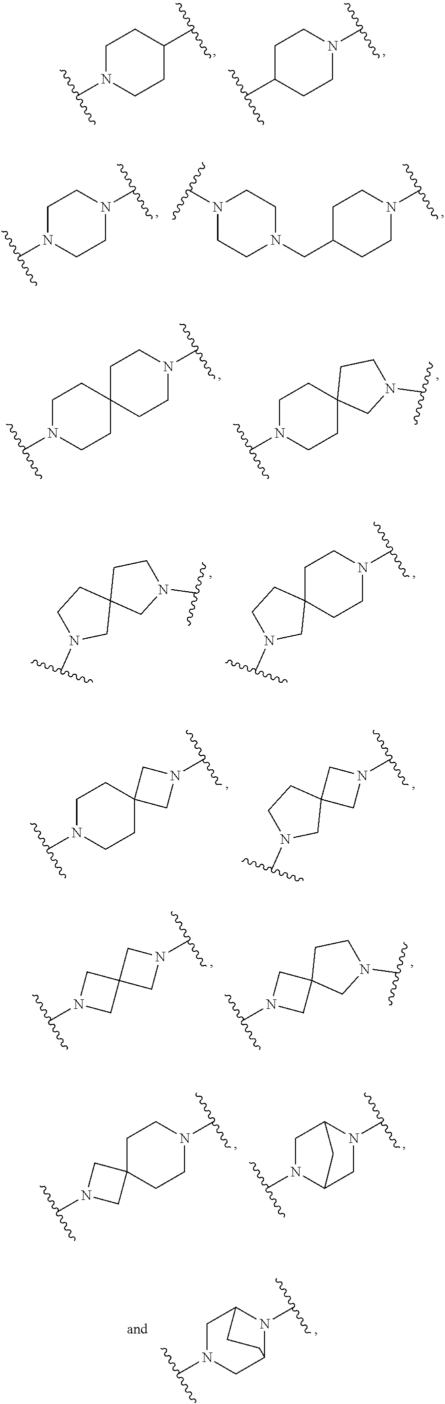

or a pharmaceutically acceptable salt thereof.

In one aspect, disclosed are methods of modulating cellular proliferation activity in a cell, the method comprising the step of contacting the cell with an effective amount of a compound having a structure represented by a formula:

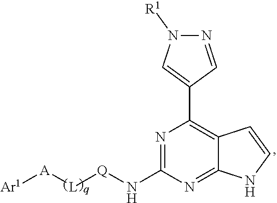

wherein q is selected from 0 and 1; wherein A is selected from —O—, —S—, —NH—, —NHCH₂—, —OCH₂C(O)NH—, —CH₂—, and a structure selected from:

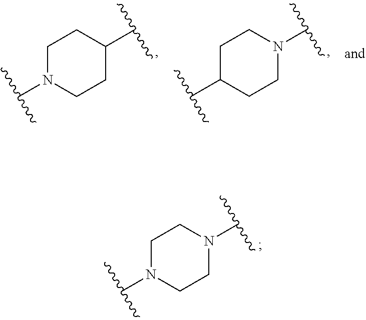

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

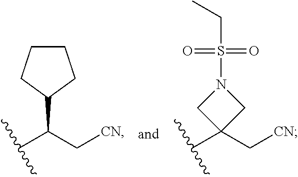 [SMILES: *[C@H](CC#N)C1CCCC1 ; *C1(CC#N)CN(S(=O)(=O)CC)C1]

and
wherein Ar¹ is a structure selected from:

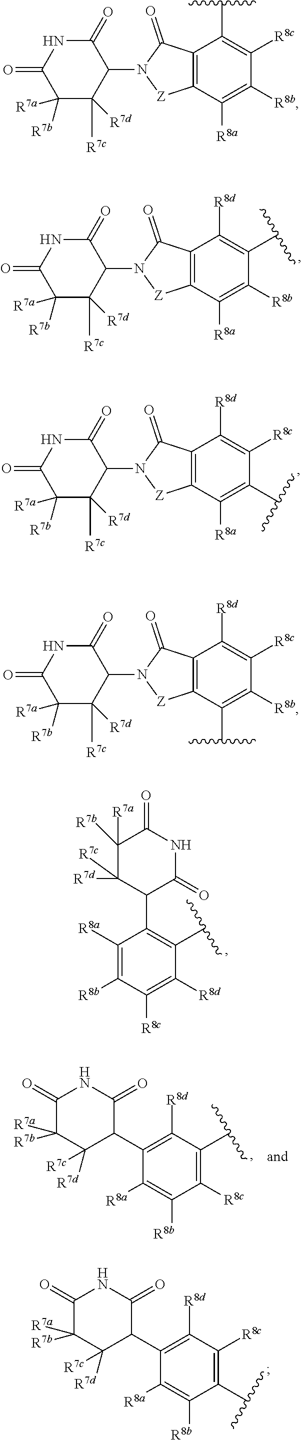

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

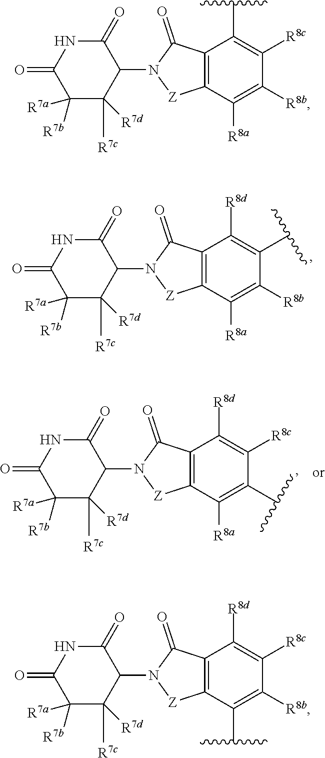

and q is 1, then A is a structure selected from:

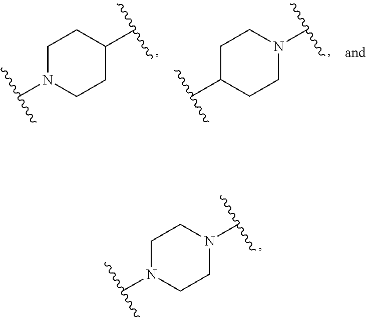 [SMILES: *C1CCN(*)CC1 ; *C1CCN(*)CC1 ; *N1CCN(*)CC1]

or a pharmaceutically acceptable salt thereof.

Also disclosed are disclosed are methods of treating a disorder associated with uncontrolled cellular proliferation, such as for example cancer, sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), in another example acute lymphoblastic leukemia activity in a subject, the method comprising administering to the subject an effective amount of a compound having a structure represented by a formula:

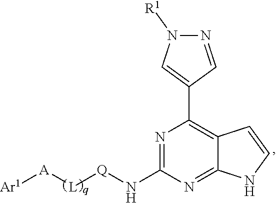

wherein q is selected from 0 and 1; wherein A is selected from —O—, —S—, —NH—, —NHCH₂—, —CH₂—, and a structure selected from:

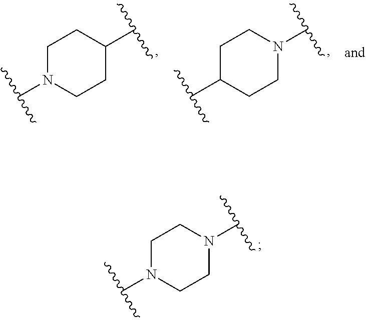

wherein L is selected from C2-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

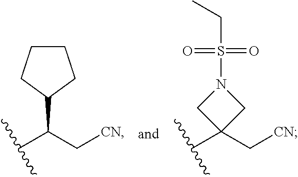 [SMILES: *[C@H](CC#N)C1CCCC1 ; *C1(CC#N)CN(S(=O)(=O)CC)C1]

and
wherein Ar¹ is a structure selected from:

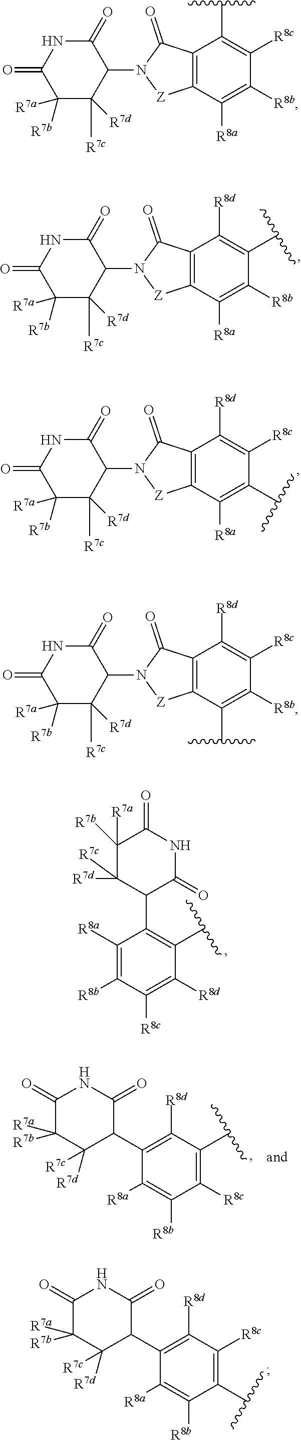

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

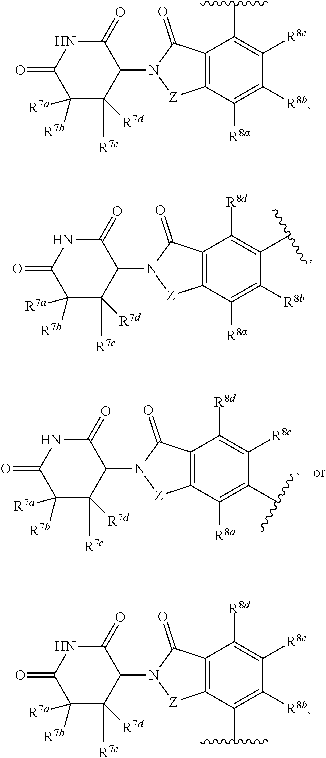

and q is 1, then A is a structure selected from:

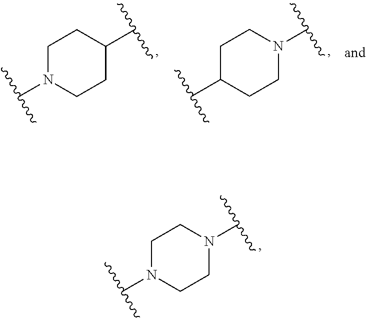 [SMILES: *C1CCN(*)CC1 ; *C1CCN(*)CC1 ; *N1CCN(*)CC1]

or a pharmaceutically acceptable salt thereof.

In one aspect, disclosed are methods of cellular proliferation activity in a cell, the method comprising the step of contacting the cell with an effective amount of at least one compound having a structure selected from:

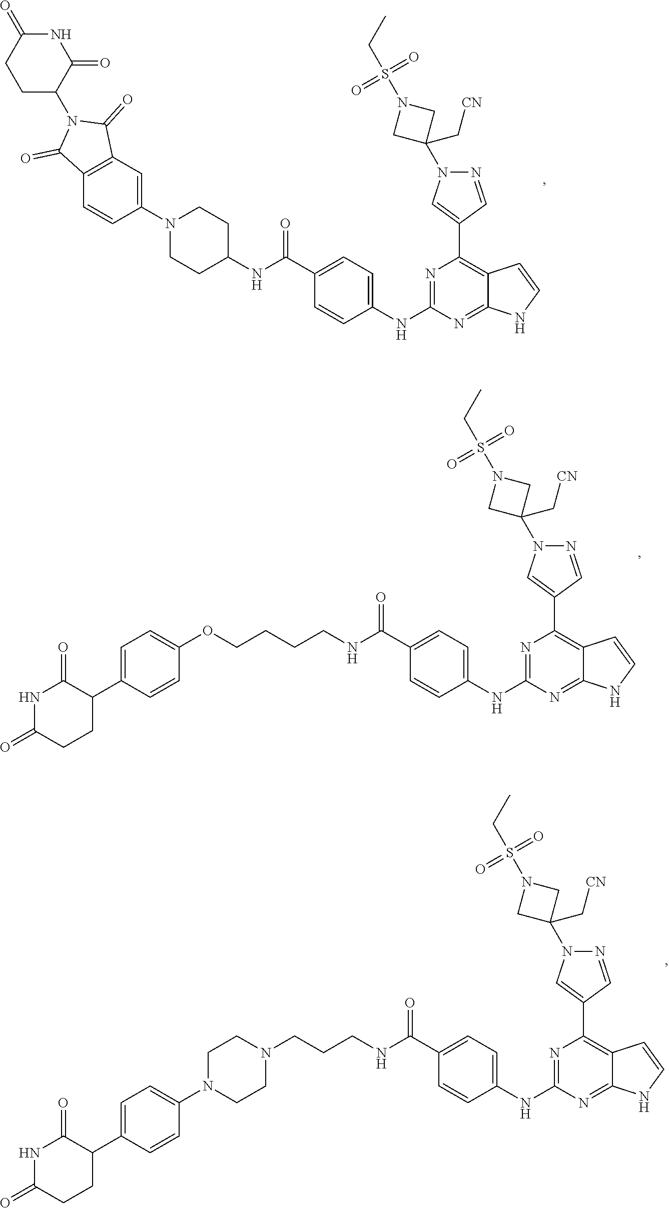 [SMILES: CCS(=O)(=O)N1CC(CC#N)(n2cc(-c3nc(Nc4ccc(C(=O)NC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCOc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(C7CCC(=O)NC7=O)cc6)CC5)cc4)nc4[nH]ccc34)cn2)C1]

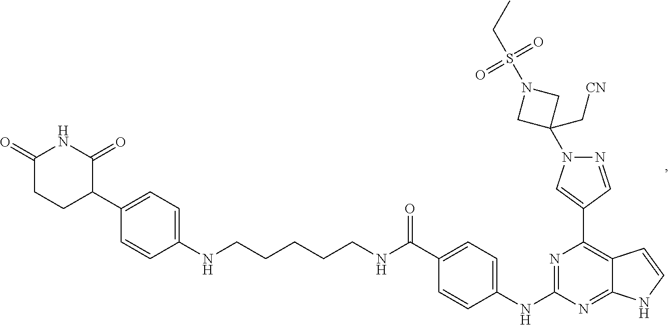 [SMILES: CCS(=O)(=O)N1CC(CC#N)(n2cc(-c3nc(Nc4ccc(C(=O)NCCCCCNc5ccc(C6CCC(=O)NC6=O)cc5)cc4)nc4[nH]ccc34)cn2)C1]

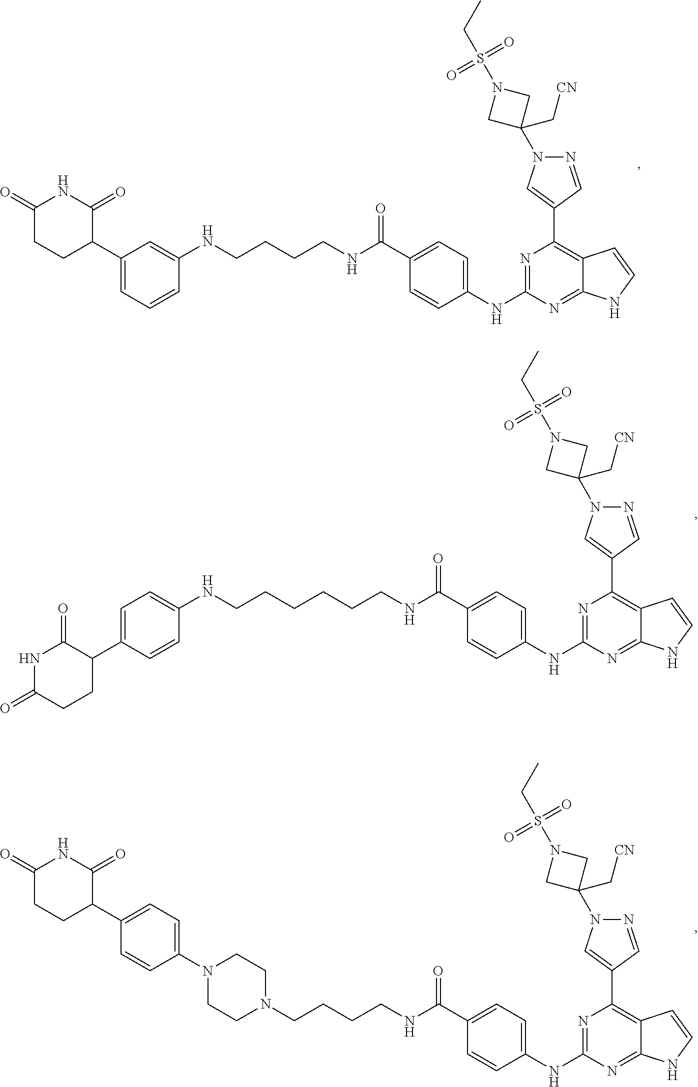

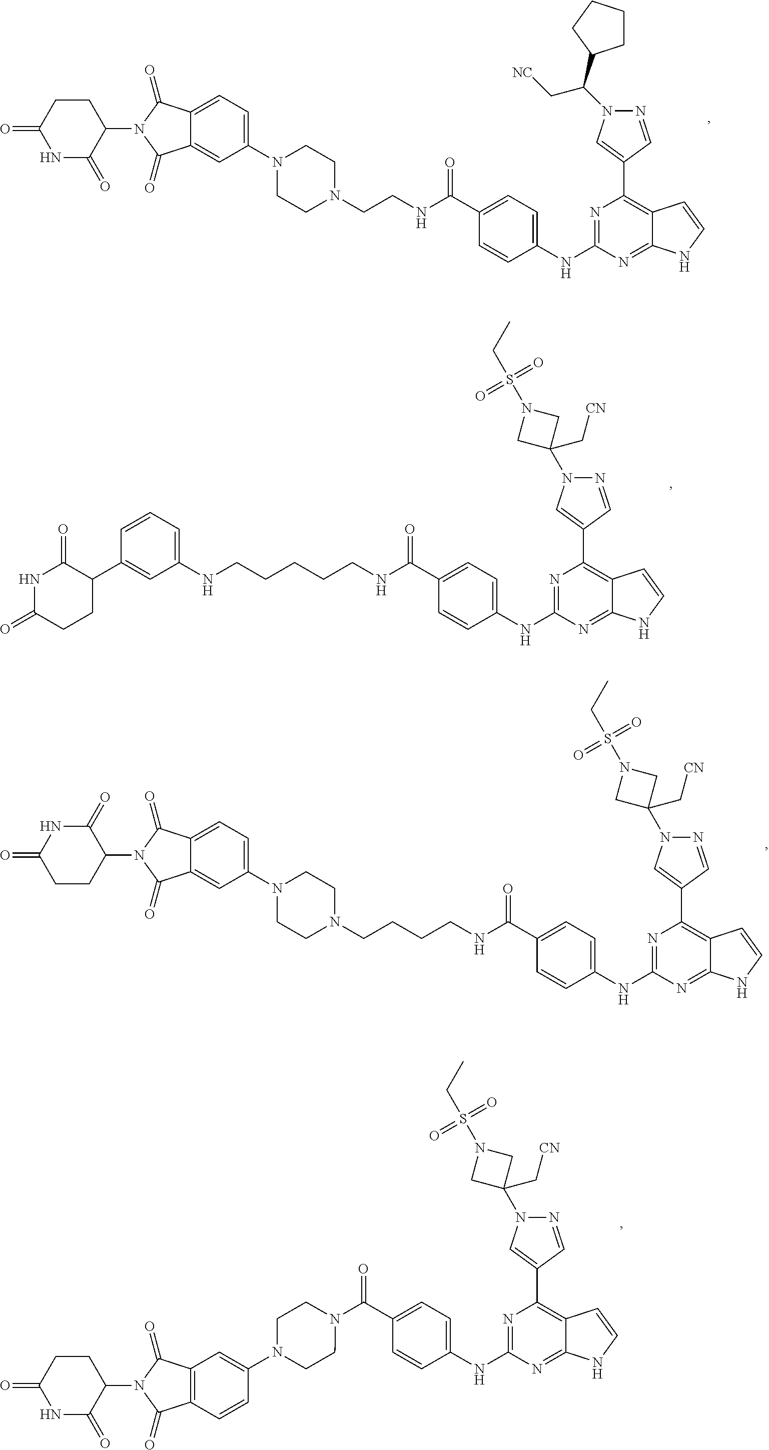 [SMILES: N#CC[C@@H](C1CCCC1)n1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCCCCNc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCCN5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)N5CCN(c6ccc7c(c6)C(=O)N(C6CCC(=O)NC6=O)C7=O)CC5)cc4)nc4[nH]ccc34)cn2)C1]

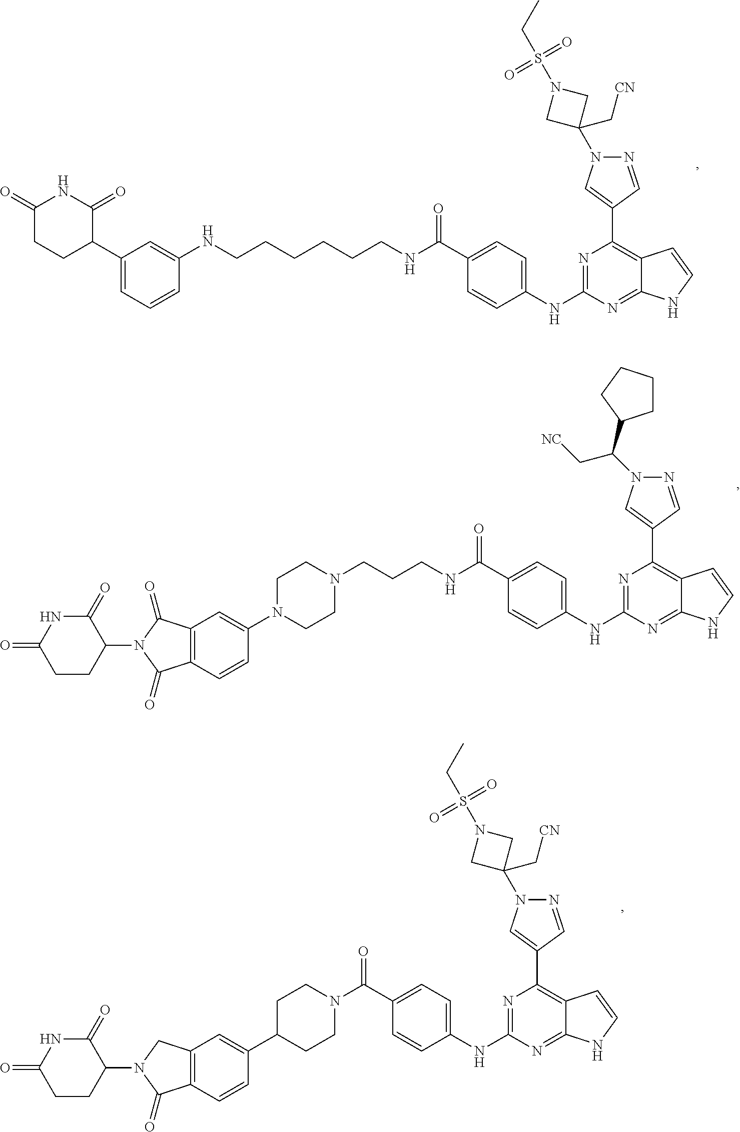 [SMILES: CCS(=O)(=O)N1CC(CC#N)(n2cc(-c3nc(Nc4ccc(C(=O)NCCCCCCNc5cccc(C6CCC(=O)NC6=O)c5)cc4)nc4[nH]ccc34)cn2)C1 ; N#CC[C@@H](C1CCCC1)n1cc(-c2nc(Nc3ccc(C(=O)NCCCN4CCN(c5ccc6c(c5)C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)N5CCC(c6ccc7c(c6)CN(C6CCC(=O)NC6=O)C7=O)CC5)cc4)nc4[nH]ccc34)cn2)C1]

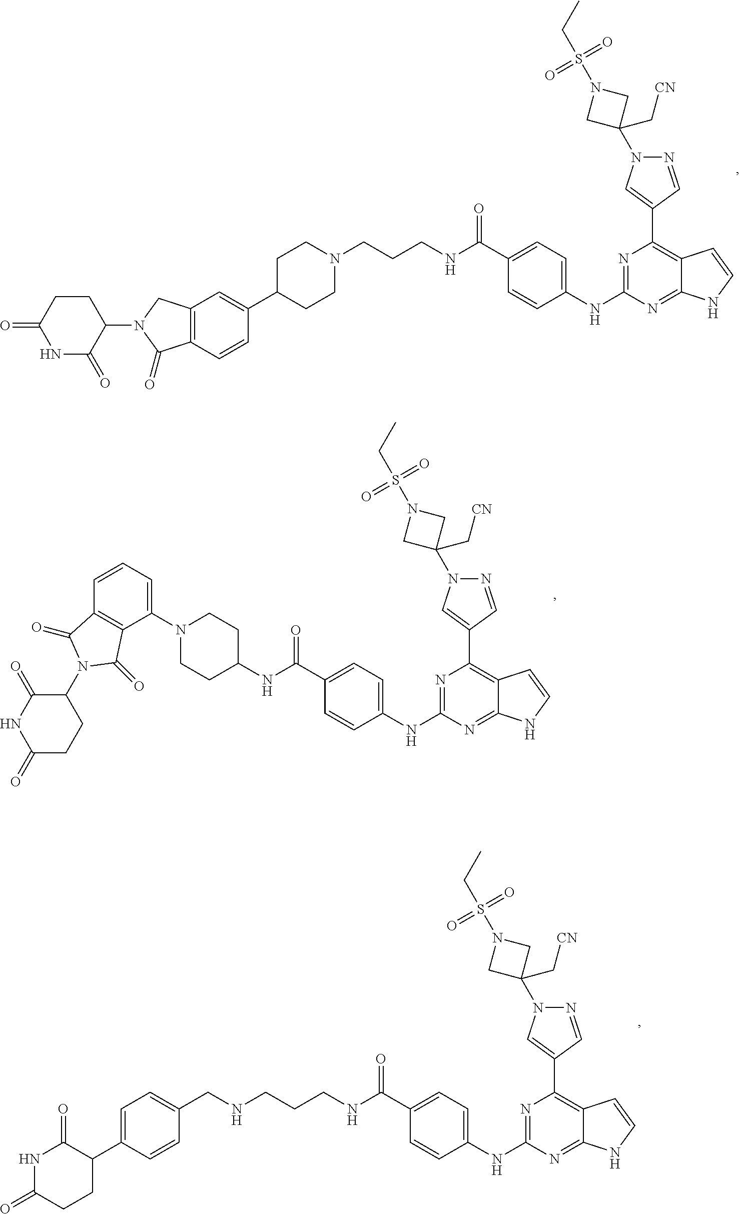 [SMILES: CCS(=O)(=O)N1CC(CC#N)(n2cc(-c3nc(Nc4ccc(C(=O)NCCCN5CCC(c6ccc7c(c6)CN(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NC5CCN(c6cccc7c6C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCNCc5ccc(C6CCC(=O)NC6=O)cc5)cc4)nc4[nH]ccc34)cn2)C1]

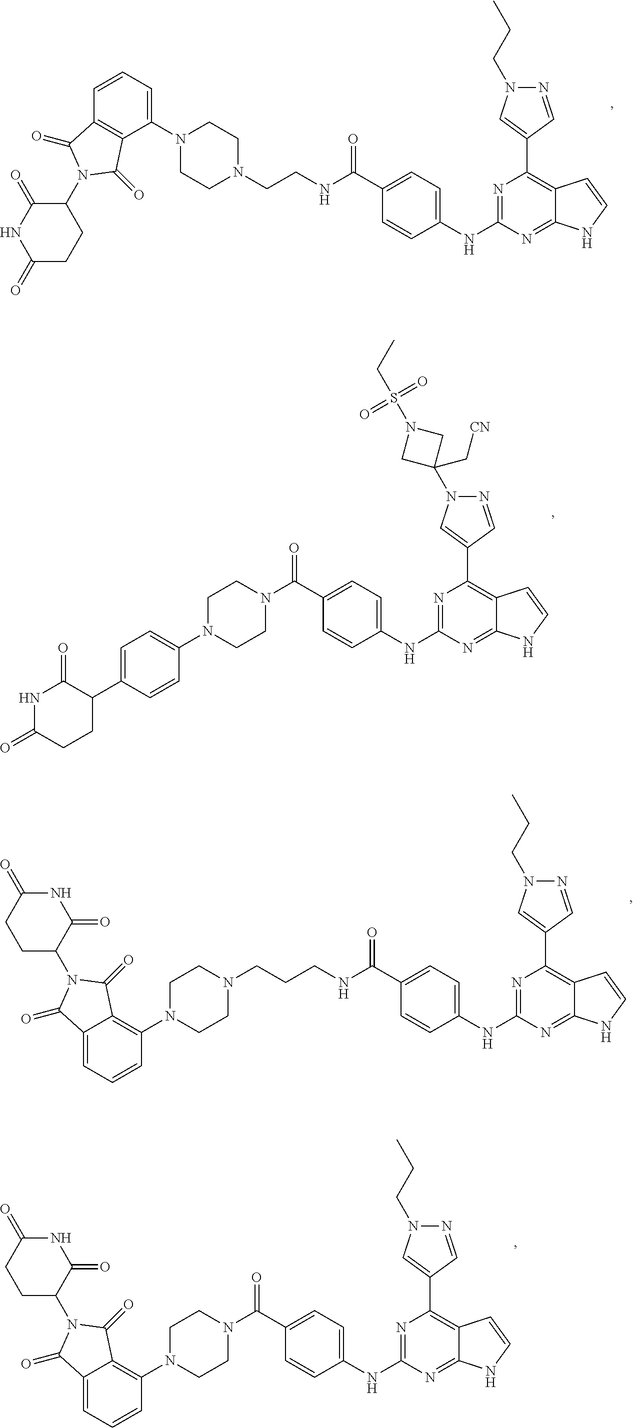 [SMILES: CCCn1cc(-c2nc(Nc3ccc(C(=O)NCCN4CCN(c5cccc6c5C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1 ; CCCn1cc(-c2nc(Nc3ccc(C(=O)NCCCN4CCN(c5cccc6c5C(=O)N(C5CCC(=O)NC5=O)C6=O)CC4)cc3)nc3[nH]ccc23)cn1 ; CCCn1cc(-c2nc(Nc3ccc(C(=O)N4CCN(c5cccc6c5C(=O)N(C5CCC(=O)NC5=O)C6=O)CC4)cc3)nc3[nH]ccc23)cn1]

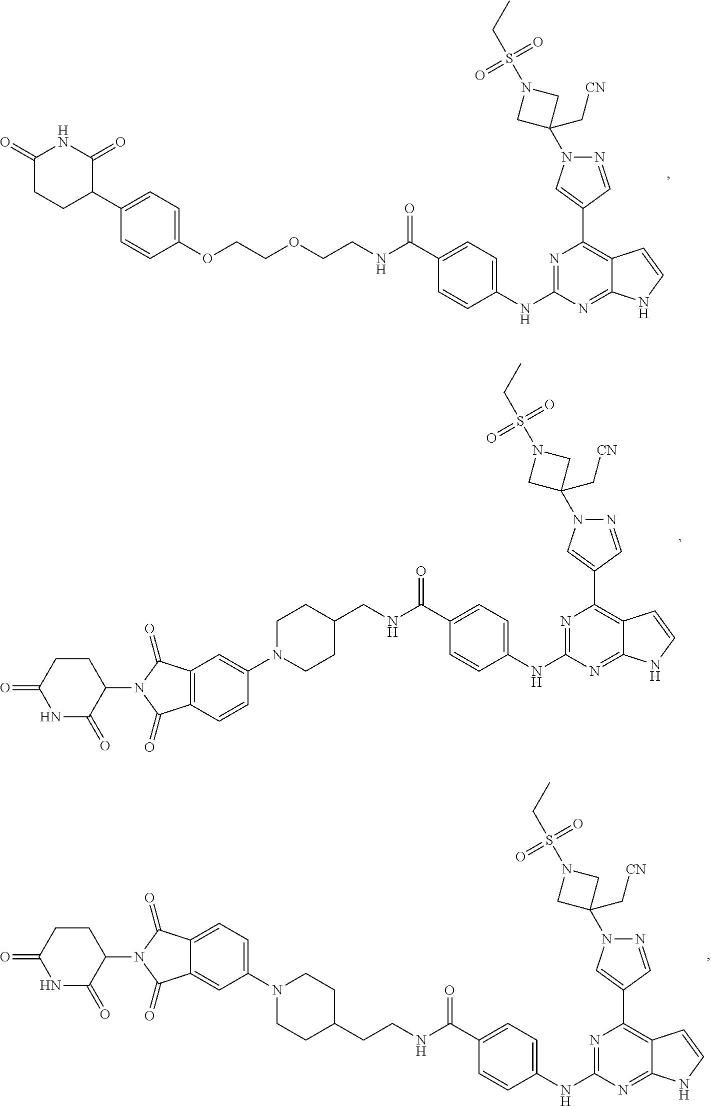 [SMILES: CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCC5CCN(c6ccc7c(c6)C(=O)N(C6CCC(=O)NC6=O)C7=O)CC5)cc4)nc4[nH]ccc34)cn2)C1]

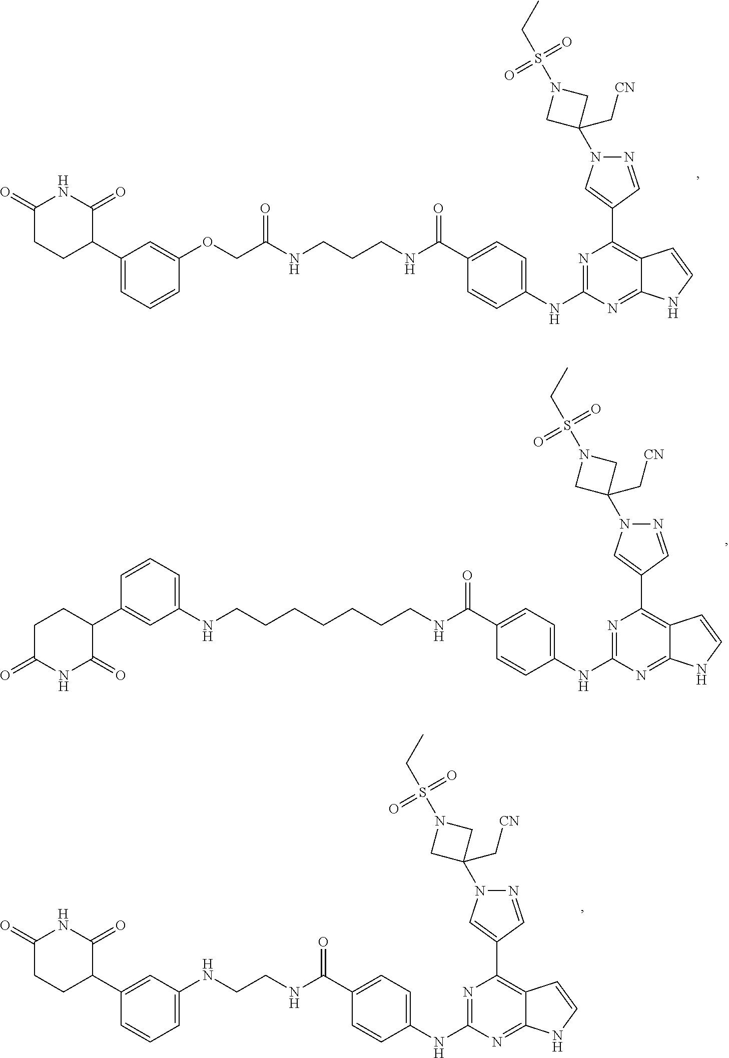 [SMILES: CCS(=O)(=O)N1CC(CC#N)(n2cc(-c3nc(Nc4ccc(C(=O)NCCCNC(=O)COc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCCCCCNc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCNc5cccc(C6CCC(=O)NC6=O)c5)cc4)nc4[nH]ccc34)cn2)C1]

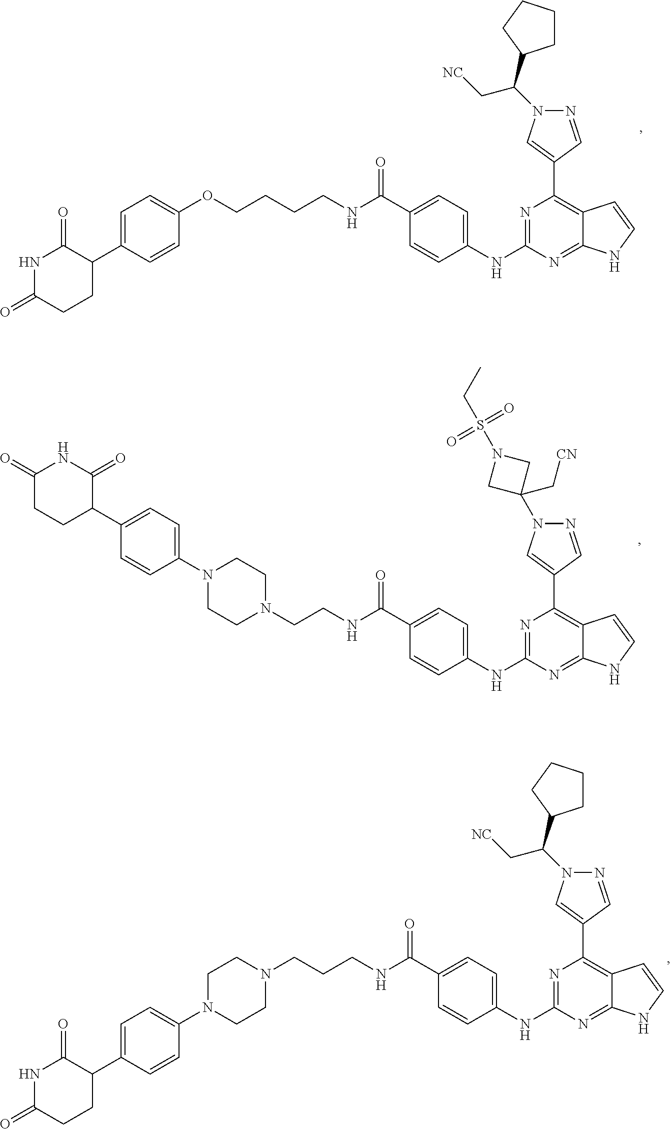 [SMILES: N#CC[C@@H](C1CCCC1)n1cc(-c2nc(Nc3ccc(C(=O)NCCCCOc4ccc(C5CCC(=O)NC5=O)cc4)cc3)nc3[nH]ccc23)cn1 ; CCS(=O)(=O)N1CC(CC#N)(n2cc(-c3nc(Nc4ccc(C(=O)NCCN5CCN(c6ccc(C7CCC(=O)NC7=O)cc6)CC5)cc4)nc4[nH]ccc34)cn2)C1 ; N#CC[C@@H](C1CCCC1)n1cc(-c2nc(Nc3ccc(C(=O)NCCCN4CCN(c5ccc(C6CCC(=O)NC6=O)cc5)CC4)cc3)nc3[nH]ccc23)cn1]

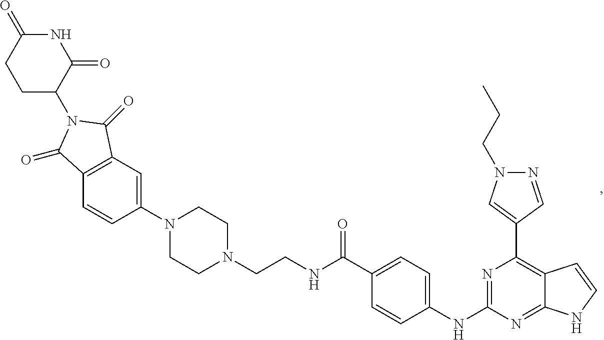 [SMILES: CCCn1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1]

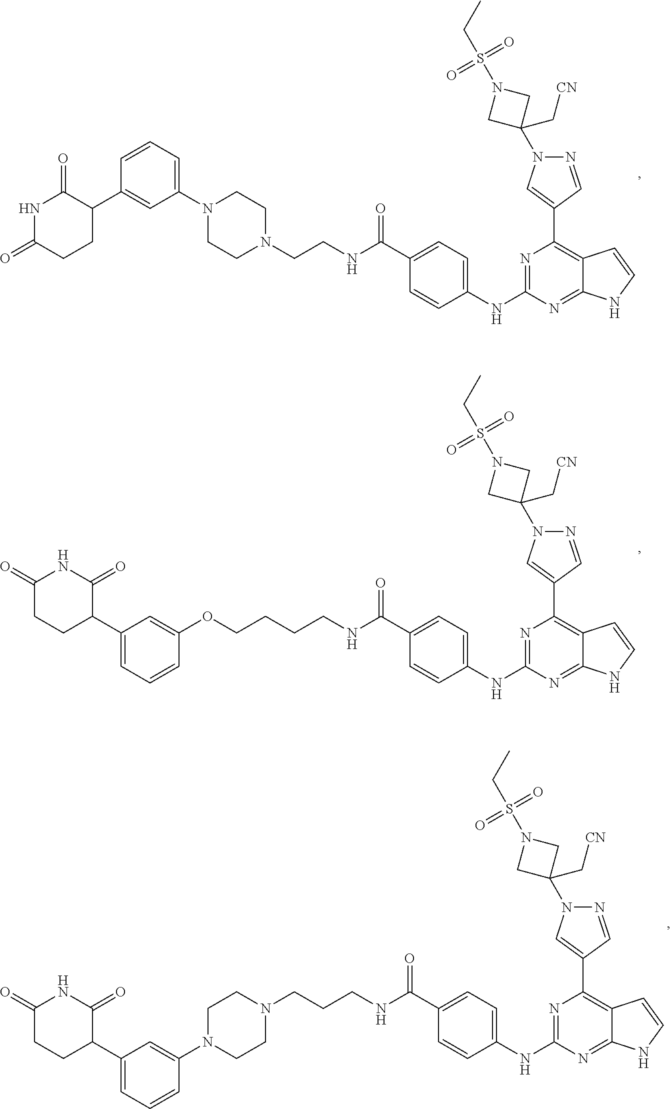 [SMILES: CCS(=O)(=O)N1CC(CC#N)(n2cc(-c3nc(Nc4ccc(C(=O)NCCN5CCN(c6cccc(C7CCC(=O)NC7=O)c6)CC5)cc4)nc4[nH]ccc34)cn2)C1 ; CCS(=O)(=O)N1CC(CC#N)(n2cc(-c3nc(Nc4ccc(C(=O)NCCCCOc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCN5CCN(c6cccc(C7CCC(=O)NC7=O)c6)CC5)cc4)nc4[nH]ccc34)cn2)C1]

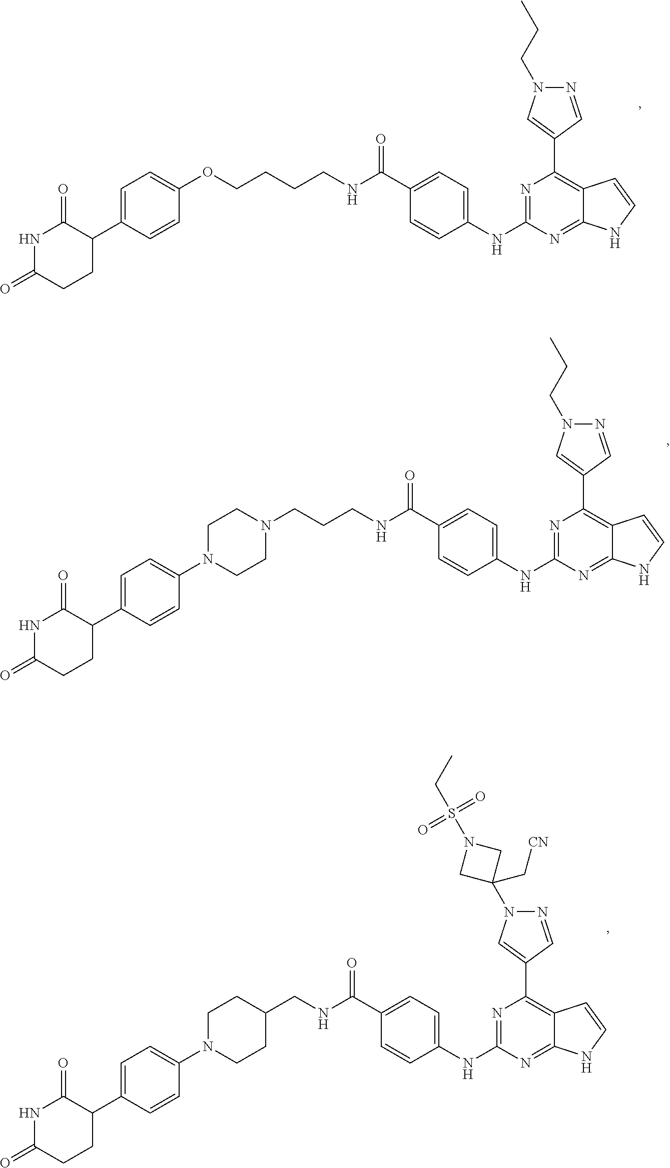 [SMILES: CCCn1cc(-c2nc(Nc3ccc(C(=O)NCCCCOc4ccc(C5CCC(=O)NC5=O)cc4)cc3)nc3[nH]ccc23)cn1 ; CCCn1cc(-c2nc(Nc3ccc(C(=O)NCCCN4CCN(c5ccc(C6CCC(=O)NC6=O)cc5)CC4)cc3)nc3[nH]ccc23)cn1 ; CCS(=O)(=O)N1CC(CC#N)(n2cc(-c3nc(Nc4ccc(C(=O)NCC5CCN(c6ccc(C7CCC(=O)NC7=O)cc6)CC5)cc4)nc4[nH]ccc34)cn2)C1]

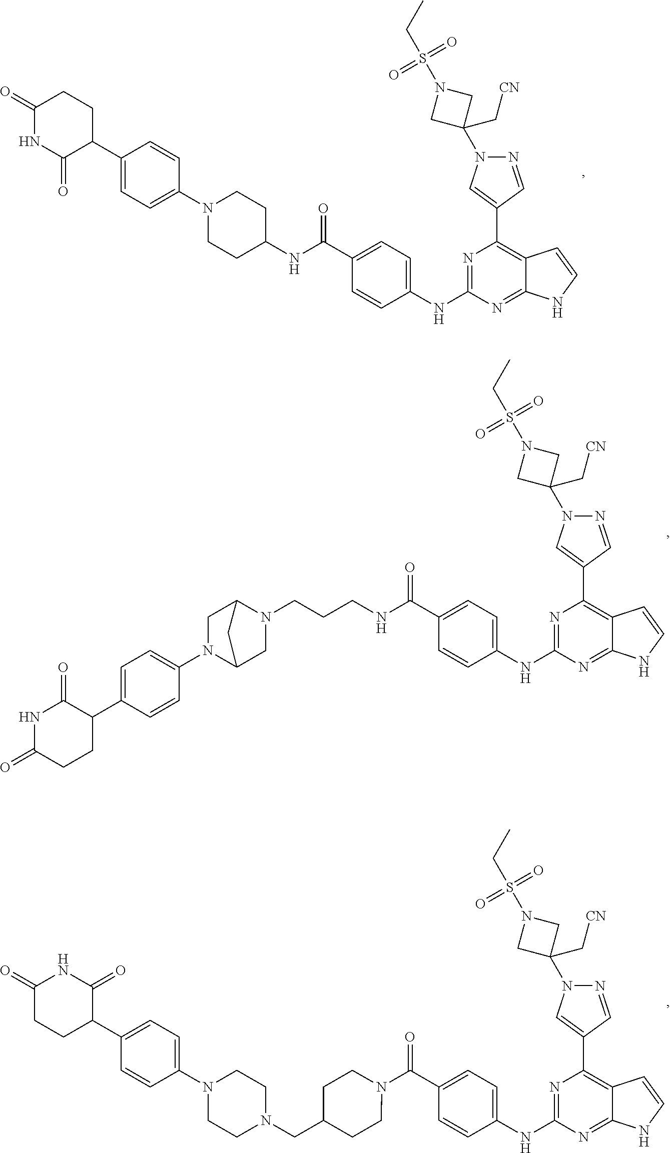 [SMILES: CCS(=O)(=O)N1CC(CC#N)(n2cc(-c3nc(Nc4ccc(C(=O)NC5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NCCCN5CC6CC5CN6c5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)N5CCC(CN6CCN(c7ccc(C8CCC(=O)NC8=O)cc7)CC6)CC5)cc4)nc4[nH]ccc34)cn2)C1]

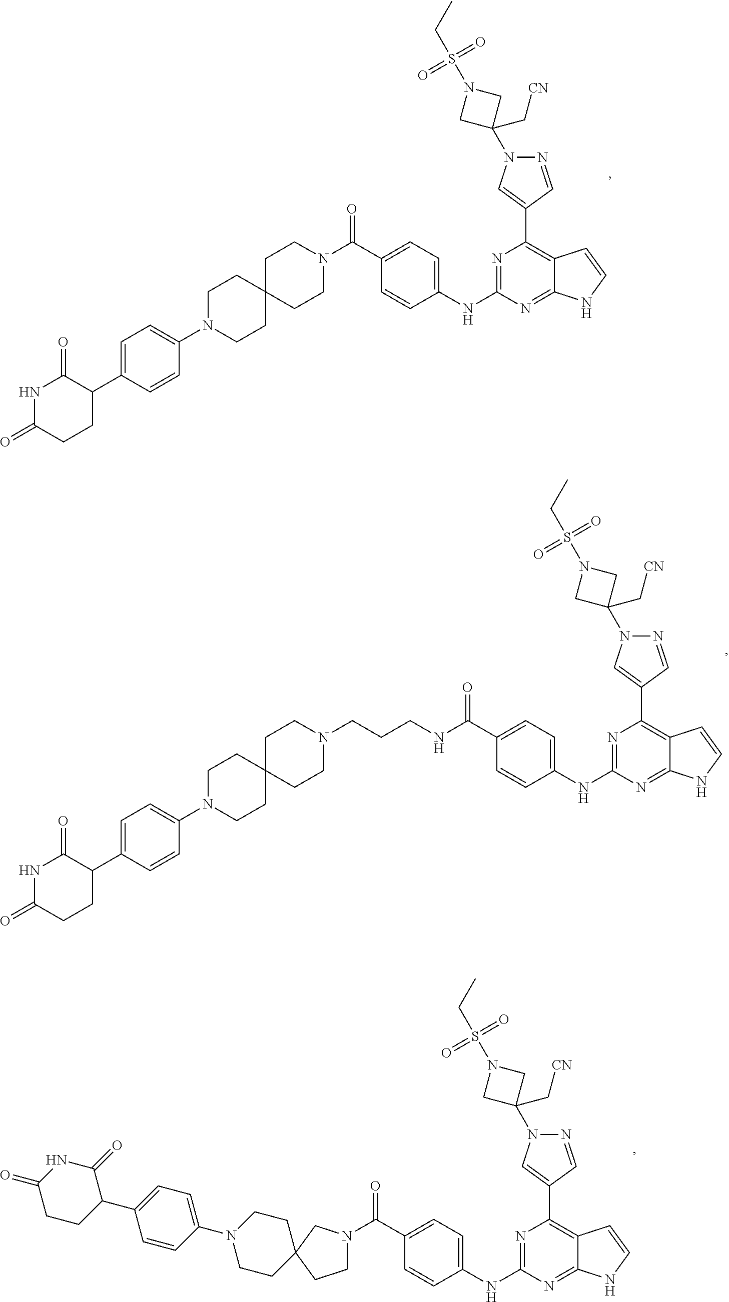 [SMILES: CCS(=O)(=O)N1CC(CC#N)(n2cc(-c3nc(Nc4ccc(C(=O)N5CCC6(CC5)CCN(c5ccc(C7CCC(=O)NC7=O)cc5)CC6)cc4)nc4[nH]ccc34)cn2)C1 ; CCS(=O)(=O)N1CC(CC#N)(n2cc(-c3nc(Nc4ccc(C(=O)NCCCN5CCC6(CC5)CCN(c5ccc(C7CCC(=O)NC7=O)cc5)CC6)cc4)nc4[nH]ccc34)cn2)C1 ; CCS(=O)(=O)N1CC(CC#N)(n2cc(-c3nc(Nc4ccc(C(=O)N5CCC6(CCN(c7ccc(C8CCC(=O)NC8=O)cc7)CC6)C5)cc4)nc4[nH]ccc34)cn2)C1]

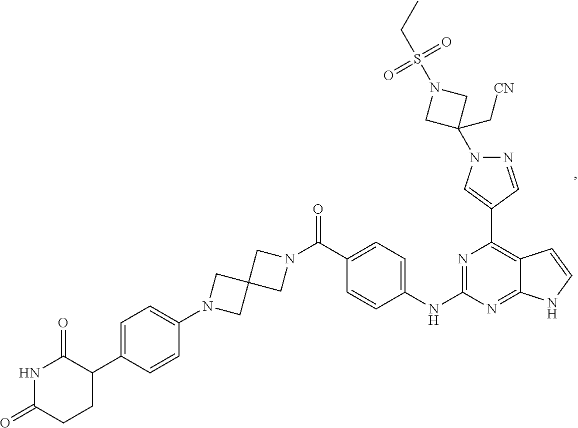 [SMILES: CCS(=O)(=O)N1CC(CC#N)(n2cc(-c3nc(Nc4ccc(C(=O)N5CC6(C5)CN(c5ccc(C7CCC(=O)NC7=O)cc5)C6)cc4)nc4[nH]ccc34)cn2)C1]

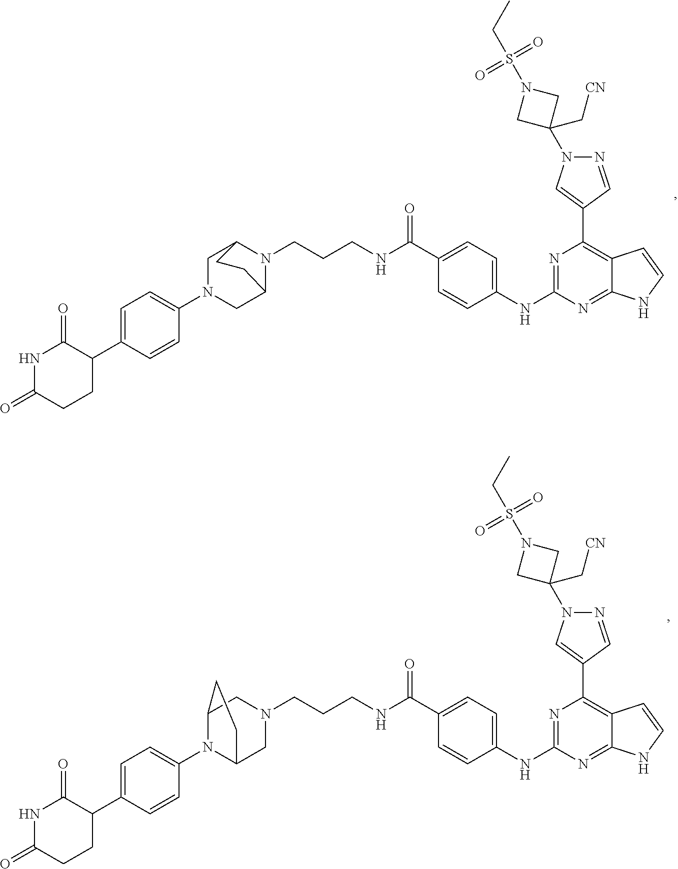 [SMILES: CCS(=O)(=O)N1CC(CC#N)(n2cc(-c3nc(Nc4ccc(C(=O)NCCCN5C6CCC5CN(c5ccc(C7CCC(=O)NC7=O)cc5)C6)cc4)nc4[nH]ccc34)cn2)C1 ; CCS(=O)(=O)N1CC(CC#N)(n2cc(-c3nc(Nc4ccc(C(=O)NCCCN5CC6CCC(C5)N6c5ccc(C6CCC(=O)NC6=O)cc5)cc4)nc4[nH]ccc34)cn2)C1]

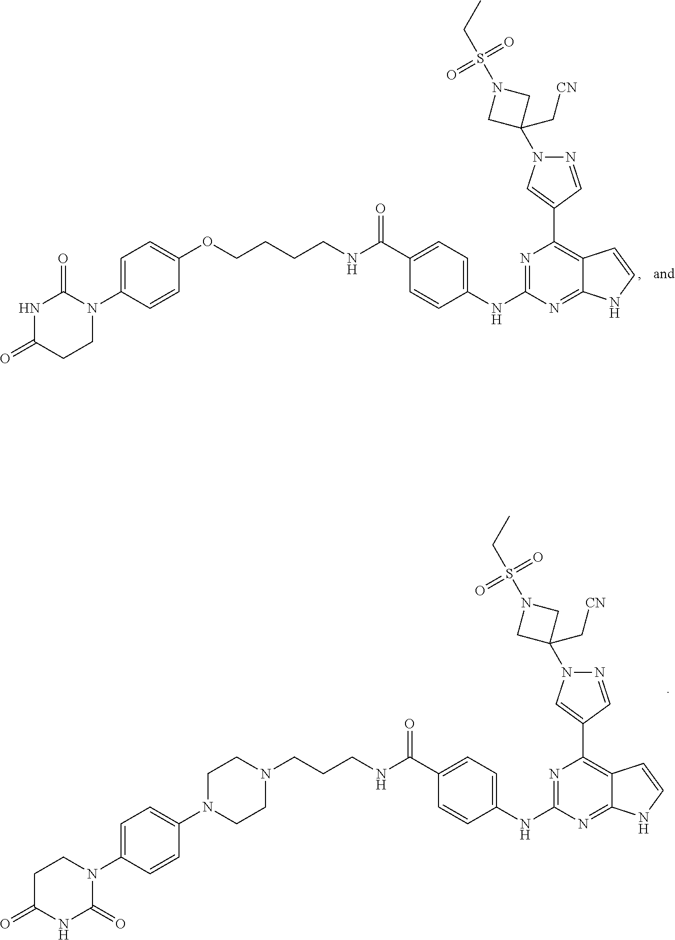 [SMILES: CCS(=O)(=O)N1CC(CC#N)(n2cc(-c3nc(Nc4ccc(C(=O)NCCCCOc5ccc(N6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(N7CCC(=O)NC7=O)cc6)CC5)cc4)nc4[nH]ccc34)cn2)C1]

or a pharmaceutically acceptable salt thereof.

In a further aspect, the cell is mammalian. In a still further aspect, the cell is human. In yet a further aspect, the cell has been isolated from a mammal prior to the contacting step.

In a further aspect, contacting is via administration to a mammal.

In a further aspect, the mammal has been diagnosed with a need for treatment of a disorder associated with cellular activity prior to the administering step. In a still further aspect, the disorder associated with cellular proliferation activity is cancer, for example a cancer selected from for example a cancer selected from sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and in another example acute lymphoblastic leukemia.

In a further aspect, the mammal has been diagnosed with a need for modulating cellular proliferation activity prior to the administering step.

### H. Additional Methods of Using the Compositions

Provided are methods of using of a disclosed composition or medicament. In one aspect, the method of use is directed to the treatment of a disorder. In a further aspect, the disclosed compounds can be used as single agents or in combination with one or more other drugs in the treatment, prevention, control, amelioration, or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

1. Manufacture of a Medicament

In one aspect, the invention relates to a method for the manufacture of a medicament for treating a disorder associated with uncontrolled cellular proliferation in a mammal, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the inhibition of uncontrolled cellular proliferation, such as cancer, and the cancer's disclosed herein. The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the disorder.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

2. Use of Compounds and Compositions

Also provided are the uses of the disclosed compounds and compositions. Thus, in one aspect, the invention relates to the uses of modulators of cellular proliferation activity.

In a further aspect, the invention relates to the use of a disclosed compound or product of a disclosed method in the manufacture of a medicament for the treatment of a disorder associated with uncontrolled cellular proliferation, for example, cancer, such as sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and in another example acute lymphoblastic leukemia.

In a further aspect, the use relates to a process for preparing a pharmaceutical composition comprising a therapeutically effective amount of a disclosed compound or a product of a disclosed method, and a pharmaceutically acceptable carrier, for use as a medicament.

In a further aspect, the use relates to a process for preparing a pharmaceutical composition comprising a therapeutically effective amount of a disclosed compound or a product of a disclosed method, wherein a pharmaceutically acceptable carrier is intimately mixed with a therapeutically effective amount of the disclosed compound or the product of a disclosed method.

In various aspects, the use relates to the treatment of uncontrolled cellular proliferation in a vertebrate animal. In a further aspect, the use relates to the treatment of uncontrolled cellular proliferation in a human subject.

In a further aspect, the use is the treatment of uncontrolled cellular proliferation, for example cancer, for example a cancer selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and in another example acute lymphoblastic leukemia.

It is understood that the disclosed uses can be employed in connection with the disclosed compounds, methods, compositions, and kits. In a further aspect, the invention relates to the use of a disclosed compound or composition of a medicament for the treatment of a disorder associated with uncontrolled cellular proliferation in a mammal.

In a further aspect, the invention relates to the use of a disclosed compound or composition in the manufacture of a medicament for the treatment of a disorder associated with uncontrolled cellular proliferation, for example cancer, such a cancer selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and in another example acute lymphoblastic leukemia.

3. Kits

In one aspect, disclosed are kits comprising a disclosed compound and one or more of: (a) an agent known to treat a disorder of uncontrolled cellular proliferation; (b) instructions for administering the compound in connection with treating a disorder of uncontrolled cellular proliferation; and (c) instructions for treating a disorder of uncontrolled cellular proliferation.

In one aspect, disclosed are kits comprising a compound having a structure represented by a formula:

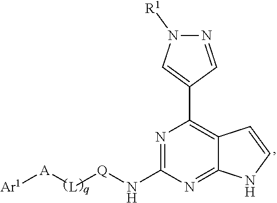

wherein q is selected from 0 and 1; wherein A is selected from —O—, —S—, —NH—, —NHCH₂—, —OCH₂C(O)NH—, —CH₂—, and a structure selected from:

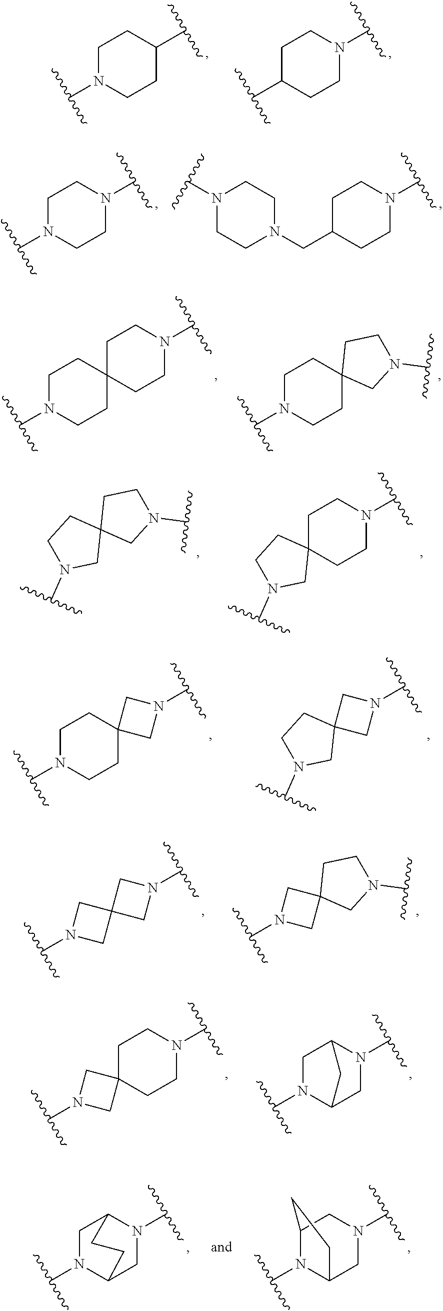

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

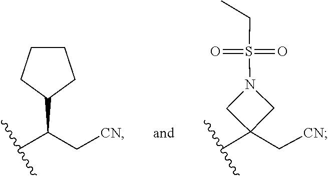 [SMILES: *[C@H](CC#N)C1CCCC1 ; "and" ; *C1(CC#N)CN(S(=O)(=O)CC)C1]

and
wherein Ar¹ is a structure selected from:

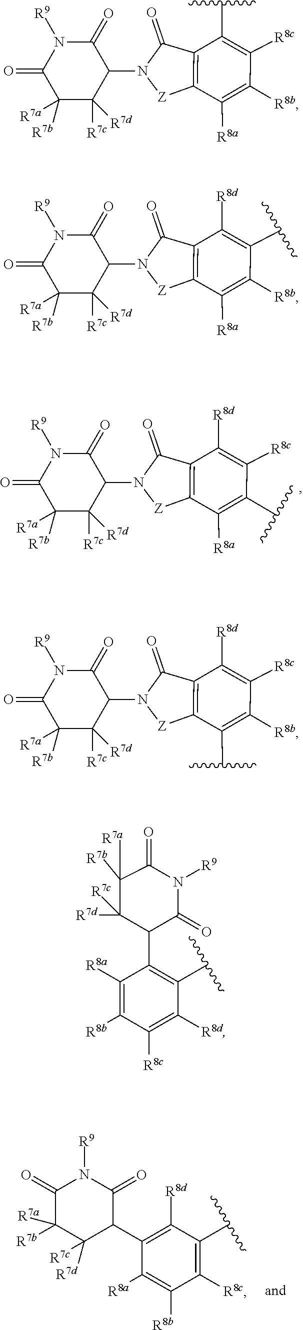

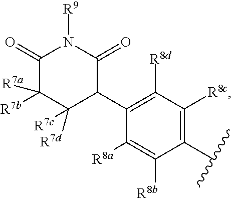

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

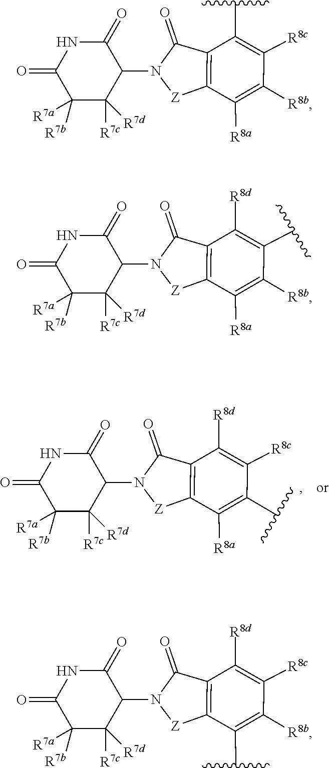

and q is 1, then A is a structure selected from:

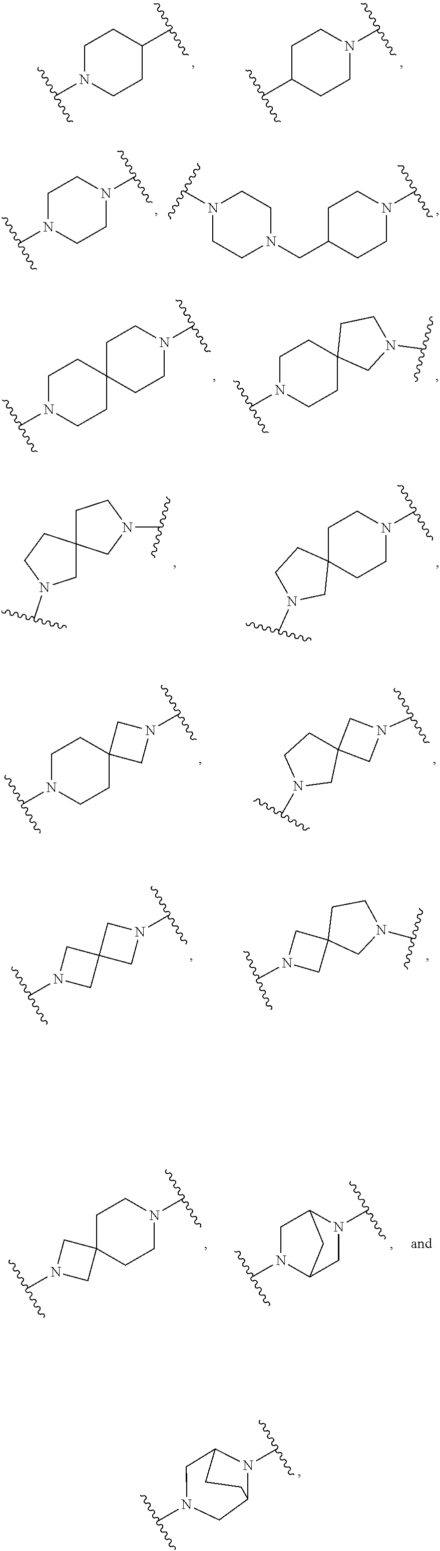

or a pharmaceutically acceptable salt thereof, and one or more of: (a) an agent known to treat a disorder of uncontrolled cellular proliferation; (b) instructions for administering the compound in connection with treating a disorder of uncontrolled cellular proliferation; and (c) instructions for treating a disorder of uncontrolled cellular proliferation.

In one aspect, disclosed are kits comprising a compound having a structure represented by a formula:

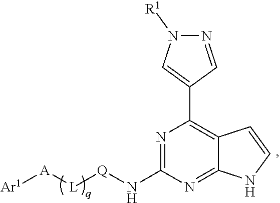

wherein q is selected from 0 and 1; wherein A is selected from —O—, —S—, —NH—, —NHCH₂—, —OCH₂C(O)NH—, —CH₂—, and a structure selected from:

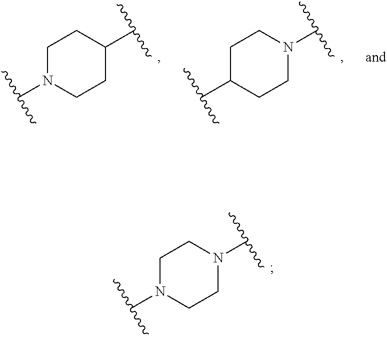

wherein L is selected from C1-C15 alkyl, —(CH₂CH₂O)ₙ—, and —(CH₂CH₂O)ₙ(C1-C4 alkyl)-; wherein n, when present, is selected from 1, 2, 3, 4, 5, 6, 7, and 8; wherein Q is selected from —C(O)(C₆H₄)—, —NHC(O)(C₆H₄)—, and —C(O)NH(C₆H₄)—; wherein R¹ is selected from C1-C8 alkyl, C1-C8 hydroxyalkyl, C1-C8 cyanoalkyl, C1-C8 aminoalkyl, and a structure selected from:

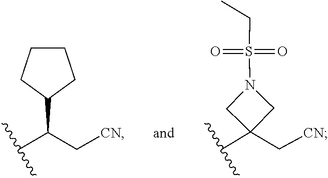 [SMILES: *[C@H](CC#N)C1CCCC1 ; "and" ; *C1(CC#N)CN(S(=O)(=O)CC)C1]

and
wherein Ar¹ is a structure selected from:

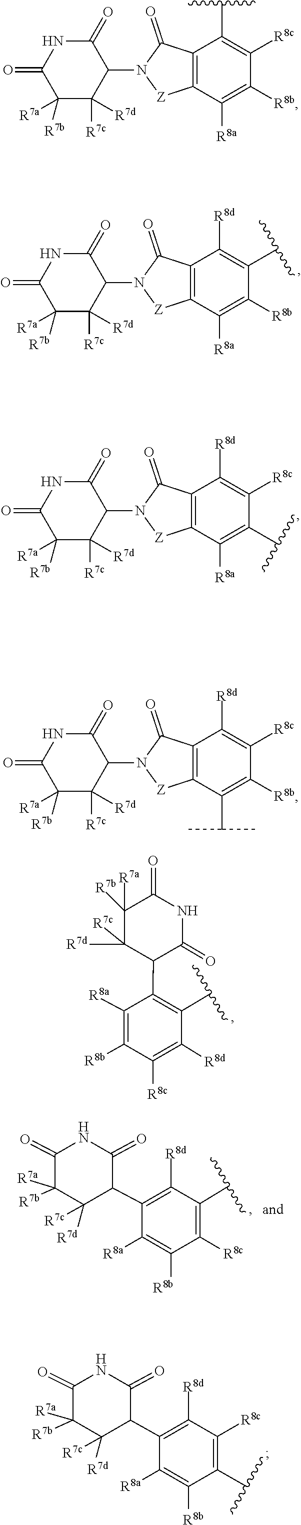

wherein Z is selected from —CH₂— and C(O); wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and wherein each of R^{8a}, R^{Bb}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl, provided that when Ar¹ is:

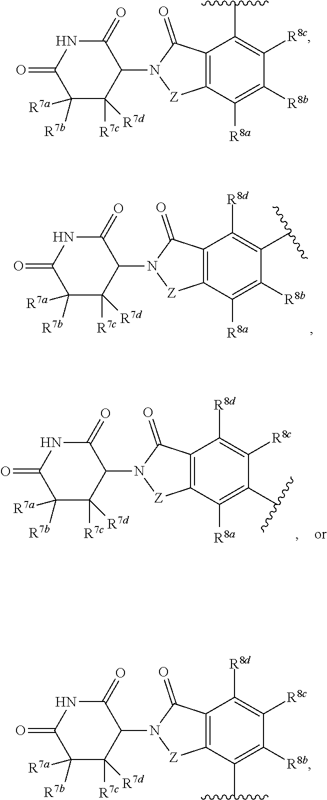

and q is 1, then A is a structure selected from:

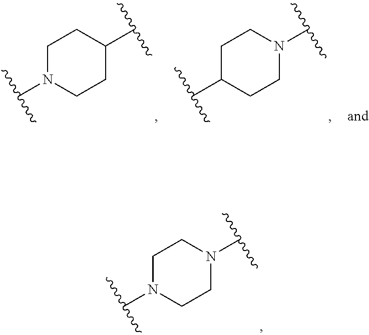 [SMILES: *C1CCN(*)CC1 ; *C1CCN(*)CC1 ; *N1CCN(*)CC1]

or a pharmaceutically acceptable salt thereof, and one or more of: (a) an agent known to treat a disorder of uncontrolled cellular proliferation; (b) instructions for administering the compound in connection with treating a disorder of uncontrolled cellular proliferation; and (c) instructions for treating a disorder of uncontrolled cellular proliferation.

In various aspects, the agents and pharmaceutical compositions described herein can be provided in a kit. The kit can also include combinations of the agents and pharmaceutical compositions described herein.

In various aspects, the informational material can be descriptive, instructional, marketing or other material that relates to the methods described herein and/or to the use of the agents for the methods described herein. For example, the informational material may relate to the use of the agents herein to treat a subject who has, or who is at risk for developing, a disorder associated with uncontrolled cellular proliferation. The kits can also include paraphernalia for administering the agents of this invention to a cell (in culture or in vivo) and/or for administering a cell to a patient.

In various aspects, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In a further aspect, the informational material can include instructions to administer the pharmaceutical composition to a suitable subject, e.g., a human having, or at risk for developing, a disorder associated with uncontrolled cellular proliferation activity.

In various aspects, the composition of the kit can include other ingredients, such as a solvent or buffer, a stabilizer, a preservative, a fragrance or other cosmetic ingredient. In such aspects, the kit can include instructions for admixing the agent and the other ingredients, or for using one or more compounds together with the other ingredients.

In a further aspect, the compound and the at least one agent known to treat a disorder of uncontrolled cellular proliferation are co-formulated. In a still further aspect, the compound and the at least one agent known to treat a disorder of uncontrolled cellular proliferation are co-packaged.

In a further aspect, the at least one agent known to treat uncontrolled cellular proliferation is a chemotherapeutic agent. The chemotherapeutic agent can be selected from an alkylating agent, an antimetabolite agent, an antineoplastic antibiotic agent, a mitotic inhibitor agent, and a mTor inhibitor agent. In one aspect, the at least one agent is an antineoplastic antibiotic agent selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof. In a further aspect, the at least one agent is an antimetabolite agent selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof. In a further aspect, the at least one agent is an alkylating agent selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof. In a further aspect, the at least one agent is a mitotic inhibitor agent selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof. In a further aspect, the at least one agent is an mTor inhibitor agent selected from everolimus, siroliumus, and temsirolimus, or a pharmaceutically acceptable salt, hydrate, solvate, or polymorph thereof.

In a further aspect, the kit further comprises a plurality of dosage forms, the plurality comprising one or more doses; wherein each dose comprises an effective amount of the compound and the at least one agent known to treat uncontrolled cellular proliferation. In a still further aspect, the effective amount is a therapeutically effective amount. In yet a further aspect, the effective amount is a prophylactically effective amount. In an even further aspect, each dose of the compound and at least one agent known to treat uncontrolled cellular proliferation are co-packaged. In a still further aspect, each dose of the compound and the at least one agent known to treat uncontrolled cellular proliferation are co-formulated.

In a further aspect, the at least one agent known to a disorder associated with uncontrolled cellular proliferation, for example cancer, such a cancer selected from a sarcoma, a carcinoma, a hematological cancer, a solid tumor, breast cancer, cervical cancer, gastrointestinal cancer, colorectal cancer, brain cancer, skin cancer, prostate cancer, ovarian cancer, non-small cell lung carcinoma, thyroid cancer, testicular cancer, pancreatic cancer, liver cancer, endometrial cancer, melanoma, glioma, leukemia, lymphoma, chronic myeloproliferative disorder, myelodysplastic syndrome, myeloproliferative neoplasm, and plasma cell neoplasm (myeloma), and in another example acute lymphoblastic leukemia.

In a further aspect, the kit further comprises a plurality of dosage forms, the plurality comprising one or more doses; wherein each dose comprises an effective amount of the compound and at least one agent known to treat uncontrolled cellular proliferation. In a still further aspect, the effective amount is a therapeutically effective amount. In yet a further aspect, the effective amount is a prophylactically effective amount. In an even further aspect, each dose of the compound and at least one agent known to treat uncontrolled cellular proliferation are co-packaged. In a still further aspect, each dose of the compound and at least one agent known to treat uncontrolled cellular proliferation are co-formulated.

4. Subjects

In various aspects, the subject of the herein disclosed methods is a vertebrate, e.g., a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a disorder associated with uncontrolled cellular proliferation prior to the administering step. In some aspects of the disclosed methods, the subject has been identified with a need for treatment prior to the administering step. In one aspect, a subject can be treated prophylactically with a compound or composition disclosed herein, as discussed herein elsewhere.

a. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

b. Routes of Administration

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### I. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Chemistry Methods

a. Synthesis of Compounds 10 and 19

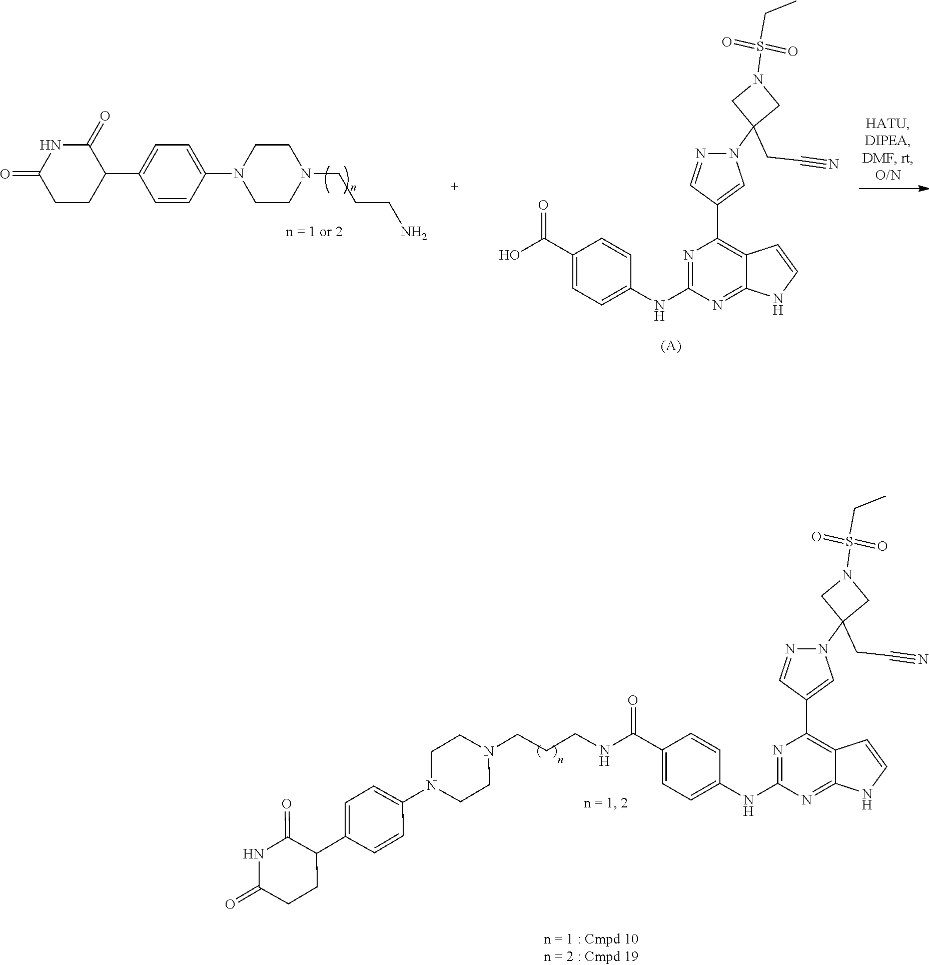

b. Synthesis of Compounds 20, 28, 29, 30, 31, and 32

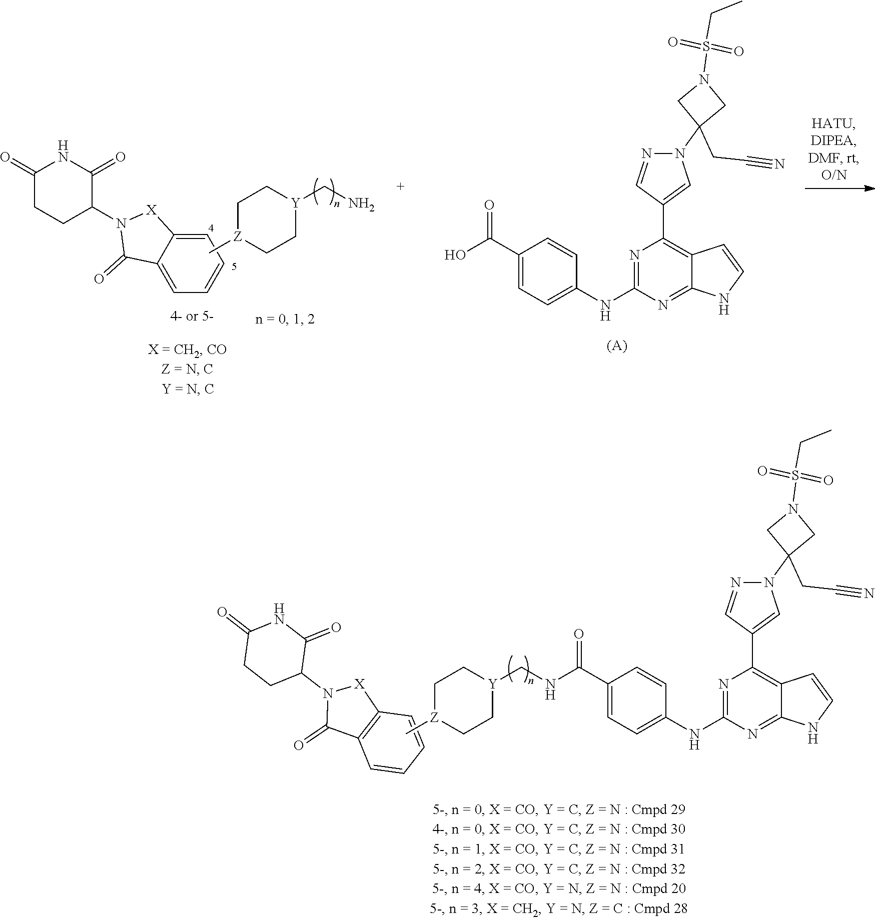

c. Synthesis of Compounds 27, 33, and 34

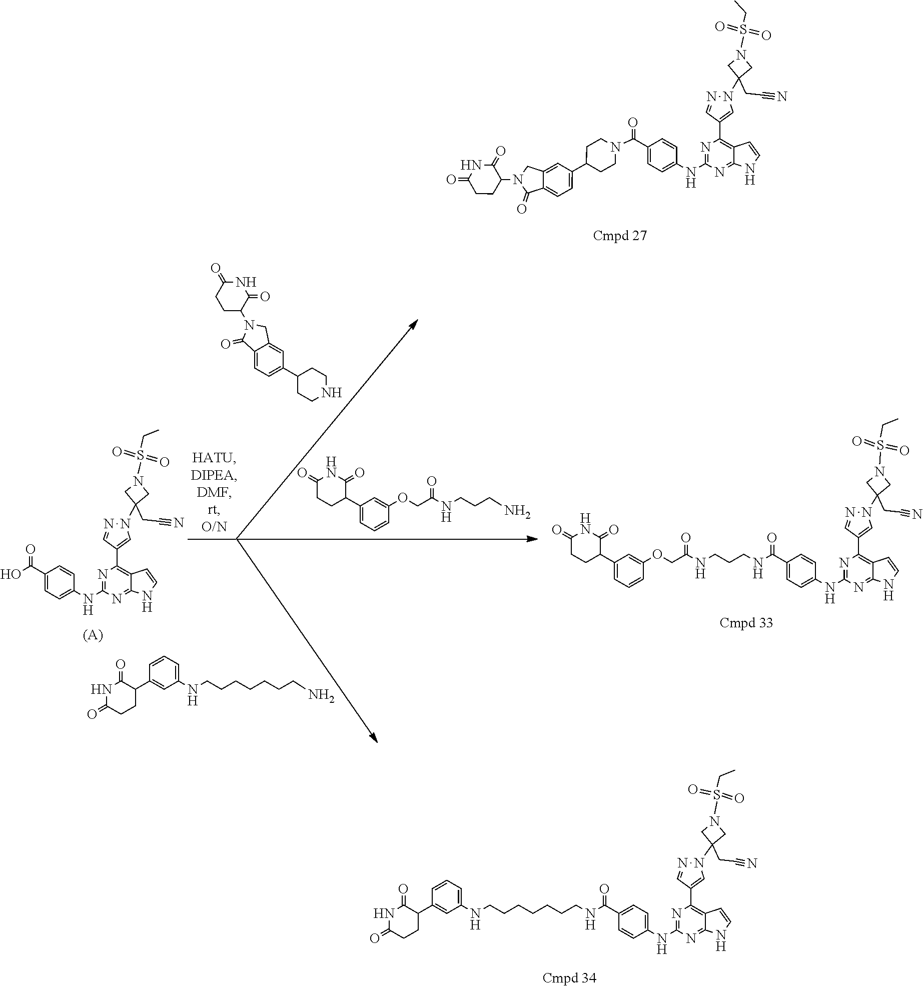

d. Synthesis of Compounds 22 and 23

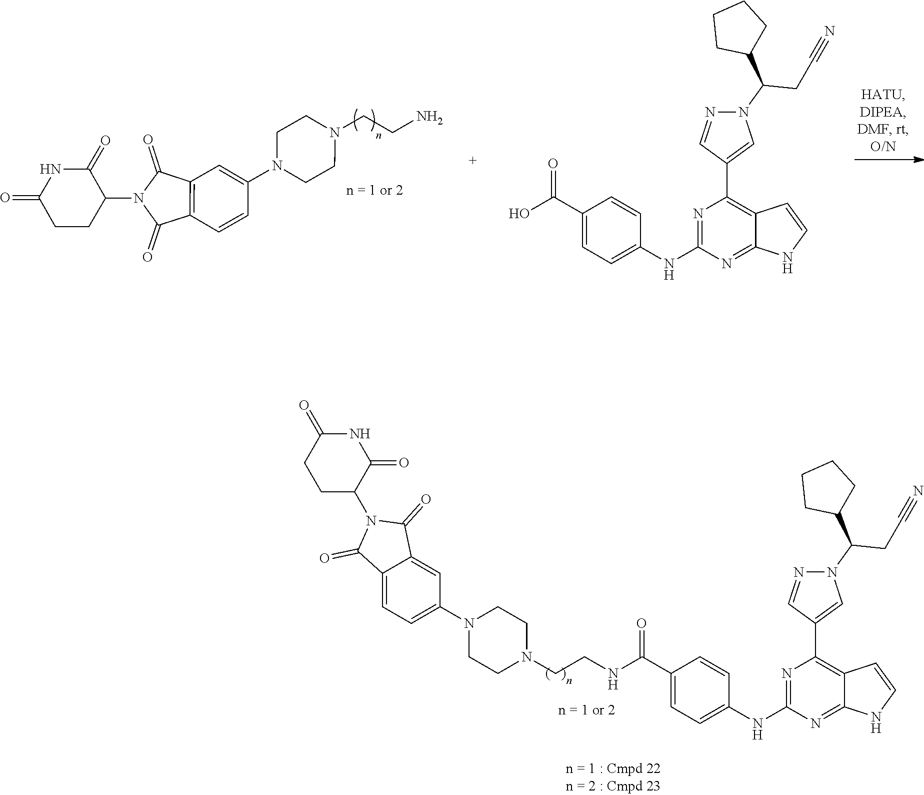

e. Synthesis of Compounds 4 and 13

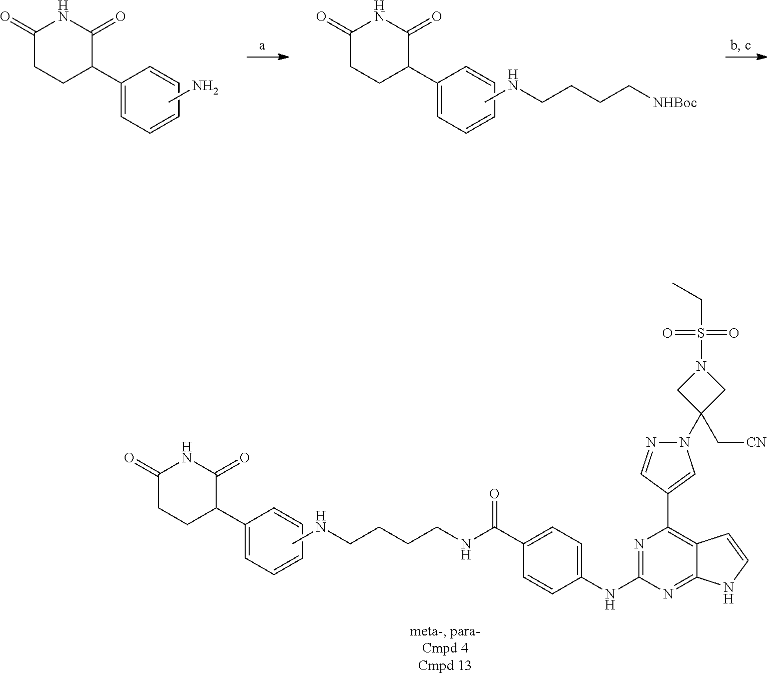

Reagents and conditions: a) tert-butyl (4-bromobutyl)carbamate, N,N-diisopropylethylamine, MeCN, 60° C., 8-40%; b) DCM:TFA (1:1), rt, 1 h; c) 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid, chloro-N,N,N′,N′-tetramethylformamidinium hexafluorophosphate, 1-methylimidazole, MeCN, rt, 16 h, 8-42%.

f. Synthesis of Compounds 15 and 16

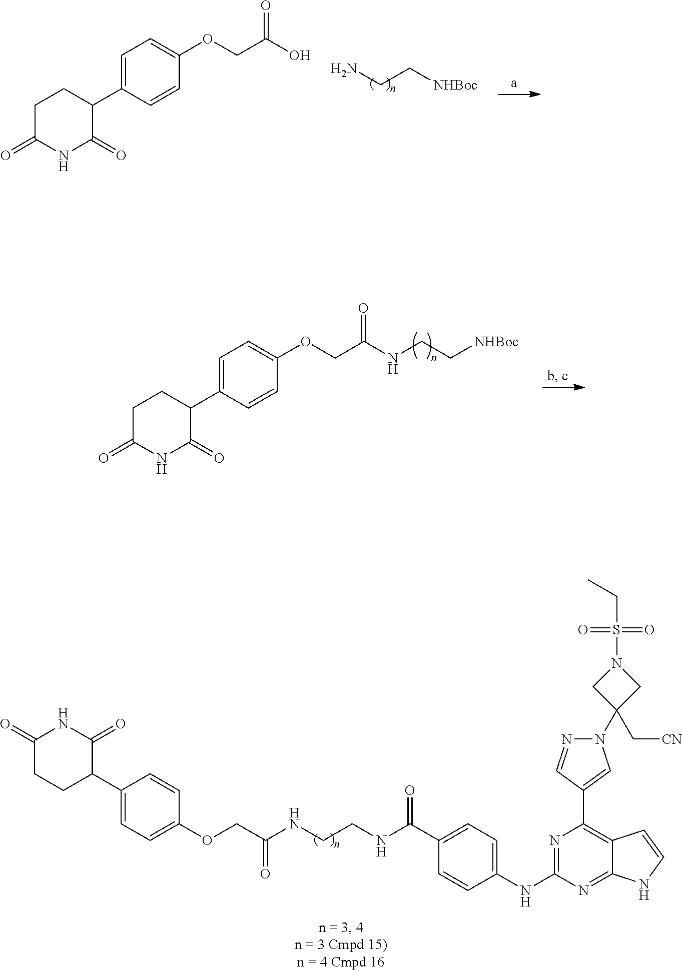

Reagents and conditions: a) chloro-N,N,N′,N′-tetramethylformamidinium hexafluorophosphate, 1-methylimidazole, MeCN, rt, 16 h, 77-93%; b) DCM:TFA (1:1), rt, 1 h; c) 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid, chloro-N,N,N′,N′-tetramethylformamidinium hexafluorophosphate, 1-methylimidazole, MeCN, rt, 16 h, 51-53%.

g. Synthesis of Compounds 8 and 14

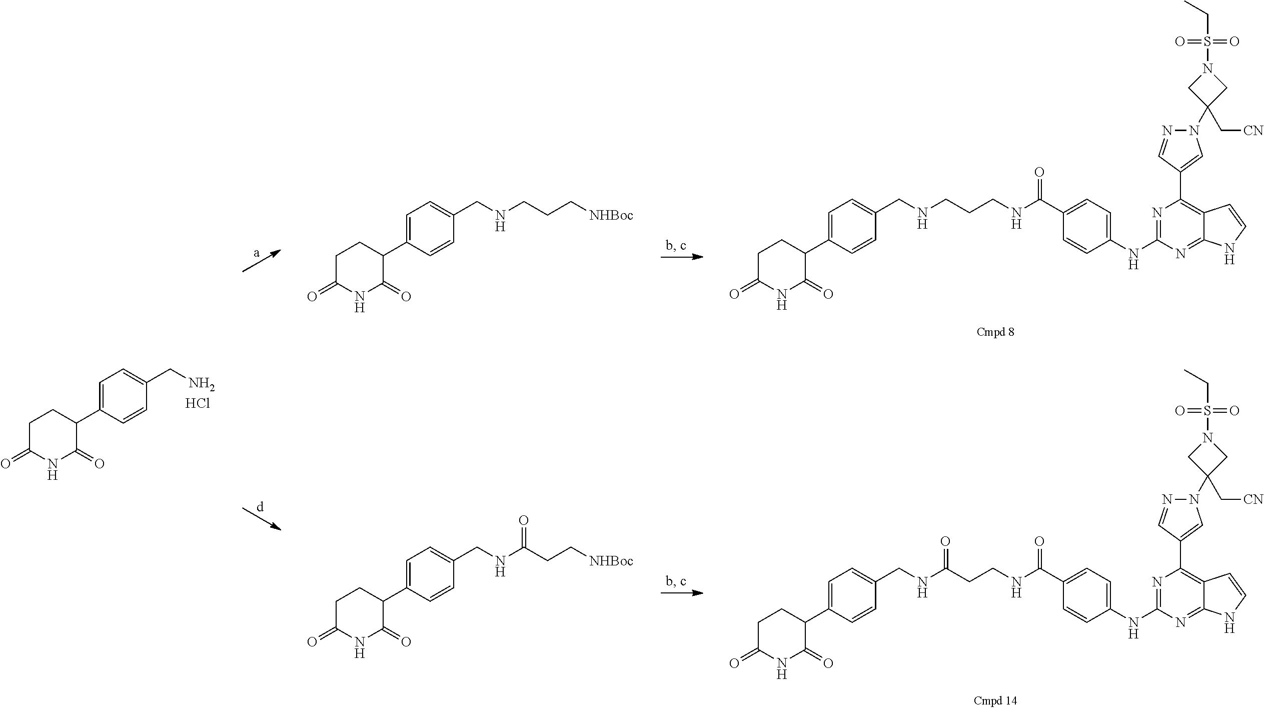 [SMILES: CC(C)(C)OC(=O)NCCCNCc1ccc(C2CCC(=O)NC2=O)cc1 ; CCS(=O)(=O)N1CC(CC#N)(n2cc(-c3nc(Nc4ccc(C(=O)NCCCNCc5ccc(C6CCC(=O)NC6=O)cc5)cc4)nc4[nH]ccc34)cn2)C1 ; NCc1ccc(C2CCC(=O)NC2=O)cc1 ; Cl ; CC(C)(C)OC(=O)NCCC(=O)NCc1ccc(C2CCC(=O)NC2=O)cc1 ; CCS(=O)(=O)N1CC(CC#N)(n2cc(-c3nc(Nc4ccc(C(=O)NCCC(=O)NCc5ccc(C6CCC(=O)NC6=O)cc5)cc4)nc4[nH]ccc34)cn2)C1]

Reagents and conditions: a) 3-((tert-butoxycarbonyl)amino)propyl 4-methylbenzenesulfonate, K2CO3, DMF, 60° C., 6 h, 35%; b) DCM:TFA (1:1), rt, 1 h; c) 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid, chloro-N,N,N′,N′-tetramethylformamidinium hexafluorophosphate, 1-methylimidazole, MeCN, rt, 16 h, 6-15%; d) 3-((tert-butoxycarbonyl)amino)propanoic acid, HATU, DIPEA, DMF, rt, 16 h, 70%.

h. Synthesis of Compounds 2, 5, 9, and 12

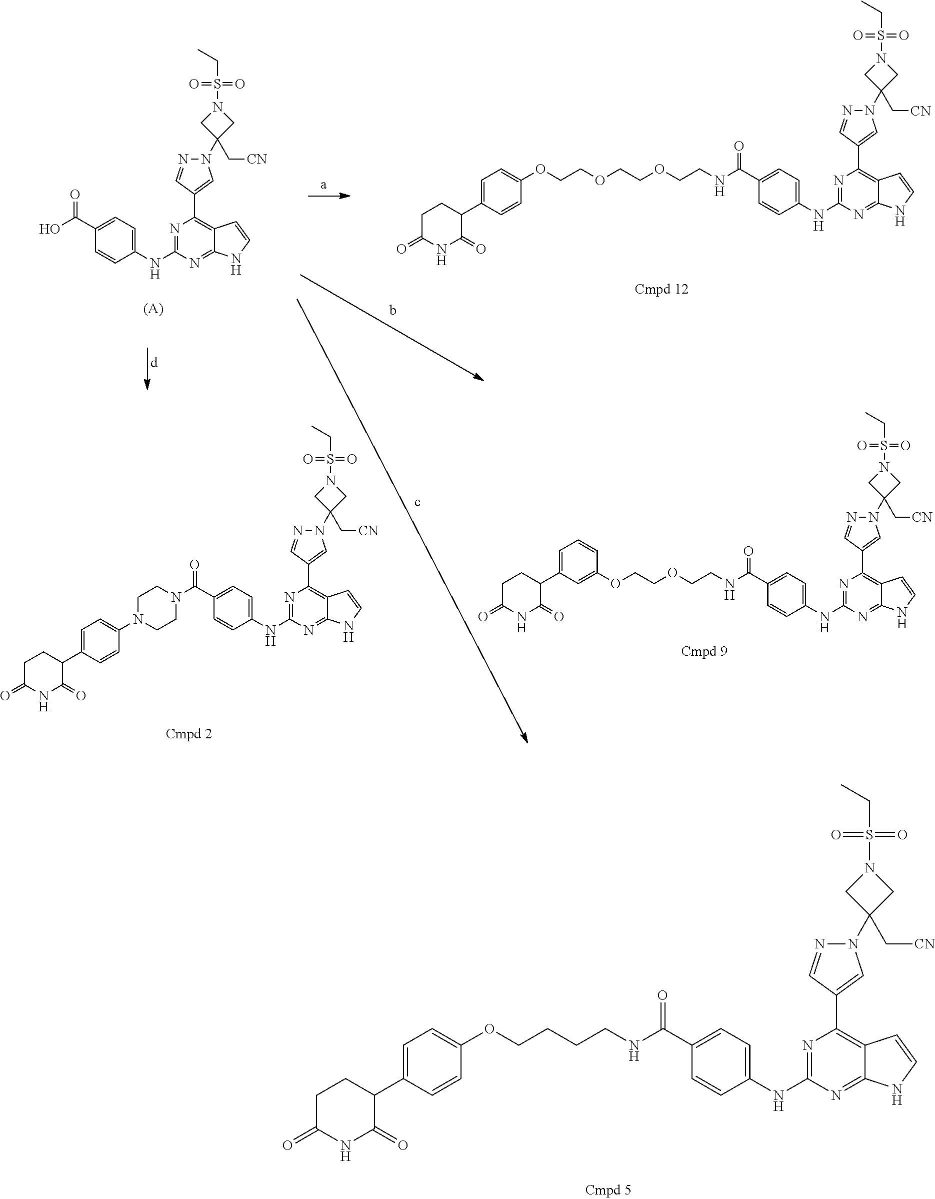 [SMILES: CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)NCCOCCOCCOc5ccc(C6CCC(=O)NC6=O)cc5)cc4)nc4[nH]ccc34)cn2)C1 ; "(A)" ; CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NCCOCCOc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCCOc5ccc(C6CCC(=O)NC6=O)cc5)cc4)nc4[nH]ccc34)cn2)C1]

Reagents and conditions: a) 3-(4-(2-(2-(2-aminoethoxy)ethoxy)ethoxy)phenyl)piperidine-2,6-dione, chloro-N,N,N′,N′-tetramethylformamidinium hexafluorophosphate, 1-methylimidazole, MeCN, rt, 16 h, 47%; b) 3-(3-(2-(2-aminoethoxy)ethoxy)phenyl)piperidine-2,6-dione, chloro-N,N,N′,N′-tetramethylformamidinium hexafluorophosphate, 1-methylimidazole, MeCN, rt, 16 h, 48%; c) 3-(4-(4-aminobutoxy)phenyl)piperidine-2,6-dione, chloro-N,N,N′,N′-tetramethylformamidinium hexafluorophosphate, 1-methylimidazole, MeCN, rt, 16 h, 21%; d) 3-(4-(piperazin-1-yl)phenyl)piperidine-2,6-dione, chloro-N,N,N′,N′-tetramethylformamidinium hexafluorophosphate, 1-methylimidazole, MeCN, rt, 16 h, 36%

i. Synthesis of Compounds 1, 3, 6, and 7

(a) Compound 1

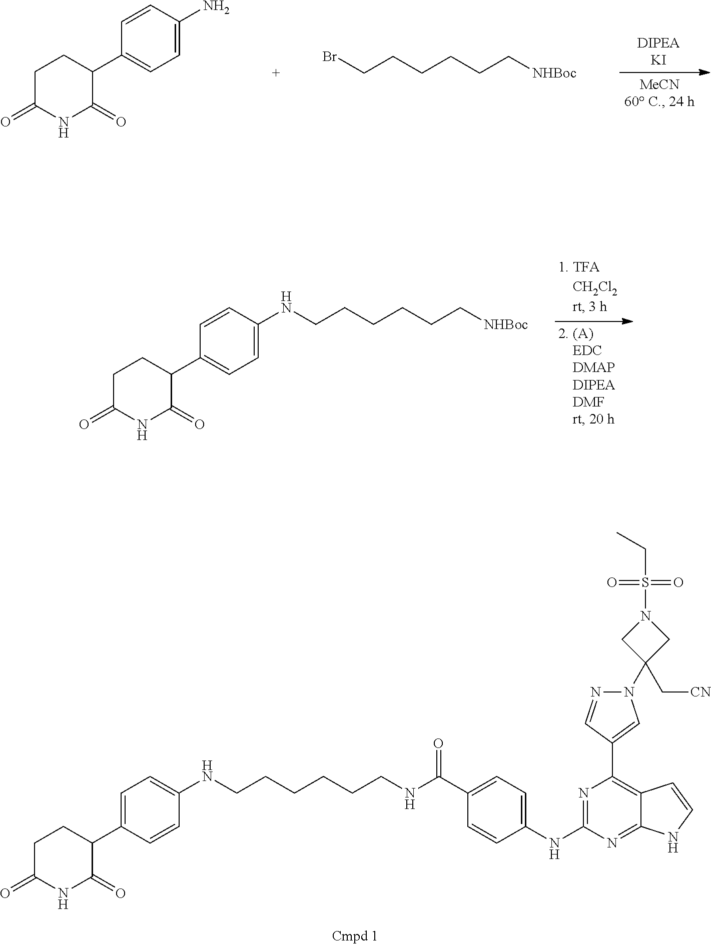 [SMILES: Nc1ccc(C2CCC(=O)NC2=O)cc1 ; CC(C)(C)OC(=O)NCCCCCCBr ; CC(C)(C)OC(=O)NCCCCCCNc1ccc(C2CCC(=O)NC2=O)cc1 ; CCS(=O)(=O)N1CC(CC#N)(n2cc(-c3nc(Nc4ccc(C(=O)NCCCCCCNc5ccc(C6CCC(=O)NC6=O)cc5)cc4)nc4[nH]ccc34)cn2)C1]

(b) Compound 3

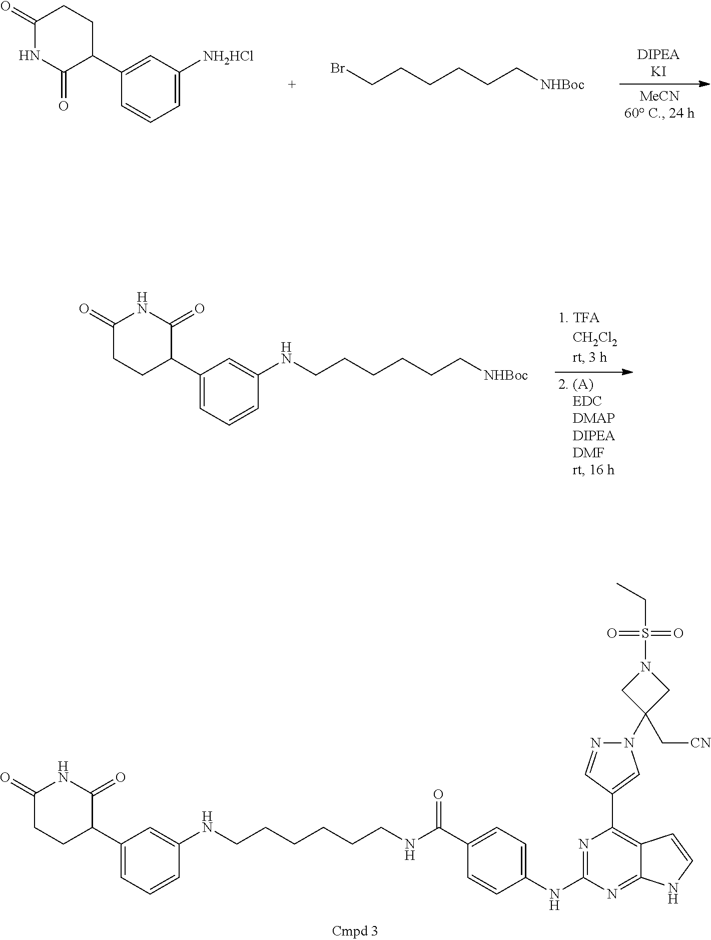

(c) Compound 6

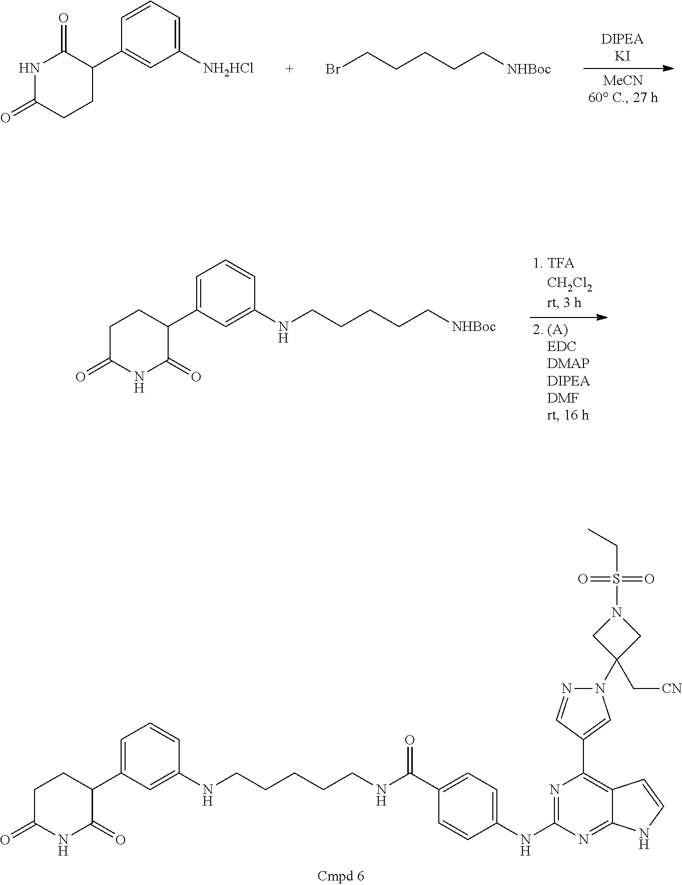

(d) Compound 7

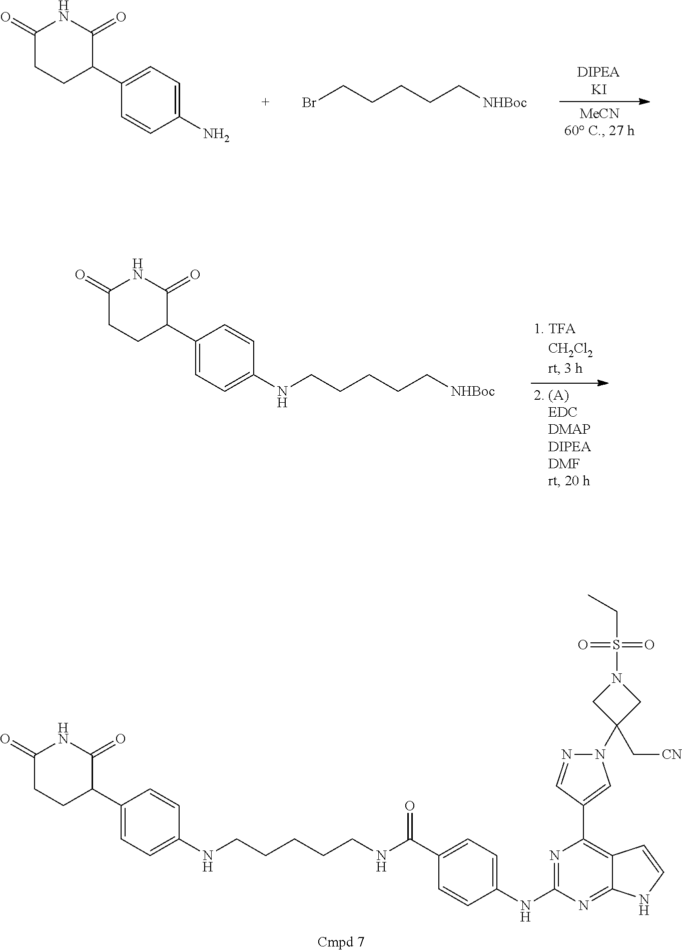 [SMILES: Nc1ccc(C2CCC(=O)NC2=O)cc1 ; CC(C)(C)OC(=O)NCCCCCBr ; CC(C)(C)OC(=O)NCCCCCNc1ccc(C2CCC(=O)NC2=O)cc1 ; CCS(=O)(=O)N1CC(CC#N)(n2cc(-c3nc(Nc4ccc(C(=O)NCCCCCNc5ccc(C6CCC(=O)NC6=O)cc5)cc4)nc4[nH]ccc34)cn2)C1]

j. Synthesis of Compounds 11, 17, and 18

(a) Compound 11

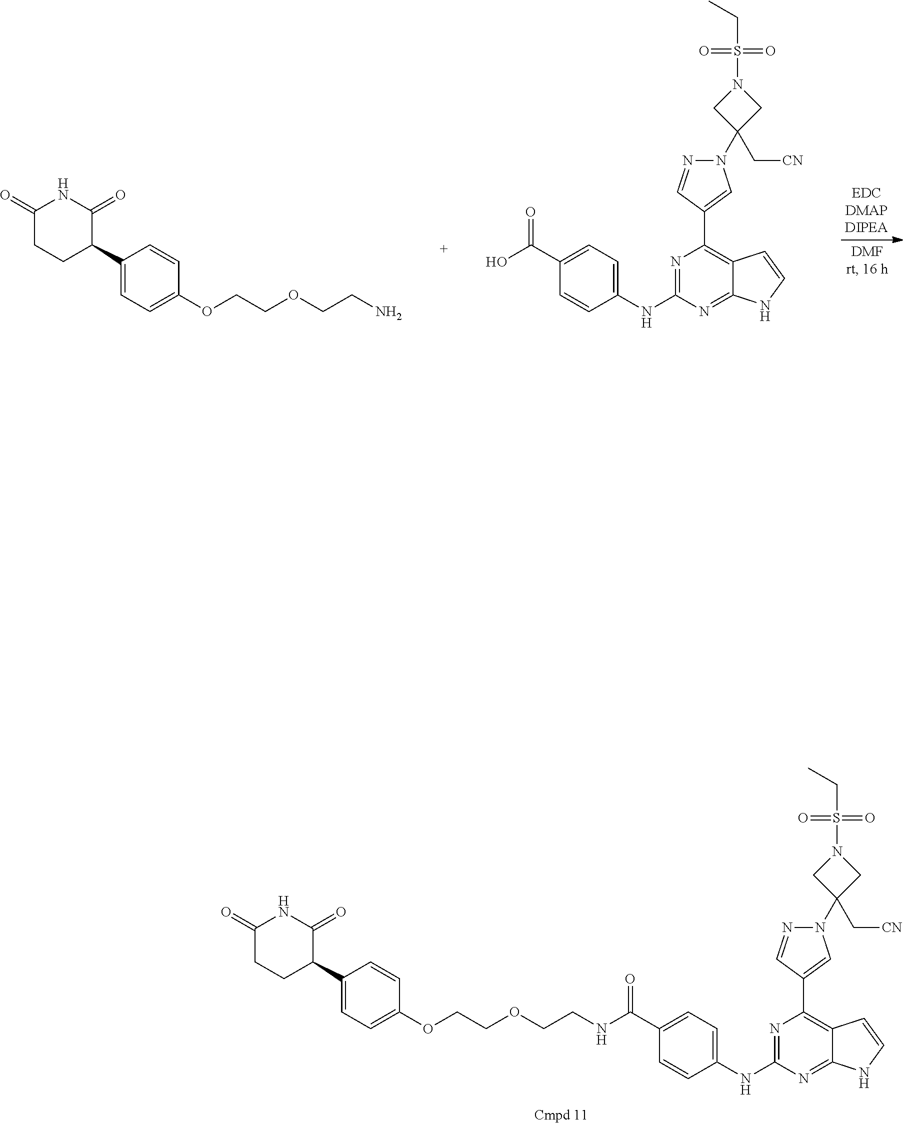 [SMILES: NCCOCCOc1ccc([C@H]2CCC(=O)NC2=O)cc1 ; CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)NCCOCCOc5ccc([C@H]6CCC(=O)NC6=O)cc5)cc4)nc4[nH]ccc34)cn2)C1]

(b) Compound 17

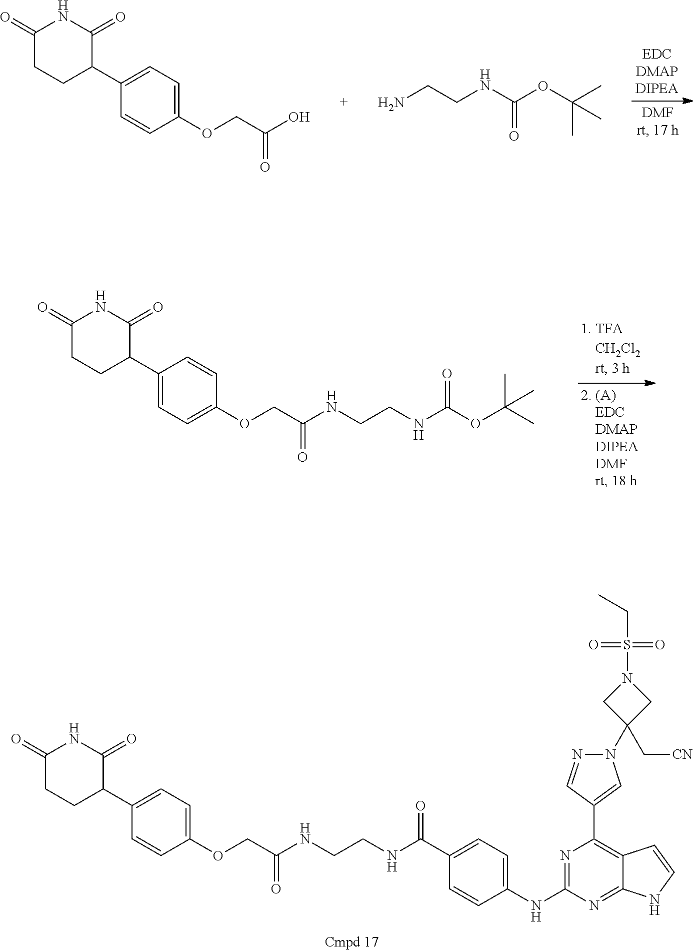 [SMILES: O=C(O)COc1ccc(C2CCC(=O)NC2=O)cc1 ; CC(C)(C)OC(=O)NCCN ; CC(C)(C)OC(=O)NCCNC(=O)COc1ccc(C2CCC(=O)NC2=O)cc1 ; CCS(=O)(=O)N1CC(CC#N)(n2cc(-c3nc(Nc4ccc(C(=O)NCCNC(=O)COc5ccc(C6CCC(=O)NC6=O)cc5)cc4)nc4[nH]ccc34)cn2)C1]

(c) Compound 18

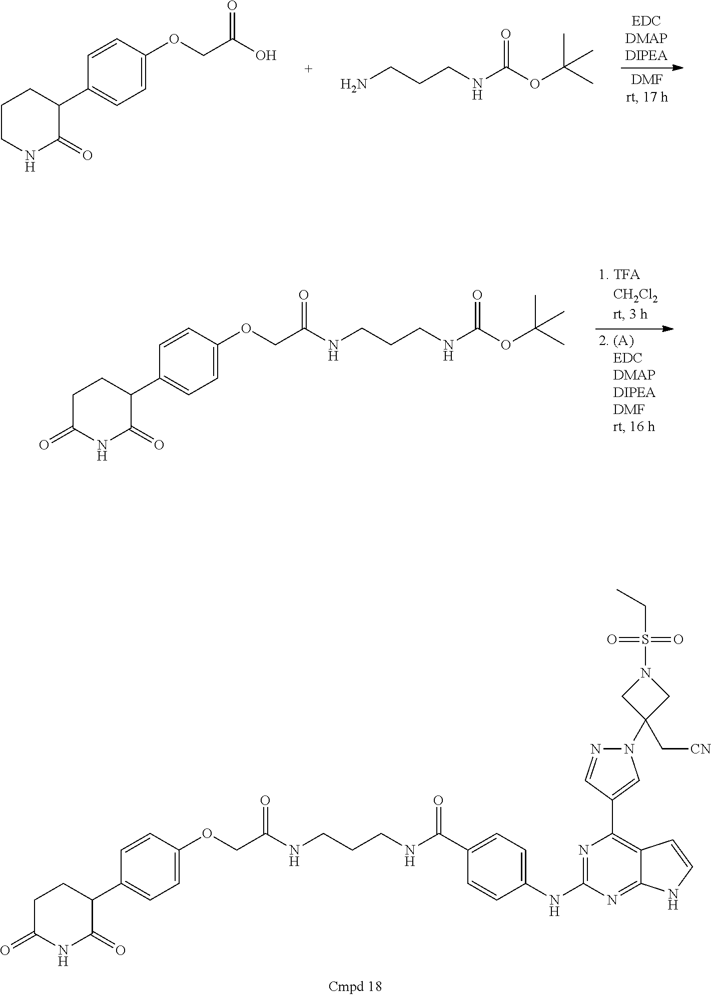 [SMILES: O=C(O)COc1ccc(C2CCCNC2=O)cc1 ; CC(C)(C)OC(=O)NCCCN ; CC(C)(C)OC(=O)NCCCNC(=O)COc1ccc(C2CCC(=O)NC2=O)cc1 ; CCS(=O)(=O)N1CC(CC#N)(n2cc(-c3nc(Nc4ccc(C(=O)NCCCNC(=O)COc5ccc(C6CCC(=O)NC6=O)cc5)cc4)nc4[nH]ccc34)cn2)C1]

k. Synthesis of Compounds 21, 24, 25, and 26

(a) Compound 21

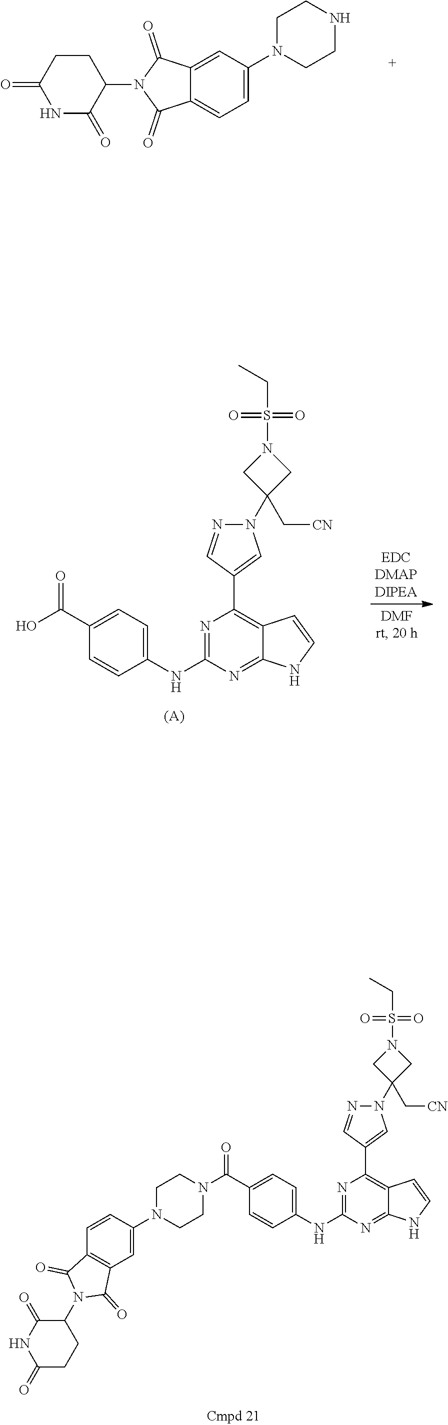

(b) Compound 24

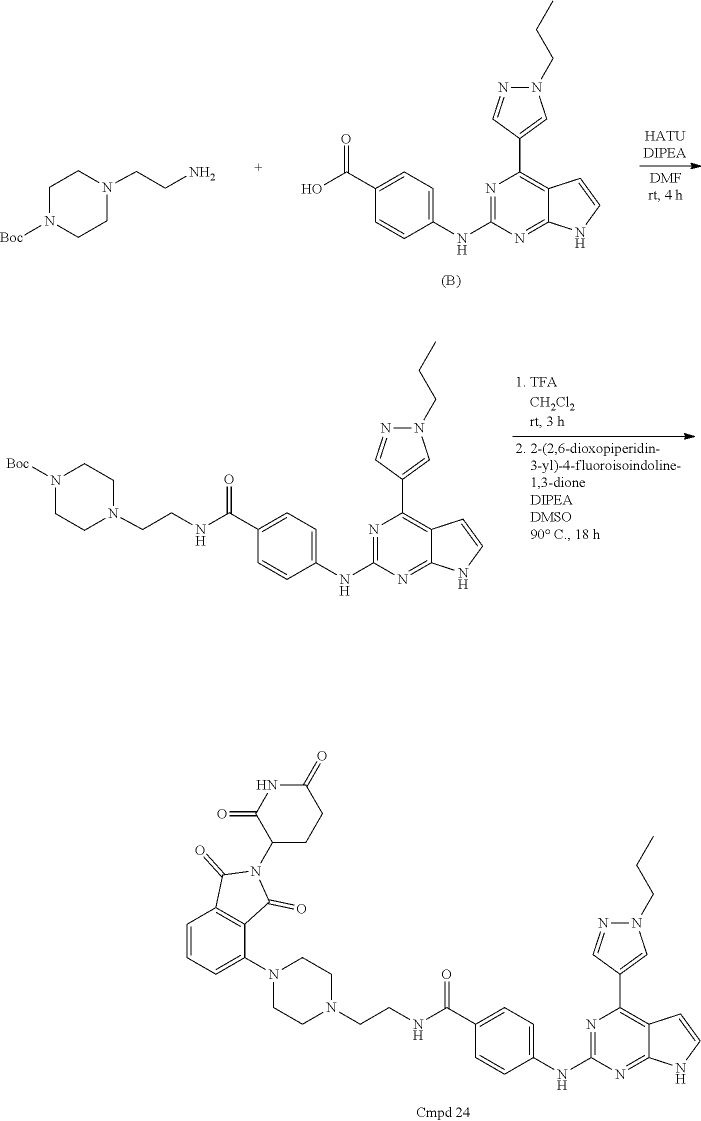

(c) Compound 25

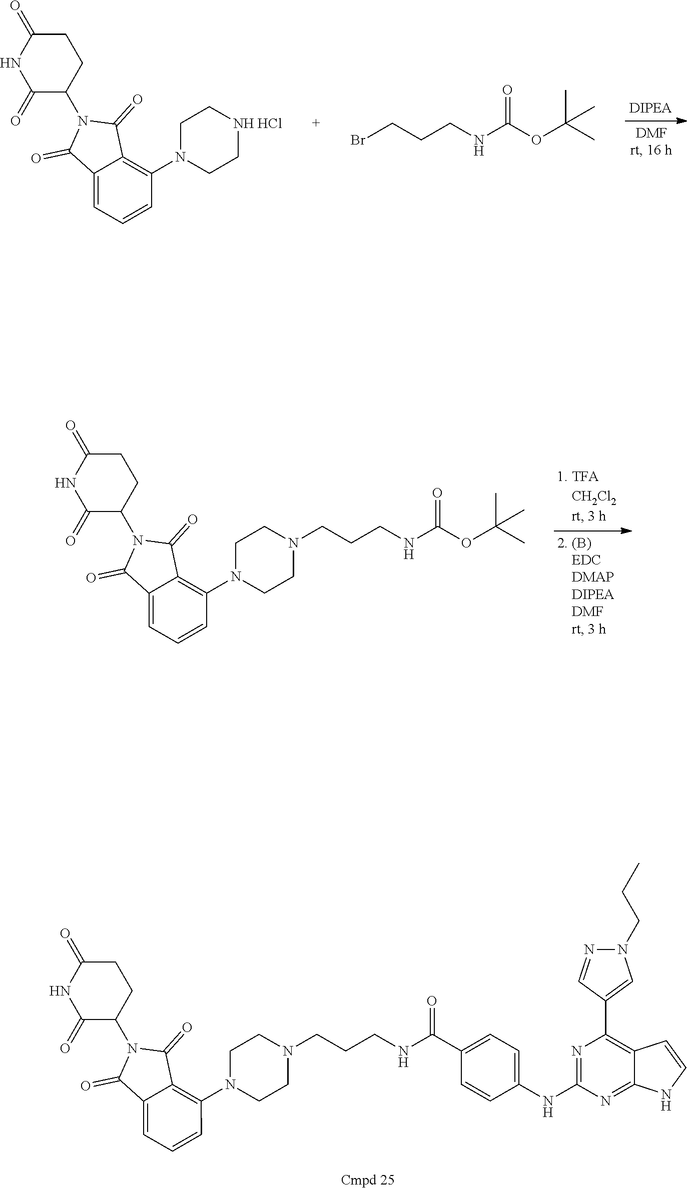

(d) Compound 26

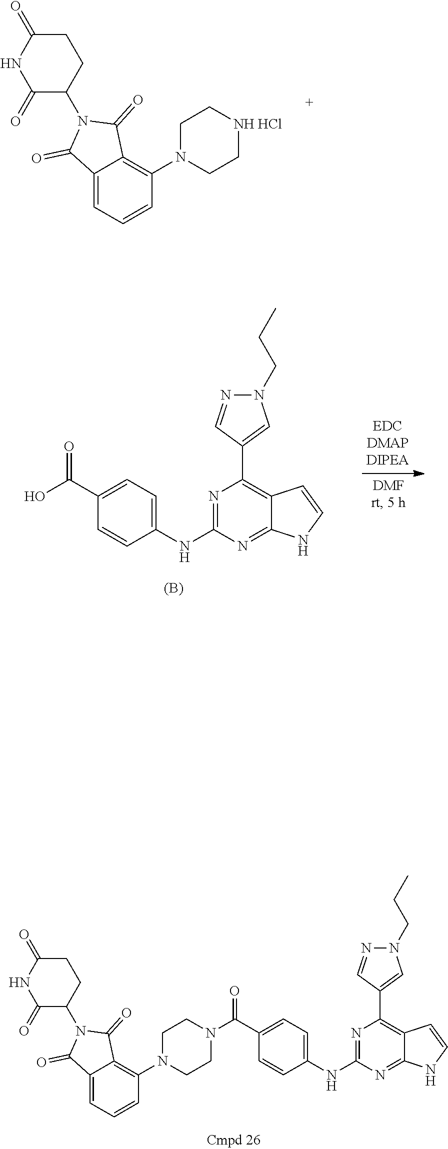

1. Synthesis of Compound 35

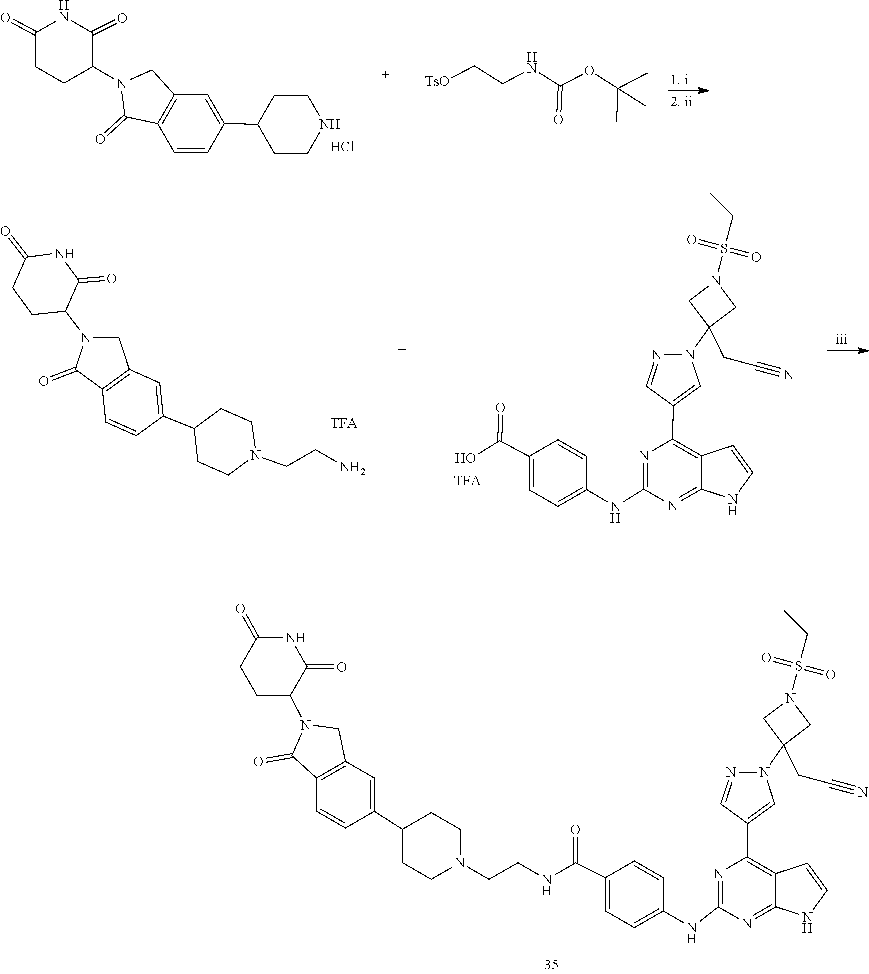 [SMILES: O=C1CCC(N2Cc3cc(C4CCNCC4)ccc3C2=O)C(=O)N1 ; Cl ; Cc1ccc(S(=O)(=O)OCCNC(=O)OC(C)(C)C)cc1 ; NCCN1CCC(c2ccc3c(c2)CN(C2CCC(=O)NC2=O)C3=O)CC1 ; CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)NCCN5CCC(c6ccc7c(c6)CN(C6CCC(=O)NC6=O)C7=O)CC5)cc4)nc4[nH]ccc34)cn2)C1]

Step i. Preparation of tert-butyl (2-(4-(2-(2,6-dioxopiperidin-3-yl)-1-oxoisoindolin-5-yl)piperidin-1-yl)ethyl)carbamate. To a flask was added 3-(1-oxo-5-(piperidin-4-yl)isoindolin-2-yl)piperidine-2,6-dione (46 mg, 0.141 mmol), 2-((tert-butoxycarbonyl)amino)ethyl 4-methylbenzenesulfonate (44.3 mg, 0.141 mmol), potassium carbonate (58.3 mg, 0.422 mmol) and dissolved in DMF (0.5 mL) and heated to 60° C. with stirring overnight. The reaction mixture was cooled, diluted with H₂O and extracted twice with EtOAc. The organic layer was collected, washed with brine, dried over NaSO₄ and evaporated under reduced pressure. The crude product was purified using biotage Sfar 11 g KP-amino D column (0-8% MeOH in DCM) to yield the compound as a solid (8.5 mg, 13%). ¹H NMR (500 MHz, CDCl₃) δ 7.91 (s, 1H), 7.75 (d, J=8.2 Hz, 1H), 7.31-7.26 (m, 2H), 5.15 (dd, J=13.3, 5.1 Hz, 1H), 4.40 (d, J=15.9 Hz, 1H), 4.26 (d, J=15.9 Hz, 1H), 3.28 (s, 2H), 2.90-2.82 (m, 1H), 2.77 (m, 1H), 2.60 (s, 2H), 2.30 (m, 1H), 2.15 (m, 1H), 1.82 (s, 2H), 1.60 (broad, 2H), 1.51 (d, J=10.9 Hz, 1H), 1.48 (d, J=3.7 Hz, 1H), 1.40 (s, 9H). ¹³C NMR (126 MHz, CDCl₃) δ 171.02, 170.98, 169.53, 169.50, 169.29, 156.12, 151.19, 141.90, 129.61, 127.45, 124.19, 121.19, 78.83, 57.09, 54.06, 51.80, 46.95, 43.07, 40.08, 33.48, 31.59, 28.48, 26.43, 23.46. LCMS (m/z) M+H=471.44.

Step ii: Preparation of 3-(5-(1-(2-aminoethyl)piperidin-4-yl)-1-oxoisoindolin-2-yl)piperidine-2,6-dione. To a flask was added tert-butyl (2-(4-(2-(2,6-dioxopiperidin-3-yl)-1-oxoisoindolin-5-yl)piperidin-1-yl)ethyl)carbamate (8.5 mg, 0.018 mmol) and dissolved in DCM (1 mL) before adding TFA (0.3 mL) with stirring at rt. The reaction was left for 1 h at rt with monitoring by LCMS. The reaction mixture was then concentrated under reduced pressure to yield the crude product as a yellow solid and used without further purification. LCMS (m/z) M+H=371.33.

Step iii: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(2-(4-(2-(2,6-dioxopiperidin-3-yl)-1-oxoisoindolin-5-yl)piperidin-1-yl)ethyl)benzamide. To a vial was added 3-(5-(1-(2-aminoethyl)piperidin-4-yl)-1-oxoisoindolin-2-yl)piperidine-2,6-dione (6.7 mg, 0.018 mmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (15 mg, 0.030 mmol), HATU (13.75 mg, 0.036 mmol) then dissolved in DMF (0.5 ml), before adding DIPEA (50 μl, 0.286 mmol) and stirring at rt overnight with monitoring by LCMS. The reaction mixture was directly purified using biotage Sfar C18 12 g column (5-50% MeCN in H₂O with 0.1% formic acid) to yield the compound as an off-white solid (5.1 mg, 33%). ¹H NMR (500 MHz, DMSO) δ 11.69 (t, J=2.2 Hz, 1H), 10.98 (s, 1H), 9.55 (s, 1H), 8.86 (s, 1H), 8.43 (s, 1H), 8.30 (s, 1H), 8.19 (t, J=5.7 Hz, 1H), 8.04-7.95 (m, 2H), 7.80 (d, J=8.8 Hz, 2H), 7.65 (d, J=7.9 Hz, 1H), 7.50 (s, 1H), 7.41 (dd, J=7.9, 1.4 Hz, 1H), 7.33 (dd, J=3.7, 2.2 Hz, 1H), 6.95 (dd, J=3.6, 1.8 Hz, 1H), 5.10 (dd, J=13.3, 5.1 Hz, 1H), 4.61 (d, J=9.1 Hz, 2H), 4.43 (d, J=17.2 Hz, 1H), 4.26 (d, J=9.4 Hz, 3H), 3.72 (s, 2H), 3.26 (q, J=7.4 Hz, 3H), 3.09-3.03 (m, 2H), 2.96 (s, 1H), 2.94-2.86 (m, 1H), 2.69-2.53 (m, 3H), 2.43-2.33 (m, 1H), 2.13 (td, J=11.7, 2.7 Hz, 2H), 1.99 (m, 1H), 1.78 (s, 2H), 1.77-1.72 (m, 1H), 1.71 (s, 1H), 1.26 (t, J=7.3 Hz, 3H). ¹³C NMR (126 MHz, DMSO) δ 173.38, 171.57, 168.51, 166.38, 164.89, 155.75, 154.07, 151.17, 150.59, 144.81, 142.95, 140.28, 130.22, 129.90, 128.32, 127.38, 126.25, 124.62, 123.40, 122.73, 122.20, 117.18, 108.51, 100.81, 59.05, 57.95, 56.54, 54.26, 52.00, 47.58, 43.80, 42.69, 41.68, 37.46, 33.55, 31.70, 27.28, 22.99, 7.91. LCMS (m/z) M+H=859.54.

m. Synthesis of Compound 36

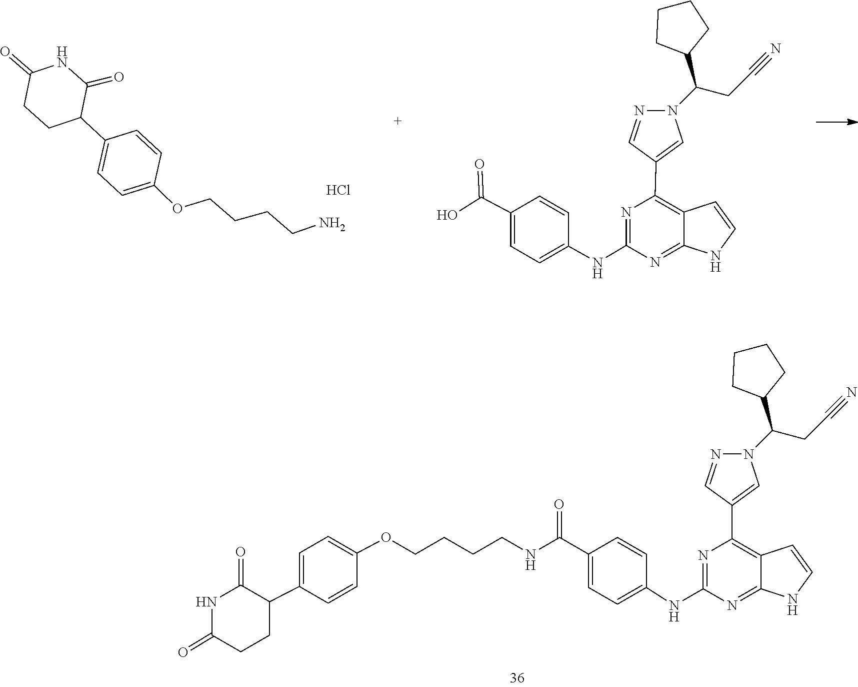 [SMILES: NCCCCOc1ccc(C2CCC(=O)NC2=O)cc1 ; Cl ; N#CC[C@H](C1CCCC1)n1cc(-c2nc(Nc3ccc(C(=O)O)cc3)nc3[nH]ccc23)cn1 ; N#CC[C@H](C1CCCC1)n1cc(-c2nc(Nc3ccc(C(=O)NCCCCOc4ccc(C5CCC(=O)NC5=O)cc4)cc3)nc3[nH]ccc23)cn1]

Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(2-(4-(2-(2,6-dioxopiperidin-3-yl)-1-oxoisoindolin-5-yl)piperidin-1-yl)ethyl)benzamide. To a vial was added (R)-4-((4-(1-(2-cyano-1-cyclopentylethyl)-1H-pyrazol-4-yl)-7H-pyrrolo[2,3-d]pyrimidin-2-yl)amino)benzoic acid (15 mg, 0.034 mmol), 3-(4-(4-aminobutoxy)phenyl)piperidine-2,6-dione (10.33 mg, 0.037 mmol), HATU (19.38 mg, 0.051 mmol) then dissolved in DMF (0.5 ml), before adding DIPEA (0.036 ml, 0.204 mmol) and stirring at rt overnight with monitoring by LCMS. The reaction mixture was directly purified using biotage Sfar C18 12 g column (5-70% MeCN in H₂O with 0.1% formic acid) to yield the compound as an off-white solid (6 mg, 25%). ¹H NMR (500 MHz, DMSO) δ 11.65 (t, J=2.1 Hz, 1H), 10.79 (s, 1H), 9.49 (s, 2H), 8.73 (s, 1H), 8.34 (s, 1H), 8.27 (q, J=4.6, 3.6 Hz, 1H), 8.06-7.90 (m, 2H), 7.89-7.73 (m, 2H), 7.30 (dd, J=3.6, 2.2 Hz, 1H), 7.17-7.04 (m, 2H), 6.98-6.75 (m, 3H), 4.58 (td, J=9.6, 4.2 Hz, 1H), 4.00 (t, J=6.3 Hz, 2H), 3.78 (dd, J=11.4, 4.9 Hz, 1H), 3.29-3.16 (m, 2H), 2.65 (ddd, J=17.1, 11.7, 5.3 Hz, 2H), 2.49-2.38 (m, 1H), 2.16 (dtd, J=13.2, 11.6, 4.4 Hz, 2H), 2.01 (dq, J=13.2, 4.9 Hz, 2H), 1.89-1.73 (m, 2H), 1.72-1.60 (m, 2H), 1.62-1.51 (m, 2H), 1.51-1.39 (m, 2H), 1.42-1.27 (m, 2H), 1.21 (dq, J=12.7, 8.3 Hz, 1H). ¹³C NMR (126 MHz, DMSO) δ 174.97, 173.92, 166.44, 158.02, 155.77, 154.01, 151.12, 144.83, 139.72, 131.45, 131.40, 130.02, 128.32, 126.26, 124.45, 121.02, 118.70, 117.12, 114.73, 108.27, 100.61, 67.63, 62.91, 46.97, 44.80, 31.83, 29.58, 26.79, 26.51, 26.48, 25.45, 24.82, 23.05. LCMS (m/z) M+H=700.35.

n. Synthesis of Compound 37

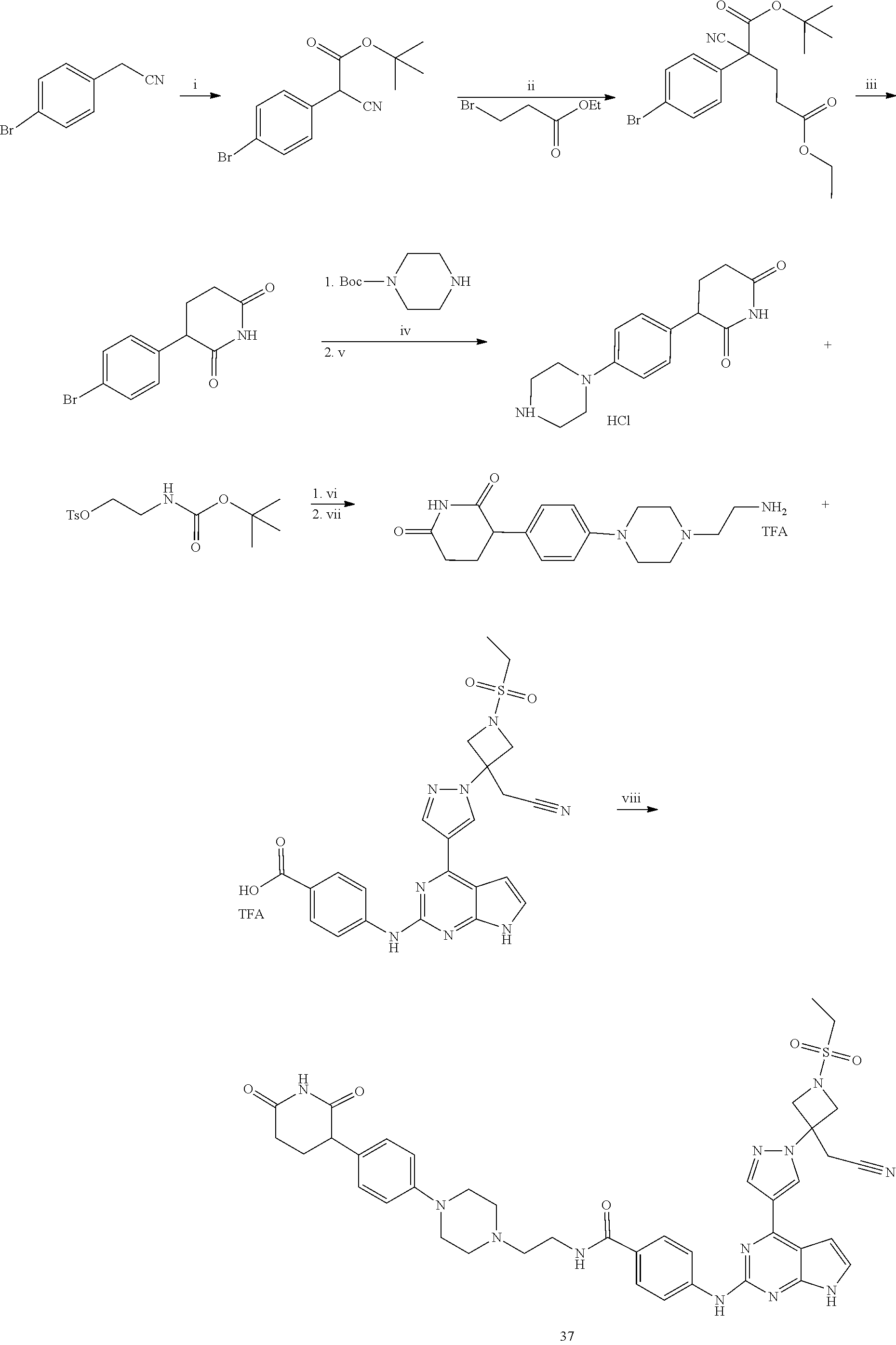

Step i: Preparation of tert-butyl 2-(4-bromophenyl)-2-cyanoacetate. To a solution of 2-(4-bromophenyl)acetonitrile (30 g, 153 mmol) in THF (200 mL) was added drop wise LDA (2 M, 191 mL) at −65° C. and stirred at −65° C. for 30 min. Then a solution of (Boc)₂O (36.7 g, 168 mmol) in THF (150 mL) was added drop wise to the reaction and the suspension was stirred at −65° C. for 1 h. The reaction solution was poured into water (500 mL) and stirred for 30 min. The aqueous phase was extracted with ethyl acetate (2×200 mL). The combined organic phase was washed with brine (150 mL), dried with anhydrous Na₂SO₄, filtered and concentrated under reduced pressure to give the crude product as a brown oil (70 g, crude) and was used without further purification. ¹H NMR (400 MHz CDCl₃) (7.50-7.54 (m, 2H), 7.29-7.32 (m, 2H), 4.52 (s, 1H), 1.44 (s, 9H).

Step ii: Preparation of 1-(tert-butyl) 5-ethyl 2-(4-bromophenyl)-2-cyanopentanedioate. To a mixture of tert-butyl 2-(4-bromophenyl)-2-cyanoacetate (70 g, 236 mmol) in MeCN (420 mL) was added K₂CO₃ (65.3 g, 473 mmol), TEBAC (5.38 g, 23.6 mmol) and ethyl 3-bromopropanoate (64.1 g, 355 mmol) and stirred at 75° C. for 6 h. The reaction suspension was cooled to 20° C. and filtered. The filtrate was concentrated under reduced pressure and the residue dissolved in EtOAc (300 mL) and washed with water (2×300 mL) and brine (300 mL). The organic phase was dried with anhydrous Na₂SO₄, filtered and concentrated under reduced pressure to yield the crude product as a brown oil (72 g). The crude was used without further purification. ¹H NMR (400 MHz CDCl₃) δ 7.54-7.57 (m, 2H), 7.41-7.43 (m, 2H), 4.13 (q, J=14.4 Hz, 2H), 2.64-2.67 (m, 1H), 2.36-2.50 (m, 4H), 1.43 (s, 9H), 1.18-1.29 (m, 6H).

Step iii: Preparation of 3-(4-bromophenyl)piperidine-2,6-dione. To a solution of 1-(tert-butyl) 5-ethyl 2-(4-bromophenyl)-2-cyanopentanedioate (33 g, 83.3 mmol) in HOAc (200 mL) was added H₂SO₄ (3.62 mL, 66.6 mmol) and stirred at 120° C. for 3 h with monitoring by LCMS. The reaction solution was cooled to 20° C., poured into water (800 mL) and stirred for 30 min. The solid was collected by filtration and washed with water (2×200 mL). The crude was triturated with MeOH (80 mL) and stirred for 30 min. The solid was collected by filtration and dried under vacuum to give the product as a white solid (14 g, 62.0%). ¹H NMR (400 MHz DMSO-d₆) δ 10.9 (s, 1H), 7.51-7.54 (m, 2H), 7.19-7.21 (m, 2H), 3.88 (dd, J=12.0 Hz, 4.8 Hz, 1H), 2.63-2.67 (m, 1H), 2.52-2.53 (m, 1H), 2.18-2.22 (m, 1H), 2.02-2.03 (m, 1H).

Step iv: Preparation of tert-butyl 4-(4-(2,6-dioxopiperidin-3-yl)phenyl)piperazine-1-carboxylate. To a solution of 3-(4-bromophenyl)piperidine-2,6-dione (14 g, 52.2 mmol) in DMF (280 mL) was added tert-butyl piperazine-1-carboxylate (19.5 g, 104 mmol), Cs₂CO₃ (68.1 g, 209 mmol) and RuPhos Pd G3 (4.37 g, 5.22 mmol). The mixture was stirred at 100° C. for 24 h under an atmosphere of N₂. The reaction solution was cooled to 20° C., poured into water (500 mL) and extracted with EtOAc (3×100 mL). The combined organic layers were washed with brine (100 mL), dried over Na₂SO₄, filtered and concentrated under reduced pressure to give a residue. The residue was purified by column chromatography (Ethyl acetate 1-5% in hexanes) to yield the product as a white solid (2.75 g, 13.4%). ¹H NMR (400 MHz DMSO-d₆) δ 10.7 (s, 1H), 7.06-7.08 (m, 2H), 6.90-6.92 (m, 2H), 3.73 (q, J=5.2 Hz, 1H), 3.44-3.46 (m, 4H), 3.06-3.08 (m, 4H), 2.60-2.63 (m, 1H), 2.48 (m, 1H), 1.99-2.02 (m, 1H), 1.42 (s, 9H).

Step v: Preparation of 3-(4-(piperazin-1-yl)phenyl)piperidine-2,6-dione. To a solution of tert-butyl 4-(4-(2,6-dioxopiperidin-3-yl)phenyl)piperazine-1-carboxylate (2.65 g, 7.10 mmol) in EtOAc (40 mL) was added HCl/EtOAc (4 M, 40 mL) at rt. The reaction suspension was stirred at rt for 12 h with monitoring by LCMS. The reaction mixture was filtered and solid collected and used without further purification as a white solid (2.3 g, 93.3%). ¹H NMR (400 MHz DMSO-d₆) (10.8 (s, 1H), 9.30 (s, 2H), 7.11-7.13 (m, 2H), 6.96-6.98 (m, 2H), 3.77 (q, J=5.2 Hz, 1H), 3.36-3.38 (m, 4H), 3.21 (s, 4H), 2.62-2.68 (m, 1H), 2.45 (m, 1H), 2.13-2.14 (m, 1H), 1.99-2.03 (m, 1H). LCMS (m/z) M+H=274.1.

Step iv: Preparation of tert-butyl (2-(4-(4-(2,6-dioxopiperidin-3-yl)phenyl)piperazin-1-yl)ethyl)carbamate. To a vial was added 3-(4-(piperazin-1-yl)phenyl)piperidine-2,6-dione (92 mg, 0.34 mmol), potassium carbonate (140 mg, 1 mmol) and 2-((tert-butoxycarbonyl)amino)ethyl 4-methylbenzenesulfonate (94 mg, 0.30 mmol) and dissolved in DMF (1 ml) and heated to 60° C. with stirring overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar 10 g silica HC D column (0-5% MeOH in DCM) to yield the compound as a white solid (11.8 mg, 8%). ¹H NMR (500 MHz, CDCl₃) δ 8.12 (s, 1H), 7.03 (d, J=8.3 Hz, 2H), 6.84 (d, J=8.7 Hz, 2H), 5.06 (s, 1H), 3.65 (dd, J=9.5, 5.2 Hz, 1H), 3.25 (s, 2H), 3.17 (s, 4H), 2.76-2.42 (m, 8H), 2.16 (m, 2H), 1.39 (s, 9H). ¹³C NMR (126 MHz, CDCl₃) δ 173.56, 172.46, 156.01, 150.49, 128.76, 127.85, 116.32, 79.37, 57.30, 52.86, 48.57, 47.07, 36.94, 30.85, 28.45, 26.33. LCMS (m/z) M+H=417.47.

Step vii: Preparation of 3-(4-(4-(2-aminoethyl)piperazin-1-yl)phenyl)piperidine-2,6-dione. To a vial was added tert-butyl (2-(4-(4-(2,6-dioxopiperidin-3-yl)phenyl)piperazin-1-yl)ethyl)carbamate (11.8 mg, 0.028 mmol) and dissolved in DCM (1 mL) before adding TFA (0.3 mL) with stirring at rt for 4 h with monitoring by LCMS. The reaction mixture was concentrated under reduced pressure and used without further purification. LCMS (m/z) M+H=317.29.

Step viii: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(2-(4-(4-(2,6-dioxopiperidin-3-yl)phenyl)piperazin-1-yl)ethyl)benzamide. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (15 mg, 0.03 mmol), 3-(4-(4-(2-aminoethyl)piperazin-1-yl)phenyl)piperidine-2,6-dione (8.9 mg, 0.028 mmol), HATU (16 mg, 0.04 mmol) and dissolved in DMF (0.5 ml) before adding DIPEA (29 ul, 0.17 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (6.5 mg, 29%). ¹H NMR (500 MHz, DMSO) δ 11.69 (s, 1H), 10.78 (s, 1H), 9.55 (s, 1H), 8.85 (s, 1H), 8.43 (s, 1H), 8.30-8.12 (m, 1H), 7.98 (d, J=8.9 Hz, 2H), 7.80 (d, J=8.8 Hz, 2H), 7.32 (dd, J=3.7, 2.2 Hz, 1H), 7.05 (d, J=8.7 Hz, 2H), 6.94 (dd, J=3.7, 1.8 Hz, 1H), 6.90 (d, J=8.8 Hz, 2H), 4.61 (d, J=9.1 Hz, 1H), 4.26 (d, J=9.1 Hz, 1H), 3.79-3.67 (m, 2H), 3.43 (m, overlapping peaks), 3.26 (q, J=7.4 Hz, overlapping peaks), 3.14 (t, J=5.1 Hz, 2H), 2.70-2.57 (m, 1H), 2.51 (m, overlapping peaks), 2.20-2.04 (m, 2H), 2.01 (m, 2H), 1.26 (t, J=7.4 Hz, 3H). ¹³C NMR (126 MHz, DMSO) δ 174.28, 173.16, 165.60, 163.49, 154.94, 153.26, 149.79, 149.63, 144.01, 139.47, 129.10, 128.86, 128.63, 127.52, 125.43, 123.81, 121.92, 116.37, 114.94, 107.70, 100.00, 58.25, 56.83, 55.73, 52.48, 47.99, 46.10, 42.98, 36.44, 30.92, 26.48, 25.65, 7.11. HRMS (ESI) Exact mass calcd for C₄₀H₄₅N₁₂O₅S [M+H]⁺ 805.3356, found 805.3333.

o. Synthesis of Compound 38

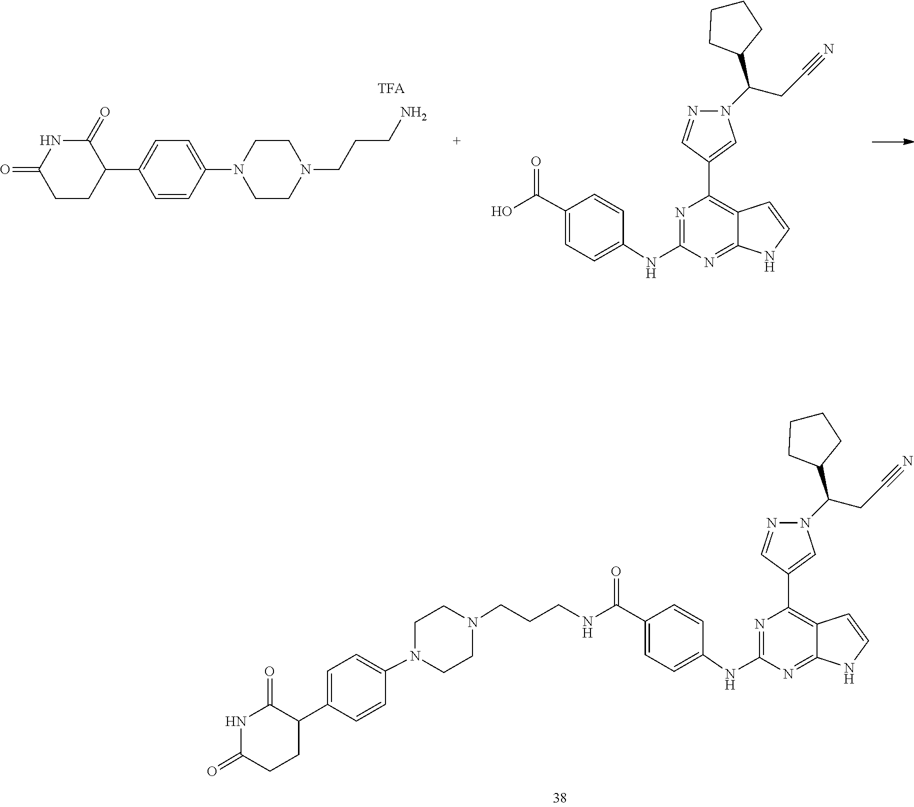 [SMILES: NCCCN1CCN(c2ccc(C3CCC(=O)NC3=O)cc2)CC1 ; N#CC[C@H](C1CCCC1)n1cc(-c2nc(Nc3ccc(C(=O)O)cc3)nc3[nH]ccc23)cn1 ; N#CC[C@H](C1CCCC1)n1cc(-c2nc(Nc3ccc(C(=O)NCCCN4CCN(c5ccc(C6CCC(=O)NC6=O)cc5)CC4)cc3)nc3[nH]ccc23)cn1]

Preparation of 4-((4-(1-((R)-2-cyano-1-cyclopentylethyl)-1H-pyrazol-4-yl)-7H-pyrrolo[2,3-d]pyrimidin-2-yl)amino)-N-(3-(4-(4-(2,6-dioxopiperidin-3-yl)phenyl)piperazin-1-yl)propyl)benzamide. To a vial was added 4-((4-(1-(2-cyano-1-cyclopentylethyl)-1H-pyrazol-4-yl)-7H-pyrrolo[2,3-d]pyrimidin-2-yl)amino)benzoic acid (15 mg, 0.034 mmol), 3-(4-(4-(3-aminopropyl)piperazin-1-yl)phenyl)piperidine-2,6-dione (15.6 mg, 0.047 mmol), HATU (19.38 mg, 0.051 mmol) and dissolved in DMF (0.5 ml) before adding DIPEA (0.036 ml, 0.204 mmol) with stirring at rt overnight with monitoring by LCMS. The crude reaction mixture was directly purified using biotage Sfar 12 g C18 column (5-50% MeCN in water (0.1% formic acid)) to yield the product as a white solid (7.8 mg, 31%). ¹H NMR (500 MHz, DMSO) δ 11.56 (t, J=2.1 Hz, 1H), 10.71 (s, 1H), 9.43 (s, 1H), 8.66 (s, 1H), 8.27 (s, 1H), 8.07 (s, 1H), 7.91 (d, J=8.7 Hz, 2H), 7.73 (d, J=8.7 Hz, 2H), 7.23 (dd, J=3.6, 2.3 Hz, 1H), 6.99 (d, J=8.2 Hz, 2H), 6.85 (d, J=8.2 Hz, 2H), 6.77 (dd, J=3.6, 1.8 Hz, 1H), 6.46 (s, 1H), 4.51 (m, 1H), 3.66 (dd, J=11.1, 4.9 Hz, 1H), 3.23-3.10 (m, 2H), 2.62-2.51 (overlapping peaks, 2H), 2.43-2.29 (overlapping peaks, 2H), 2.12-1.99 (m, 2H), 1.92 (m, 2H), 1.76 (m, 3H), 1.69 (s, 1H), 1.63-1.50 (m, 2H), 1.50-1.42 (m, 2H), 1.42-1.33 (m, 2H), 1.26 (m, 2H), 1.15 (m, 2H). ¹³C NMR (126 MHz, DMSO) δ 175.05, 173.94, 166.58, 163.52, 155.75, 154.00, 151.11, 144.92, 139.72, 131.45, 129.51, 128.33, 126.12, 124.48, 121.01, 118.71, 117.13, 115.94, 108.28, 100.62, 62.92, 46.91, 44.80, 31.75, 29.59, 26.43, 25.45, 24.82, 23.05. LCMS (m/z) M+H=754.11.

p. Synthesis of Compound 39

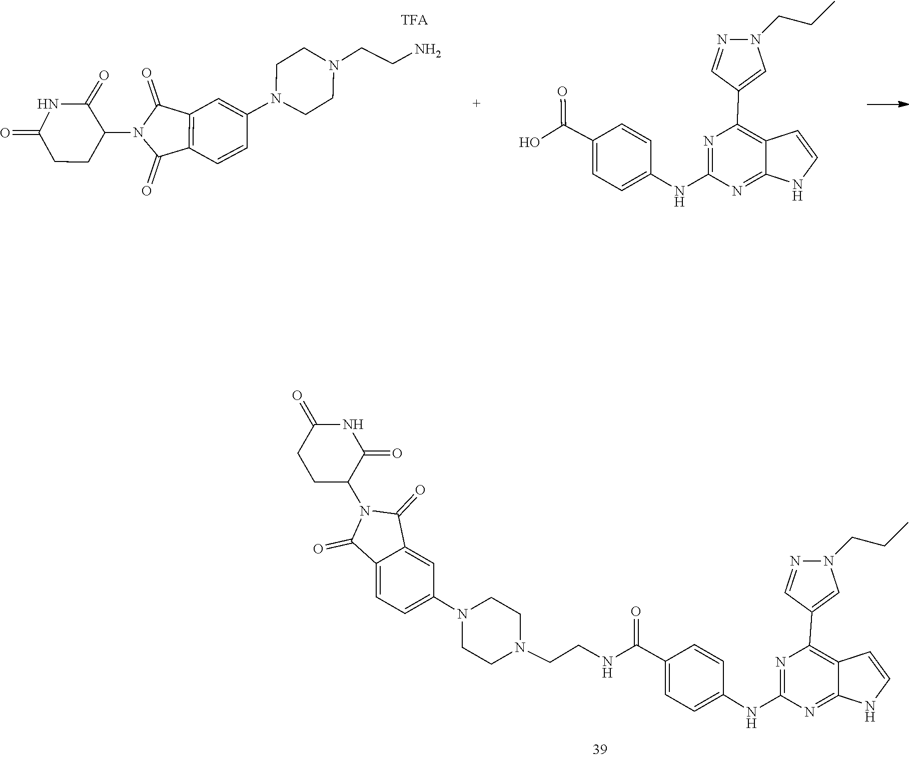 [SMILES: NCCN1CCN(c2ccc3c(c2)C(=O)N(C2CCC(=O)NC2=O)C3=O)CC1 ; CCCn1cc(-c2nc(Nc3ccc(C(=O)O)cc3)nc3[nH]ccc23)cn1 ; CCCn1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1]

Preparation of N-(2-(4-(2-(2,6-dioxopiperidin-3-yl)-1,3-dioxoisoindolin-5-yl)piperazin-1-yl)ethyl)-4-((4-(1-propyl-1H-pyrazol-4-yl)-7H-pyrrolo[2,3-d]pyrimidin-2-yl)amino)benzamide. To a vial was added 5-(4-(2-aminoethyl)piperazin-1-yl)-2-(2,6-dioxopiperidin-3-yl)isoindoline-1,3-dione (15.95 mg, 0.041 mmol), 4-((4-(1-propyl-1H-pyrazol-4-yl)-7H-pyrrolo[2,3-d]pyrimidin-2-yl)amino)benzoic acid (15 mg, 0.041 mmol), HATU (23.61 mg, 0.062 mmol) then dissolved in DMF (0.5 ml), before adding DIPEA (0.043 ml, 0.248 mmol) and stirring at rt overnight with monitoring by LCMS. The reaction mixture was directly purified using biotage Sfar C18 12 g column (5-50% MeCN in H₂O with 0.1% formic acid) to yield the compound as an off-white solid (13.7 mg, 45%). ¹H NMR (500 MHz, DMSO) δ 11.60 (d, J=2.2 Hz, 1H), 11.08 (s, 1H), 9.47 (s, 1H), 8.59 (s, 1H), 8.22 (d, J=13.6 Hz, 2H), 7.97 (d, J=8.6 Hz, 2H), 7.79 (d, J=8.6 Hz, 2H), 7.67 (d, J=8.5 Hz, 1H), 7.35 (d, J=2.3 Hz, 1H), 7.29-7.23 (m, 2H), 6.81 (dd, J=3.7, 1.8 Hz, 1H), 5.07 (dd, J=12.8, 5.4 Hz, 1H), 4.19 (t, J=7.0 Hz, 2H), 3.44 (dd, J=15.9, 8.5 Hz, 4H), 2.96-2.82 (m, 1H), 2.62-2.50 (m, 6H), 2.01 (ddq, J=13.0, 5.6, 3.5, 2.5 Hz, 1H), 1.87 (h, J=7.2 Hz, 2H), 0.87 (t, J=7.3 Hz, 3H). ¹³C NMR (126 MHz, DMSO) δ 172.70, 169.98, 167.45, 166.87, 165.83, 155.15, 153.35, 150.77, 144.35, 138.48, 133.74, 130.76, 127.91, 127.72, 125.49, 125.37, 124.77, 123.71, 120.52, 118.18, 117.66, 116.53, 107.79, 107.62, 100.06, 56.91, 53.02, 52.23, 48.64, 46.79, 39.99, 39.90, 39.82, 39.73, 39.66, 39.57, 39.49, 39.40, 39.32, 39.23, 39.07, 38.90, 36.58, 30.86, 23.05, 22.06, 10.80, 10.73. ¹³C NMR (126 MHz, DMSO) δ 172.70, 169.98, 167.45, 166.87, 165.83, 155.15, 153.35, 150.77, 144.35, 138.48, 133.74, 130.76, 127.91, 127.72, 125.49, 125.37, 124.77, 123.71, 120.52, 118.18, 117.66, 116.53, 107.79, 107.62, 100.06, 56.91, 53.02, 52.23, 48.64, 46.79, 36.58, 30.86, 23.05, 22.06, 10.80. LCMS (m/z) M+H=730.24.

q. Synthesis of Compound 40

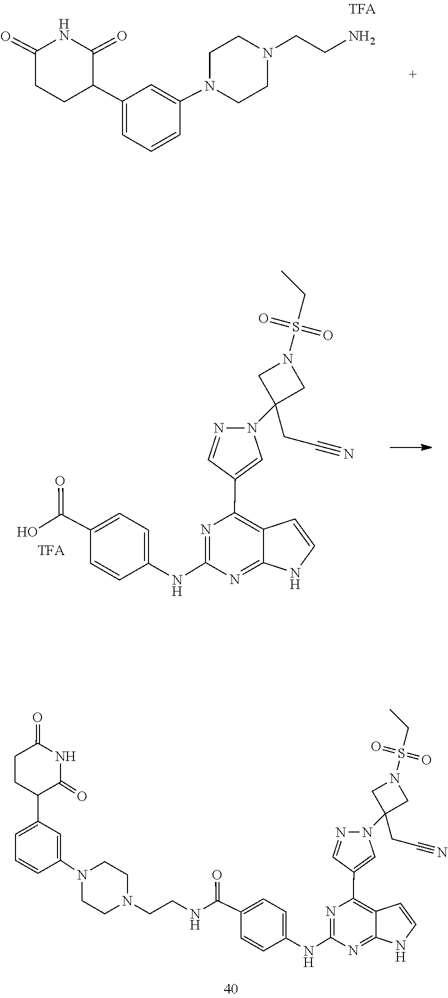 [SMILES: NCCN1CCN(c2cccc(C3CCC(=O)NC3=O)c2)CC1 ; CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)NCCN5CCN(c6cccc(C7CCC(=O)NC7=O)c6)CC5)cc4)nc4[nH]ccc34)cn2)C1]

Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(2-(4-(3-(2,6-dioxopiperidin-3-yl)phenyl)piperazin-1-yl)ethyl)benzamide. To a vial was added 3-(3-(4-(2-aminoethyl)piperazin-1-yl)phenyl)piperidine-2,6-dione hydrochloride (20 mg, 0.057 mmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (28.7 mg, 0.057 mmol), HATU (32.3 mg, 0.085 mmol) then dissolved in DMF (0.5 ml), before adding DIPEA (0.099 ml, 0.567 mmol) and stirring at rt overnight with monitoring by LCMS. The reaction mixture was directly purified using biotage Sfar C18 12 g column (5-50% MeCN in H₂O with 0.1% formic acid) to yield the compound as an off-white solid (8.9 mg, 20%). ¹H NMR (500 MHz, DMSO) δ 11.61 (s, 1H), 10.74 (s, 1H), 9.52 (s, 1H), 8.79 (s, 1H), 8.35 (s, 1H), 8.07 (s, 1H), 7.93 (d, J=8.3 Hz, 2H), 7.75 (d, J=8.5 Hz, 2H), 7.35-7.22 (m, 1H), 7.12 (t, J=7.7 Hz, 1H), 6.88 (dd, J=3.6, 1.9 Hz, 1H), 6.81 (d, J=14.3 Hz, 2H), 6.61 (s, 1H), 6.46 (s, 1H), 4.54 (d, J=9.2 Hz, 2H), 4.29-4.08 (m, 2H), 3.70 (dd, J=11.4, 5.0 Hz, 2H), 3.64 (s, 2H), 3.45-3.28 (m, 2H), 3.18 (q, J=7.4 Hz, 2H), 3.11-2.84 (m, 3H), 2.74-2.47 (m, 3H), 2.21-2.08 (m, 1H), 2.08-1.86 (m, 2H), 1.19 (t, J=7.3 Hz, 3H). ¹³C NMR (126 MHz, DMSO) δ 174.76, 173.93, 163.52, 155.69, 154.06, 150.56, 140.58, 140.28, 129.91, 129.40, 128.47, 124.70, 122.71, 117.17, 116.62, 108.57, 100.82, 59.04, 56.55, 48.16, 43.81, 31.82, 27.30, 26.48, 7.92. LCMS (m/z) M+H=805.15.

r. Synthesis of Compound 41

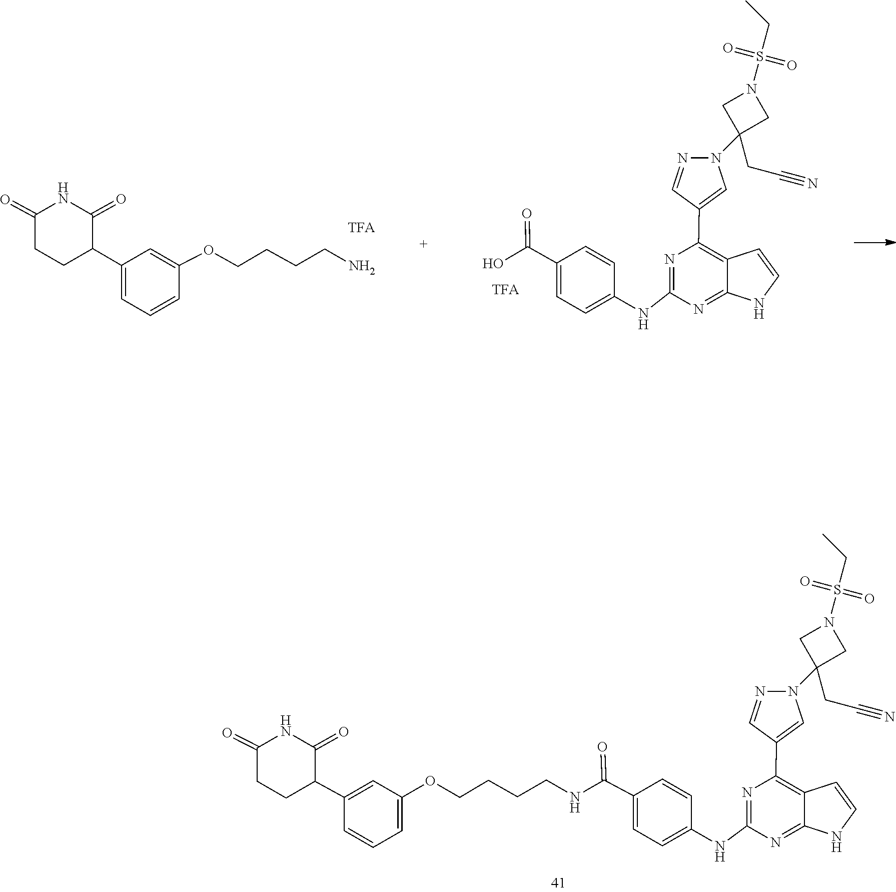 [SMILES: NCCCCOc1cccc(C2CCC(=O)NC2=O)c1 ; CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)NCCCCOc5cccc(C6CCC(=O)NC6=O)c5)cc4)nc4[nH]ccc34)cn2)C1]

Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(4-(3-(2,6-dioxopiperidin-3-yl)phenoxy)butyl)benzamide. To a vial was added 3-(3-(4-aminobutoxy)phenyl)piperidine-2,6-dione (20 mg, 0.072 mmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (36.7 mg, 0.072 mmol), HATU (41.3 mg, 0.109 mmol) and dissolved in DMF (0.5 ml) before adding DIPEA (0.126 ml, 0.724 mmol) with stirring at rt overnight with monitoring by LCMS. The crude reaction mixture was directly purified using biotage Sfar C18 12 g column (5-60% MeCN in H₂O with 0.1% formic acid) to yield the product as a white solid (10.4 mg, 19%). Purity 99% (UV/ELSD). NMR: ¹H NMR (500 MHz, DMSO) δ 11.68 (s, 1H), 10.82 (s, 1H), 9.53 (s, 1H), 8.85 (s, 1H), 8.42 (s, 1H), 8.27 (t, J=5.7 Hz, 1H), 7.97 (d, J=8.9 Hz, 2H), 7.80 (d, J=8.9 Hz, 2H), 7.32 (dd, J=3.6, 2.2 Hz, 1H), 7.23 (t, J=7.9 Hz, 1H), 6.94 (dd, J=3.6, 1.8 Hz, 1H), 6.84 (dd, J=7.8, 2.5 Hz, 1H), 6.81-6.72 (m, 2H), 4.60 (d, J=9.1 Hz, 1H), 4.25 (d, J=9.1 Hz, 1H), 3.99 (t, J=6.4 Hz, 2H), 3.81 (dd, J=11.4, 4.9 Hz, 1H), 3.71 (s, 1H), 3.25 (q, J=7.4 Hz, 2H), 2.64 (m, 2H), 2.20 (m, 2H), 2.02 (m, 2H), 1.76 (m, 2H), 1.68 (m, 2H), 1.25 (t, J=7.3 Hz, 3H). ¹³C NMR (126 MHz, DMSO) δ 174.14, 173.41, 165.95, 165.16, 158.61, 155.28, 153.61, 150.11, 144.29, 140.66, 139.80, 129.43, 129.31, 127.85, 125.87, 124.13, 122.26, 120.71, 116.71, 116.69, 115.00, 112.70, 108.03, 100.34, 67.08, 58.58, 56.07, 47.36, 43.31, 31.33, 26.81, 26.33, 26.04, 25.85, 7.44. HRMS (ESI) Exact mass calcd for C₃₈H₄₁N₁₀O₆S [M+H]⁺ 765.2931, found 765.2921.

s. Synthesis of Compound 42

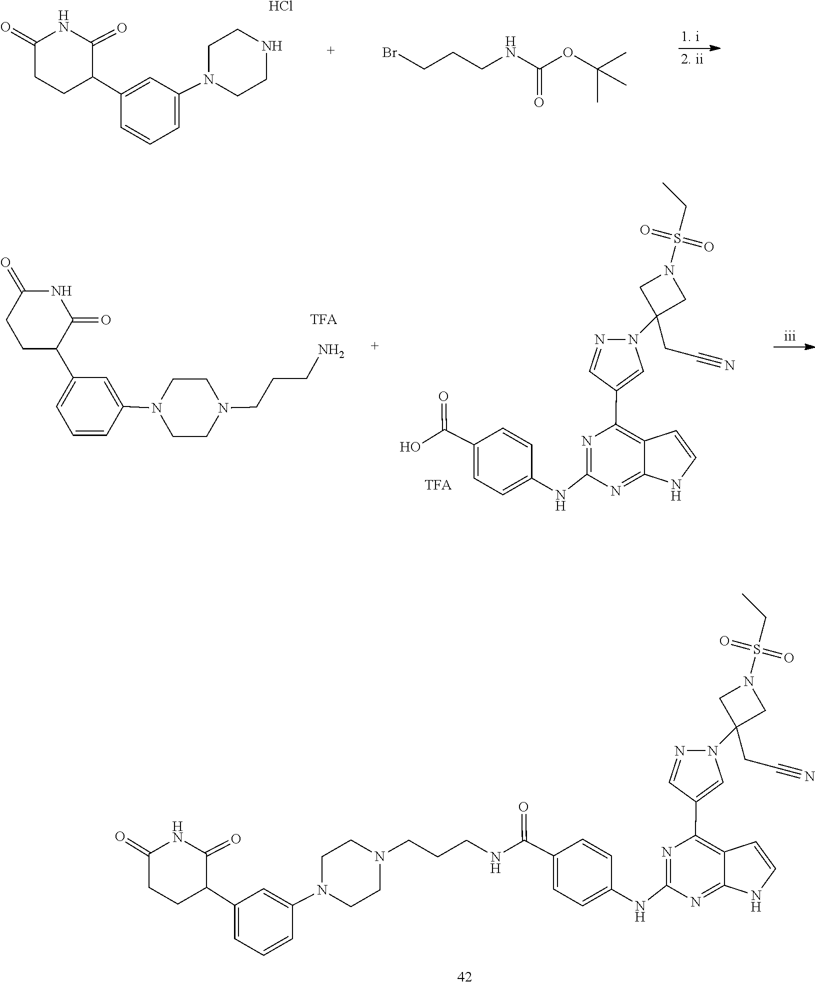 [SMILES: Cl ; O=C1CCC(c2cccc(N3CCNCC3)c2)C(=O)N1 ; CC(C)(C)OC(=O)NCCCBr ; NCCCN1CCN(c2cccc(C3CCC(=O)NC3=O)c2)CC1 ; CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)NCCCN5CCN(c6cccc(C7CCC(=O)NC7=O)c6)CC5)cc4)nc4[nH]ccc34)cn2)C1]

Step i: Preparation of tert-butyl (3-(4-(3-(2,6-dioxopiperidin-3-yl)phenyl)piperazin-1-yl)propyl)carbamate. To a vial was added 3-(3-(piperazin-1-yl)phenyl)piperidine-2,6-dione hydrochloride (30 mg, 0.097 mmol) and tert-butyl (3-bromopropyl)carbamate (23.06 mg, 0.097 mmol) and dissolved in DMF (1 ml) before adding DIPEA (0.068 ml, 0.387 mmol) with stirring and heating to 60° C. overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar 25 g silica HC D column (0-5% MeOH in DCM) to yield the compound as a brown solid (18.2 mg, 44%). ¹H NMR (500 MHz, CDCl₃) δ 8.24 (s, 1H), 7.26 (d, J=7.9 Hz, 1H), 6.88 (dd, J=8.3, 2.4 Hz, 1H), 6.76 (t, J=2.2 Hz, 1H), 6.71 (dd, J=7.4, 1.5 Hz, 1H), 5.30 (s, 1H), 3.76 (dd, J=9.2, 5.2 Hz, 1H), 3.24 (broad, 6H), 2.81-2.57 (m, 6H), 2.53 (t, J=7.0 Hz, 2H), 2.41-2.18 (m, 2H), 1.75 (p, J=6.6 Hz, 2H), 1.46 (s, 9H). ¹³C NMR (126 MHz, CDCl₃) δ 173.22, 172.40, 156.12, 151.59, 137.97, 129.69, 119.15, 115.96, 115.32, 79.02, 60.42, 56.54, 53.01, 48.78, 48.18, 39.63, 30.77, 28.47, 26.42. LCMS (m/z) M+H=431.33.

Step ii: Preparation of 3-(3-(4-(3-aminopropyl)piperazin-1-yl)phenyl)piperidine-2,6-dione. To a vial was added tert-butyl (3-(4-(3-(2,6-dioxopiperidin-3-yl)phenyl)piperazin-1-yl)propyl)carbamate (18.2 mg, 0.042 mmol) and dissolved in DCM (2 mL) before adding TFA (0.5 mL) with stirring at rt for 1 h with monitoring by LCMS. The reaction mixture was concentrated under reduced pressure and used without further purification. LCMS (m/z) M+H=331.22.

Step iii: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(3-(4-(3-(2,6-dioxopiperidin-3-yl)phenyl)piperazin-1-yl)propyl)benzamide. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (21.31 mg, 0.042 mmol), 3-(3-(4-(3-aminopropyl)piperazin-1-yl)phenyl)piperidine-2,6-dione (13.9 mg, 0.042 mmol), HATU (23.99 mg, 0.063 mmol) and dissolved in DMF (0.5 ml) before adding DIPEA (0.073 ml, 0.421 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (8.4 mg, 24%). ¹H NMR (500 MHz, DMSO) δ 11.68 (s, 1H), 10.80 (s, 1H), 9.55 (s, 1H), 8.86 (s, 1H), 8.43 (s, 1H), 8.14 (s, 1H), 7.98 (d, J=8.8 Hz, 2H), 7.80 (d, J=8.9 Hz, 2H), 7.33 (dd, J=3.6, 2.2 Hz, 1H), 7.17 (t, J=7.8 Hz, 1H), 6.95 (dd, J=3.7, 1.9 Hz, 1H), 6.90-6.75 (m, 1H), 6.64 (d, J=7.5 Hz, 1H), 6.54 (s, 1H), 4.61 (d, J=9.1 Hz, 2H), 4.26 (d, J=9.1 Hz, 1H), 3.84-3.60 (m, 2H), 3.30-3.04 (overlapping peaks, 4H), 2.82-2.54 (overlapping peaks, 4H), 2.46 (overlapping peaks, 2H), 2.36-2.09 (m, 2H), 2.09-1.91 (m, 1H), 1.78 (s, 2H), 1.26 (t, J=7.4 Hz, 3H). ¹³C NMR (126 MHz, DMSO) δ 174.79, 173.92, 166.52, 163.53, 155.74, 154.08, 150.57, 144.82, 140.45, 140.27, 129.90, 129.33, 128.33, 126.24, 124.62, 122.73, 117.16, 116.43, 114.39, 108.51, 100.81, 59.05, 56.54, 48.17, 43.79, 31.79, 27.29, 26.48, 7.91. LCMS (m/z) M+H=819.29.

t. Synthesis of Compound 43

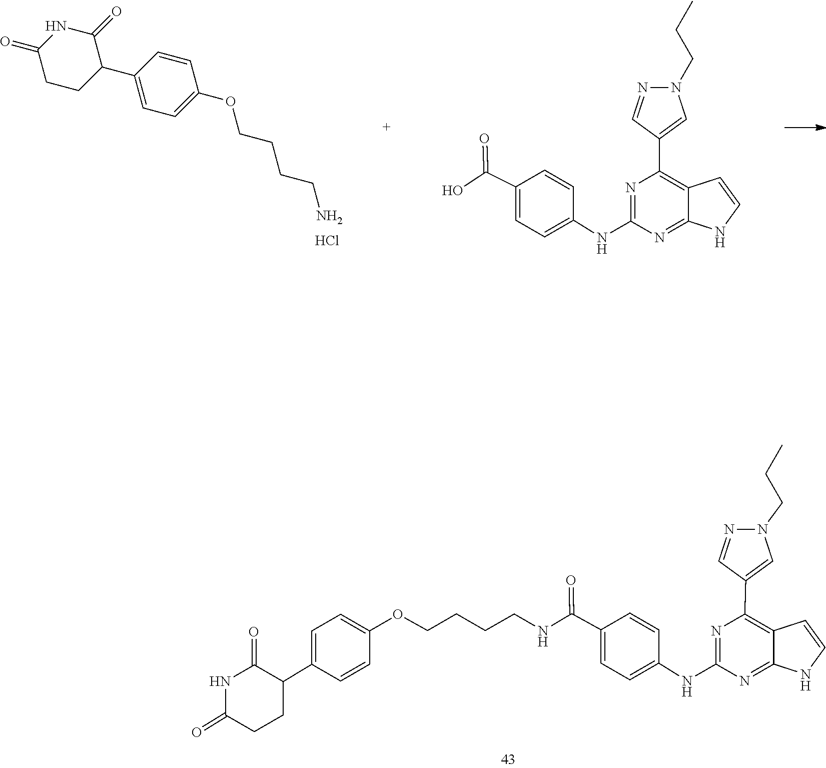 [SMILES: NCCCCOc1ccc(C2CCC(=O)NC2=O)cc1 ; CCCn1cc(-c2nc(Nc3ccc(C(=O)O)cc3)nc3[nH]ccc23)cn1 ; Cl ; CCCn1cc(-c2nc(Nc3ccc(C(=O)NCCCCOc4ccc(C5CCC(=O)NC5=O)cc4)cc3)nc3[nH]ccc23)cn1]

Preparation of N-(4-(4-(2,6-dioxopiperidin-3-yl)phenoxy)butyl)-4-((4-(1-propyl-1H-pyrazol-4-yl)-7H-pyrrolo[2,3-d]pyrimidin-2-yl)amino)benzamide. To a vial was added 3-(4-(4-aminobutoxy)phenyl)piperidine-2,6-dione (11.44 mg, 0.041 mmol), 4-((4-(1-propyl-1H-pyrazol-4-yl)-7H-pyrrolo[2,3-d]pyrimidin-2-yl)amino)benzoic acid (15 mg, 0.041 mmol), HATU (23.61 mg, 0.062 mmol) and dissolved in DMF (0.5 ml) before adding DIPEA (0.043 ml, 0.248 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (13.2 mg, 51%). ¹H NMR (500 MHz, DMSO) δ 11.55 (s, 1H), 10.72 (s, 1H), 9.39 (s, 1H), 8.53 (s, 1H), 8.19 (d, J=13.6 Hz, 2H), 7.93-7.87 (m, 2H), 7.76-7.70 (m, 2H), 7.20 (dd, J=3.6, 2.2 Hz, 1H), 7.08-7.02 (m, 2H), 6.85-6.79 (m, 2H), 6.75 (dd, J=3.6, 1.8 Hz, 1H), 4.13 (t, J=7.0 Hz, 2H), 3.93 (t, J=6.3 Hz, 2H), 3.71 (dd, J=11.4, 4.9 Hz, 1H), 3.25 (q, J=6.6 Hz, 2H), 2.57 (m, 1H), 2.43-2.35 (m, 1H), 2.08 (m, 1H), 1.93 (m, 1H), 1.81 (h, J=7.3 Hz, 2H), 1.70 (m, 2H), 1.61 (p, J=6.9 Hz, 2H), 0.81 (t, J=7.4 Hz, 3H). ¹³C NMR (126 MHz, DMSO) δ 174.97, 173.93, 166.43, 158.02, 155.75, 153.96, 151.35, 144.86, 139.07, 131.40, 131.34, 130.02, 128.30, 126.21, 124.29, 121.12, 117.09, 114.72, 108.20, 100.64, 67.62, 53.61, 46.97, 31.84, 26.78, 26.51, 26.47, 23.64, 11.40, 11.32. LCMS (m/z) M+H=621.18.

u. Synthesis of Compound 44

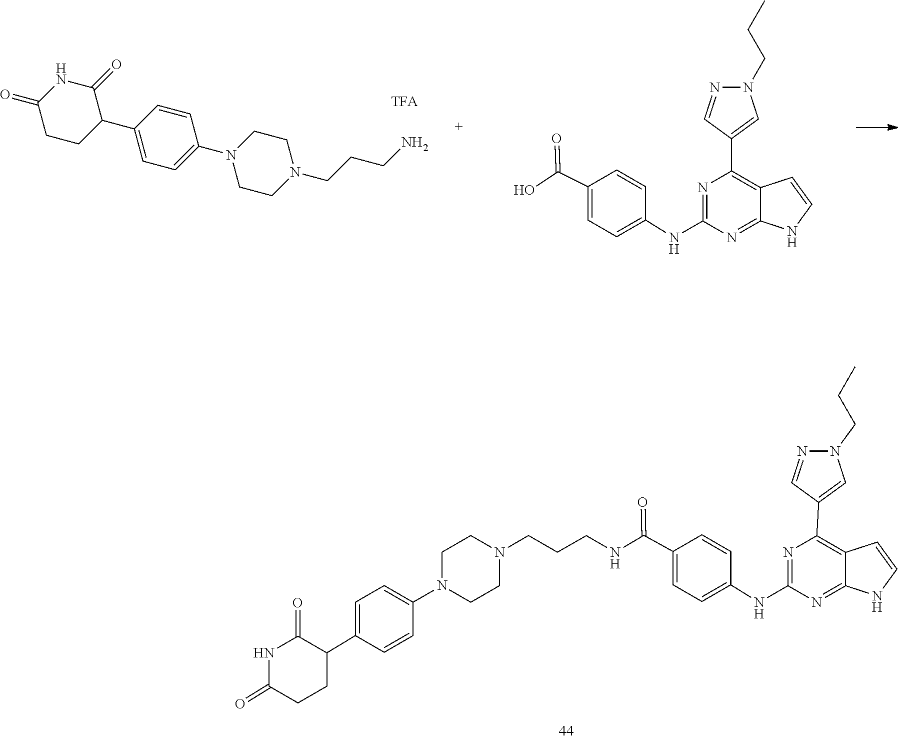 [SMILES: NCCCN1CCN(c2ccc(C3CCC(=O)NC3=O)cc2)CC1 ; CCCn1cc(-c2nc(Nc3ccc(C(=O)O)cc3)nc3[nH]ccc23)cn1 ; CCCn1cc(-c2nc(Nc3ccc(C(=O)NCCCN4CCN(c5ccc(C6CCC(=O)NC6=O)cc5)CC4)cc3)nc3[nH]ccc23)cn1]

Preparation of N-(3-(4-(4-(2,6-dioxopiperidin-3-yl)phenyl)piperazin-1-yl)propyl)-4-((4-(1-propyl-JH-pyrazol-4-yl)-7H-pyrrolo[2,3-d]pyrimidin-2-yl)amino)benzamide. To a vial was added 3-(4-(4-(3-aminopropyl)piperazin-1-yl)phenyl)piperidine-2,6-dione (20 mg, 0.061 mmol), 4-((4-(1-propyl-1H-pyrazol-4-yl)-7H-pyrrolo[2,3-d]pyrimidin-2-yl)amino)benzoic acid (15 mg, 0.041 mmol), HATU (34.5 mg, 0.091 mmol) and dissolved in DMF (0.5 ml) before adding DIPEA (0.063 ml, 0.363 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (13.7 mg, 34%). ¹H NMR (500 MHz, DMSO) δ 11.53 (s, 1H), 10.71 (s, 1H), 9.40 (s, 1H), 8.53 (s, 1H), 8.22 (t, J=5.6 Hz, 1H), 8.18 (s, 1H), 8.07 (s, 1H), 7.91 (d, J=2.0 Hz, 1H), 7.89 (d, J=2.0 Hz, 1H), 7.77-7.70 (m, 2H), 7.20 (dd, J=3.6, 2.3 Hz, 1H), 7.01-6.95 (m, 2H), 6.88-6.80 (m, 2H), 6.75 (dd, J=3.7, 1.8 Hz, 1H), 6.47 (s, 1H), 4.13 (t, J=7.0 Hz, 2H), 3.65 (dd, J=11.1, 4.9 Hz, 1H), 3.08 (s, 4H), 2.56 (m, 5H), 2.42-2.34 (m, 1H), 2.05 (m, 1H), 1.92 (m, 1H), 1.81 (h, J=7.2 Hz, 2H), 1.69 (p, J=7.0 Hz, 2H), 0.81 (t, J=7.4 Hz, 3H). ¹³C NMR (126 MHz, DMSO) δ 175.07, 173.95, 166.48, 163.55, 155.74, 153.95, 151.35, 150.25, 144.90, 139.07, 131.34, 129.82, 129.45, 128.50, 128.29, 126.17, 125.96, 124.30, 121.11, 117.10, 115.82, 108.20, 100.64, 56.02, 53.61, 53.07, 48.54, 46.90, 38.08, 31.73, 26.73, 26.43, 23.64, 11.40. LCMS (m/z) M+H=675.36.

v. Synthesis of Compound 45

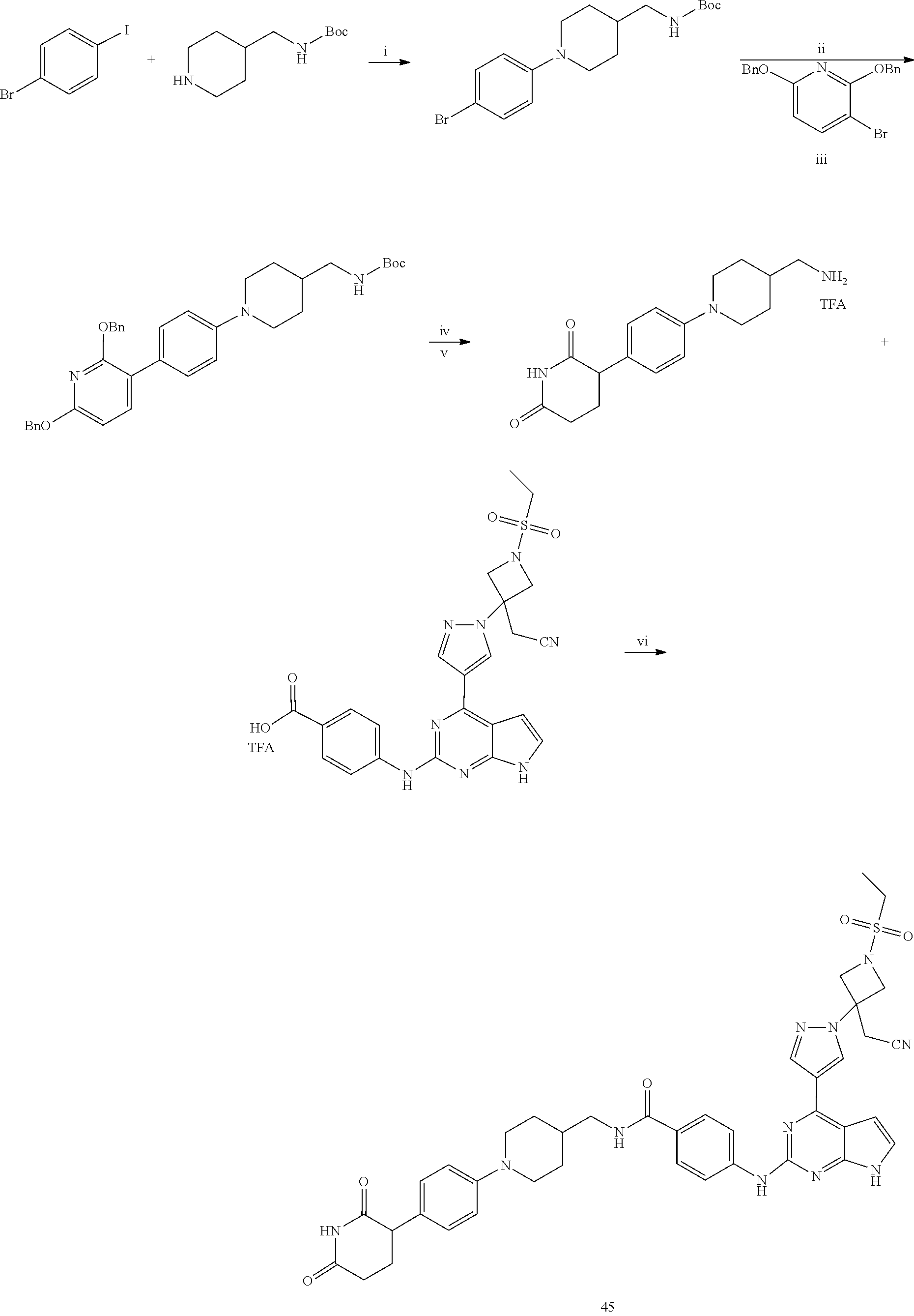

Step i. Preparation of tert-butyl ((1-(4-bromophenyl)piperidin-4-yl)methyl)carbamate. To a vial was added Cesium carbonate (461 mg, 1.414 mmol), tert-butyl (piperidin-4-ylmethyl)carbamate (152 mg, 0.707 mmol), 1-bromo-4-iodobenzene (200 mg, 0.707 mmol), 9,9-Dimethyl-4,5-bis(diphenylphosphino)xanthene (82 mg, 0.141 mmol), and Tris(dibenzylideneacetone)dipalladium(0) (64.7 mg, 0.071 mmol) and sealed then degassed and flushed with N₂ three times with stirring. A degassed solution of dioxane (8 ml) was then added and the reaction mixture degassed and flushed with N₂ before heating to 100° C. overnight with monitoring by LCMS. After cooling the reaction mixture was diluted in EtOAc and filtered through a bed of celite and the collected eluant washed with water (15 mL) then extracted with EtOAc (2×10 mL). The combined organic layers were washed with saturated brine, dried over anhydrous Na₂SO₄, filtered, and concentrated. The crude mixture was purified by flash column chromatography using Biotage 25 g Sfar HC D column, 0-20% Hex:EtOAc) to give the product as an oily light brown solid (61 mg, 23%). LCMS (m/z) M+H=369.09.

Step ii: Preparation of tert-butyl ((1-(4-(2,6-bis(benzyloxy)pyridin-3-yl)phenyl)piperidin-4-yl)methyl)carbamate. To a microwave vial was added tert-butyl ((1-(4-bromophenyl)piperidin-4-yl)methyl)carbamate (83.4 mg, 0.226 mmol), 2-Dicyclohexylphosphino-2′,6′-di-i-propoxy-1,1′-biphenyl (21.08 mg, 0.045 mmol), Methanesulfonato(2-dicyclohexylphosphino-2′,6′-di-i-propoxy-1,1′-biphenyl)(2-amino-1,1′-biphenyl-2-yl)palladium(II) (18.89 mg, 0.023 mmol), Tetrahydroxydiboron (60.7 mg, 0.677 mmol), and Potassium acetate (66.5 mg, 0.677 mmol) and sealed then degassed and flushed with N₂ three times with stirring. A degassed solution of EtOH (2 ml) was then added and the reaction mixture degassed and flushed with N₂ before heating to 80° C. for 1.5 h with monitoring by LCMS. During this time the reaction mixture became orange.

Step iii: After formation of the boronic acid, degassed Potassium carbonate (0.376 mL, 1.8M, aqueous solution) was added followed by 2,6-bis(benzyloxy)-3-bromopyridine (92 mg, 0.248 mmol) in degassed EtOH (0.5 mL) and the reaction mixture degassed and flushed with N₂ with heating to 80° C. overnight with monitoring by LCMS. After cooling the reaction mixture was diluted in EtOAc and filtered through a bed of celite and the collected eluant washed with water (15 mL) then extracted with EtOAc (2×10 mL). The combined organic layers were washed with saturated brine, dried over anhydrous Na₂SO₄, filtered, and concentrated. The crude mixture was purified by flash column chromatography using Biotage 10 g Sfar HC D column, 0-30% Hex:EtOAc) to give the product as an oily light brown solid (23 mg, 18%). ¹H NMR (500 MHz, CDCl₃) δ 7.51 (d, J=8.0 Hz, 2H), 7.46-7.36 (m, 3H), 7.37-7.21 (m, 9H), 6.89 (s, 2H), 6.37 (d, J=8.0 Hz, 1H), 5.35 (s, 2H), 5.28 (s, 2H), 4.57 (s, 1H), 3.66 (dt, J=12.8, 3.3 Hz, 2H), 3.00 (t, J=6.4 Hz, 2H), 2.66 (d, J=13.3 Hz, 2H), 1.74 (d, J=12.6 Hz, 2H), 1.51 (s, 2H), 1.38 (s, 9H). ¹³C NMR (126 MHz, CDCl₃) δ 160.75, 158.22, 156.10, 141.32, 138.04, 137.69, 129.63, 128.48, 128.34, 127.78, 127.76, 127.38, 127.28, 116.11, 102.32, 79.26, 67.82, 67.55, 49.53, 46.12, 36.53, 29.71, 28.45. LCMS (m/z) M+H=580.29.

Step iv: Preparation of tert-butyl ((1-(4-(2,6-dioxopiperidin-3-yl)phenyl)piperidin-4-yl)methyl)carbamate. To a vial was added Pd/C (4.22 mg, 3.97 μmol) and sealed then degassed and flushed with N₂ three times. In a separate vial, tert-butyl ((1-(4-(2,6-bis(benzyloxy)pyridin-3-yl)phenyl)piperidin-4-yl)methyl)carbamate (23 mg, 0.040 mmol) was dissolved in EtOH (3 ml) and sealed then degassed and flushed with N₂ three times before adding this solution to the Pd/C, followed by TES Triethylsilane (0.032 ml, 0.198 mmol) with stirring at rt overnight with monitoring by LCMS. After 2 h the reaction mixture was diluted in EtOAc and filtered through a bed of celite and the collected eluant evaporated under reduced pressure. The residue was dissolved in EtOAc (5 mL) and washed with water (10 mL) then extracted with EtOAc (2×5 mL). The combined organic layers were washed with saturated brine, dried over anhydrous Na₂SO₄, filtered, and concentrated. The crude mixture was purified using biotage Sfar C18 12 g column (5-50% MeCN in H₂O with 0.1% formic acid) to yield the product as an off-white solid (3.8 mg, 24%). ¹H NMR (500 MHz, CDCl₃) δ 7.93 (s, 1H), 6.99 (d, J=8.7 Hz, 2H), 6.89-6.79 (m, 2H), 4.56 (s, 1H), 3.70-3.50 (m, 2H), 2.98 (t, J=6.6 Hz, 2H), 2.70-2.49 (m, 3H), 2.23-2.09 (m, 1H), 1.96 (s, 2H), 1.75-1.67 (m, 2H), 1.29 (overlapping peaks, 2H). LCMS (m/z) M+H=402.19.

Step v: Preparation of 3-(4-(4-(aminomethyl)piperidin-1-yl)phenyl)piperidine-2,6-dione. To a solution of tert-butyl ((1-(4-(2,6-dioxopiperidin-3-yl)phenyl)piperidin-4-yl)methyl)carbamate (3.8 mg, 9.46 μmol) in DCM (1 mL) was added TFA (300 μL) and stirred for 1 h. The reaction mixture was concentrated under reduced pressure and used without further purification. LCMS (m/z) M+H=302.36.

Step vi: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-((1-(4-(2,6-dioxopiperidin-3-yl)phenyl)piperidin-4-yl)methyl)benzamide. To a vial was added 3-(4-(4-(aminomethyl)piperidin-1-yl)phenyl)piperidine-2,6-dione (2.8 mg, 9.29 μmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (4.71 mg, 9.29 μmol), HATU (5.30 mg, 0.014 mmol) and dissolved in DMF (0.5 ml) before adding DIPEA (0.016 ml, 0.093 mmol) with stirring at rt overnight with monitoring by LCMS. The crude reaction mixture was directly purified using biotage Sfar C18 12 g column (5-60% MeCN in H₂O with 0.1% formic acid) to yield the product as a white solid (1.1 mg, 15%). ¹H NMR (500 MHz, DMSO) δ 11.64 (s, 1H), 10.70 (s, 1H), 9.47 (s, 1H), 8.79 (s, 1H), 8.35 (d, J=2.5 Hz, 2H), 8.23 (t, J=5.8 Hz, 1H), 7.91 (d, J=8.8 Hz, 2H), 7.75 (d, J=8.8 Hz, 2H), 7.25 (dd, J=3.6, 2.2 Hz, 1H), 6.99-6.94 (m, 2H), 6.87 (dd, J=3.7, 1.9 Hz, 1H), 6.87-6.80 (m, 2H), 6.23 (s, 1H), 4.54 (d, J=9.1 Hz, 2H), 4.19 (d, J=9.1 Hz, 2H), 3.64 (s, 2H), 3.63 (td, J=14.2, 12.5, 4.0 Hz, 3H), 3.22-3.10 (overlapping peaks, 3H), 2.94-2.86 (m, 1H), 2.55 (td, J=12.9, 12.5, 3.1 Hz, 2H), 2.42-2.34 (m, 1H), 2.05 (tdd, J=13.4, 7.9, 3.5 Hz, 1H), 1.94 (dq, J=13.1, 4.9 Hz, 1H), 1.75-1.68 (m, 2H), 1.19 (q, J=7.3, 6.5 Hz, 3H). LCMS (m/z) M+H=790.33.

w. Synthesis of Cmpd 46

Step i: Preparation of (4-(2,6-bis(benzyloxy)pyridin-3-yl)phenyl)boronic acid. To a vial was added 2,6-bis(benzyloxy)-3-bromopyridine (60 mg, 0.162 mmol), 1,4-phenylenediboronic acid (81 mg, 0.486 mmol), tripotassium phosphate (68.8 mg, 0.324 mmol), Pd(dppf)Cl₂ (17.79 mg, 0.024 mmol) and sealed then degassed and flushed with N₂ three times with stirring. A degassed solution of dioxane (1 ml) and water (0.333 ml) was then added and the reaction mixture degassed and flushed with N₂ before heating to 80° C. overnight with monitoring by LCMS. After cooling the reaction mixture was diluted in EtOAc and filtered through a bed of celite and the collected eluant evaporated under reduced pressure. The residue was dissolved in EtOAc (10 mL) and washed with water (15 mL) then extracted with EtOAc (2×10 mL). The combined organic layers were washed with saturated brine, dried over anhydrous Na₂SO₄, filtered, and concentrated. The crude mixture was purified by flash column chromatography (Biotage Isolera, 10 g Sfar column, 0-80% Hex:EtOAc) to give the product as an oily light brown solid (23.4 mg, 35%). LCMS (m/z) M+H=412.40.

Step ii: Preparation of tert-butyl (1-(4-(2,6-bis(benzyloxy)pyridin-3-yl)phenyl)piperidin-4-yl)carbamate. To a vial was added tert-butyl piperidin-4-ylcarbamate (11.40 mg, 0.057 mmol), (4-(2,6-bis(benzyloxy)pyridin-3-yl)phenyl)boronic acid (23.4 mg, 0.057 mmol), Cupric acetate (10.33 mg, 0.057 mmol) and dissolved in DCM (1 ml) before adding TEA (23.03 mg, 0.228 mmol) with stirring at rt overnight with monitoring by LCMS. The reaction mixture was added to water (10 mL) and extracted with EtOAc (2×10 mL). The combined organic layers were washed with saturated brine, dried over anhydrous Na₂SO₄, filtered, and concentrated. The crude mixture was purified by flash column chromatography (Biotage Isolera, 10 g Sfar column, 0-20% Hex:EtOAc) to give the product as a white solid (11.2 mg, 35%). ¹H NMR (500 MHz, CDCl₃) δ 7.58 (d, J=8.1 Hz, 1H), 7.49 (d, J=8.8 Hz, 2H), 7.45-7.27 (m, 10H), 7.00 (s, 2H), 6.45 (d, J=8.0 Hz, 1H), 5.42 (s, 2H), 5.35 (s, 2H), 4.52-4.48 (m, 1H), 3.65 (d, J=12.5 Hz, 2H), 2.90 (t, J=11.9 Hz, 2H), 2.17-1.99 (m, 2H), 1.68-1.53 (m, 2H), 1.46 (s, 9H). ¹³C NMR (126 MHz, CDCl₃) δ 160.84, 158.23, 155.22, 141.34, 137.99, 137.67, 129.72, 128.48, 128.35, 127.78, 127.41, 127.31, 116.37, 115.70, 102.36, 79.46, 67.83, 67.58, 48.83, 47.66, 32.19, 28.44. LCMS (m/z) M+H=566.30.

Step iii: Preparation of tert-butyl (1-(4-(2,6-dioxopiperidin-3-yl)phenyl)piperidin-4-yl)carbamate. To a vial was added Pd/C (2.107 mg, 1.980 μmol) and sealed then degassed and flushed with N₂ three times. In a separate vial, tert-butyl (1-(4-(2,6-bis(benzyloxy)pyridin-3-yl)phenyl)piperidin-4-yl)carbamate (11.2 mg, 0.020 mmol) was dissolved in EtOH (1 ml) and sealed then degassed and flushed with N₂ three times before adding this solution to the Pd/C, followed by TES (0.016 ml, 0.099 mmol) with stirring at rt overnight with monitoring by LCMS. After 2 h the reaction mixture was diluted in EtOAc and filtered through a bed of celite and the collected eluant evaporated under reduced pressure. The residue was dissolved in EtOAc (5 mL) and washed with water (10 mL) then extracted with EtOAc (2×5 mL). The combined organic layers were washed with saturated brine, dried over anhydrous Na₂SO₄, filtered, and concentrated. The crude mixture was purified using biotage Sfar C18 12 g column (5-50% MeCN in H₂O with 0.1% formic acid) to yield the product as an off-white solid (3.3 mg, 43%). LCMS (m/z) M+H=388.46.

Step iv: Preparation of 3-(4-(4-aminopiperidin-1-yl)phenyl)piperidine-2,6-dione. To a solution of tert-butyl (1-(4-(2,6-dioxopiperidin-3-yl)phenyl)piperidin-4-yl)carbamate (3.3 mg, 8.52 μmol) in DCM (1 mL) was added TFA (300 μL) and stirred for 1 h. The reaction mixture was concentrated under reduced pressure and used without further purification. LCMS (m/z) M+H=288.37.

Step v: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(1-(4-(2,6-dioxopiperidin-3-yl)phenyl)piperidin-4-yl)benzamide. To a vial was added 3-(4-(4-aminopiperidin-1-yl)phenyl)piperidine-2,6-dione (2.5 mg, 8.70 μmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (4.41 mg, 8.70 μmol), HATU (4.96 mg, 0.013 mmol) and dissolved in DMF (0.5 ml) before adding DIPEA (0.015 ml, 0.087 mmol) with stirring at rt overnight with monitoring by LCMS. The crude reaction mixture was directly purified using biotage Sfar C18 12 g column (5-60% MeCN in H₂O with 0.1% formic acid) to yield the product as a white solid (2.2 mg, 33%). Purity 99% (UV/ELSD). ¹H NMR (500 MHz, DMSO) δ 11.63 (s, 1H), 10.71 (s, 1H), 9.46 (s, 1H), 8.78 (s, 1H), 8.40 (s, 1H), 8.35 (s, 1H), 7.98 (d, J=7.8 Hz, 1H), 7.90 (d, J=8.5 Hz, 2H), 7.75 (d, J=8.6 Hz, 2H), 7.27-7.23 (m, 1H), 6.99 (d, J=8.4 Hz, 2H), 6.86 (d, J=8.6 Hz, 3H), 4.53 (d, J=9.1 Hz, 2H), 4.19 (d, J=9.2 Hz, 2H), 3.92 (m, 1H), 3.71-3.62 (m, 5H), 3.18 (q, J=7.3 Hz, 2H), 2.75 (m, 2H), 2.13-1.99 (m, 1H), 1.94 (m, 1H), 1.79 (m, 2H), 1.60 (m, 2H), 1.18 (t, J=7.4 Hz, 3H). ¹³C NMR (126 MHz, DMSO) δ 174.50, 173.34, 165.69, 165.17, 155.11, 153.46, 149.91, 149.43, 144.17, 139.64, 129.25, 128.89, 128.54, 127.85, 125.66, 124.00, 122.11, 116.54, 116.45, 115.41, 107.88, 100.16, 58.42, 55.91, 47.78, 46.51, 46.26, 43.15, 31.09, 30.79, 26.65, 25.86, 7.28. HRMS (ESI) Exact mass calcd for C₃₉H₄₂N₁₁O₅S [M+H]⁺ 776.3091, found 776.3068.

x. Synthesis of Compound 47

Step i: Preparation of tert-butyl (4-(4-(2,6-dioxopiperidin-3-yl)phenoxy)butyl)carbamate. To a vial was added 3-(4-(4-aminobutoxy)phenyl)piperidine-2,6-dione (100 mg, 0.362 mmol) and dissolved in DCM (1 ml) followed by addition of Triethylamine (0.101 ml, 0.724 mmol) before adding Di-tert-butyl dicarbonate (0.125 ml, 0.543 mmol) with stirring at rt overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar 10 g silica HC D column (0-10% MeOH in DCM) to yield the compound as a white solid (111 mg, 81%). ¹H NMR (500 MHz, CDCl₃) δ 7.94 (s, 1H), 7.15-7.08 (m, 2H), 6.92-6.85 (m, 2H), 4.60 (s, 1H), 3.97 (t, J=6.2 Hz, 2H), 3.73 (dd, J=9.7, 5.2 Hz, 1H), 3.19 (t, J=7.0 Hz, 2H), 2.73 (m, 1H), 2.64 (m, 1H), 2.32-2.14 (m, 2H), 1.81 (m, 2H), 1.71-1.63 (m, 2H), 1.45 (s, 8H). ¹³C NMR (126 MHz, CDCl₃) δ 173.45, 172.35, 158.60, 156.18, 129.23, 129.02, 115.04, 79.38, 67.66, 47.33, 40.49, 31.07, 28.57, 26.99, 26.66, 26.56. LCMS (m/z) M-boc+H=277.09.

Step ii: Preparation of tert-butyl (4-(4-(1-methyl-2,6-dioxopiperidin-3-yl)phenoxy)butyl)carbamate. To a vial was added tert-butyl (4-(4-(2,6-dioxopiperidin-3-yl)phenoxy)butyl)carbamate (111 mg, 0.295 mmol) and potassium carbonate (48.9 mg, 0.354 mmol) and dissolved in DMF (1 ml) before adding Iodomethane (0.022 ml, 0.354 mmol) with stirring at rt overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar Kamino D 12 g column (40-80% EtOAc in hexane) to yield the compound as a yellow oil (47 mg, 41%). ¹H NMR (500 MHz, CDCl₃) δ 7.10-7.03 (m, 2H), 6.90-6.83 (m, 2H), 4.62 (s, 1H), 3.95 (t, J=6.2 Hz, 2H), 3.77 (dd, J=9.4, 5.0 Hz, 1H), 3.21 (s, 3H), 3.17 (t, J=7.0 Hz, 2H), 2.77 (m, 1H), 2.67 (m, 1H), 2.26-2.09 (m, 2H), 1.80 (m, 2H), 1.70-1.60 (m, 2H), 1.44 (s, 9H). ¹³C NMR (126 MHz, CDCl₃) δ 173.93, 172.68, 158.40, 156.14, 130.03, 129.84, 129.11, 114.90, 79.32, 67.62, 47.93, 40.46, 31.55, 28.54, 26.96, 26.64, 25.43. LCMS (m/z) M-boc+H=291.27.

Step iii: Preparation 3-(4-(4-aminobutoxy)phenyl)-1-methylpiperidine-2,6-dione. To a vial was added tert-butyl (4-(4-(1-methyl-2,6-dioxopiperidin-3-yl)phenoxy)butyl)carbamate (47 mg, 0.120 mmol) and dissolve in DCM (1 ml) before adding TFA (0.3 mL) with stirring at rt for 1 h with monitoring by LCMS. The reaction mixture was concentrated under reduced pressure and used without further purification. LCMS (m/z) M+H=291.21.

Step iv: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(4-(4-(1-methyl-2,6-dioxopiperidin-3-yl)phenoxy)butyl)benzamide. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (30 mg, 0.059 mmol), HATU (33.8 mg, 0.089 mmol), 3-(4-(4-aminobutoxy)phenyl)-1-methylpiperidine-2,6-dione (19 mg, 0.065 mmol) and dissolved in DMF (0.5 ml) before adding DIPEA (0.053 ml, 0.296 mmol) with stirring at rt overnight with monitoring by LCMS. The crude reaction mixture was directly purified using biotage Sfar C18 12 g column (5-70% MeCN in H₂O with 0.1% formic acid) to yield the product as an off-white solid (11 mg, 24%). Purity % (UV/ELSD). ¹H NMR (500 MHz, MeOD) δ 8.71 (s, 1H), 8.41 (s, 1H), 7.93 (d, J=8.8 Hz, 2H), 7.79 (d, J=8.8 Hz, 2H), 7.20 (d, J=3.6 Hz, 1H), 7.09 (d, J=8.7 Hz, 2H), 6.89 (d, J=8.7 Hz, 2H), 6.81 (d, J=3.7 Hz, 1H), 4.66 (d, J=9.1 Hz, 2H), 4.30 (d, J=9.1 Hz, 2H), 4.04 (t, J=6.0 Hz, 2H), 3.83 (dd, J=10.3, 5.4 Hz, 1H), 3.60 (s, 2H), 3.47 (t, J=6.7 Hz, 2H), 3.18 (q, J=7.4 Hz, 2H), 3.13 (s, 3H), 2.78-2.64 (m, 2H), 2.20-2.06 (m, 2H), 1.92-1.77 (m, 4H), 1.37 (t, J=7.3 Hz, 3H). ¹³C NMR (126 MHz, MeOD) δ 174.89, 173.45, 168.76, 158.25, 155.58, 153.93, 150.57, 144.77, 140.27, 130.90, 129.05, 128.73, 127.72, 125.65, 123.54, 123.06, 116.93, 115.57, 114.33, 108.59, 99.83, 67.25, 58.53, 56.14, 44.97, 39.20, 31.23, 26.47, 26.37, 25.86, 25.63, 25.30, 6.64. HRMS (ESI) Exact mass calcd for C₃₉H₄₃N₁₀O₆S [M+H]⁺ 779.3087, found 779.3082.

y. Synthesis of Compound 48

Step i: Preparation of tert-butyl 5-[4-(2-methoxy-2-oxoethyl)phenyl]-2,5-diazabicyclo[2.2.1]heptane-2-carboxylate. To a solution of methyl 2-(4-bromophenyl)acetate (1.11 g, 4.842 mmol, 1.2 equiv.) and tert-butyl 2,5-diazabicyclo[2.2.1]heptane-2-carboxylate (800 mg, 4.035 mmol, 1.0 equiv.) in toluene (8 mL) were added Cs₂CO₃ (2.63 g, 8.070 mmol, 2 equiv.) and RuPhos Pd G2 (0.31 g, 0.404 mmol, 0.1 equiv.) at room temperature under nitrogen atmosphere. The reaction was stirred at 110° C. for 16 hours. The resulting mixture was concentrated under reduced pressure, dissolved with EA (50 mL) and filtered. The filtrate was concentrated under reduced pressure and the residue was purified by silica gel column chromatography, eluted with 0˜18% EA in PE. The fractions containing desired product were combined and concentrated under reduced pressure to afford tert-butyl 5-[4-(2-methoxy-2-oxoethyl)phenyl]-2,5-diazabicyclo[2.2.1]heptane-2-carboxylate (1.1 g, 78.75% yield) as a light yellow solid. MS ESI (m/z)=347.10 [M+H]⁺. ¹H-NMR (400 MHz, Chloroform-d): δ 7.24-7.22 (m, 2H), 6.54-6.51 (m, 2H), 4.69-4.48 (m, 1H), 4.36 (s, 1H), 3.68 (s, 3H), 3.61-3.34 (m, 5H), 3.21-3.10 (m, 1H), 2.00-1.84 (m, 2H), 1.45-1.41 (m, 9H).

Step ii: Preparation of tert-butyl 5-[4-(2,6-dioxopiperidin-3-yl)phenyl]-2,5-diazabicyclo[2.2.1]heptane-2-carboxylate. To a solution of tert-butyl 5-[4-(2-methoxy-2-oxoethyl)phenyl]-2,5-diazabicyclo[2.2.1]heptane-2-carboxylate (1.1 g, 3.144 mmol, 1.0 equiv.) and prop-2-enamide (201.09 mg, 2.830 mmol, 0.9 equiv) in THF (11 mL) was added t-BuOK (1 M in THF, 3.458 mL, 3.458 mmol, 1.1 equiv) at 0° C. under nitrogen atmosphere. The reaction was stirred at 50° C. for 2 hours. The resulting reaction was quenched with saturated aq. NH₄Cl (50 mL) and extracted with EA (3×50 mL). The combined organic layer was washed with brine (100 mL), dried over anhydrous Na₂SO₄ and filtered. The filtrate was concentrated under reduced pressure and the residue was purified by silica gel column chromatography, eluted with 0˜5% MeOH in DCM. The fractions containing desired product were combined and concentrated under reduced pressure to afford tert-butyl 5-[4-(2,6-dioxopiperidin-3-yl)phenyl]-2,5-diazabicyclo[2.2.1]heptane-2-carboxylate (500 mg, 39.20% yield) as a pink solid. MS ESI (m/z)=386.15 [M+H]⁺. ¹H-NMR (400 MHz, Chloroform-d): δ 8.00 (s, 1H), 7.07-7.00 (m, 2H), 6.57-6.55 (m, 2H), 4.64-4.43 (m, 1H), 4.37 (s, 1H), 3.71-3.68 (m, 1H), 3.61-3.55 (m, 1H), 3.49-3.35 (m, 2H), 3.27-3.11 (m, 1H), 2.87-2.55 (m, 2H), 2.30-2.21 (m, 2H), 2.00-1.89 (m, 2H), 1.45-1.42 (m, 9H).

Step iii: Preparation of 3-(4-(2,5-diazabicyclo[2.2.1]heptan-2-yl)phenyl)piperidine-2,6-dione hydrochloride. To a solution of tert-butyl 5-[4-(2,6-dioxopiperidin-3-yl)phenyl]-2,5-diazabicyclo[2.2.1]heptane-2-carboxylate (500 mg, 1.232 mmol, 1.0 equiv.) in DCM (5 mL) was added HCl in 1,4-dioxane (4 M, 5 mL) at room temperature. The reaction was stirred at room temperature for 2 hours. The resulting reaction was concentrated under reduced pressure and the residue was diluted with H₂O (4 mL). The pH of the solution was adjusted to 8 with 1 M aq. NH₄HCO₃ (4 mL) and purified by RP-Flash with the following conditions: Column: Flash C¹⁸ (80 g); Mobile Phase A: water, Mobile Phase B: ACN; Flow rate: 60 mL/min; Gradient: 0% B to 0% B in 10 min; 0% B to 50% B in 10 min; Detector: UV 210 nm; RT: 15 min. The fractions containing desired product were directly lyophilized to afford 3-(4-{2,5-diazabicyclo[2.2.1]heptan-2-yl}phenyl)piperidine-2,6-dione hydrochloride (117.7 mg, 29.38% yield) as an off-white solid. MS ESI (m/z)=286.10 [M−HCl+H]⁺. ¹H-NMR (300 MHz, DMSO-d₆): δ 10.77 (s, 1H), 9.17 (br, 2H), 7.07-7.04 (m, 2H), 6.63-6.60 (m, 2H), 4.58 (s, 1H), 4.41 (s, 1H), 3.74-3.69 (m, 1H), 3.60-3.56 (m, 1H), 3.27-3.11 (m, 3H), 2.73-2.59 (m, 1H), 2.44-2.41 (m, 1H), 2.21-1.90 (m, 4H).

Step iv: Preparation of tert-butyl (3-(5-(4-(2,6-dioxopiperidin-3-yl)phenyl)-2,5-diazabicyclo[2.2.1]heptan-2-yl)propyl)carbamate. To a vial was added 3-(4-(2,5-diazabicyclo[2.2.1]heptan-2-yl)phenyl)piperidine-2,6-dione hydrochloride (25 mg, 0.078 mmol) and tert-butyl (3-bromopropyl)carbamate (18.50 mg, 0.078 mmol) and dissolved in DMF (1 ml) before adding DIPEA (0.079 mL, 0.466 mmol) with stirring and heating to 60° C. overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar 10 g silica HC D column (0-15% MeOH in DCM) to yield the compound as a solid (16.4 mg, 48%). LCMS (m/z) M+H=443.47.

Step v: Preparation of 3-(4-(5-(3-aminopropyl)-2,5-diazabicyclo[2.2.1]heptan-2-yl)phenyl)piperidine-2,6-dione. To a vial was added tert-butyl (3-(5-(4-(2,6-dioxopiperidin-3-yl)phenyl)-2,5-diazabicyclo[2.2.1]heptan-2-yl)propyl)carbamate (9.1 mg, 0.021 mmol) and dissolved in DCM (1 mL) before adding TFA (0.3 mL) with stirring at rt for 1 h with monitoring by LCMS. The reaction mixture was concentrated under reduced pressure and used without further purification. LCMS (m/z) M+H=343.46.

Step iv: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(3-(5-(4-(2,6-dioxopiperidin-3-yl)phenyl)-2,5-diazabicyclo[2.2.1]heptan-2-yl)propyl)benzamide. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (10.35 mg, 0.020 mmol), 3-(4-(5-(3-aminopropyl)-2,5-diazabicyclo[2.2.1]heptan-2-yl)phenyl)piperidine-2,6-dione (7 mg, 0.020 mmol), HATU (7.77 mg, 0.020 mmol) and dissolved in DMF (0.5 ml) before adding DIPEA (0.017 mL, 0.102 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (11.5 mg, 68%). ¹H NMR (500 MHz, DMSO) δ 11.69 (d, J=2.2 Hz, 1H), 10.75 (s, 1H), 9.52 (s, 1H), 8.85 (s, 1H), 8.42 (s, 1H), 8.28-8.21 (m, 2H), 7.95 (d, J=8.7 Hz, 2H), 7.75 (d, J=8.8 Hz, 2H), 7.32 (dd, J=3.6, 2.3 Hz, 1H), 6.98 (d, J=8.5 Hz, 2H), 6.94 (dd, J=3.7, 1.8 Hz, 1H), 6.53 (d, J=8.4 Hz, 2H), 4.61 (d, J=9.1 Hz, 2H), 4.26 (d, J=9.3 Hz, 3H), 3.71 (s, 2H), 3.70-3.63 (m, 1H), 3.57 (s, 1H), 3.34-3.19 (m, 5H), 3.13 (d, J=9.1 Hz, 1H), 2.86 (dd, J=9.5, 2.1 Hz, 1H), 2.61 (td, J=11.5, 11.1, 5.7 Hz, 1H), 2.50-2.39 (m, 3H), 2.16-2.04 (m, 1H), 1.99 (dq, J=13.8, 4.9 Hz, 1H), 1.84 (d, J=9.1 Hz, 1H), 1.77 (d, J=8.9 Hz, 1H), 1.60 (p, J=7.3 Hz, 2H), 1.26 (t, J=7.4 Hz, 3H). ¹³C NMR (126 MHz, DMSO) δ 174.84, 173.52, 165.85, 164.11, 155.28, 153.61, 150.11, 146.00, 144.24, 139.80, 129.42, 129.02, 127.76, 125.90, 125.86, 125.81, 124.13, 122.26, 116.70, 112.37, 108.03, 100.34, 60.87, 58.58, 56.89, 56.07, 52.57, 51.51, 46.42, 46.39, 43.32, 37.64, 35.72, 31.22, 28.75, 28.72, 26.81, 26.13, 26.03, 7.44. LCMS (m/z) M+H=831.67.

z. Synthesis of Compound 49

Step i: Preparation of benzyl 4-((1-(tert-butoxycarbonyl)piperidin-4-yl)methyl)piperazine-1-carboxylate. To a stirred solution of benzyl piperazine-1-carboxylate (10 g, 45.398 mmol, 1.0 equiv.) and tert-butyl 4-formylpiperidine-1-carboxylate (11.62 g, 54.478 mmol, 1.2 equiv.) in DCE (100 mL) were added MgSO₄ (54.65, 453.980 mmol, 10 equiv.) and TEA (31.55 mL, 226.990 mmol, 5 equiv.) at room temperature. The reaction suspension was stirred at room temperature for 30 min then NaBH(OAc)₃ (28.87 g, 136.194 mmol, 3 equiv.) was added to the solution. The reaction mixture was stirred at room temperature for 16 hours. The solid was filtered out and the filtrate was concentrated under reduced pressure. The residue was purified by silica gel column chromatography, eluted with 0˜60% EA in PE to afford benzyl 4-{[1-(tert-butoxycarbonyl)piperidin-4-yl]methyl}piperazine-1-carboxylate (15.8 g, 79.18% yield) as a light yellow viscous oil. MS ESI (m/z)=418.25 [M+H]⁺. ¹H-NMR (400 MHz, Chloroform-d): δ 7.38-7.30 (m, 5H), 5.13 (s, 2H), 4.11-4.09 (m, 2H), 3.51-3.49 (m, 3H), 2.71-2.65 (m, 2H), 2.38 (s, 4H), 2.24-2.18 (m, 2H), 2.07 (s, 1H), 1.74-1.50 (m, 3H), 1.45 (s, 9H), 1.12-1.02 (m, 2H).

Step ii: Preparation of tert-butyl 4-(piperazin-1-ylmethyl)piperidine-1-carboxylate. To a stirred solution of benzyl 4-((1-(tert-butoxycarbonyl)piperidin-4-yl)methyl)piperazine-1-carboxylate (15.8 g, 37.840 mmol, 1.0 equiv.) in THF (200 mL) was added 10% wt Pd/C (8.05 g, 7.568 mmol, 0.2 equiv.) at room temperature under nitrogen atmosphere. The reaction mixture was degassed with hydrogen for three times and stirred at room temperature for 3 hours under hydrogen atmosphere (1.5 atm). The resulting solution was filtered and the filtrate was concentrated under reduced pressure to afford tert-butyl 4-(piperazin-1-ylmethyl)piperidine-1-carboxylate (11 g, crude) as a light yellow solid. MS ESI (m/z)=284.15 [M+H]⁺.

Step iii: Preparation of tert-butyl 4-({4-[4-(2-methoxy-2-oxoethyl)phenyl]piperazin-1-yl}methyl)piperidine-1-carboxylate. To a solution of tert-butyl 4-(piperazin-1-ylmethyl)piperidine-1-carboxylate (11 g, 38.812 mmol, 1 equiv.) (crude) and methyl 2-(4-bromophenyl)acetate (10.67 g, 46.574 mmol, 1.2 equiv.) in toluene (110 mL) were added Cs₂CO₃ (25.29 g, 77.624 mmol, 2 equiv.) and RuPhos Pd G2 (3.01 g, 3.881 mmol, 0.1 equiv.) at room temperature under nitrogen atmosphere. The reaction was stirred at 110° C. for 16 hours. The resulting reaction was concentrated under reduced pressure, re-dissolved with EA (400 mL) and filtered. The filtrate was concentrated under reduced pressure and the residue was purified by silica gel column chromatography, eluted with 0˜40% EA in PE to afford tert-butyl 4-({4-[4-(2-methoxy-2-oxoethyl)phenyl]piperazin-1-yl}methyl)piperidine-1-carboxylate (10.6 g, 60.12% yield) as a yellow solid. MS ESI (m/z)=432.15 [M+H]⁺. ¹H-NMR (300 MHz, Chloroform-d): δ 7.18-7.15 (m, 2H), 6.90-6.86 (m, 2H), 4.11-4.07 (m, 2H), 3.68 (s, 3H), 3.54 (s, 2H), 3.17 (s, 4H), 2.74-2.55 (m, 6H), 2.22 (s, 2H), 1.78-1.67 (m, 3H), 1.46 (s, 9H), 1.16-1.04 (m, 2H).

Step iv: Preparation of tert-butyl 4-({4-[4-(2,6-dioxopiperidin-3-yl)phenyl]piperazin-1-yl}methyl)piperidine-1-carboxylate. To a solution of tert-butyl 4-({4-[4-(2-methoxy-2-oxoethyl)phenyl]piperazin-1-yl}methyl)piperidine-1-carboxylate (10.6 g, 24.561 mmol, 1 equiv.) in THF (106 mL) were added prop-2-enamide (1.57 g, 22.105 mmol, 0.9 equiv.) and Potassium tert-butoxide (1 M in THF, 27.01 mL, 27.017 mmol, 1.1 equiv.) at 0° C. under nitrogen atmosphere. The reaction was stirred at 50° C. for 2 hours. The reaction mixture was quenched with saturated aq. NH₄Cl (200 mL) and extracted with EA (3×300 mL). The combined organic layer was washed with brine (3×300 mL), dried over anhydrous Na₂SO₄ and filtered. The filtrate was concentrated under reduced pressure and the residue was purified by silica gel column chromatography, eluted with 0˜8% MeOH in DCM to afford tert-butyl 4-({4-[4-(2,6-dioxopiperidin-3-yl)phenyl]piperazin-1-yl}methyl)piperidine-1-carboxylate (4.1 g, 33.70% yield) as a red solid. MS ESI (m/z)=471.20 [M+H]⁺. ¹H-NMR (300 MHz, Chloroform-d): δ 8.00 (s, 1H), 7.11-7.08 (m, 2H), 6.93-6.88 (m, 2H), 4.12-4.08 (m, 2H), 3.74-3.69 (m, 1H), 3.19 (s, 4H), 2.77-2.56 (m, 8H), 2.30-2.18 (m, 4H), 1.78-1.73 (m, 3H), 1.46 (s, 9H), 1.16-1.08 (m, 2H).

Step v: Preparation of 3-(4-(4-(piperidin-4-ylmethyl)piperazin-1-yl)phenyl)piperidine-2,6-dione hydrochloride. To a stirred solution of tert-butyl 4-({4-[4-(2,6-dioxopiperidin-3-yl)phenyl]piperazin-1-yl}methyl)piperidine-1-carboxylate (3.8 g, 8.075 mmol, 1 equiv.) in DCM (38 mL) was added HCl in 1,4-dioxane (4 M, 38 mL) at 0° C. The reaction mixture was stirred at room temperature for 2 hours. The resulting reaction were concentrated under reduced pressure and diluted with H₂O (10 mL). The resulting solution was adjusted pH to 8 with 1 M aq. NH₄HCO₃ and the residue was purified by RP-Flash Column: Flash C¹⁸ Column (330 g); Mobile Phase A: water; Mobile Phase B: ACN; Flow rate: 100 mL/min; Gradient: 0%˜0% in 10 min; 0%˜50% in 10 min; 50%˜50% in 5 min; Detector: UV 210 nm. RT: 20 min. The fractions containing desired product were directly lyophilized to afford 3-{4-[4-(piperidin-4-ylmethyl)piperazin-1-yl]phenyl}piperidine-2,6-dione hydrochloride (1.1686 g, 35.10% yield) as a light yellow solid. MS ESI (m/z)=371.20 [M−HCl+H]⁺. ¹H-NMR (300 MHz, DMSO-d₆): δ 10.79 (s, 1H), 9.10-8.70 (m, 2H), 7.10-7.07 (m, 2H), 6.95-6.93 (m, 2H), 3.78-3.72 (m, 2H), 3.27-3.00 (m, 8H), 2.85-2.80 (m, 3H), 2.73-2.59 (m, 2H), 2.45-2.42 (m, 1H), 2.21-1.80 (m, 6H), 1.50-1.40 (m, 2H).

Step vi: Preparation of 2-(3-(4-(2-((4-(4-((4-(4-(2,6-dioxopiperidin-3-yl)phenyl)piperazin-1-yl)methyl)piperidine-1-carbonyl)phenyl)amino)-7H-pyrrolo[2,3-d]pyrimidin-4-yl)-1H-pyrazol-1-yl)-1-(ethylsulfonyl)azetidin-3-yl)acetonitrile. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (18.67 mg, 0.037 mmol), 3-(4-(4-(piperidin-4-ylmethyl)piperazin-1-yl)phenyl)piperidine-2,6-dione hydrochloride (15 mg, 0.037 mmol), HATU (21.02 mg, 0.055 mmol) and dissolved in DMF (0.5 ml) before adding DIPEA (0.033 mL, 0.184 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (12.7 mg, 40%). ¹H NMR (500 MHz, DMSO) δ 11.66 (d, J=2.1 Hz, 1H), 10.78 (s, 1H), 9.49 (s, 1H), 8.85 (s, 1H), 8.43 (s, 1H), 8.19 (s, 1H), 8.00-7.94 (m, 2H), 7.34 (d, J=8.4 Hz, 2H), 7.31 (dd, J=3.6, 2.2 Hz, 1H), 7.05 (d, J=8.6 Hz, 2H), 6.93 (dd, J=3.6, 1.9 Hz, 1H), 6.89 (d, J=8.7 Hz, 2H), 6.59 (s, 1H), 4.61 (d, J=9.1 Hz, 2H), 4.26 (d, J=9.1 Hz, 2H), 3.72 (s, 2H), 3.25 (q, J=7.4 Hz, 2H), 3.11 (t, J=4.7 Hz, 4H), 2.68-2.58 (m, 1H), 2.51-2.42 (overlapping peaks, 2H), 2.22 (d, J=7.1 Hz, 2H), 2.19-2.07 (m, 1H), 2.01 (dq, J=13.3, 5.0 Hz, 1H), 1.90-1.82 (m, 2H), 1.76 (s, 2H), 1.26 (t, J=7.3 Hz, 3H), 1.10 (qd, J=12.4, 3.9 Hz, 2H). ¹³C NMR (126 MHz, DMSO) δ 174.28, 173.15, 168.95, 163.36, 155.09, 153.33, 149.78, 149.63, 142.56, 139.49, 129.09, 128.87, 128.62, 127.49, 127.13, 123.69, 121.96, 116.67, 116.38, 114.95, 107.61, 99.98, 63.49, 58.27, 55.72, 52.85, 48.03, 46.10, 42.99, 32.60, 30.92, 26.45, 25.64, 7.11. LCMS (m/z) M+H=859.71.

aa. Synthesis of Compound 50

Step i: Preparation of tert-butyl 9-(4-(2-methoxy-2-oxoethyl)phenyl)-3,9-diazaspiro[5.5]undecane-3-carboxylate. To a solution of methyl 2-(4-bromophenyl)acetate (9.73 g, 42.457 mmol, 1.2 equiv.) and tert-butyl 3,9-diazaspiro[5.5]undecane-3-carboxylate (9.00 g, 35.381 mmol, 1.0 equiv.) in toluene (100 mL) were added Cs₂CO₃ (23.06 g, 70.762 mmol, 2 equiv.) and RuPhos Pd G2 (2.75 g, 3.538 mmol, 0.1 equiv.) at room temperature under nitrogen atmosphere. The reaction was stirred at 110° C. for 16 hours. The resulting reaction was concentrated under reduced pressure, re-dissolved with EA (200 mL) and filtered. The filtrate was concentrated under reduced pressure and the residue was purified by silica gel column chromatography, eluted with 0˜20% EA in PE. The fractions containing desired product were combined and concentrated under reduced pressure to afford tert-butyl 9-(4-(2-methoxy-2-oxoethyl)phenyl)-3,9-diazaspiro[5.5]undecane-3-carboxylate (8.6 g, 54.95% yield) as a light yellow solid. MS ESI (m/z)=403.20 [M+H]⁺. ¹H-NMR (300 MHz, Chloroform-d): δ 7.21-7.09 (m, 2H), 6.92-6.89 (m, 2H), 3.68 (s, 3H), 3.54 (s, 2H), 3.42-3.31 (m, 4H), 3.18-3.14 (m, 4H), 1.68-1.64 (m, 4H), 1.50-1.46 (m, 13H).

Step ii: Preparation of tert-butyl 9-(4-(2,6-dioxopiperidin-3-yl)phenyl)-3,9-diazaspiro[5.5]undecane-3-carboxylate. To a solution of tert-butyl 9-(4-(2-methoxy-2-oxoethyl)phenyl)-3,9-diazaspiro[5.5]undecane-3-carboxylate (8.6 g, 19.442 mmol, 1.0 equiv.) and prop-2-enamide (1.24 g, 17.51 mmol, 0.9 equiv.) in THF (90 mL) was added t-BuOK (1 M in THF, 21.386 mL, 21.386 mmol, 1.1 equiv.) at 0° C. under nitrogen atmosphere. The reaction was stirred at 50° C. for 2 hours. The resulting reaction was quenched with saturated aq. NH₄Cl (200 mL) and extracted with EA (3×200 mL). The combined organic layer was washed with brine (400 mL), dried over anhydrous Na₂SO₄ and filtered. The filtrate was concentrated under reduced pressure and the residue was purified by silica gel column chromatography, eluted with 0˜5% MeOH in DCM. The fractions containing desired product were combined and concentrated under reduced pressure to afford tert-butyl 9-(4-(2,6-dioxopiperidin-3-yl)phenyl)-3,9-diazaspiro[5.5]undecane-3-carboxylate (4 g, 46.13% yield) as a pink solid. MS ESI (m/z)=442.15 [M+H]⁺. ¹H-NMR (300 MHz, Chloroform-d): δ 8.00 (s, 1H), 7.22-6.74 (m, 4H), 3.74-3.70 (m, 1H), 3.51-3.39 (m, 4H), 3.20-3.16 (m, 4H), 2.77-2.56 (m, 2H), 2.32-2.14 (m, 2H), 1.68-1.46 (m, 17H).

Step iii: Preparation of 3-(4-(3,9-diazaspiro[5.5]undecan-3-yl)phenyl)piperidine-2,6-dione hydrochloride. To a solution of tert-butyl 9-(4-(2,6-dioxopiperidin-3-yl)phenyl)-3,9-diazaspiro[5.5]undecane-3-carboxylate (4 g, 8.968 mmol, 1.0 equiv.) in DCM (40 mL) was added HCl in 1,4-dioxane (4 M, 40 mL) at room temperature. The reaction was stirred at room temperature for 2 hours. The resulting reaction was concentrated under reduced pressure and the residue was diluted with H₂O (15 mL). The pH of the suspension was adjusted to 8 with 1 M aq. NH₄HCO₃ (15 mL) and purified by RP-CombiFlash with the following conditions: Column: Flash C¹⁸ Column (330 g); Mobile Phase A: water, Mobile Phase B: ACN; Flow rate: 100 mL/min; Gradient: 0% B to 0% B in 10 min; 0% B to 50% B in 10 min; Detector: UV 210 nm; RT: 20 min. The fractions containing desired product were directly lyophilized to afford 3-(4-{3,9-diazaspiro[5.5]undecan-3-yl}phenyl)piperidine-2,6-dione hydrochloride (1.2069 g, 35.26% yield) as an off-white solid. MS ESI (m/z)=342.20 [M−HCl+H]⁺. ¹H-NMR (300 MHz, DMSO-d₆): δ 10.78 (s, 1H), 8.87 (s, 2H), 7.04-6.91 (m, 4H), 3.76-3.71 (m, 1H), 3.25-3.04 (m, 8H), 2.73-2.41 (m, 2H), 2.20-1.95 (m, 2H), 1.64-1.48 (m, 8H).

Step iv: Preparation of 2-(3-(4-(2-((4-(9-(4-(2,6-dioxopiperidin-3-yl)phenyl)-3,9-diazaspiro[5.5]undecane-3-carbonyl)phenyl)amino)-7H-pyrrolo[2,3-d]pyrimidin-4-yl)-1H-pyrazol-1-yl)-1-(ethylsulfonyl)azetidin-3-yl)acetonitrile. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (20.11 mg, 0.040 mmol), 3-(4-(3,9-diazaspiro[5.5]undecan-3-yl)phenyl)piperidine-2,6-dione hydrochloride (15 mg, 0.040 mmol), HATU (22.64 mg, 0.060 mmol) and dissolved in DMF (0.5 ml) before adding DIPEA (0.035 mL, 0.198 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (13.7 mg, 42%). ¹H NMR (500 MHz, DMSO) δ 11.65 (s, 1H), 10.76 (s, 1H), 9.48 (s, 1H), 8.84 (s, 1H), 8.42 (s, 1H), 7.96 (d, J=8.4 Hz, 2H), 7.35 (d, J=8.3 Hz, 2H), 7.30 (t, J=3.0 Hz, 1H), 7.03 (d, J=8.4 Hz, 2H), 6.92 (dd, J=3.6, 1.8 Hz, 1H), 6.89 (d, J=8.4 Hz, 2H), 6.78 (s, 1H), 4.60 (d, J=9.1 Hz, 2H), 4.25 (d, J=9.1 Hz, 2H), 3.72 (d, J=8.5 Hz, 2H), 3.53 (overlapping peaks, 4H), 3.24 (q, J=7.3 Hz, 2H), 3.17-3.11 (m, 4H), 2.67-2.57 (m, 1H), 2.48-2.40 (m, 1H), 2.16-2.05 (m, 1H), 2.00 (dq, J=13.8, 5.1 Hz, 1H), 1.62 (broad, 4H), 1.49 (broad, 4H), 1.25 (t, J=7.4 Hz, 3H). ¹³C NMR (126 MHz, DMSO) δ 174.80, 173.66, 169.44, 155.58, 153.83, 150.32, 150.27, 143.08, 139.99, 129.59, 129.08, 128.80, 128.03, 127.53, 124.20, 122.46, 117.17, 116.89, 115.53, 108.11, 100.48, 58.77, 56.22, 46.56, 44.18, 43.50, 35.31, 34.83, 31.37, 29.94, 26.94, 26.15, 7.61. LCMS (m/z) M+H=830.41.

Bb. Synthesis of Compound 51

Step i: Preparation of tert-butyl (3-(9-(4-(2,6-dioxopiperidin-3-yl)phenyl)-3,9-diazaspiro[5.5]undecan-3-yl)propyl)carbamate. To a vial was added 3-(4-(3,9-diazaspiro[5.5]undecan-3-yl)phenyl)piperidine-2,6-dione hydrochloride (42 mg, 0.111 mmol) and tert-butyl (3-bromopropyl)carbamate (26.5 mg, 0.111 mmol) and dissolved in DMF (1 ml) before adding DIPEA (0.099 mL, 0.556 mmol) with stirring and heating to 60° C. overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar 10 g silica HC D column (0-15% MeOH in DCM) to yield the compound as a solid (14.4 mg, 26%). ¹H NMR (500 MHz, MeOD) δ 7.12 (d, J=8.4 Hz, 2H), 7.03-6.92 (m, 2H), 3.77 (dd, J=8.9, 6.4 Hz, 1H), 3.23-3.04 (m, 8H), 2.98 (t, J=8.0 Hz, 2H), 2.76-2.49 (m, 2H), 2.28-2.12 (m, 2H), 1.93 (s, 2H), 1.91-1.80 (m, 2H), 1.77 (t, J=5.8 Hz, 4H), 1.71 (t, J=5.7 Hz, 4H), 1.44 (s, 9H). ¹³C NMR (126 MHz, MeOD) δ 177.12, 175.50, 174.38, 157.41, 150.77, 129.37, 129.30, 128.67, 116.46, 79.00, 54.39, 45.02, 37.18, 32.74, 30.57, 28.48, 27.33, 26.40, 24.97, 21.61. LCMS (m/z) M+H=499.56.

Step ii: Preparation of 3-(4-(9-(3-aminopropyl)-3,9-diazaspiro[5.5]undecan-3-yl)phenyl)piperidine-2,6-dione. To a vial was tert-butyl (3-(9-(4-(2,6-dioxopiperidin-3-yl)phenyl)-3,9-diazaspiro[5.5]undecan-3-yl)propyl)carbamate (14.4 mg, 0.029 mmol) and dissolved in DCM (1 mL) before adding TFA (0.3 mL) with stirring at rt for 1 h with monitoring by LCMS. The reaction mixture was concentrated under reduced pressure and used without further purification. LCMS (m/z) M+H=399.48.

Step iii: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(3-(9-(4-(2,6-dioxopiperidin-3-yl)phenyl)-3,9-diazaspiro[5.5]undecan-3-yl)propyl)benzamide. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (14.62 mg, 0.029 mmol), 3-(4-(9-(3-aminopropyl)-3,9-diazaspiro[5.5]undecan-3-yl)phenyl)piperidine-2,6-dione (11.5 mg, 0.029 mmol), HATU (16.46 mg, 0.043 mmol) and dissolved in DMF (0.5 ml) before adding DIPEA (0.026 mL, 0.144 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (6.6 mg, 26%). ¹H NMR (500 MHz, DMSO) δ 11.68 (d, J=2.2 Hz, 1H), 10.76 (s, 1H), 9.54 (s, 1H), 8.85 (s, 1H), 8.42 (s, 1H), 8.33 (t, J=5.6 Hz, 1H), 8.23 (s, 1H), 8.02-7.94 (m, 2H), 7.83-7.75 (m, 2H), 7.31 (dd, J=3.6, 2.3 Hz, 1H), 7.01 (d, J=8.7 Hz, 2H), 6.93 (dd, J=3.7, 1.8 Hz, 1H), 6.86 (d, J=8.7 Hz, 2H), 4.60 (d, J=9.1 Hz, 2H), 4.25 (d, J=9.2 Hz, 2H), 3.70 (s, 2H), 3.27 (overlapping peaks, 4H), 3.12-3.06 (m, 4H), 2.67-2.56 (m, 1H), 2.48-2.37 (m, 7H), 2.11 (dtd, J=13.3, 11.1, 4.6 Hz, 1H), 1.99 (dq, J=13.2, 5.1 Hz, 1H), 1.69 (p, J=6.9 Hz, 2H), 1.53 (t, J=5.7 Hz, 4H), 1.48 (t, J=5.6 Hz, 4H), 1.25 (t, J=7.4 Hz, 3H). ¹³C NMR (126 MHz, DMSO) δ 174.81, 173.66, 166.06, 164.10, 155.45, 153.78, 150.39, 150.27, 144.48, 139.97, 129.60, 129.06, 128.76, 127.96, 126.06, 124.32, 122.43, 116.86, 115.50, 108.21, 100.50, 58.75, 56.37, 56.24, 48.87, 46.56, 44.23, 43.49, 38.21, 35.20, 31.38, 29.04, 26.98, 26.46, 26.15, 7.61. LCMS (m/z) M+H=887.44.

cc. Synthesis of Compound 52

Step a: Preparation of tert-butyl 8-[4-(2-methoxy-2-oxoethyl)phenyl]-2,8-diazaspiro[4.5]decane-2-carboxylate. To a solution of methyl 2-(4-bromophenyl)acetate (5.72 g, 24.964 mmol, 1.2 equiv.) and tert-butyl 2,8-diazaspiro[4.5]decane-2-carboxylate (5 g, 20.803 mmol, 1.0 equiv.) in toluene (50 mL) were added Cs₂CO₃ (13.56 g, 41.606 mmol, 2 equiv.) and RuPhos Pd G2 (1.62 g, 2.080 mmol, 0.1 equiv.) at room temperature under nitrogen atmosphere. The reaction was stirred at 110° C. for 16 hours. The resulting reaction was concentrated under reduced pressure, dissolved with EA (200 mL) and filtered. The filtrate was concentrated under reduced pressure and the residue was purified by silica gel column chromatography, eluted with 0˜22% EA in PE. The fractions containing desired product were combined and concentrated under reduced pressure to afford tert-butyl 8-[4-(2-methoxy-2-oxoethyl)phenyl]-2,8-diazaspiro[4.5]decane-2-carboxylate (6 g, 70.53% yield) as a light yellow oil. MS ESI (m/z)=389.15 [M+H]⁺. ¹H-NMR (300 MHz, Chloroform-d): δ 7.19-7.16 (m, 2H), 6.92 (bs, 2H), 3.68 (d, J=1.5 Hz, 3H), 3.55 (s, 2H), 3.47-3.36 (m, 2H), 3.27-3.11 (m, 6H), 1.78-1.71 (m, 6H), 1.47 (d, J=1.5 Hz, 9H).

Step b: Preparation of tert-butyl 8-[4-(2,6-dioxopiperidin-3-yl)phenyl]-2,8-diazaspiro[4.5]decane-2-carboxylate. To a solution of tert-butyl 8-[4-(2-methoxy-2-oxoethyl)phenyl]-2,8-diazaspiro[4.5]decane-2-carboxylate (6 g, 15.444 mmol, 1.0 equiv.) in THF (60 mL) were added prop-2-enamide (0.99 g, 13.900 mmol, 0.9 equiv.) and t-BuOK (1 M in THF, 16.988 mL, 16.988 mmol, 1.1 equiv.) at 0° C. under nitrogen atmosphere. The reaction was stirred at 50° C. for 2 hours. The resulting reaction was quenched with saturated aq. NH₄Cl (200 mL) and extracted with EA (3×200 mL). The combined organic layer was washed with brine (400 mL), dried over anhydrous Na₂SO₄ and filtered. The filtrate was concentrated under reduced pressure and the residue was purified by silica gel column chromatography, eluted with 0˜5% MeOH in DCM. The fractions containing desired product were combined and concentrated under reduced pressure to afford tert-butyl 8-[4-(2,6-dioxopiperidin-3-yl)phenyl]-2,8-diazaspiro[4.5]decane-2-carboxylate (2.4 g, 34.53% yield) as a pink solid. MS ESI (m/z)=450.25 [M+Na]⁺. ¹H-NMR (300 MHz, Chloroform-d): δ 8.02 (s, 1H), 7.21-6.92 (m, 4H), 3.875-3.61 (m, 1H), 3.47-3.13 (m, 8H), 2.77-2.57 (m, 2H), 2.32-2.14 (m, 2H), 1.78-1.68 (m, 6H), 1.49-1.47 (m, 9H).

Step c: Preparation of 3-(4-(2,8-diazaspiro[4.5]decan-8-yl)phenyl)piperidine-2,6-dione hydrochloride. To a solution of tert-butyl 8-[4-(2,6-dioxopiperidin-3-yl)phenyl]-2,8-diazaspiro[4.5]decane-2-carboxylate (2.4 g, 8.968 mmol, 1.0 equiv.) in DCM (24 mL) was added HCl in 1,4-dioxane (4 M, 24 mL) at room temperature. The reaction was stirred at room temperature for 2 hours. The resulting reaction was concentrated under reduced pressure and the residue was diluted with H₂O (10 mL). The pH of the solution was adjusted to 8 with 1 M aq. NH₄HCO₃ (10 mL) and purified by RP-Flash with the following conditions: Column: Flash C¹⁸ Column (330 g); Mobile Phase A: water, Mobile Phase B: ACN; Flow rate: 100 mL/min; Gradient: 0% B to 0% B in 10 min; 0% B to 50% B in 10 min; Detector: UV 210 nm; RT: 15 min. The fractions containing desired product were directly lyophilized to afford 3-(4-{2,8-diazaspiro[4.5]decan-8-yl}phenyl)piperidine-2,6-dione hydrochloride (1.1221 g, 54.38% yield) as an off-white solid. MS ESI (m/z)=328.15 [M−HCl+H]⁺. ¹H-NMR (300 MHz, DMSO-d₆): δ 10.79 (s, 1H), 9.38 (s, 2H), 7.20-6.80 (m, 4H), 3.76-3.74 (m, 1H), 3.28-3.02 (m, 8H), 2.73-2.59 (m, 1H), 2.49-2.42 (m, 1H), 2.21-1.95 (m, 2H), 1.90-1.70 (m, 6H).

Step d: Preparation of 2-(3-(4-(2-((4-(8-(4-(2,6-dioxopiperidin-3-yl)phenyl)-2,8-diazaspiro[4.5]decane-2-carbonyl)phenyl)amino)-7H-pyrrolo[2,3-d]pyrimidin-4-yl)-1H-pyrazol-1-yl)-1-(ethylsulfonyl)azetidin-3-yl)acetonitrile. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (22.83 mg, 0.045 mmol), 3-(4-(2,8-diazaspiro[4.5]decan-8-yl)phenyl)piperidine-2,6-dione hydrochloride (16.4 mg, 0.045 mmol), HATU (25.7 mg, 0.068 mmol) and dissolved in DMF (0.5 ml) before adding DIPEA (0.040 mL, 0.225 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (19.8 mg, 54%). ¹H NMR (500 MHz, DMSO) δ 11.66 (s, 1H), 10.77 (d, J=12.7 Hz, 1H), 9.51 (s, 1H), 8.85 (s, 1H), 8.43 (s, 1H), 7.97 (d, J=8.5 Hz, 2H), 7.52 (t, J=10.1 Hz, 2H), 7.31 (t, J=2.8 Hz, 1H), 7.08-6.98 (m, 2H), 6.95-6.84 (m, 3H), 4.60 (d, J=9.1 Hz, 2H), 4.25 (d, J=9.1 Hz, 2H), 3.71 (s, 2H), 3.60 (d, J=22.7 Hz, 2H), 3.41 (d, J=14.4 Hz, 3H), 3.25 (q, J=7.3 Hz, 2H), 3.13 (d, J=28.1 Hz, 3H), 2.12 (s, 1H), 2.00 (s, 1H), 1.81 (d, J=17.5 Hz, 2H), 1.70 (s, 2H), 1.59 (s, 2H), 1.25 (t, J=7.4 Hz, 3H). ¹³C NMR (126 MHz, DMSO) δ 174.61, 173.48, 155.37, 153.65, 150.09, 143.29, 139.82, 129.42, 128.92, 128.25, 127.84, 124.07, 122.29, 116.73, 115.71, 107.98, 100.31, 58.90, 58.61, 56.06, 54.93, 46.40, 46.18, 45.93, 43.33, 35.96, 33.89, 33.21, 31.22, 28.99, 26.77, 25.98, 7.44. LCMS (m/z) M+H=816.61.

dd. Synthesis of Compound 53

Step a: Preparation of tert-butyl 6-[4-(2-methoxy-2-oxoethyl)phenyl]-2,6-diazaspiro[3.3]heptane-2-carboxylate. To a solution of tert-butyl 2,6-diazaspiro[3.3]heptane-2-carboxylate (600 mg, 3.026 mmol, 1 equiv.) and methyl 2-(4-bromophenyl)acetate (831.88 mg, 3.631 mmol, 1.2 equiv.) in toluene (6 mL) were added Cs₂CO₃ (1.97 g, 6.052 mmol, 2 equiv.) and RuPhos Pd G2 (235.06 mg, 0.303 mmol, 0.1 equiv.) at room temperature under nitrogen atmosphere. The reaction was stirred at 110° C. for 16 hours. The resulting reaction was concentrated under reduced pressure, re-dissolved with EA (100 mL) and filtered. The filtrate was concentrated under reduced pressure and the residue was purified by silica gel column chromatography, eluted with 0˜30% EA in PE to afford tert-butyl 6-[4-(2-methoxy-2-oxoethyl)phenyl]-2,6-diazaspiro[3.3]heptane-2-carboxylate (860 mg, 77.93% yield) as a yellow solid. MS ESI (m/z)=347.10 [M+H]⁺. ¹H-NMR (300 MHz, Chloroform-d): δ 7.15-7.13 (m, 2H), 6.49-6.46 (m, 2H), 4.09 (s, 4H), 3.99 (s, 4H), 3.67 (s, 3H), 3.53 (s, 2H), 1.44 (s, 9H).

Step b: Preparation of tert-butyl 6-(4-(2,6-dioxopiperidin-3-yl)phenyl)-2,6-diazaspiro[3.3]heptane-2-carboxylate. To a solution of tert-butyl 6-[4-(2-methoxy-2-oxoethyl)phenyl]-2,6-diazaspiro[3.3]heptane-2-carboxylate (860 mg, 2.482 mmol, 1 equiv.) in THF (9 mL) were added prop-2-enamide (158.81 mg, 2.234 mmol, 0.9 equiv.) and potassium tert-butoxide (1 M in THF, 2.73 mL, 2.730 mmol, 1.1 equiv.) at 0° C. under nitrogen atmosphere. The reaction was stirred at 50° C. for 2 hours. The reaction mixture was quenched with saturated aq. NH₄Cl (15 mL) and extracted with EA (3×80 mL). The combined organic layer was washed with brine (3×80 mL), dried over anhydrous Na₂SO₄ and filtered. The filtrate was concentrated under reduced pressure and the residue was purified by silica gel column chromatography, eluted with 0%˜8% MeOH in DCM to afford tert-butyl 6-[4-(2,6-dioxopiperidin-3-yl)phenyl]-2,6-diazaspiro[3.3]heptane-2-carboxylate (306 mg, 30.38% yield) as a red solid. MS ESI (m/z)=386.15 [M+H]⁺. ¹H-NMR (300 MHz, Chloroform-d): δ 7.92 (s, 1H), 7.09-7.06 (m, 2H), 6.55-6.52 (m, 2H), 4.10 (s, 4H), 4.02 (s, 4H), 3.72-3.67 (m, 1H), 2.76-2.57 (m, 2H), 2.30-2.17 (m, 2H), 1.45 (s, 9H).

Step c: Preparation of 3-(4-(2,6-diazaspiro[3.3]heptan-2-yl)phenyl)piperidine-2,6-dione. To a stirred solution of tert-butyl 6-(4-(2,6-dioxopiperidin-3-yl)phenyl)-2,6-diazaspiro[3.3]heptane-2-carboxylate (250 mg, 0.649 mmol, 1 equiv.) in DCM (2.5 mL) was added TFA (0.5 mL) at 0° C. The reaction mixture was stirred at room temperature for 2 hours. The resulting reaction were concentrated under reduced pressure and diluted with H₂O (5 mL). The resulting solution was adjusted pH to 8 with 1 M aq. NH₄HCO₃ and the residue was purified by RP Flash (Column: Flash C¹⁸ Column (80 g); Mobile Phase A: water; Mobile Phase B: ACN; Flow rate: 55 mL/min; Gradient: 0%˜0% in 10 min; 0%˜50% in 10 min; 50%˜50% in 5 min; Detector: UV 210 nm. RT: 18 min. The fractions containing desired product were directly lyophilized to afford 3-(4-(2,6-diazaspiro[3.3]heptan-2-yl)phenyl)piperidine-2,6-dione 2,2,2-trifluoroacetate (153.6 mg, 58.11% yield) as an off white solid. MS ESI (m/z)=286.10 [M−TFA+H]⁺. ¹H-NMR (300 MHz, DMSO-d₆): δ 10.76 (s, 1H), 8.79 (s, 2H), 7.03-7.00 (m, 2H), 6.43-6.40 (m, 2H), 4.16 (s, 4H), 3.95 (s, 4H), 3.73-3.67 (m, 1H), 2.68-2.57 (m, 1H), 2.47-2.40 (m, 1H), 2.17-1.91 (m, 2H).

Step d: Preparation of 2-(3-(4-(2-((4-(6-(4-(2,6-dioxopiperidin-3-yl)phenyl)-2,6-diazaspiro[3.3]heptane-2-carbonyl)phenyl)amino)-7H-pyrrolo[2,3-d]pyrimidin-4-yl)-1H-pyrazol-1-yl)-1-(ethylsulfonyl)azetidin-3-yl)acetonitrile. To a vial was added 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (20.88 mg, 0.041 mmol), 3-(4-(2,6-diazaspiro[3.3]heptan-2-yl)phenyl)piperidine-2,6-dione (15.8 mg, 0.041 mmol), HATU (23.51 mg, 0.062 mmol) and dissolved in DMF (0.5 ml) before adding DIPEA (0.037 mL, 0.206 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (13.2 mg, 41%). ¹H NMR (500 MHz, DMSO) δ 11.70 (s, 1H), 10.76 (s, 1H), 9.60 (s, 1H), 8.85 (s, 1H), 8.43 (s, 1H), 7.99 (d, J=8.6 Hz, 2H), 7.61 (d, J=8.6 Hz, 2H), 7.34-7.29 (m, 1H), 7.02 (d, J=8.2 Hz, 2H), 6.94 (m, 1H), 6.79 (s, 1H), 6.41 (d, J=8.2 Hz, 2H), 4.60 (d, J=9.1 Hz, 2H), 4.56 (s, 2H), 4.25 (d, J=9.1 Hz, 2H), 4.23 (s, 2H), 3.96 (s, 4H), 3.71 (overlapping peaks, 3H), 3.27-3.20 (m, 2H), 2.67-2.57 (m, 1H), 2.16-2.06 (m, 1H), 1.98 (dq, J=13.7, 5.0 Hz, 1H), 1.25 (t, J=7.3 Hz, 3H). ¹³C NMR (126 MHz, DMSO) δ 174.69, 173.49, 168.87, 165.28, 155.22, 153.60, 150.53, 150.10, 144.34, 139.83, 129.44, 128.85, 128.73, 127.85, 124.22, 124.05, 122.25, 116.79, 116.72, 111.62, 108.09, 100.33, 61.93, 58.61, 56.07, 54.92, 46.56, 43.34, 33.60, 31.27, 26.77, 26.12, 7.44. LCMS (m/z) M+H=774.58.

ee. Synthesis of Compound 54

Step i: Preparation of tert-butyl 5-[4-(2-methoxy-2-oxoethyl)phenyl]-2,5-diazabicyclo[2.2.2]octane-2-carboxylate. To a solution of methyl 2-(4-bromophenyl)acetate (776.91 mg, 3.391 mmol, 1.2 equiv.) and tert-butyl 2,5-diazabicyclo[2.2.2]octane-2-carboxylate (600 mg, 2.826 mmol, 1 equiv.) in toluene (6 mL) were added Cs₂CO₃ (1.84 g, 5.652 mmol, 2 equiv.) and RuPhos Pd G2 (219.52 mg, 0.283 mmol, 0.1 equiv.) at room temperature under nitrogen atmosphere. The reaction was stirred at 110° C. for 16 hours. The resulting reaction was concentrated under reduced pressure, dissolved with EA (50 mL) and filtered. The filtrate was concentrated under reduced pressure and the residue was purified by silica gel column chromatography, eluted with 0˜20% EA in PE. The fractions containing desired product were combined and concentrated under reduced pressure to afford tert-butyl 5-[4-(2-methoxy-2-oxoethyl)phenyl]-2,5-diazabicyclo[2.2.2]octane-2-carboxylate (800 mg, 74.60% yield) as a light yellow oil. MS ESI (m/z)=361.10 [M+H]⁺. ¹H-NMR (400 MHz, Chloroform-d): δ 7.18-7.16 (m, 2H), 6.68-6.66 (m, 2H), 4.39-4.24 (m, 1H), 4.02-3.93 (m, 1H), 3.86-3.64 (m, 5H), 3.53 (s, 2H), 3.49-3.37 (m, 2H), 2.14-1.98 (m, 2H), 1.83-1.70 (m, 2H), 1.47-1.46 (m, 9H).

Step ii: Preparation of tert-butyl 5-[4-(2,6-dioxopiperidin-3-yl)phenyl]-2,5-diazabicyclo[2.2.2]octane-2-carboxylate. To a solution of tert-butyl 5-[4-(2-methoxy-2-oxoethyl)phenyl]-2,5-diazabicyclo[2.2.2]octane-2-carboxylate (800 mg, 2.108 mmol, 1.0 equiv.) and prop-2-enamide (134.88 mg, 1.897 mmol, 0.9 equiv.) in THF (8 mL) was added t-BuOK (1 M in THF, 2.319 mL, 2.319 mmol, 1.1 equiv.) at 0° C. under nitrogen atmosphere. The reaction was stirred at 50° C. for 2 hours. The resulting reaction was quenched with saturated aq. NH₄Cl (50 mL) and extracted with EA (3×50 mL). The combined organic layer was washed with brine (100 mL), dried over anhydrous Na₂SO₄ and filtered. The filtrate was concentrated under reduced pressure and the residue was purified by silica gel column chromatography, eluted with 0˜4% MeOH in DCM. The fractions containing desired product were combined and concentrated under reduced pressure to afford tert-butyl 5-[4-(2,6-dioxopiperidin-3-yl)phenyl]-2,5-diazabicyclo[2.2.2]octane-2-carboxylate (250 mg, 28.49% yield) as a pink solid. MS ESI (m/z)=400.10 [M+H]⁺. ¹H-NMR (400 MHz, Chloroform-d): δ 7.94 (s, 1H), 7.09-7.06 (m, 2H), 6.65-6.63 (m, 2H), 4.40-4.24 (m, 1H), 4.05-4.01 (m, 1H), 3.71-3.62 (m, 3H), 3.54-3.35 (m, 2H), 2.77-2.59 (m, 2H), 2.30-2.20 (m, 2H), 2.19-1.98 (m, 2H), 1.87-1.70 (m, 2H), 1.47-1.46 (m, 9H).

Step iii: Preparation of 3-(4-(2,5-diazabicyclo[2.2.2]octan-2-yl)phenyl)piperidine-2,6-dione hydrochloride. To a solution of tert-butyl 5-[4-(2,6-dioxopiperidin-3-yl)phenyl]-2,5-diazabicyclo[2.2.2]octane-2-carboxylate (250 mg, 1.232 mmol, 1.0 equiv.) in DCM (2.5 mL) was added HCl in 1,4-dioxane (4 M, 2.5 mL) at room temperature. The reaction was stirred at room temperature for 2 hours. The resulting reaction was concentrated under reduced pressure and the residue was diluted with H₂O (2 mL). The pH of the suspension was adjusted to 8 with 1 M aq. NH₄HCO₃ (2 mL) and purified by RP-Flash with the following conditions: Column: Flash C¹⁸ Column (80 g); Mobile Phase A: water, Mobile Phase B: ACN; Flow rate: 60 mL/min; Gradient: 0% B to 0% B in 10 min; 0% B to 50% B in 10 min; Detector: UV 210 nm; RT: 15 min. The fractions containing desired product were lyophilized to afford 3-(4-{2,5-diazabicyclo[2.2.2]octan-2-yl}phenyl)piperidine-2,6-dione hydrochloride (126.7 mg, 62.17% yield) as a grey solid. MS ESI (m/z)=300.15 [M−HCl+H]⁺. ¹H-NMR (300 MHz, DMSO-d₆): δ 10.76 (s, 1H), 9.54-9.41 (m, 2H), 7.06-7.03 (m, 2H), 6.68-6.65 (m, 2H), 4.15 (s, 1H), 3.77-3.57 (m, 3H), 3.36-3.29 (m, 3H), 2.69-2.58 (m, 1H), 2.50-2.41 (m, 1H), 2.19-1.71 (m, 6H).

Step iv: Preparation of tert-butyl (3-(5-(4-(2,6-dioxopiperidin-3-yl)phenyl)-2,5-diazabicyclo[2.2.2]octan-2-yl)propyl)carbamate. To a vial was added 3-(4-(2,5-diazabicyclo[2.2.2]octan-2-yl)phenyl)piperidine-2,6-dione hydrochloride (24 mg, 0.071 mmol) and tert-butyl (3-bromopropyl)carbamate (17.02 mg, 0.071 mmol) and dissolved in DMF (1 ml) before adding DIPEA (0.061 ml, 0.357 mmol) with stirring and heating to 60° C. overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar 10 g silica HC D column (0-15% MeOH in DCM) to yield the compound as a solid (18 mg, 55%). ¹H NMR (500 MHz, CDCl₃) δ 8.15 (s, 1H), 7.09-7.02 (m, 2H), 6.64-6.57 (m, 2H), 5.53 (s, 1H), 3.84 (t, J=3.1 Hz, 1H), 3.75 (m, 1H), 3.68 (m, 1H), 3.28-3.09 (m, 4H), 3.01 (s, 1H), 2.93-2.85 (m, 1H), 2.77-2.67 (m, 3H), 2.62 (m, 1H), 2.20 (m, 3H), 2.02-1.93 (m, 1H), 1.83 (m, 1H), 1.76-1.57 (m, 3H), 1.42 (s, 9H). ¹³C NMR (126 MHz, CDCl₃) δ 174.00, 172.72, 156.30, 147.67, 129.03, 124.01, 111.54, 79.04, 56.90, 54.38, 51.44, 49.07, 47.10, 45.23, 39.81, 31.00, 27.01, 26.51, 26.48, 24.76, 24.64. LCMS (m/z) M+H=457.53.

Step v: Preparation of 3-(4-(5-(3-aminopropyl)-2,5-diazabicyclo[2.2.2]octan-2-yl)phenyl)piperidine-2,6-dione. To a vial was added tert-butyl (3-(5-(4-(2,6-dioxopiperidin-3-yl)phenyl)-2,5-diazabicyclo[2.2.2]octan-2-yl)propyl)carbamate (18 mg, 0.039 mmol) and dissolved in DCM (1 mL) before adding TFA (0.3 mL) with stirring at rt for 1 h with monitoring by LCMS. The reaction mixture was concentrated under reduced pressure and used without further purification. LCMS (m/z) M+H=357.38.

Step vi: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(3-(5-(4-(2,6-dioxopiperidin-3-yl)phenyl)-2,5-diazabicyclo[2.2.2]octan-2-yl)propyl)benzamide. To a vial was added 3-(4-(5-(3-aminopropyl)-2,5-diazabicyclo[2.2.2]octan-2-yl)phenyl)piperidine-2,6-dione (14.05 mg, 0.039 mmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (19.96 mg, 0.039 mmol), HATU (22.48 mg, 0.059 mmol) and dissolved in DMF (0.5 ml) before adding DIPEA (25.5 mg, 0.197 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (17.7 mg, 53%). ¹H NMR (500 MHz, DMSO) δ 11.67 (s, 1H), 10.74 (s, 1H), 9.51 (s, 1H), 8.84 (s, 1H), 8.41 (s, 1H), 8.28 (q, J=4.9 Hz, 1H), 8.16 (s, 1H), 7.94 (d, J=8.5 Hz, 2H), 7.77 (d, J=8.5 Hz, 2H), 7.31 (t, J=3.0 Hz, 1H), 6.97 (d, J=8.3 Hz, 2H), 6.93 (m, 1H), 6.59 (d, J=8.6 Hz, 2H), 4.60 (d, J=9.1 Hz, 2H), 4.25 (d, J=9.1 Hz, 2H), 3.88 (d, J=3.6 Hz, 1H), 3.70 (s, 2H), 3.65 (m, 1H), 3.62-3.57 (m, 1H), 3.24 (q, J=7.4 Hz, 2H), 3.17-3.11 (m, 1H), 2.94 (d, J=9.6 Hz, 2H), 2.82 (m, 1H), 2.62 (m, 2H), 2.44 (m, 1H), 2.36 (p, J=1.8 Hz, 2H), 2.15-2.03 (m, 1H), 1.98 (m, 2H), 1.88-1.70 (m, 2H), 1.65 (p, J=7.0 Hz, 2H), 1.57 (m, 1H), 1.25 (t, J=7.3 Hz, 3H). ¹³C NMR (126 MHz, DMSO) δ 174.84, 173.53, 165.90, 163.34, 155.28, 153.61, 150.11, 147.37, 144.26, 139.80, 129.42, 129.04, 127.77, 125.92, 125.47, 125.45, 124.13, 122.27, 116.71, 111.01, 108.03, 100.34, 58.58, 56.41, 56.07, 53.59, 50.65, 50.00, 46.35, 44.42, 43.32, 37.78, 31.19, 27.56, 26.82, 26.07, 24.66, 24.08, 7.44. LCMS (m/z) M+H=845.66.

Ff. Synthesis of Compound 55

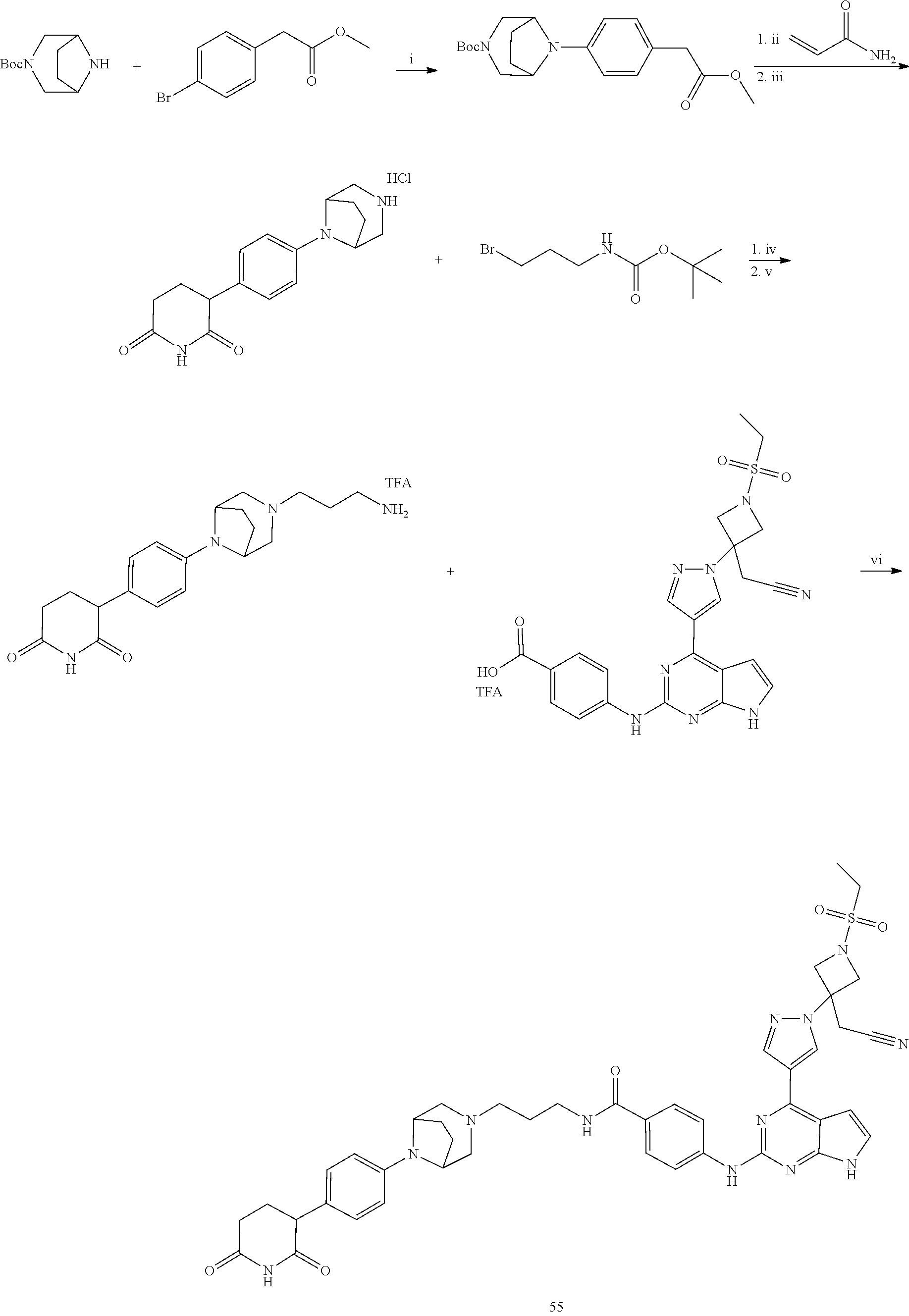

Step i. Preparation of tert-butyl 8-[4-(2-methoxy-2-oxoethyl)phenyl]-3,8-diazabicyclo[3.2.1]octane-3-carboxylate. To a solution of tert-butyl 3,8-diazabicyclo[3.2.1]octane-3-carboxylate (800 mg, 3.768 mmol, 1 equiv.) and methyl 2-(4-bromophenyl)acetate (1.04 g, 4.522 mmol, 1.2 equiv.) in toluene (8 mL) were added Cs₂CO₃ (2.45 g, 7.520 mmol, 2 equiv.) and RuPhos Pd G2 (292.8 mg, 0.377 mmol, 0.1 equiv.) at room temperature under nitrogen atmosphere. The reaction was stirred at 110° C. for 16 hours. The resulting reaction was concentrated under reduced pressure, re-dissolved with EA (100 mL) and filtered. The filtrate was concentrated under reduced pressure and the residue was purified by silica gel column chromatography, eluted with 0˜30% EA in PE to afford tert-butyl 8-[4-(2-methoxy-2-oxoethyl)phenyl]-3,8-diazabicyclo[3.2.1]octane-3-carboxylate (1.2 g, 83.93% yield) as a light yellow oil. MS ESI (m/z)=361.10 [M+H]⁺. ¹H NMR (300 MHz, Chloroform-d): δ 7.16-7.12 (m, 2H), 6.77-6.74 (m, 2H), 4.18-4.11 (m, 2H), 3.74-3.70 (m, 1H), 3.68 (s, 3H), 3.60-3.56 (m, 1H), 3.52 (s, 2H), 3.33-3.19 (m, 2H), 2.04-1.92 (m, 2H), 1.85-1.80 (m, 2H), 1.44 (s, 9H).

Step ii: Preparation of tert-butyl 8-[4-(2,6-dioxopiperidin-3-yl)phenyl]-3,8-diazabicyclo[3.2.1]octane-3-carboxylate. To a solution of tert-butyl 8-[4-(2-methoxy-2-oxoethyl)phenyl]-3,8-diazabicyclo[3.2.1]octane-3-carboxylate (1.2 g, 3.329 mmol, 1 equiv.) in THF (12 mL) were added prop-2-enamide (210 mg, 2.996 mmol, 0.9 equiv.) and potassium tert-butoxide (1 M in THF, 3.66 mL, 3.654 mmol, 1.1 equiv.) at 0° C. under nitrogen atmosphere. The reaction was stirred at 50° C. for 2 hours. The reaction mixture was quenched with saturated aq. NH₄Cl (20 mL) and extracted with EA (2×80 mL). The combined organic layer was washed with brine (200 mL), dried over anhydrous Na₂SO₄ and filtered. The filtrate was concentrated under reduced pressure and the residue was purified by silica gel column chromatography, eluted with 0˜8% MeOH in DCM to afford tert-butyl 8-[4-(2,6-dioxopiperidin-3-yl)phenyl]-3,8-diazabicyclo[3.2.1]octane-3-carboxylate (505 mg, 34.0% yield) as a red solid. MS ESI (m/z)=400.10 [M+H]⁺. ¹H-NMR (300 MHz, Chloroform-d): δ 7.95 (s, 1H), 7.10-7.07 (m, 2H), 6.83-6.80 (d, J=8.1 Hz, 2H), 4.20-4.15 (m, 2H), 3.77-3.59 (m, 3H), 3.36-3.22 (m, 2H), 2.76-2.57 (m, 2H), 2.28-2.18 (m, 2H), 2.02-1.99 (m, 2H), 1.88-1.82 (m, 2H), 1.45 (s, 9H).

Step iii: Preparation of 3-(4-(3,8-diazabicyclo[3.2.1]octan-8-yl)phenyl)piperidine-2,6-dione hydrochloride. To a stirred solution of tert-butyl 8-[4-(2,6-dioxopiperidin-3-yl)phenyl]-3,8-diazabicyclo[3.2.1]octane-3-carboxylate (250 mg, 0.626 mmol, 1.0 equiv.) in DCM (2.5 mL) was added HCl in 1,4-dioxane (4 M, 2.5 mL) at 0° C. The reaction mixture was stirred at room temperature for 2 hours. The resulting reaction were concentrated under reduced pressure and diluted with H₂O (5 mL). The resulting solution was adjusted pH to 8 with 1 M aq. NH₄HCO₃ and purified by RP-Flash (Column: Flash C¹⁸ Column (80 g); Mobile Phase A: water; Mobile Phase B: ACN; Flow rate: 55 mL/min; Gradient: 0%˜0% in 10 min; 0%˜50% in 10 min; 50%˜50% in 5 min; Detector: UV 210 nm. RT: 20 min. The fractions containing desired product were directly lyophilized to afford 3-(4-{3,8-diazabicyclo[3.2.1]octan-8-yl}phenyl)piperidine-2,6-dione hydrochloride (119.8 mg, 56.43% yield) as an off white solid. MS ESI (m/z)=300.15 [M−HCl+H]⁺. ¹H-NMR (300 MHz, DMSO-d₆): δ 10.78 (s, 1H), 9.47 (s, 1H), 9.16 (s, 1H), 7.11-7.08 (m, 2H), 6.88-6.85 (m, 2H), 4.37 (s, 2H), 3.76-3.71 (m, 1H), 3.07-2.93 (m, 4H), 2.70-2.59 (m, 1H), 2.45-2.42 (m, 1H), 2.21-2.08 (m, 6H).

Step iv: Preparation of tert-butyl (3-(8-(4-(2,6-dioxopiperidin-3-yl)phenyl)-3,8-diazabicyclo[3.2.1]octan-3-yl)propyl)carbamate. To a vial was added 3-(4-(3,8-diazabicyclo[3.2.1]octan-8-yl)phenyl)piperidine-2,6-dione hydrochloride (24 mg, 0.071 mmol) and tert-butyl (3-bromopropyl)carbamate (17.02 mg, 0.071 mmol) and dissolved in DMF (1 ml) before adding DIPEA (0.073 mL, 0.429 mmol) with stirring and heating to 60° C. overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar 10 g silica HC D column (0-15% MeOH in DCM) to yield the compound as a solid (11.7 mg, 36%). ¹H NMR (500 MHz, CDCl₃) δ 8.04 (s, 1H), 7.05 (d, J=8.4 Hz, 2H), 6.74 (d, J=2.0 Hz, 2H), 6.18 (s, 1H), 4.17 (d, J=4.6 Hz, 2H), 3.71 (dd, J=9.1, 5.0 Hz, 1H), 3.21 (q, J=5.8 Hz, 2H), 2.81-2.66 (m, 2H), 2.61 (m, 1H), 2.45 (broad, 3H), 2.31-2.14 (m, 2H), 2.11 (overlapping peaks, 1H), 1.98 (d, J=9.5 Hz, 2H), 1.64 (s, 2H), 1.44 (s, 9H). ¹³C NMR (126 MHz, CDCl₃) δ 173.54, 172.35, 156.02, 129.31, 128.99, 115.74, 115.43, 78.66, 57.19, 54.96, 46.78, 30.51, 28.34, 27.47, 26.26, 24.47, 20.43. LCMS (m/z) M+H=457.53.

Step v: Preparation of 3-(4-(3-(3-aminopropyl)-3,8-diazabicyclo[3.2.1]octan-8-yl)phenyl)piperidine-2,6-dione. To a vial was added tert-butyl (3-(8-(4-(2,6-dioxopiperidin-3-yl)phenyl)-3,8-diazabicyclo[3.2.1]octan-3-yl)propyl)carbamate (11.7 mg, 0.026 mmol) and dissolved in DCM (1 mL) before adding TFA (0.3 mL) with stirring at rt for 1 h with monitoring by LCMS. The reaction mixture was concentrated under reduced pressure and used without further purification. LCMS (m/z) M+H=357.45.

Step vi: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(3-(8-(4-(2,6-dioxopiperidin-3-yl)phenyl)-3,8-diazabicyclo[3.2.1]octan-3-yl)propyl)benzamide. To a vial was added 3-(4-(3-(3-aminopropyl)-3,8-diazabicyclo[3.2.1]octan-8-yl)phenyl)piperidine-2,6-dione (9.13 mg, 0.026 mmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (12.97 mg, 0.026 mmol), HATU (14.61 mg, 0.038 mmol) and dissolved in DMF (0.5 ml) before adding DIPEA (16.55 mg, 0.128 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (7.1 mg, 33%). ¹H NMR (500 MHz, DMSO) δ 11.68 (s, 1H), 10.76 (s, 1H), 9.53 (s, 1H), 8.85 (s, 1H), 8.42 (s, 1H), 8.28 (s, 1H), 8.18 (t, J=5.6 Hz, 1H), 7.96 (d, J=8.6 Hz, 2H), 7.78 (d, J=8.6 Hz, 2H), 7.34-7.29 (m, 1H), 6.99 (d, J=8.4 Hz, 2H), 6.94 (m, 1H), 6.76 (d, J=8.5 Hz, 2H), 4.60 (d, J=9.1 Hz, 2H), 4.26 (d, J=9.1 Hz, 2H), 4.20 (d, J=4.6 Hz, 2H), 3.73-3.65 (m, 3H), 3.26 (p, J=7.1 Hz, 3H), 2.67-2.53 (m, 3H), 2.49-2.41 (m, 1H), 2.30 (d, J=10.4 Hz, 2H), 2.25 (t, J=6.9 Hz, 2H), 2.16-2.05 (m, 1H), 2.01 (m, 1H), 1.91 (t, J=5.9 Hz, 2H), 1.80 (m, 2H), 1.63 (p, J=7.0 Hz, 2H), 1.25 (t, J=7.3 Hz, 3H). ¹³C NMR (126 MHz, DMSO) δ 174.74, 173.51, 165.91, 164.25, 155.29, 153.61, 150.11, 145.82, 144.25, 139.80, 129.43, 129.29, 127.82, 126.82, 125.93, 124.13, 122.27, 116.71, 116.68, 115.02, 108.03, 100.34, 58.58, 56.07, 54.80, 54.64, 54.58, 46.36, 43.32, 37.38, 31.21, 27.75, 26.82, 26.58, 26.02, 7.45. LCMS (m/z) M+H=845.66.

gg. Synthesis of Compound 56

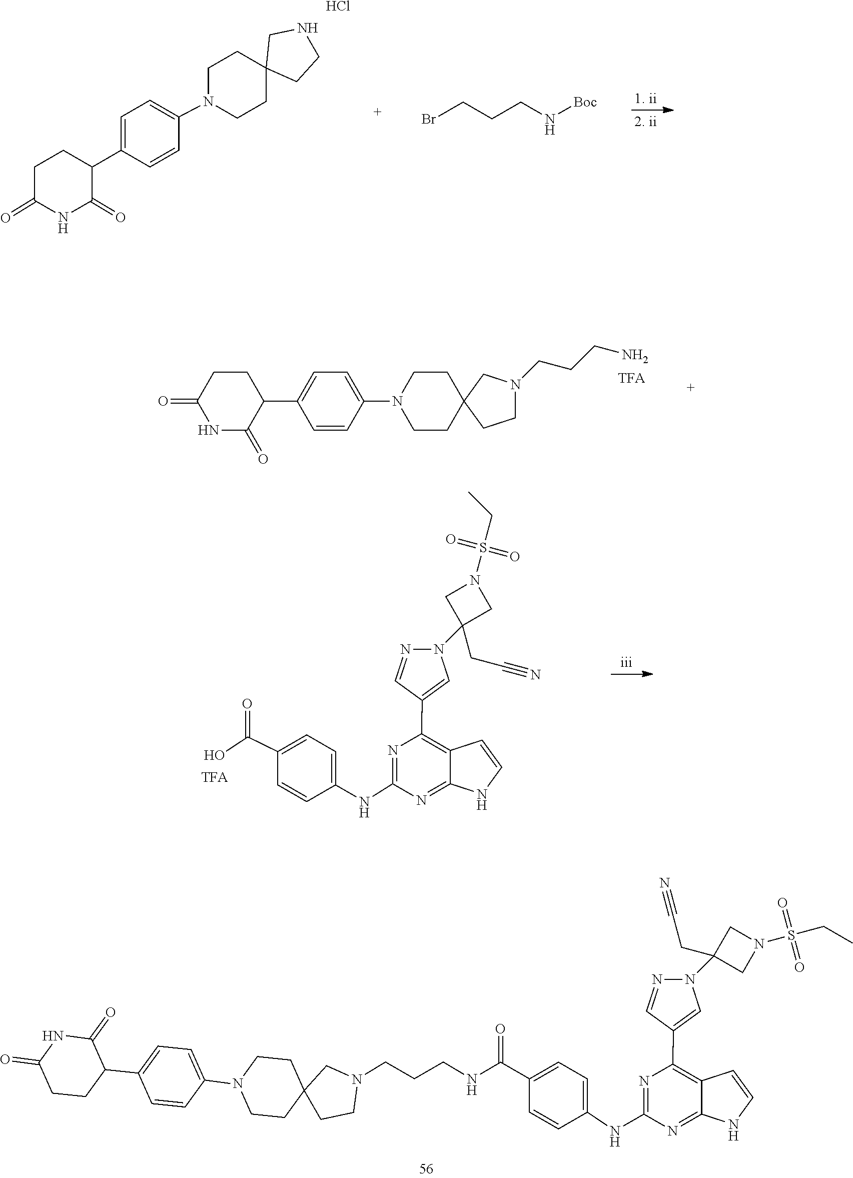 [SMILES: Cl ; O=C1CCC(c2ccc(N3CCC4(CCNC4)CC3)cc2)C(=O)N1 ; CC(C)(C)OC(=O)NCCCBr ; NCCCN1CCC2(CCN(c3ccc(C4CCC(=O)NC4=O)cc3)CC2)C1 ; CCS(=O)(=O)N1CC(CC#N)(n2cc(-c3nc(Nc4ccc(C(=O)O)cc4)nc4[nH]ccc34)cn2)C1 ; CCS(=O)(=O)N1CC(CC#N)(n2cc(-c3nc(Nc4ccc(C(=O)NCCCN5CCC6(CCN(c7ccc(C8CCC(=O)NC8=O)cc7)CC6)C5)cc4)nc4[nH]ccc34)cn2)C1]

Step i. Preparation of tert-butyl (3-(8-(4-(2,6-dioxopiperidin-3-yl)phenyl)-2,8-diazaspiro[4.5]decan-2-yl)propyl)carbamate. To a vial was added 3-(4-(2,8-diazaspiro[4.5]decan-8-yl)phenyl)piperidine-2,6-dione hydrochloride (25 mg, 0.069 mmol) and tert-butyl (3-bromopropyl)carbamate (16.36 mg, 0.069 mmol) and dissolved in DMF (1 ml) before adding DIPEA (0.070 mL, 0.412 mmol) with stirring and heating to 60° C. overnight with monitoring by LCMS. The reaction mixture was cooled, diluted with H₂O and extracted twice with EtOAc. The organic layer was collected, washed with brine, dried over NaSO₄ and evaporated under reduced pressure. The crude product was purified using biotage Sfar 10 g silica HC D column (0-15% MeOH in DCM) to yield the compound as a solid (14.1 mg, 42%). ¹H NMR (500 MHz, CDCl₃) δ 8.11 (s, 1H), 7.10-7.05 (m, 2H), 6.94-6.88 (m, 2H), 5.69 (s, 1H), 3.71 (dd, J=9.3, 5.1 Hz, 1H), 3.28-3.14 (m, 4H), 3.11 (ddd, J=12.3, 7.7, 4.1 Hz, 2H), 2.76 (s, 2H), 2.74-2.48 (overlapping peaks, 5H), 2.31-2.14 (m, 2H), 1.73 (overlapping peaks, 7H), 1.43 (s, 9H). ¹³C NMR (126 MHz, CDCl₃) δ 173.70, 172.57, 156.28, 151.10, 128.78, 127.39, 116.68, 79.01, 65.17, 54.88, 53.49, 47.15, 47.09, 40.00, 39.86, 37.18, 35.87, 30.90, 28.59, 27.41, 26.44, 22.71. LCMS (m/z) M+H=485.51.

Step ii: Preparation of 3-(4-(2-(3-aminopropyl)-2,8-diazaspiro[4.5]decan-8-yl)phenyl)piperidine-2,6-dione. To a vial was tert-butyl (3-(8-(4-(2,6-dioxopiperidin-3-yl)phenyl)-2,8-diazaspiro[4.5]decan-2-yl)propyl)carbamate (14.1 mg, 0.029 mmol) and dissolved in DCM (1 mL) before adding TFA (0.3 mL) with stirring at rt for 1 h with monitoring by LCMS. The reaction mixture was concentrated under reduced pressure and used without further purification. LCMS (m/z) M+H=385.43.

Step iii: Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(3-(8-(4-(2,6-dioxopiperidin-3-yl)phenyl)-2,8-diazaspiro[4.5]decan-2-yl)propyl)benzamide. To a vial was added 3-(4-(2-(3-aminopropyl)-2,8-diazaspiro[4.5]decan-8-yl)phenyl)piperidine-2,6-dione (11.19 mg, 0.029 mmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (14.74 mg, 0.029 mmol), HATU (16.60 mg, 0.044 mmol) and dissolved in DMF (0.5 ml) before adding DIPEA (18.81 mg, 0.146 mmol) with stirring at rt overnight with monitoring by LCMS. The crude reaction mixture was directly purified using biotage Sfar C18 12 g column (5-50% MeCN in H₂O with 0.1% formic acid) to yield the product as a white solid (11.8 mg, 46%). ¹H NMR (500 MHz, DMSO) δ 11.68 (d, J=2.4 Hz, 1H), 10.76 (s, 1H), 9.54 (s, 1H), 8.84 (s, 1H), 8.42 (s, 1H), 8.28 (t, J=5.5 Hz, 1H), 8.23 (s, 1H), 7.97 (d, J=8.7 Hz, 2H), 7.77 (d, J=8.6 Hz, 2H), 7.34-7.29 (m, 1H), 6.99 (d, J=8.4 Hz, 2H), 6.94 (m, 1H), 6.86 (d, J=8.4 Hz, 2H), 4.60 (d, J=9.1 Hz, 2H), 4.25 (d, J=9.1 Hz, 2H), 3.70 (s, 2H), 3.26 (overlapping peaks, 3H), 3.08 (m, 4H), 2.58 (overlapping peaks, 2H), 2.49-2.37 (overlapping peaks, 4H), 2.14-2.04 (m, 1H), 1.98 (m, 1H), 1.74-1.61 (m, 2H), 1.59 (m, 5H), 1.25 (t, J=7.4 Hz, 3H). ¹³C NMR (126 MHz, DMSO) δ 174.62, 173.49, 165.93, 163.88, 155.29, 153.61, 150.15, 150.11, 144.28, 139.80, 129.42, 128.89, 128.71, 127.79, 125.94, 124.14, 122.27, 116.71, 116.68, 115.59, 108.03, 100.33, 65.43, 58.58, 56.06, 53.90, 53.07, 46.39, 46.31, 43.32, 37.96, 36.96, 35.92, 31.20, 28.12, 26.81, 25.96, 7.44. LCMS (m/z) M+H=873.70.

hh. Synthesis of Compound 57

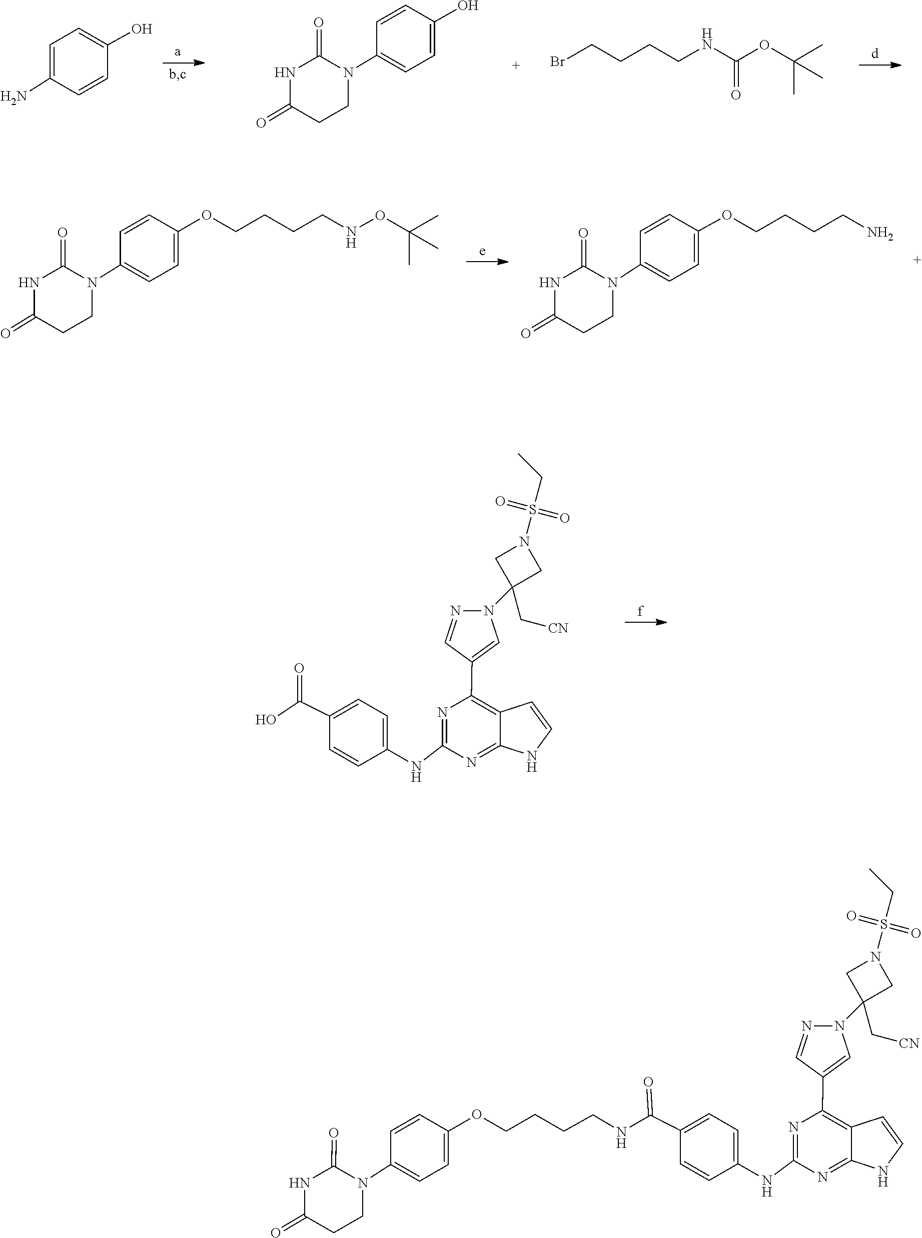

Reagents and conditions: a) methyl acrylate, hydroquinone, acetic acid, isopropanol, 85° C., 18 h; b) urea, acetic acid, 155° C., 18 h; c) HCl, 0° C.; d) K₂CO₃, DMF, 60° C., 20 h; e) TFA, CH₂Cl₂, rt, 3 h; f) N,N-diisopropylethylamine, EDC HCl, DMAP, DMF, rt, 2 h.

Step a: A mixture of 4-aminophenol (1.1 g, 10 mmol), methyl acrylate (1.0 mL, 11 mmol), acetic acid (0.1 mL, 1.8 mmol), and hydroquinone (0.004 g, 0.040 mmol) in isopropanol (10 mL) was refluxed at 85° C. for 18 h. The reaction mixture was cooled to room temperature, then concentrated in vacuo. To the crude was added 20% aqueous HCl solution (4 mL) and the mixture was stirred at room temperature for 1 h, then refluxed for 16 h. The mixture was cooled to room temperature, concentrated in vacou, and used without purification.

Step b: To the crude from Step (a) was added urea (1.19 g, 19.87 mmol) and acetic acid (4 mL). The reaction mixture was stirred at 115° C. in for 18 h, then was cooled to 0° C.

Step c: Preparation of 1-(4-hydroxyphenyl)dihydropyrimidine-2,4(1H,3H)-dione. To the crude from Step (b) at 0° C. was added 10% aqueous HCl (10 mL). The resulting mixture was filtered to obtain the title compound a black solid (0.70 g, 34%). LC-MS (ESI) m/z: 207.00 [M+H]⁺. ¹H NMR (500 MHz, DMSO-d6) δ 10.26 (s, 1H), 9.47 (br s, 1H), 7.10 (d, J=8.7 Hz, 2H), 6.76 (d, J=8.7 Hz, 2H), 3.68 (t, J=6.7 Hz, 2H), 2.68 (t, J=6.7 Hz, 2H). ¹³C NMR (126 MHz, DMSO) δ 171.16, 156.03, 152.79, 133.93, 127.42, 115.63, 45.52, 31.60.

Step d: Preparation of 1-(4-(4-(tert-butoxyamino)butoxy)phenyl)dihydropyrimidine-2,4(1H,3H)-dione. To a solution of 4-(Boc-amino)butyl bromide (0.15 g, 0.58 mmol) in DMF (1.5 mL) under nitrogen at room temperature was added 1-(4-hydroxyphenyl)dihydropyrimidine-2,4(1H,3H)-dione (0.10 g, 0.49 mmol) followed by potassium carbonate (0.20 g, 1.5 mmol). The reaction mixture was stirred at 60° C. for (s—2:00) 20 h then was diluted with water (15 mL) and ethyl acetate (15 mL) and stirred at room temperature for 5 min. The phases were then separated and the aqueous phase was extracted into ethyl acetate (3×10 mL). The combined organics were dried over sodium sulfate, filtered, and concentrated in vacuo. Purification using automated flash chromatography (MeOH/DCM) was followed by evaporation to obtain the title compound as a purple solid (0.051 g, 28%. LC-MS (ESI) m/z: 376.35 [M−H]⁻. ¹H NMR (500 MHz, CDCl₃) δ 7.65 (s, 1H), 7.21 (d, J=8.9 Hz, 2H), 6.93 (d, J=8.9 Hz, 2H), 4.65 (s, 1H), 3.99 (t, J=6.2 Hz, 2H), 3.83 (t, J=6.7 Hz, 2H), 3.20 (t, J=6.7 Hz, 2H), 2.84 (t, J=6.7 Hz, 2H), 1.83 (dt, J=8.4, 6.3 Hz, 2H), 1.68 (dd, J=8.7, 6.1 Hz, 2H), 1.47 (s, 9H). ¹³C NMR (126 MHz, CDCl₃) δ 169.50, 157.76, 156.03, 151.92, 133.82, 126.62, 115.10, 79.22, 67.78, 45.61, 40.26, 31.46, 28.44, 26.84, 26.49.

Step e: Preparation of 1-(4-(4-aminobutoxy)phenyl)dihydropyrimidine-2,4(1H,3H)-dione. To 1-(4-(4-(tert-butoxyamino)butoxy)phenyl)dihydropyrimidine-2,4(1H,3H)-dione (0.051 g, 0.14 mmol) in CH₂Cl₂ (5 mL) at room temperature was added TFA (0.52 mL, 6.8 mmol) and the reaction mixture was stirred at room temperature for 3 h. The reaction mixture was concentrated in vacuo to obtain the title compound as a dark purple oil, which was used without purification. LC-MS (ESI) m/z: 278.31 [M+H]⁺. ¹H NMR (500 MHz, MeOD) δ 7.24 (d, J=8.8 Hz, 2H), 6.95 (d, J=8.9 Hz, 2H), 4.04 (t, J=5.5 Hz, 2H), 3.80 (t, J=6.8 Hz, 2H), 3.01 (t, J=7.1 Hz, 2H), 2.79 (t, J=6.7 Hz, 2H), 1.89-1.82 (m, 4H). ¹³C NMR (126 MHz, MeOD) δ 172.95, 159.03, 154.84, 135.98, 128.32, 115.93, 68.46, 46.91, 40.57, 32.12, 27.21, 25.66.

Step f Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(4-(4-(2,4-dioxotetrahydropyrimidin-1(2H)-yl)phenoxy)butyl)benzamide. A mixture of 1-(4-(4-aminobutoxy)phenyl)dihydropyrimidine-2,4(1H,3H)-dione (0.019 g, 0.069 mmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (0.038 g, 0.0.075 mmol), N,N-diisopropylethylamine (0.048 mL, 0.27 mmol), EDC HCl (0.016 g, 0.082 mmol), DMAP (0.0017 g, 0.014 mmol) was stirred at room temperature in DMF (0.5 mL) for 2 h, then was purified using automated flash chromatography (MeOH/DCM). Evaporation gave the title compound as a white solid (0.016 g, 31%). LC-MS (ESI) m/z: 766.52 [M+H]⁺. ¹H NMR (500 MHz, DMSO-d6) δ 11.68 (s, 1H), 10.30 (s, 1H), 9.54 (s, 1H), 8.86 (s, 1H), 8.43 (s, 1H), 8.28 (t, J=5.8 Hz, 1H), 7.98 (d, J=8.6 Hz, 2H), 7.80 (d, J=8.6 Hz, 2H), 7.32 (dd, J=3.6, 2.2 Hz, 1H), 7.27-7.17 (m, 2H), 7.00-6.89 (m, 3H), 4.61 (d, J=9.1 Hz, 2H), 4.26 (d, J=9.1 Hz, 2H), 4.02 (t, J=6.3 Hz, 2H), 3.78-3.65 (m, 4H), 3.33-3.31 (m, 2H), 3.26 (q, J=7.3 Hz, 2H), 2.69 (t, J=6.7 Hz, 2H), 1.78 (dq, J=11.8, 6.5 Hz, 2H), 1.69 (q, J=7.0 Hz, 2H), 1.26 (t, J=7.3 Hz, 3H). ¹³C NMR (126 MHz, DMSO) δ 170.66, 165.95, 156.65, 155.28, 153.60, 152.30, 150.11, 144.29, 139.80, 134.83, 129.42, 127.85, 126.85, 125.86, 124.13, 122.26, 116.71, 116.68, 114.43, 108.03, 100.33, 67.42, 58.58, 56.07, 44.92, 43.31, 38.72, 31.10, 26.81, 26.27, 26.01, 7.44.

ii. Synthesis of Compound 58

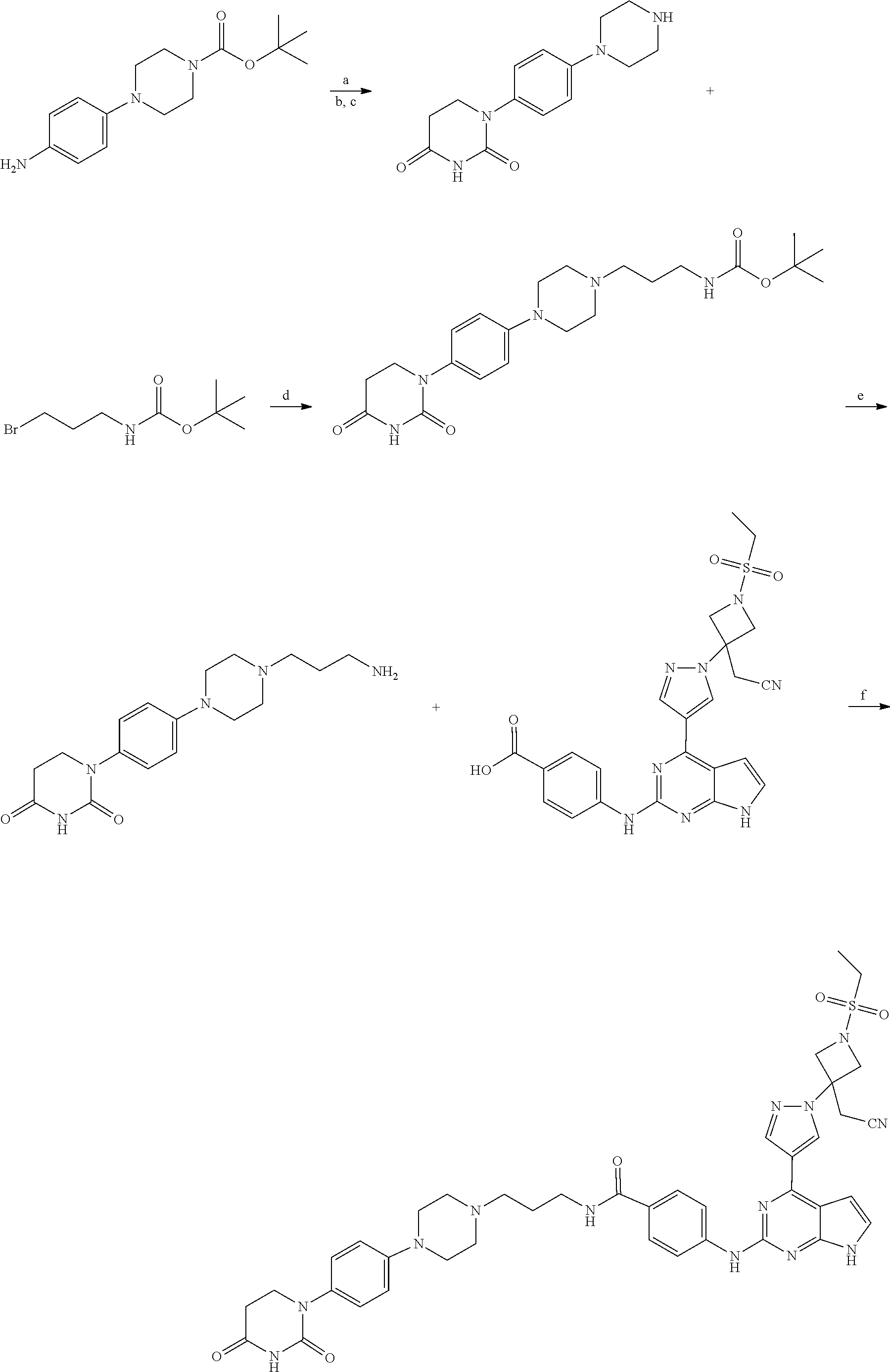

Reagents and conditions: a) acrylic acid, toluene, 115° C. 18 h; b) urea, acetic acid, 120° C., 19 h; c) HCl, 50° C., 16 h; d) N,N-diisopropylethylamine, DMF, 60° C., 26 h; e) TFA, CH₂Cl₂, rt, 4 h; f) N,N-diisopropylethylamine, EDC HCl, DMAP, DMF, rt, 4 h.

Step a: A mixture of 1-Boc-4-(4-aminophenyl)piperazine (1.00 g, 3.61 mmol) and acrylic acid (0.371 mL, 5.41 mmol) in toluene (20 mL) was stirred at 110° C. for 18 h. The reaction mixture was cooled to room temperature, then concentrated in vacuo to obtain a black solid which was used without purification.

Step b: To the crude from Step a was added urea (0.65 g, 10.8 mmol) and acetic acid (10 mL). The reaction mixture was stirred at 120° C. for (s—2:20) 19 h, then was cooled to room temperature. The mixture was concentrated in vacuo to give a black oil which was used without purification.

Step c: Preparation of 1-(4-(piperazin-1-yl)phenyl)dihydropyrimidine-2,4(1H,3H)-dione. To the crude from Step b was added 6 N HCl (11.4 mL, 68.4 mmol). The reaction mixture was stirred at 50° C. for 16 h, then cooled to room temperature. The mixture was concentrated in vacuo to give the title compound as a black solid, which was used without purification. LC-MS (ESI) m/z: 275.07 [M+H]⁺.

Step d: Preparation of tert-butyl (3-(4-(4-(2,4-dioxotetrahydropyrimidin-1(2H)-yl)phenyl)piperazin-1-yl)propyl)carbamate. A mixture of 1-(4-(piperazin-1-yl)phenyl)dihydropyrimidine-2,4(1H,3H)-dione (0.10 g, 0.32 mmol), 3-(Boc-amino)propyl bromide (0.077 g, 0.32 mmol), and N,N-diisopropylethylamine (0.34 mL, 1.9 mmol) was stirred in DMF at 60° C. for 26 h. The reaction mixture was the diluted with ethyl acetate (10 mL) and washed sequentially with brine (2×10 mL), then 5% aqueous LiCl solution (10 mL). The organic phase was dried over sodium sulfate, filtered, and concentrated in vacuo. Purification using automated flash chromatography (MeOH/DCM) followed by evaporation gave the title compound as a purple oil (0.008 g, 6%). LC-MS (ESI) m/z: 432.47 [M+H]⁺. ¹H NMR (500 MHz, MeOD) δ 7.21 (d, J=9.0 Hz, 2H), 7.00 (d, J=9.0 Hz, 2H), 3.80 (t, J=6.8 Hz, 2H), 3.22 (t, J=5.0 Hz, 4H), 3.10 (t, J=6.8 Hz, 2H), 2.79 (t, J=6.7 Hz, 2H), 2.64 (t, J=5.0 Hz, 4H), 2.51-2.39 (m, 2H), 1.72 (p, J=6.9 Hz, 2H), 1.44 (s, 9H). ¹³C NMR (126 MHz, MeOD) δ 173.02, 158.53, 154.79, 151.48, 134.96, 127.66, 117.53, 79.93, 57.15, 54.17, 49.96, 46.88, 39.73, 32.13, 28.77, 27.79.

Step e: Preparation of 1-(4-(4-(3-aminopropyl)piperazin-1-yl)phenyl)dihydropyrimidine-2,4(1H,3H)-dione. To tert-butyl (3-(4-(4-(2,4-dioxotetrahydropyrimidin-1(2H)-yl)phenyl)piperazin-1-yl)propyl)carbamate (0.008 g, 0.019 mmol) in CH₂Cl₂ (1 mL) at room temperature was added TFA (0.071 mL, 0.93 mmol) and the reaction mixture was stirred at room temperature for 4 h. The reaction mixture was concentrated in vacuo to obtain the title compound as a tan solid, which was used without purification. LC-MS (ESI) m/z: 332.25 [M+H]⁺. 1H NMR (500 MHz, MeOD) δ 7.28 (d, J=8.9 Hz, 2H), 7.07 (d, J=8.9 Hz, 2H), 3.82 (t, J=6.7 Hz, 2H), 3.76-3.31 (br m, 10H), 3.07 (t, J=7.7 Hz, 2H), 2.80 (t, J=6.8 Hz, 2H), 2.22-2.13 (m, 2H). 13C NMR (126 MHz, MeOD) δ 172.92, 154.78, 149.78, 136.35, 127.92, 118.33, 54.70, 53.17, 47.88, 46.75, 37.81, 32.10, 23.32.

Step f Preparation of 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)-N-(3-(4-(4-(2,4-dioxotetrahydropyrimidin-1(2H)-yl)phenyl)piperazin-1-yl)propyl)benzamide. A mixture of 1-(4-(4-(3-aminopropyl)piperazin-1-yl)phenyl)dihydropyrimidine-2,4(1H,3H)-dione (0.012 g, 0.036 mmol), 4-((4-(1-(3-(cyanomethyl)-1-(ethylsulfonyl)azetidin-3-yl)-1H-pyrazol-4-yl)-7H-pyrrolo[2,3-d]pyrimidin-2-yl)amino)benzoic acid (0.020 g, 0.040 mmol), N,N-diisopropylethylamine (0.025 mL, 0.15 mmol), EDC HCl (0.008 g, 0.043 mmol), DMAP (0.001 g, 0.007 mmol) was stirred at room temperature in DMF (0.5 mL) for 4 h. Purification by automated C18 flash chromatography (MeCN/water+0.1% formic acid) followed by evaporation gave the title compound as a white solid (0.012 g, 40%). LC-MS (ESI) m/z: 820.46 [M+H]⁺. ¹H NMR (500 MHz, DMSO-d6) δ 11.68 (s, 1H), 10.26 (s, 1H), 9.54 (s, 1H), 8.85 (s, 1H), 8.43 (s, 1H), 8.25 (d, J=32.0 Hz, 1H), 7.97 (d, J=8.5 Hz, 2H), 7.80 (d, J=8.4 Hz, 2H), 7.38-7.27 (m, 1H), 7.14 (d, J=8.3 Hz, 2H), 6.99-6.90 (m, 3H), 4.61 (d, J=9.1 Hz, 2H), 4.26 (d, J=9.0 Hz, 2H), 3.76-3.64 (m, 4H), 3.29 (d, J=9.0 Hz, 2H), 3.26 (d, J=7.3 Hz, 2H), 3.14 (t, J=5.0 Hz, 4H), 2.67 (s, 2H), 2.54 (s, 4H), 2.41 (t, J=7.1 Hz, 2H), 1.72 (dt, J=20.4, 7.0 Hz, 2H), 1.26 (t, J=7.3 Hz, 3H). ¹³C NMR (126 MHz, DMSO) δ 171.14, 166.41, 164.00, 155.75, 154.08, 152.75, 150.58, 149.68, 144.76, 140.28, 133.85, 129.90, 128.29, 126.68, 126.37, 124.60, 122.73, 117.17, 115.79, 108.50, 100.80, 59.05, 56.54, 53.21, 48.86, 45.38, 43.79, 38.23, 31.59, 27.28, 26.97, 7.91.

2. Biology Methods

Cytotoxicity assay: MHH-CALL-4 cells were maintained in RPMI1640 medium (Invitrogen) supplemented with 10% FBS (Hyclone), Penicillin/Streptomycin (100 units/mL) and Glutamine (100 μM). 2×105 cells were seeded in 100 μl medium/well in 96 well assay plates (Corning 3603). Assays were performed in triplicate; compounds to be screened were added to assay plates from DMSO stock solutions by pin transfer using 50SS pins (V&P Scientific). The assay plates incubated at 37° C. in 5% CO₂ for 72 hours. Cells were then incubated for four hours with resazurin (Sigma) solution and read on a Synergy HT plate reader (Biotek, Winooski, VT). High-throughput assay data was analyzed using our in-house Robust Interpretation of Screening Experiments (RISE) application written in Pipeline Pilot (Biovia, v. 17.2.0) and the R program (R Core Team, 2013). All the AUC (area under the dose-response curve) per compound calculated from its dose response curve by RISE protocol. Selected data were plotted and analyzed by GraphPad Prism software v7 using non-linear regression curve fitting.

Western blot assay: MHH-CALL-4 Cells were washed twice with ice-cold PBS and lysed in RIPA lysis buffer (Sigma) freshly supplemented with Halt™ Protease Inhibitor Cocktail (ThermoFisher) for 15 minutes on ice. The cell pellet was removed by centrifugation at 13,000 g at 4° C. for 15 minutes. Protein concentration was measured by BCA assay (Thermo Fisher Scientific). Proteins were denatured in NuPAGE LDS 4× sample buffer supplemented with reducing agents (Invitrogen). Typically, 10-20 μg of total protein was loaded per lane on a 4%-12% NUPAGE Bis-Tris gradient gel (Invitrogen) and analyzed by standard immunoblotting and imaged using Li—COR Odyssey CLx (LI-COR Biotechnology, Lincoln, NE). To access the protein degradation, the signal of target protein was normalized by the signal of ACBT and the signal of vehicle control was set as 100%. Primary antibodies used were: JAK1 (Cell Signaling #3332S), JAK2 (#3230S), JAK3 (#8827S), TYK2 (#9312S), GSPT1 (#14980S), CRBN (#71810S), IKZF1 (Santa Cruz Biotechnology, SC-398265) and ACBT (Santa Cruz Biotechnology, SC-47778).

3. Evaluation of Proteolysis-Targeting Chimeras (PROTACS)

The structures of the exemplary compounds evaluated for activity are shown in Table 1, and their accompanying data are shown in Table 2.

TABLE 1

| No. | Structure |
|---|---|
| 1 | 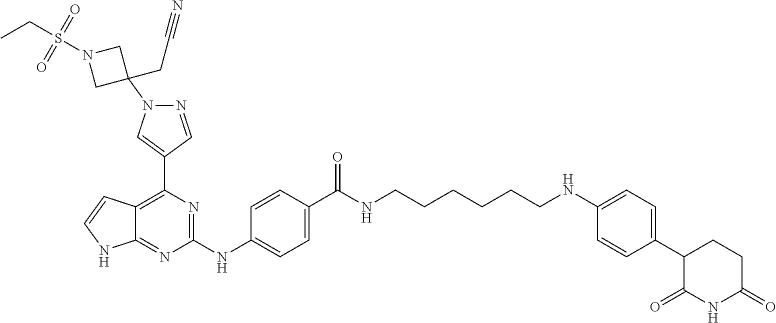 [SMILES: CCS(=O)(=O)N1CC(CC#N)(n2cc(-c3nc(Nc4ccc(C(=O)NCCCCCCNc5ccc(C6CCC(=O)NC6=O)cc5)cc4)nc4[nH]ccc34)cn2)C1] |
| 2 | 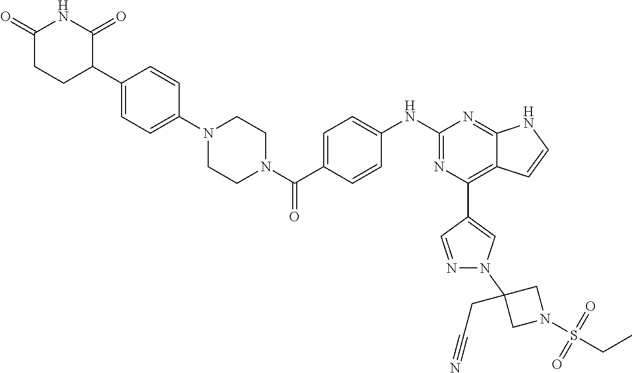 [SMILES: CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1] |
| 3 | 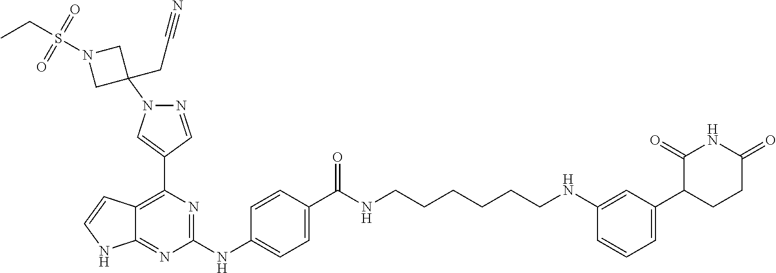 [SMILES: CCS(=O)(=O)N1CC(CC#N)(n2cc(-c3nc(Nc4ccc(C(=O)NCCCCCCNc5cccc(C6CCC(=O)NC6=O)c5)cc4)nc4[nH]ccc34)cn2)C1] |
| 4 | 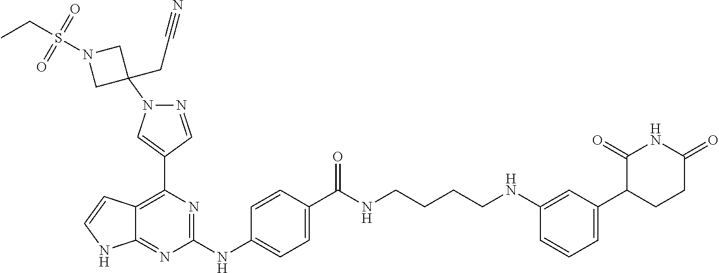 [SMILES: CCS(=O)(=O)N1CC(CC#N)(n2cc(-c3nc(Nc4ccc(C(=O)NCCCCNc5cccc(C6CCC(=O)NC6=O)c5)cc4)nc4[nH]ccc34)cn2)C1] |
| 5 | 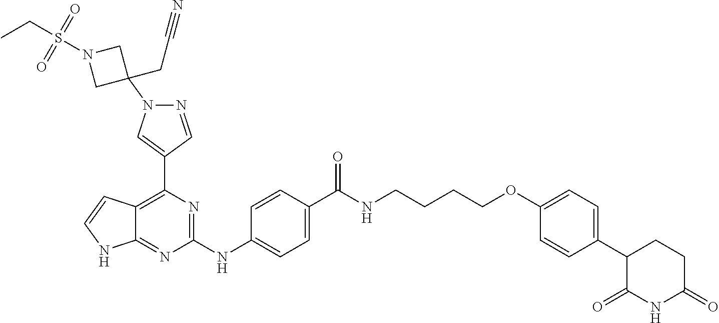 [SMILES: CCS(=O)(=O)N1CC(CC#N)(n2cc(-c3nc(Nc4ccc(C(=O)NCCCCOc5ccc(C6CCC(=O)NC6=O)cc5)cc4)nc4[nH]ccc34)cn2)C1] |
| 6 | 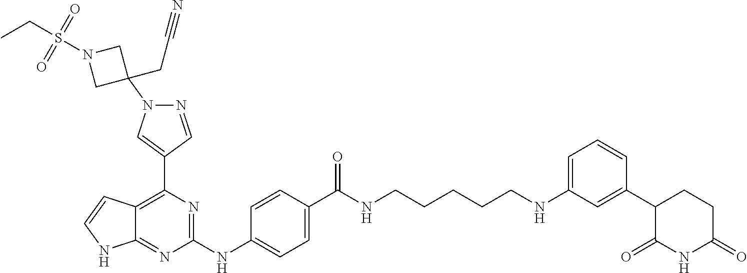 [SMILES: CCS(=O)(=O)N1CC(CC#N)(n2cc(-c3nc(Nc4ccc(C(=O)NCCCCCNc5cccc(C6CCC(=O)NC6=O)c5)cc4)nc4[nH]ccc34)cn2)C1] |
| 7 | 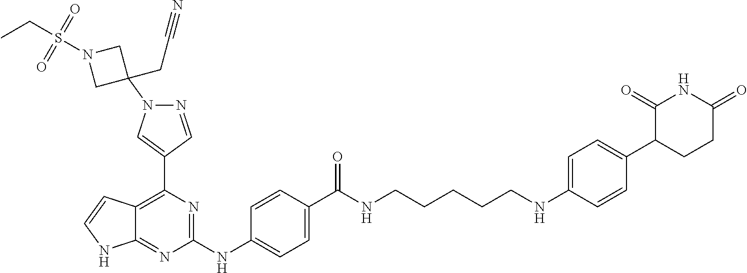 [SMILES: CCS(=O)(=O)N1CC(CC#N)(n2cc(-c3nc(Nc4ccc(C(=O)NCCCCCNc5ccc(C6CCC(=O)NC6=O)cc5)cc4)nc4[nH]ccc34)cn2)C1] |
| 8 | 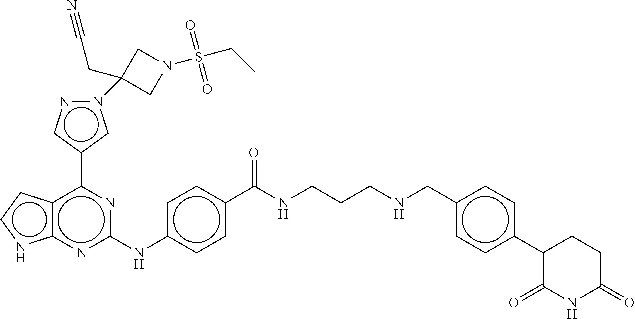 |
| 9 | 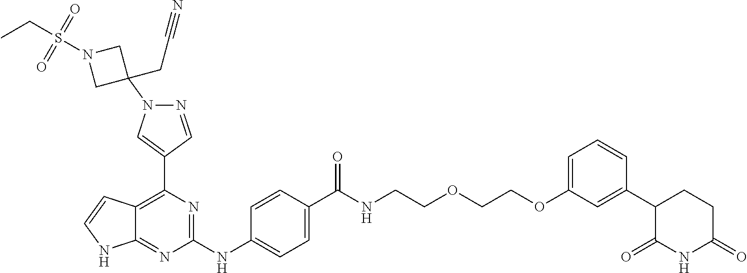 [SMILES: CCS(=O)(=O)N1CC(CC#N)(n2cc(-c3nc(Nc4ccc(C(=O)NCCOCCOc5cccc(C6CCC(=O)NC6=O)c5)cc4)nc4[nH]ccc34)cn2)C1] |
| 10 | 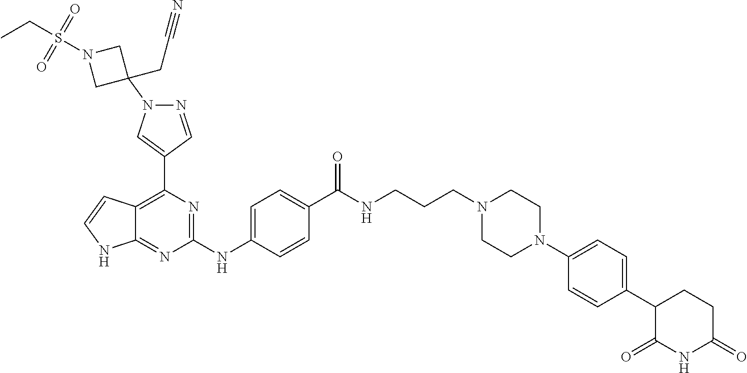 [SMILES: CCS(=O)(=O)N1CC(CC#N)(n2cc(-c3nc(Nc4ccc(C(=O)NCCCN5CCN(c6ccc(C7CCC(=O)NC7=O)cc6)CC5)cc4)nc4[nH]ccc34)cn2)C1] |
| 11 | 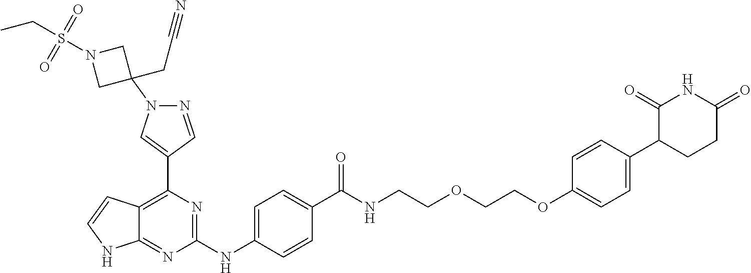 [SMILES: CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1] |
| 12 | 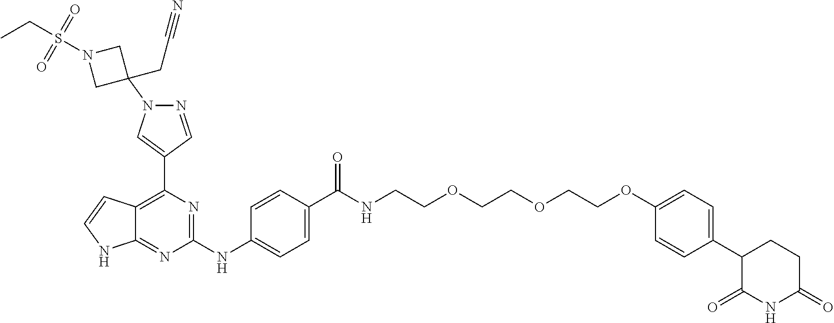 [SMILES: CCS(=O)(=O)N1CC(CC#N)(n2cc(-c3nc(Nc4ccc(C(=O)NCCOCCOCCOc5ccc(C6CCC(=O)NC6=O)cc5)cc4)nc4[nH]ccc34)cn2)C1] |
| 13 | 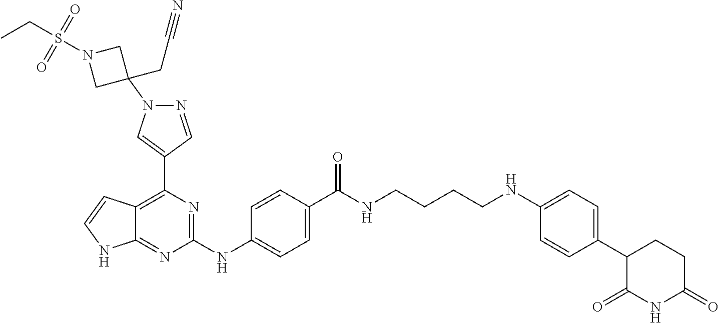 [SMILES: CCS(=O)(=O)N1CC(CC#N)(n2cc(-c3nc(Nc4ccc(C(=O)NCCCCNc5ccc(C6CCC(=O)NC6=O)cc5)cc4)nc4[nH]ccc34)cn2)C1] |
| 14 | 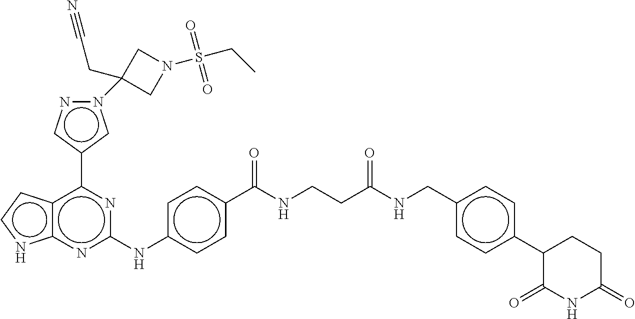 |
| 15 | 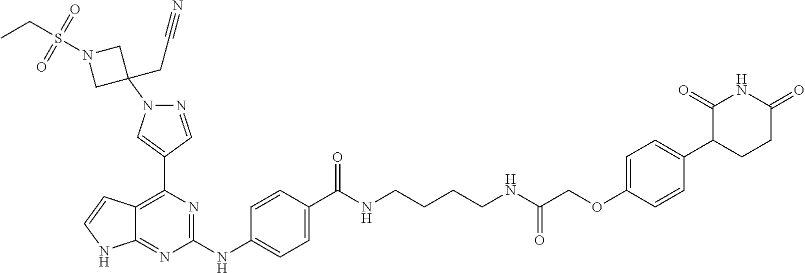 [SMILES: CCS(=O)(=O)N1CC(CC#N)(n2cc(-c3nc(Nc4ccc(C(=O)NCCCCNC(=O)COc5ccc(C6CCC(=O)NC6=O)cc5)cc4)nc4[nH]ccc34)cn2)C1] |
| 16 | 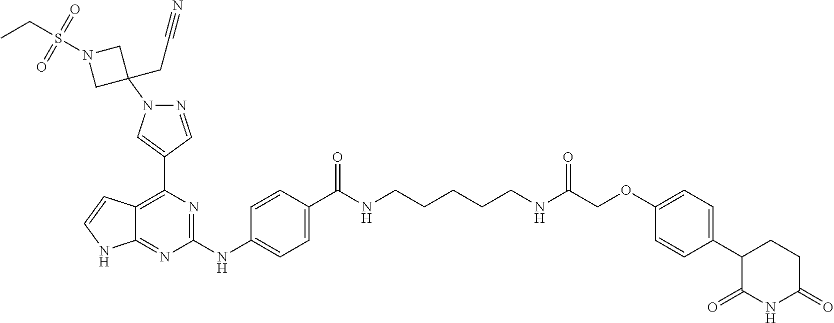 [SMILES: CCS(=O)(=O)N1CC(CC#N)(n2cc(-c3nc(Nc4ccc(C(=O)NCCCCCNC(=O)COc5ccc(C6CCC(=O)NC6=O)cc5)cc4)nc4[nH]ccc34)cn2)C1] |
| 17 | 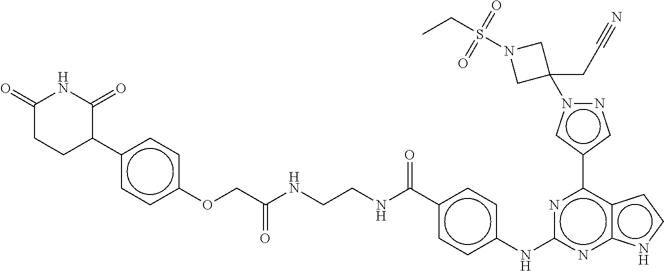 |
| 18 | 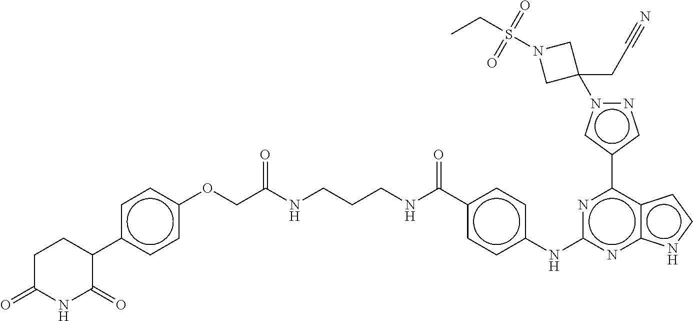 |
| 19 | 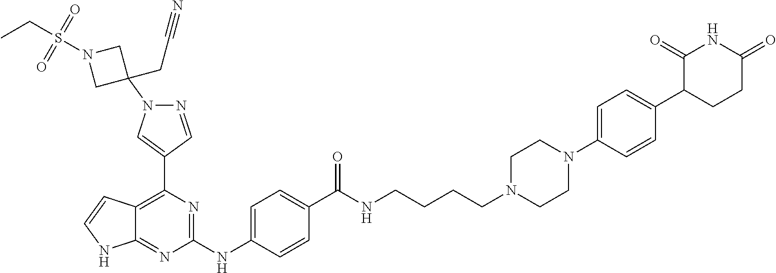 [SMILES: CCS(=O)(=O)N1CC(CC#N)(n2cc(-c3nc(Nc4ccc(C(=O)NCCCCN5CCN(c6ccc(C7CCC(=O)NC7=O)cc6)CC5)cc4)nc4[nH]ccc34)cn2)C1] |
| 20 | 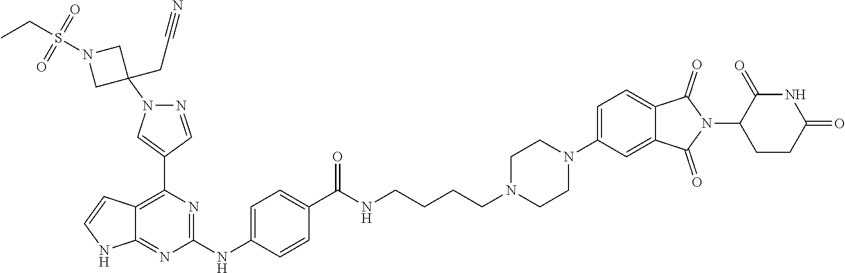 [SMILES: CCS(=O)(=O)N1CC(CC#N)(n2cc(-c3nc(Nc4ccc(C(=O)NCCCCN5CCN(c6ccc7c(c6)C(=O)N(C6CCC(=O)NC6=O)C7=O)CC5)cc4)nc4[nH]ccc34)cn2)C1] |
| 21 | 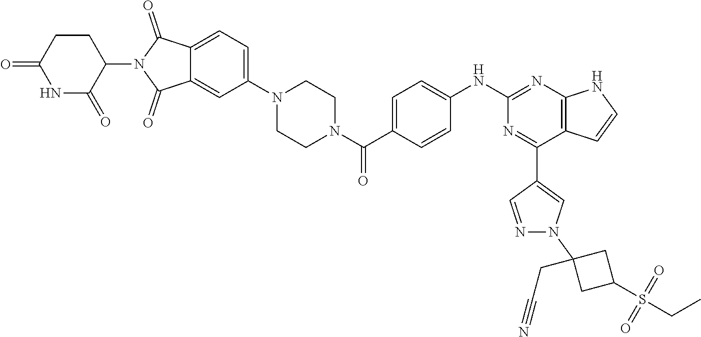 [SMILES: CCS(=O)(=O)C1CC(CC#N)(n2cc(-c3nc(Nc4ccc(C(=O)N5CCN(c6ccc7c(c6)C(=O)N(C6CCC(=O)NC6=O)C7=O)CC5)cc4)nc4[nH]ccc34)cn2)C1] |
| 22 | 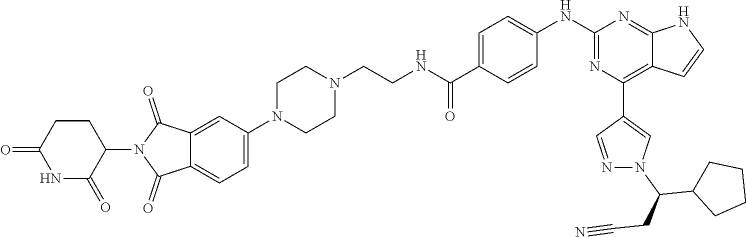 [SMILES: N#CC[C@H](C1CCCC1)n1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1] |
| 23 | 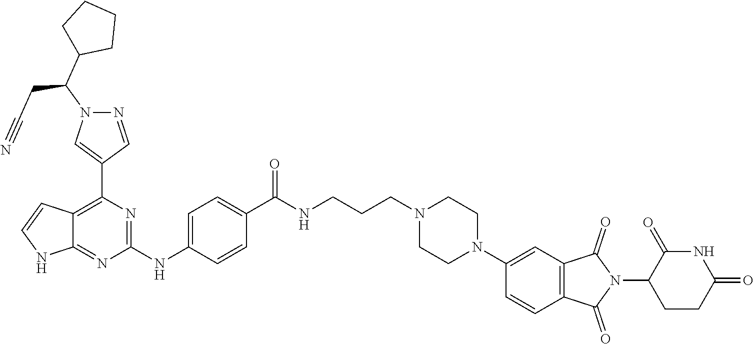 [SMILES: N#CC[C@@H](C1CCCC1)n1cc(-c2nc(Nc3ccc(C(=O)NCCCN4CCN(c5ccc6c(c5)C(=O)N(C5CCC(=O)NC5=O)C6=O)CC4)cc3)nc3[nH]ccc23)cn1] |
| 24 | 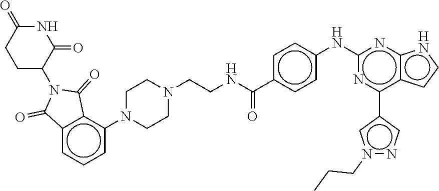 |
| 25 | 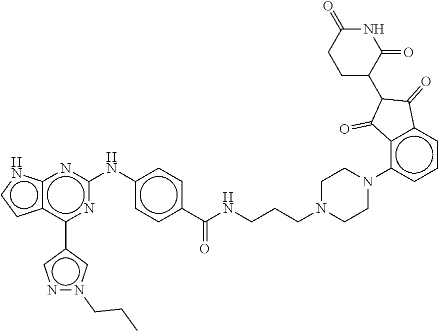 |
| 26 | 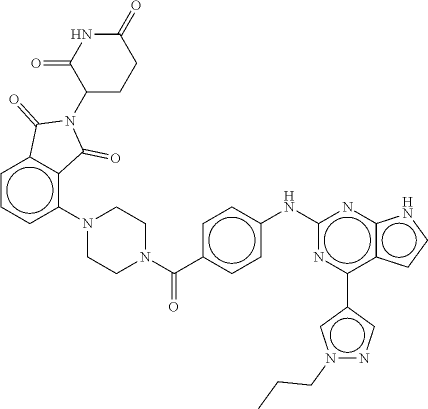 |
| 27 | 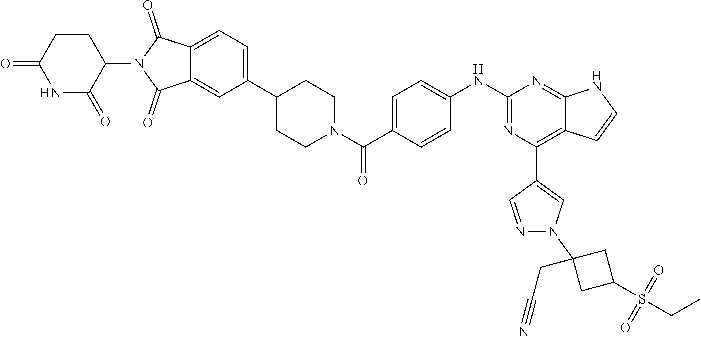 [SMILES: CCS(=O)(=O)C1CC(CC#N)(n2cc(-c3nc(Nc4ccc(C(=O)N5CCC(c6ccc7c(c6)C(=O)N(C6CCC(=O)NC6=O)C7=O)CC5)cc4)nc4[nH]ccc34)cn2)C1] |
| 28 | 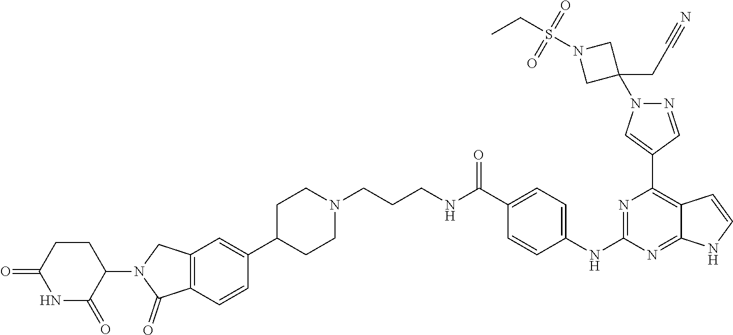 [SMILES: CCS(=O)(=O)N1CC(CC#N)(n2cc(-c3nc(Nc4ccc(C(=O)NCCCN5CCC(c6ccc7c(c6)CN(C6CCC(=O)NC6=O)C7=O)CC5)cc4)nc4[nH]ccc34)cn2)C1] |
| 29 | 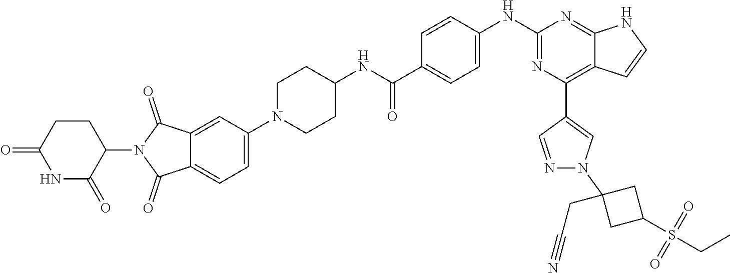 [SMILES: CCS(=O)(=O)C1CC(CC#N)(n2cc(-c3nc(Nc4ccc(C(=O)NC5CCN(c6ccc7c(c6)C(=O)N(C6CCC(=O)NC6=O)C7=O)CC5)cc4)nc4[nH]ccc34)cn2)C1] |
| 30 | 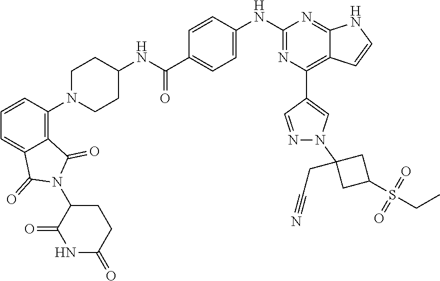 [SMILES: CCS(=O)(=O)C1CC(CC#N)(n2cc(-c3nc(Nc4ccc(C(=O)NC5CCN(c6cccc7c6C(=O)N(C6CCC(=O)NC6=O)C7=O)CC5)cc4)nc4[nH]ccc34)cn2)C1] |
| 31 | 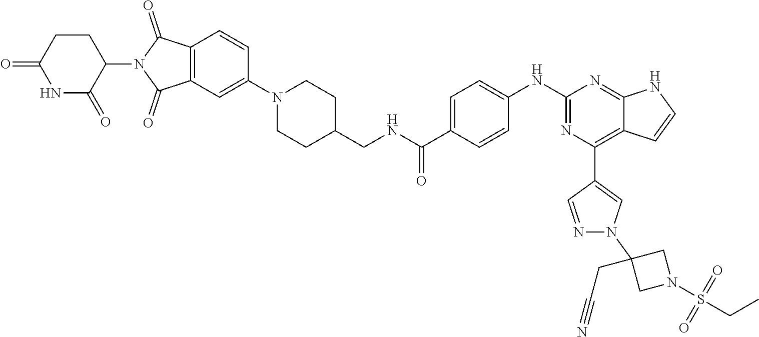 [SMILES: CCS(=O)(=O)N1CC(CC#N)(n2cc(-c3nc(Nc4ccc(C(=O)NCC5CCN(c6ccc7c(c6)C(=O)N(C6CCC(=O)NC6=O)C7=O)CC5)cc4)nc4[nH]ccc34)cn2)C1] |
| 32 | 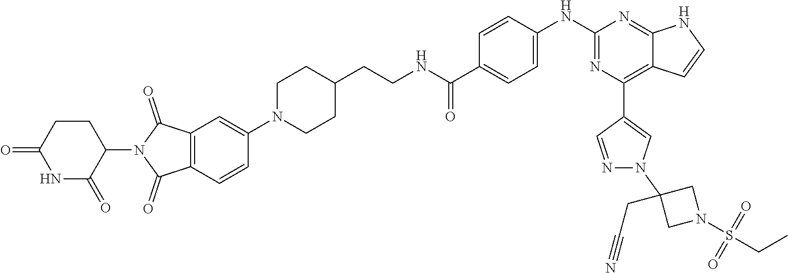 [SMILES: CCS(=O)(=O)N1CC(CC#N)(n2cc(-c3nc(Nc4ccc(C(=O)NCCC5CCN(c6ccc7c(c6)C(=O)N(C6CCC(=O)NC6=O)C7=O)CC5)cc4)nc4[nH]ccc34)cn2)C1] |
| 33 | 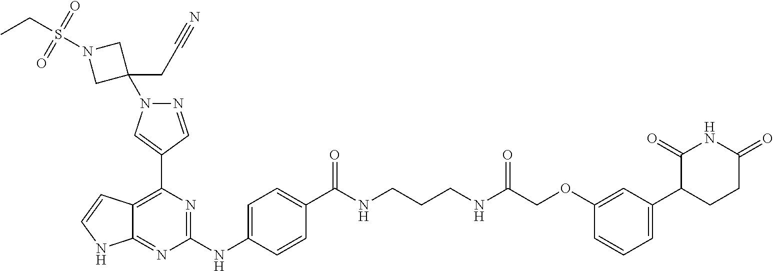 [SMILES: CCS(=O)(=O)N1CC(CC#N)(n2cc(-c3nc(Nc4ccc(C(=O)NCCCNC(=O)COc5cccc(C6CCC(=O)NC6=O)c5)cc4)nc4[nH]ccc34)cn2)C1] |
| 34 | 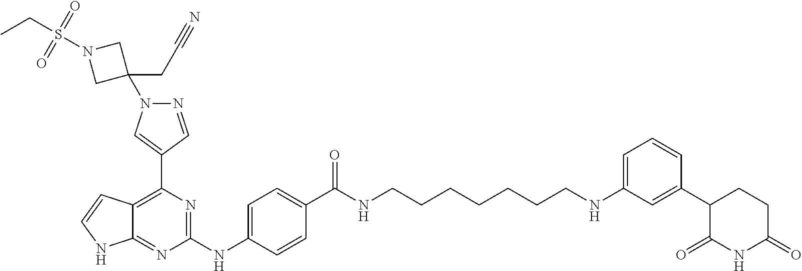 [SMILES: CCS(=O)(=O)N1CC(CC#N)(n2cc(-c3nc(Nc4ccc(C(=O)NCCCCCCCNc5cccc(C6CCC(=O)NC6=O)c5)cc4)nc4[nH]ccc34)cn2)C1] |
| 35 | 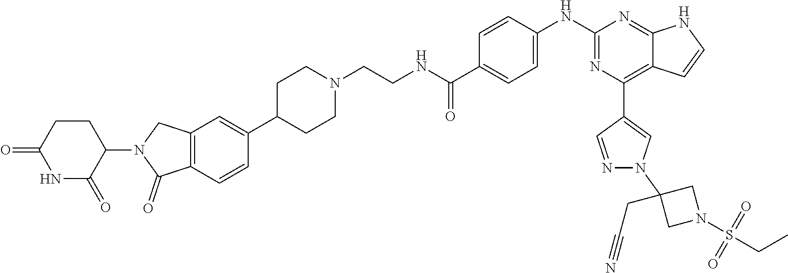 [SMILES: CCS(=O)(=O)N1CC(CC#N)(n2cc(-c3nc(Nc4ccc(C(=O)NCCN5CCC(c6ccc7c(c6)CN(C6CCC(=O)NC6=O)C7=O)CC5)cc4)nc4[nH]ccc34)cn2)C1] |
| 36 | 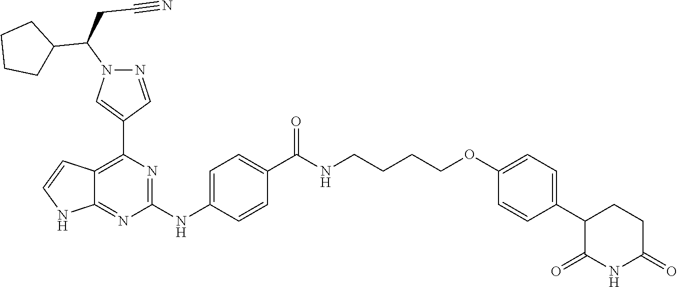 [SMILES: N#CC[C@H](C1CCCC1)n1cc(-c2nc(Nc3ccc(C(=O)NCCCCOc4ccc(C5CCC(=O)NC5=O)cc4)cc3)nc3[nH]ccc23)cn1] |
| 37 | 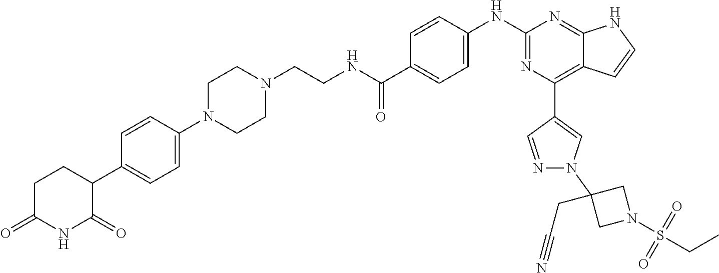 [SMILES: CCS(=O)(=O)N1CC(CC#N)(n2cc(-c3nc(Nc4ccc(C(=O)NCCN5CCN(c6ccc(C7CCC(=O)NC7=O)cc6)CC5)cc4)nc4[nH]ccc34)cn2)C1] |
| 38 | 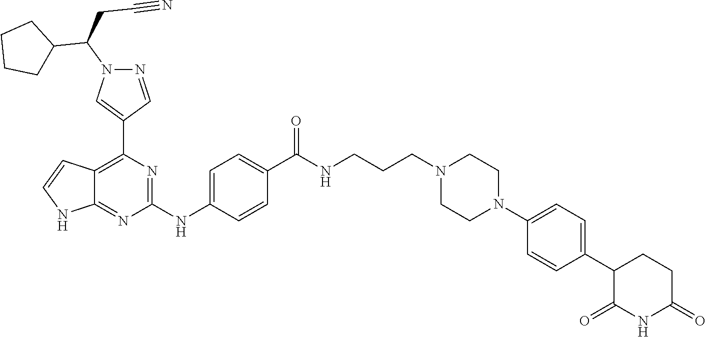 [SMILES: N#CC[C@H](C1CCCC1)n1cc(-c2nc(Nc3ccc(C(=O)NCCCN4CCN(c5ccc(C6CCC(=O)NC6=O)cc5)CC4)cc3)nc3[nH]ccc23)cn1] |
| 39 | 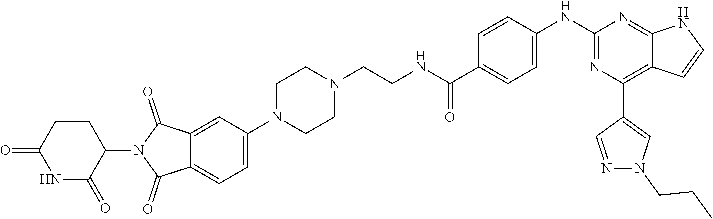 [SMILES: CCCn1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1] |
| 40 | 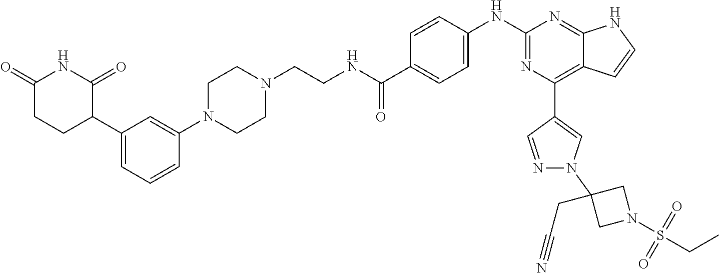 [SMILES: CCS(=O)(=O)N1CC(CC#N)(n2cc(-c3nc(Nc4ccc(C(=O)NCCN5CCN(c6cccc(C7CCC(=O)NC7=O)c6)CC5)cc4)nc4[nH]ccc34)cn2)C1] |
| 41 | 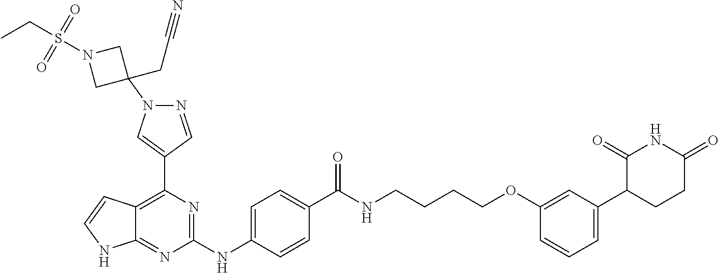 [SMILES: CCS(=O)(=O)N1CC(CC#N)(n2cc(-c3nc(Nc4ccc(C(=O)NCCCCOc5cccc(C6CCC(=O)NC6=O)c5)cc4)nc4[nH]ccc34)cn2)C1] |
| 42 | 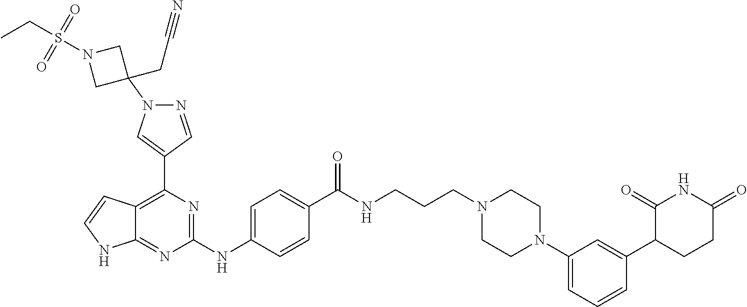 [SMILES: CCS(=O)(=O)N1CC(CC#N)(n2cc(-c3nc(Nc4ccc(C(=O)NCCCN5CCN(c6cccc(C7CCC(=O)NC7=O)c6)CC5)cc4)nc4[nH]ccc34)cn2)C1] |
| 43 | 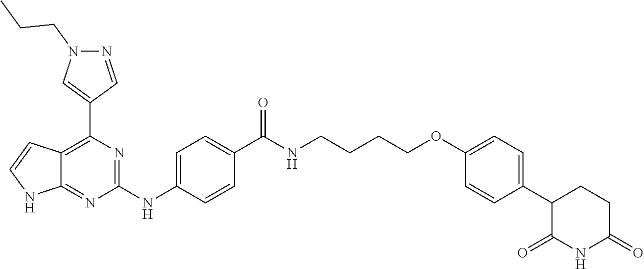 [SMILES: CCCn1cc(-c2nc(Nc3ccc(C(=O)NCCCCOc4ccc(C5CCC(=O)NC5=O)cc4)cc3)nc3[nH]ccc23)cn1] |
| 44 | 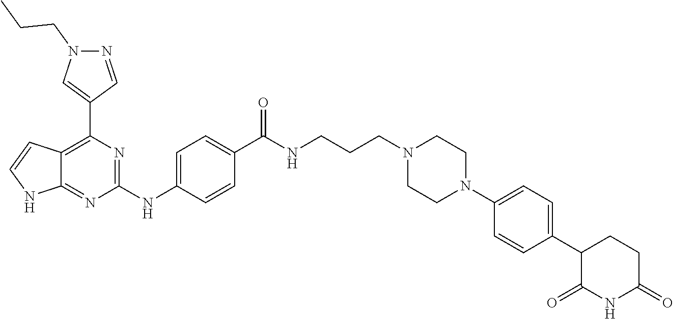 [SMILES: CCCn1cc(-c2nc(Nc3ccc(C(=O)NCCCN4CCN(c5ccc(C6CCC(=O)NC6=O)cc5)CC4)cc3)nc3[nH]ccc23)cn1] |
| 45 | 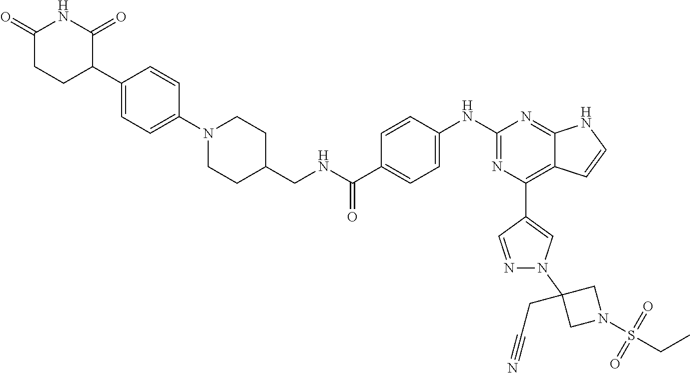 [SMILES: CCS(=O)(=O)N1CC(CC#N)(n2cc(-c3nc(Nc4ccc(C(=O)NCC5CCN(c6ccc(C7CCC(=O)NC7=O)cc6)CC5)cc4)nc4[nH]ccc34)cn2)C1] |
| 46 | 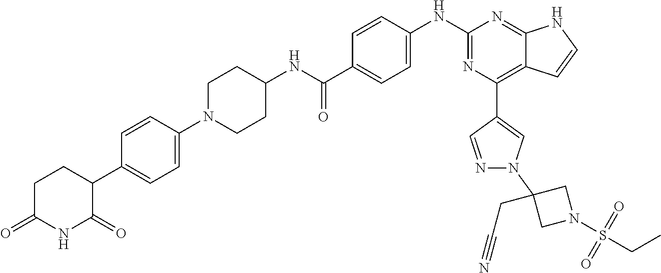 [SMILES: CCS(=O)(=O)N1CC(CC#N)(n2cc(-c3nc(Nc4ccc(C(=O)NC5CCN(c6ccc(C7CCC(=O)NC7=O)cc6)CC5)cc4)nc4[nH]ccc34)cn2)C1] |
| 47 | 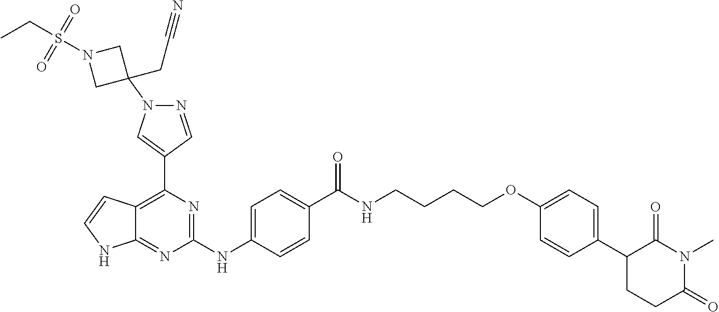 [SMILES: CCS(=O)(=O)N1CC(CC#N)(n2cc(-c3nc(Nc4ccc(C(=O)NCCCCOc5ccc(C6CCC(=O)N(C)C6=O)cc5)cc4)nc4[nH]ccc34)cn2)C1] |
| 48 | 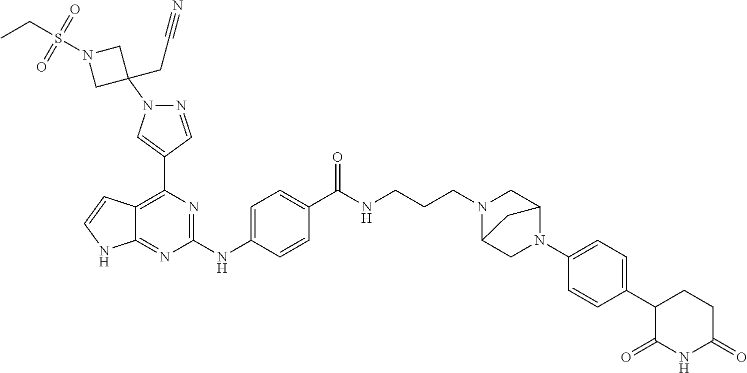 [SMILES: CCS(=O)(=O)N1CC(CC#N)(n2cc(-c3nc(Nc4ccc(C(=O)NCCCN5CC6CC5CN6c5ccc(C6CCC(=O)NC6=O)cc5)cc4)nc4[nH]ccc34)cn2)C1] |
| 49 | 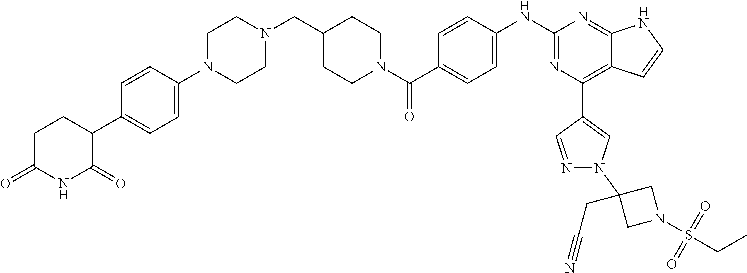 [SMILES: CCS(=O)(=O)N1CC(CC#N)(n2cc(-c3nc(Nc4ccc(C(=O)N5CCC(CN6CCN(c7ccc(C8CCC(=O)NC8=O)cc7)CC6)CC5)cc4)nc4[nH]ccc34)cn2)C1] |
| 50 | 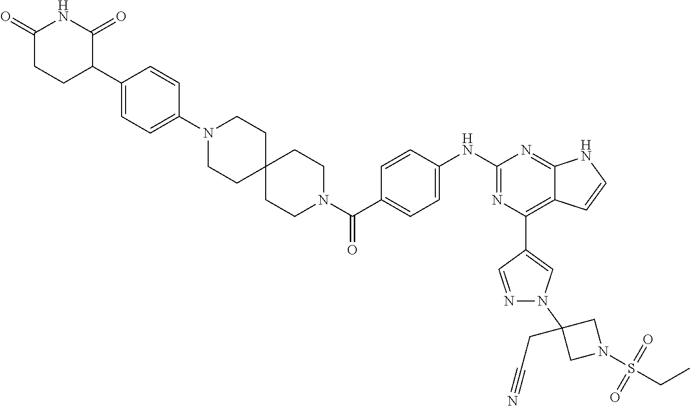 [SMILES: CCS(=O)(=O)N1CC(CC#N)(n2cc(-c3nc(Nc4ccc(C(=O)N5CCC6(CC5)CCN(c5ccc(C7CCC(=O)NC7=O)cc5)CC6)cc4)nc4[nH]ccc34)cn2)C1] |
| 51 | 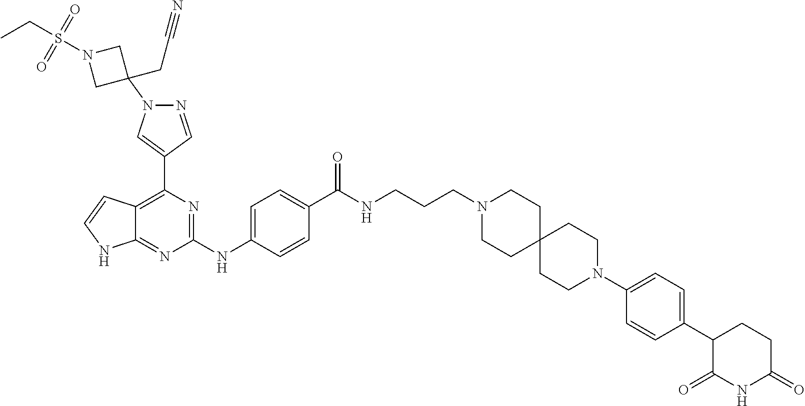 [SMILES: CCS(=O)(=O)N1CC(CC#N)(n2cc(-c3nc(Nc4ccc(C(=O)NCCCN5CCC6(CC5)CCN(c5ccc(C7CCC(=O)NC7=O)cc5)CC6)cc4)nc4[nH]ccc34)cn2)C1] |
| 52 | 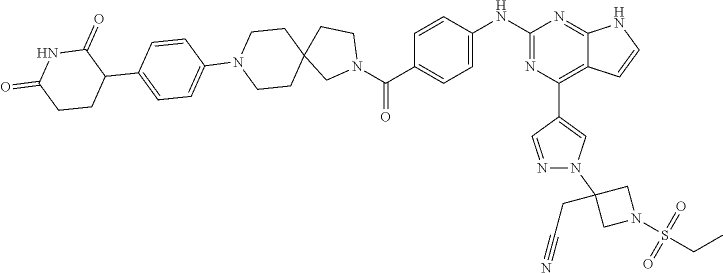 [SMILES: CCS(=O)(=O)N1CC(CC#N)(n2cc(-c3nc(Nc4ccc(C(=O)N5CCC6(CCN(c7ccc(C8CCC(=O)NC8=O)cc7)CC6)C5)cc4)nc4[nH]ccc34)cn2)C1] |
| 53 | 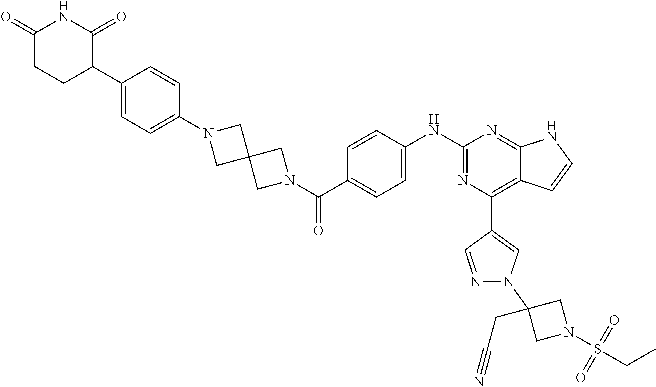 [SMILES: CCS(=O)(=O)N1CC(CC#N)(n2cc(-c3nc(Nc4ccc(C(=O)N5CC6(C5)CN(c5ccc(C7CCC(=O)NC7=O)cc5)C6)cc4)nc4[nH]ccc34)cn2)C1] |
| 54 | 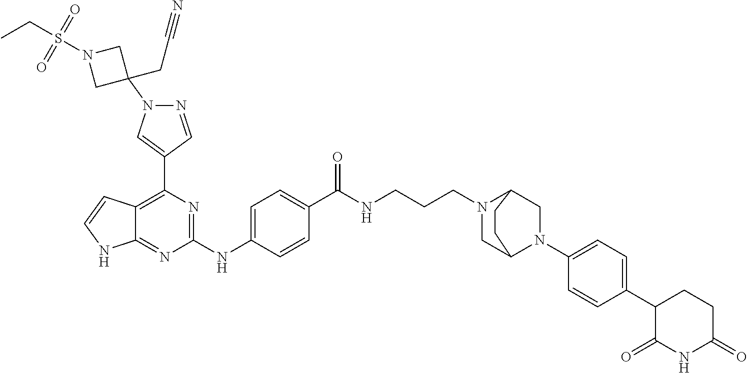 [SMILES: CCS(=O)(=O)N1CC(CC#N)(n2cc(-c3nc(Nc4ccc(C(=O)NCCCN5CC6CCC5CN6c5ccc(C6CCC(=O)NC6=O)cc5)cc4)nc4[nH]ccc34)cn2)C1] |
| 55 | 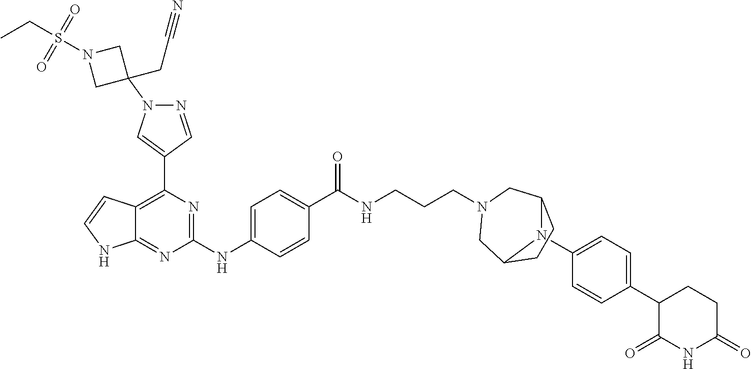 [SMILES: CCS(=O)(=O)N1CC(CC#N)(n2cc(-c3nc(Nc4ccc(C(=O)NCCCN5CC6CCC(C5)N6c5ccc(C6CCC(=O)NC6=O)cc5)cc4)nc4[nH]ccc34)cn2)C1] |
| 56 | 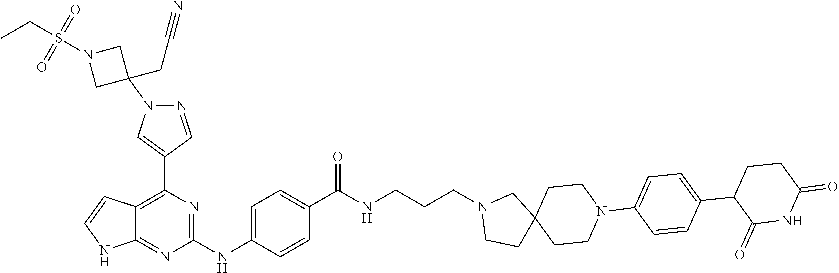 [SMILES: CCS(=O)(=O)N1CC(CC#N)(n2cc(-c3nc(Nc4ccc(C(=O)NCCCN5CCC6(CCN(c7ccc(C8CCC(=O)NC8=O)cc7)CC6)C5)cc4)nc4[nH]ccc34)cn2)C1] |
| 57 | 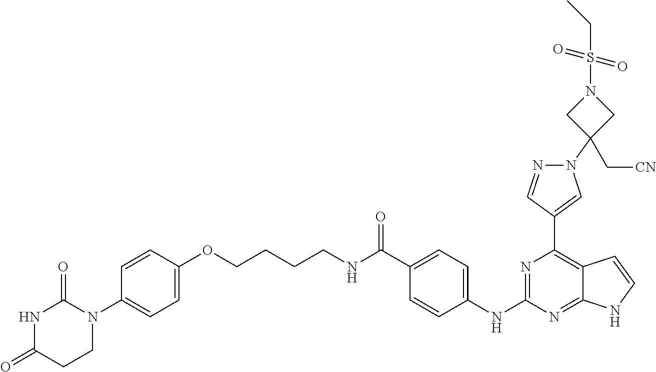 [SMILES: CCS(=O)(=O)N1CC(CC#N)(n2cc(-c3nc(Nc4ccc(C(=O)NCCCCOc5ccc(N6CCC(=O)NC6=O)cc5)cc4)nc4[nH]ccc34)cn2)C1] |
| 58 | 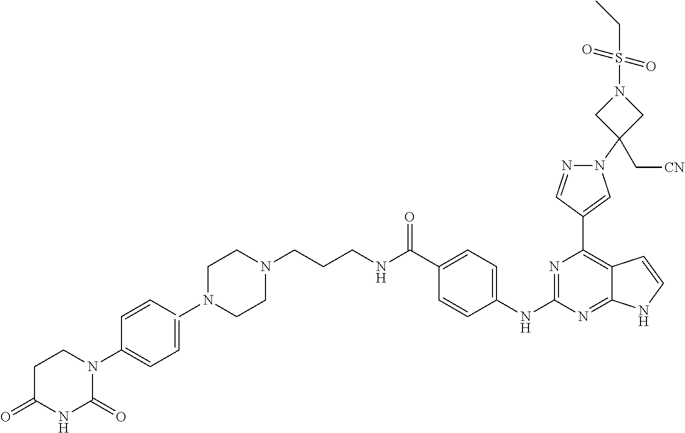 [SMILES: CCS(=O)(=O)N1CC(CC#N)(n2cc(-c3nc(Nc4ccc(C(=O)NCCCN5CCN(c6ccc(N7CCC(=O)NC7=O)cc6)CC5)cc4)nc4[nH]ccc34)cn2)C1] |

TABLE 2

| | | | | | | | Neosubstrate | | |
|---|---|---|---|---|---|---|---|---|---|
| | | | | | | | Degradation | | |
| | | | Target Protein Degradation | | | | (% remaining at | | |
| | EC₅₀ | | (% remaining at 100 nM, 24 h) | | | | 100 nM, 24 h) | | CRBN |
| No. | (nM) | AUC | JAK1 | JAK2 | JAK3 | TYK2 | GSPT1 | IKZF1 | ligand |
| 1 | 0.80 | 231 | 128 | 34 | 37 | | 84 | | PG |
| 2 | 1.00 | 167 | | None | | | None | | PG |
| 3 | 1.70 | 248 | 94 | 56 | 79 | | 84 | | PG |
| 4 | 2.10 | 120 | 17 | 30 | 44 | | 124 | | PG |
| 5 | 2.20 | 124 | 124 | 22 | 65 | 142 | 71 | 79 | PG |
| 6 | 5.90 | 136 | 44 | 37 | 54 | | 1511 | | PG |
| 7 | 13.90 | 248 | 78 | 29 | 50 | | 63 | | PG |
| 8 | 21.53 | 122 | 140 | 89 | 106 | | 89 | | PG |
| 9 | 872.80 | 132 | | | | | | | PG |
| 10 | 2581.20 | 114 | 86 | 25 | 46 | 118 | 109 | 87 | PG |
| 11 | >10,000 | | 110 | 90 | 70 | | 130 | | PG |
| 12 | >5000 | 116 | | | | | | | PG |
| 13 | >5000 | 93 | | | | | | | PG |
| 14 | >5061.59 | 11 | 82 | 140 | 100 | 84 | 100 | 99 | PG |
| 15 | >5500 | 156 | | 29 | | | 78 | | PG |
| 16 | >5700 | 136 | | | | | | | PG |
| 17 | >783.88 | NA | 105 | 120 | 102 | 88 | 110 | 96 | PG |
| 18 | >811.87 | 95 | 83 | 82 | 80 | 122 | 117 | 105 | PG |
| 19 | 1452 | 225 | 87 | 34 | 112 | | 129 | | PG |
| 20 | 13.60 | 123 | 138 | 40 | 102 | | 128 | | Pom- |
| | | | | | | | | | Pipz |
| 21 | 13.90 | 118 | | 53 | | | | | Pom- |
| | | | | | | | | | Pipz |
| 22 | 18.10 | 141 | 72 | 36 | 61 | | 103 | | Pom- |
| | | | | | | | | | Pipz |
| 23 | 30.90 | 79 | 145 | 67 | 109 | | 155 | | Pom- |
| | | | | | | | | | Pipz |
| 24 | 83.57 | 208 | | 93 | | | 0 | | Pom- |
| | | | | | | | | | Pipz |
| 25 | 169.45 | 250 | | None | | | Medium | | Pom- |
| | | | | | | | | | Pipz |
| 26 | 296.85 | 217 | | None | | | Medium | | Pom- |
| | | | | | | | | | Pipz |
| 27 | 289.10 | 143 | 107 | 68 | 92 | | 118 | | Lena- |
| | | | | | | | | | Pipd |
| 28 | 16.80 | 223.00 | 112 | 83 | 97 | | 127 | | Lena- |
| | | | | | | | | | Pipd |
| 29 | 4.30 | 262 | 64 | 14 | 62 | | 51 | | Pom- |
| | | | | | | | | | Pipd |
| 30 | 0.30 | 215.00 | 105 | 88 | 77 | | 140 | | Pom- |
| | | | | | | | | | Pipd |
| 31 | 2.1 | 260 | 58 | 18 | 54 | | 61 | | Pom- |
| | | | | | | | | | Pipd |
| 32 | 1.3 | 251 | 69 | 21 | 56 | | 46 | | Pom- |
| | | | | | | | | | Pipd |
| 33 | 0.7 | 185 | 83 | 74 | 71 | | 89 | | PG |
| 34 | 31 | 210 | 90 | 71 | 69 | | 74 | | PG |
| 35 | 22.00 | 223 | | 33 | 51 | | 116 | | Pipd |
| 36 | 5.40 | 129 | 118 | 32 | 63 | | 94 | | O |
| 37 | 2.70 | 149 | 120 | 26 | 58 | 121 | 99 | | Pipz |
| 38 | 2.60 | 122 | | 48 | 41 | | 99 | | Pipz |
| 39 | 33.10 | 221 | 126 | 61 | 53 | | 84 | | Pipz |
| 40 | 1.90 | 139 | 114 | 36 | 56 | | 88 | | Pipz |
| 41 | 51.20 | 202 | 97 | 28 | 46 | 125 | 88 | | O |
| 42 | 836.50 | 139 | 128 | 48 | 71 | | 76 | | Pipz |
| 43 | | | 123 | 76 | 79 | | 37 | | O |
| 44 | | | 104 | 64 | 74 | | 81 | | Pipz |
| 45 | | | 107 | 21 | 29 | | 101 | | Pipd |
| 46 | 9.25 | | 98 | 20 | 28 | 119 | 87 | | Pipd |
| 47 | 3.35 | | 114 | 128 | 101 | 89 | 86 | | O |
| 48 | 27.3 | 80 | | 76 | | | 126 | | Pipz |
| 49 | 4 | 199 | 59 | 13 | 11 | | 74 | | Pipz |
| 50 | 9 | 202 | 85 | 11 | 28 | | 137 | | Spiro |
| | | | | | | | | | pipd |
| 51 | 226 | 105 | | 67 | | | 126 | | Spiro |
| | | | | | | | | | pipd |
| 52 | 9 | 180 | 126 | 16 | 27 | | 72 | | Spiro |
| | | | | | | | | | pipd |
| 53 | 1.7 | 142 | | 36 | | | 102 | | Spiro |
| | | | | | | | | | pipd |
| 54 | 6.3 | 67 | | 62 | | | 125 | | Pipz |
| 55 | 596.5 | 149 | 113 | 19 | 44 | | 75 | | Pipz |
| 56 | 2649 | 172 | | 58 | | | 102 | | Spiro |
| | | | | | | | | | Pipd |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 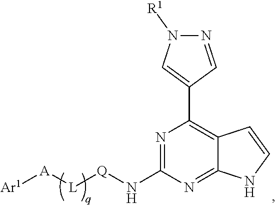
wherein q is selected from 0 and 1;
wherein A is selected from —O—, —S—, —NH—, —NHCH₂—, —OCH₂C(O)NH—, —CH₂—, and a structure selected from: 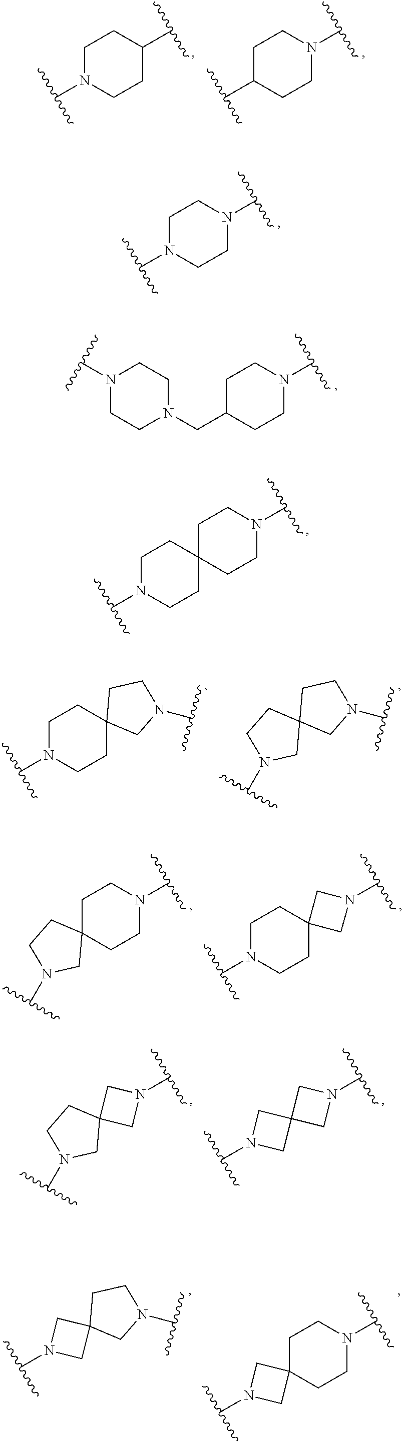 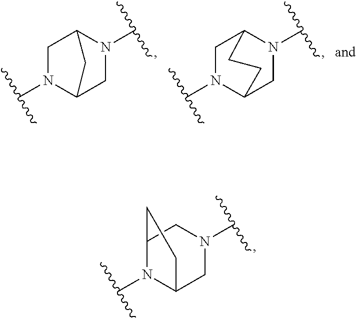
wherein L is selected from C1-C15 alkyl, —(CH₂CH₂O)ₙ—, and —(CH₂CH₂O)ₙ(C1-C4 alkyl)-;
wherein n, when present, is selected from 1, 2, 3, 4, 5, 6, 7, and 8;
wherein Q is selected from —C(O)(C₆H₄)—, —NHC(O)(C₆H₄)—, and —C(O)NH(C₆H₄)—;
wherein R¹ is selected from C1-C8 alkyl, C1-C8 hydroxyalkyl, C1-C8 cyanoalkyl, C1-C8 aminoalkyl, and a structure selected from: 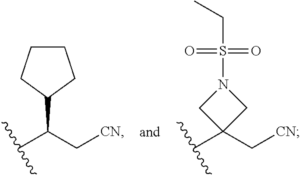 [SMILES: *[C@H](CC#N)C1CCCC1 ; *C1(CC#N)CN(S(=O)(=O)CC)C1]
and
wherein Ar¹ is a structure selected from: 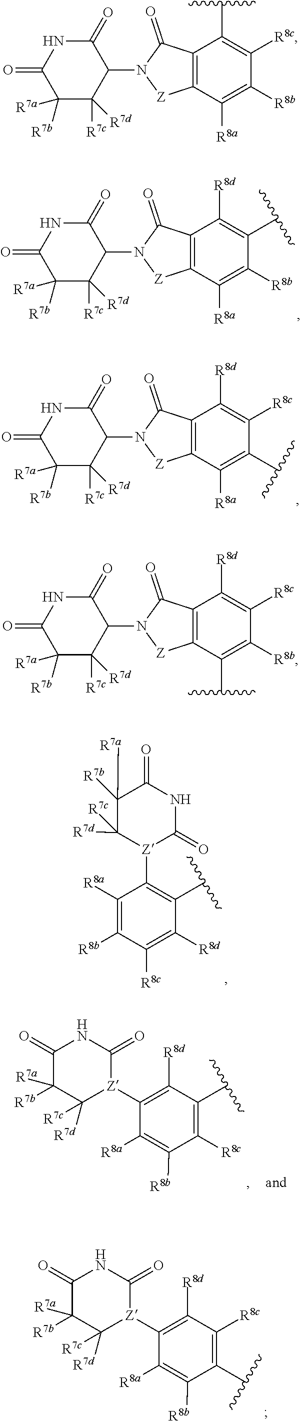
wherein Z is selected from —CH₂— and C(O);
wherein Z′ is selected from —CH— and —N—;
wherein each of R^{7a}, R^{7b}, R^{7c}, and R^{7d} is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; and
wherein each of R^{8a}, R^{8b}, R^{8c}, and R^{8d}, when present, is independently selected from hydrogen, halogen, —NH₂, —OH, —NO₂, —CN, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl,
provided that when Ar¹ is: 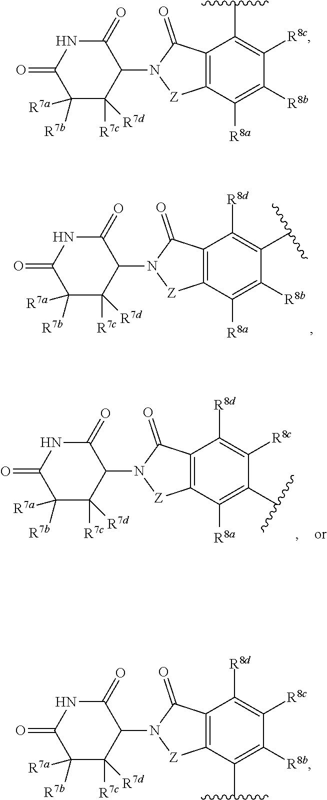
and q is 1, then A is a structure selected from: 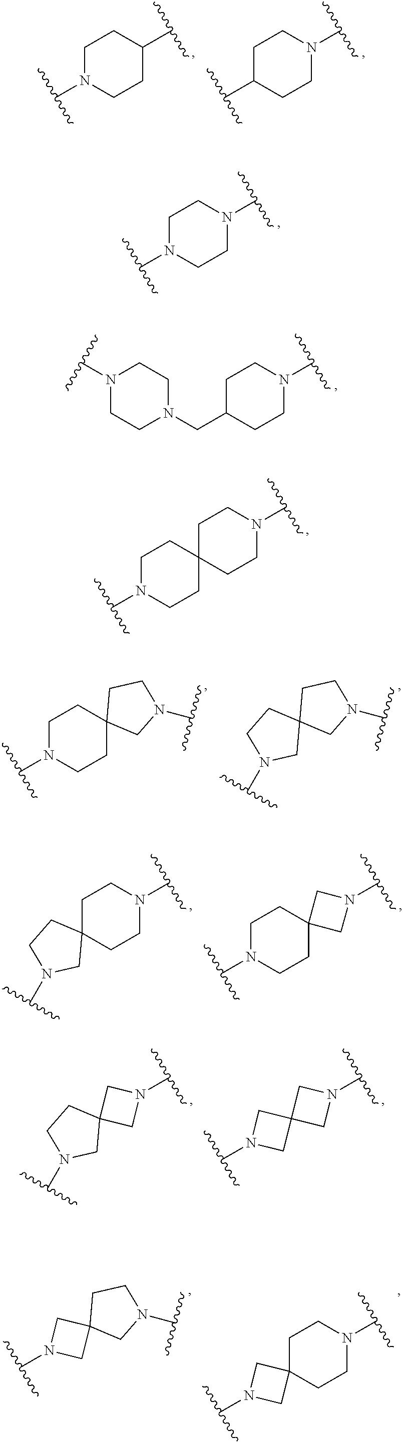 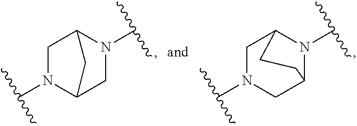 [SMILES: *N1CC2CC1CN2* ; *N1CC2CCC(C1)N2*]
or a pharmaceutically acceptable salt thereof.

2. The compound of claim 1, wherein q is 0.

3-6. (canceled)

7. The compound of claim 1, wherein A is a structure selected from:

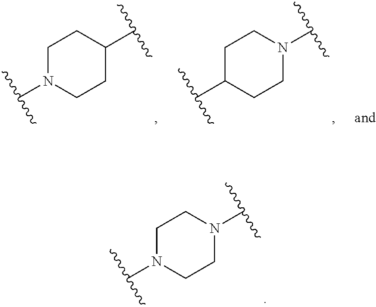 [SMILES: *C1CCN(*)CC1 ; *C1CCN(*)CC1 ; *N1CCN(*)CC1]

8-12. (canceled)

13. The compound of claim 1, Q is —C(O)(C₆H₄)—.

14. The compound of claim 1, wherein Q is a structure:

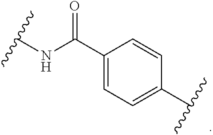 [SMILES: *NC(=O)c1ccc(*)cc1]

15-18. (canceled)

19. The compound of claim 1, wherein R¹ is:

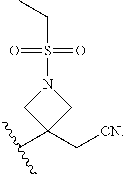 [SMILES: *C1(CC#N)CN(S(=O)(=O)CC)C1]

20. The compound of claim 1, wherein Ar¹ is a structure selected from:

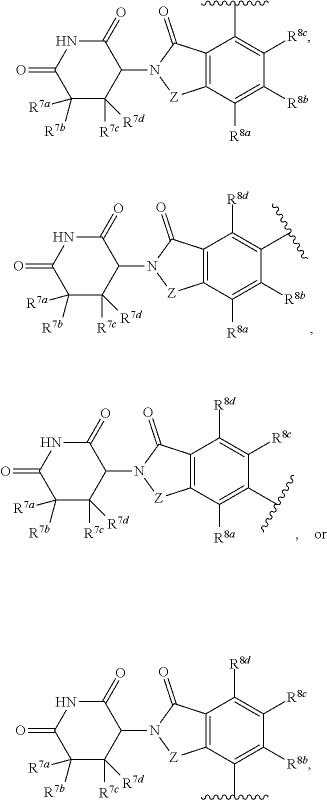

21-22. (canceled)

23. The compound of claim 1, wherein Ar¹ is a structure:

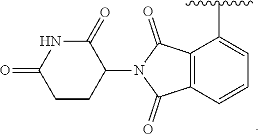 [SMILES: *c1cccc2c1C(=O)N(C1CCC(=O)NC1=O)C2=O]

24. The compound of claim 1, wherein Ar¹ is a structure selected from:

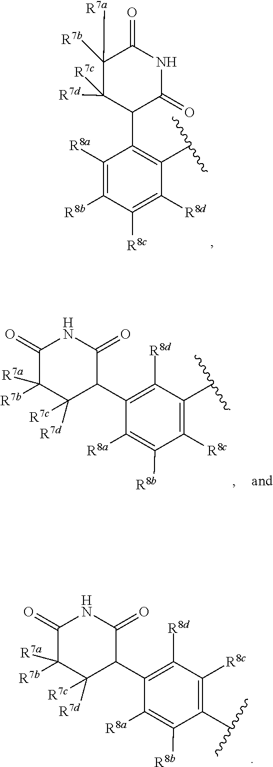

25. (canceled)

26. The compound of claim 1, wherein Ar¹ is a structure selected from:

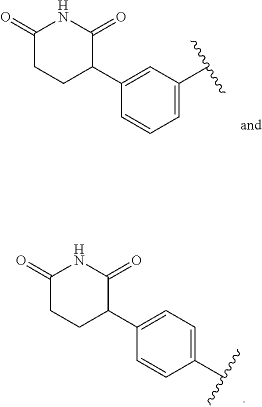 [SMILES: *c1cccc(C2CCC(=O)NC2=O)c1 ; *c1ccc(C2CCC(=O)NC2=O)cc1]

27-28. (canceled)

29. The compound of claim 1, wherein Z is —C(O)—.

30. The compound of claim 1, wherein each of R^{7a}, R^{7b}, R^{7c}, and R^{7d} is hydrogen.

31. The compound of claim 1, wherein each of R^{8a}, R^{8b}, R^{8c}, and R^{8d}, when present, is hydrogen.

32. The compound of claim 1, wherein the compound is selected from:

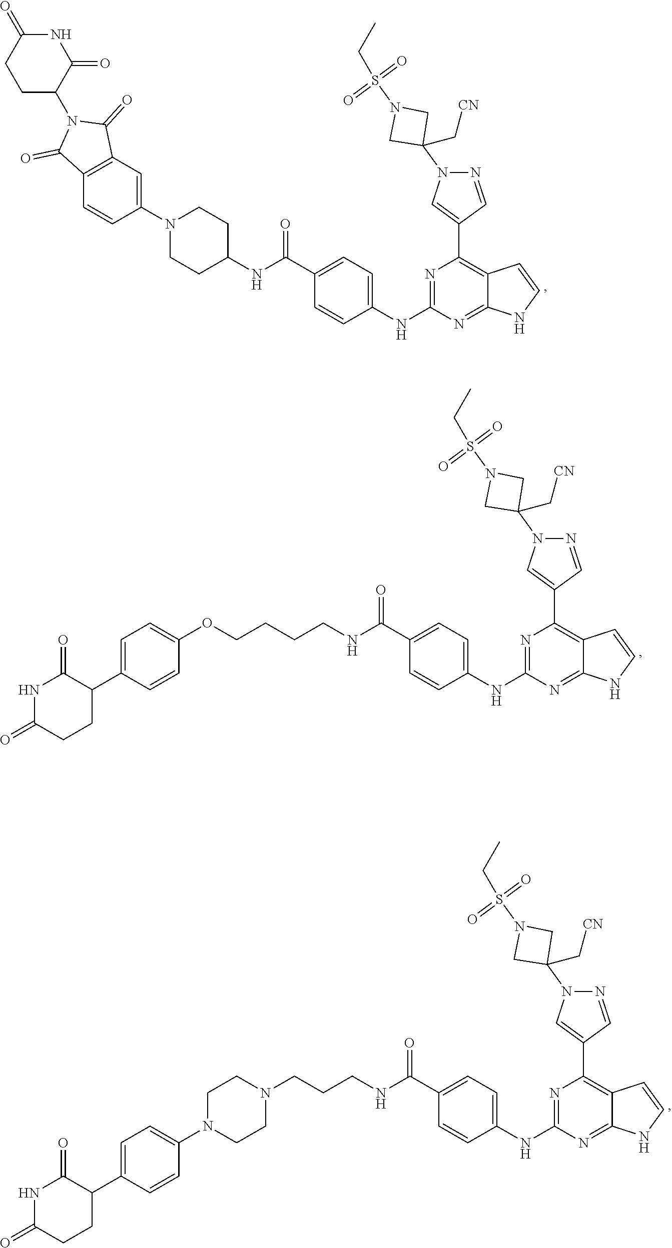 [SMILES: CCS(=O)(=O)N1CC(CC#N)(n2cc(-c3nc(Nc4ccc(C(=O)NC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCOc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCN5CCN(c6ccc(C7CCC(=O)NC7=O)cc6)CC5)cc4)nc4[nH]ccc34)cn2)C1]

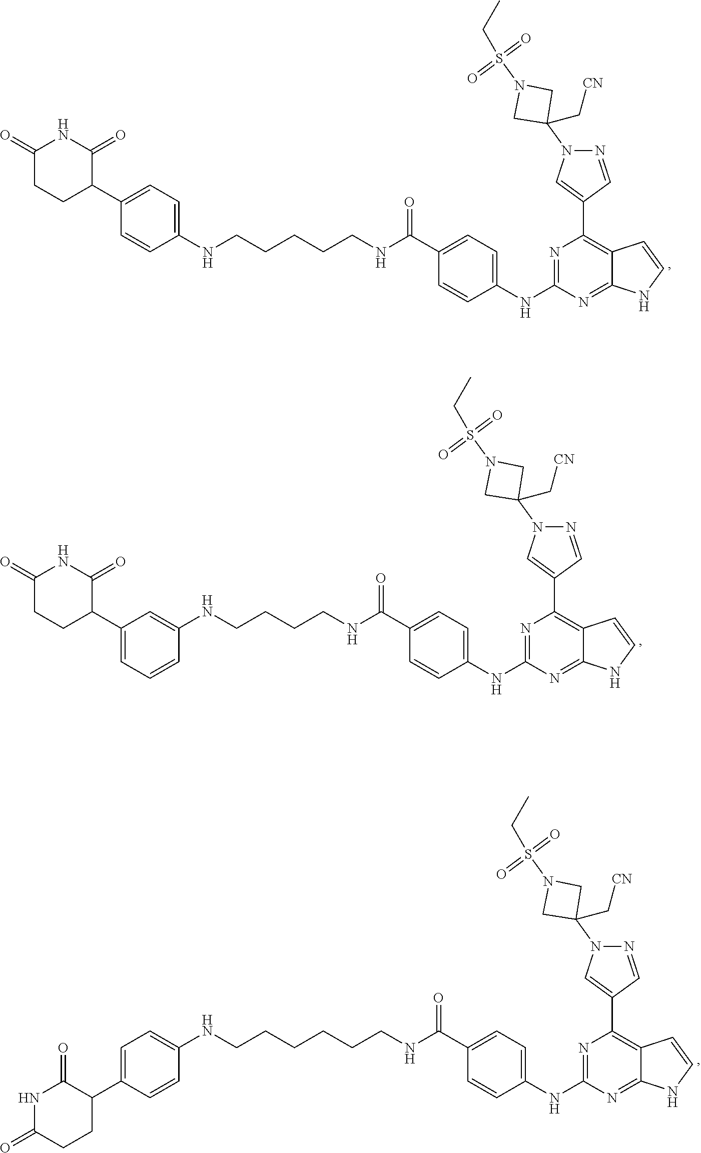 [SMILES: CCS(=O)(=O)N1CC(CC#N)(n2cc(-c3nc(Nc4ccc(C(=O)NCCCCCNc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCNc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCCCCNc5ccc(C6CCC(=O)NC6=O)cc5)cc4)nc4[nH]ccc34)cn2)C1]

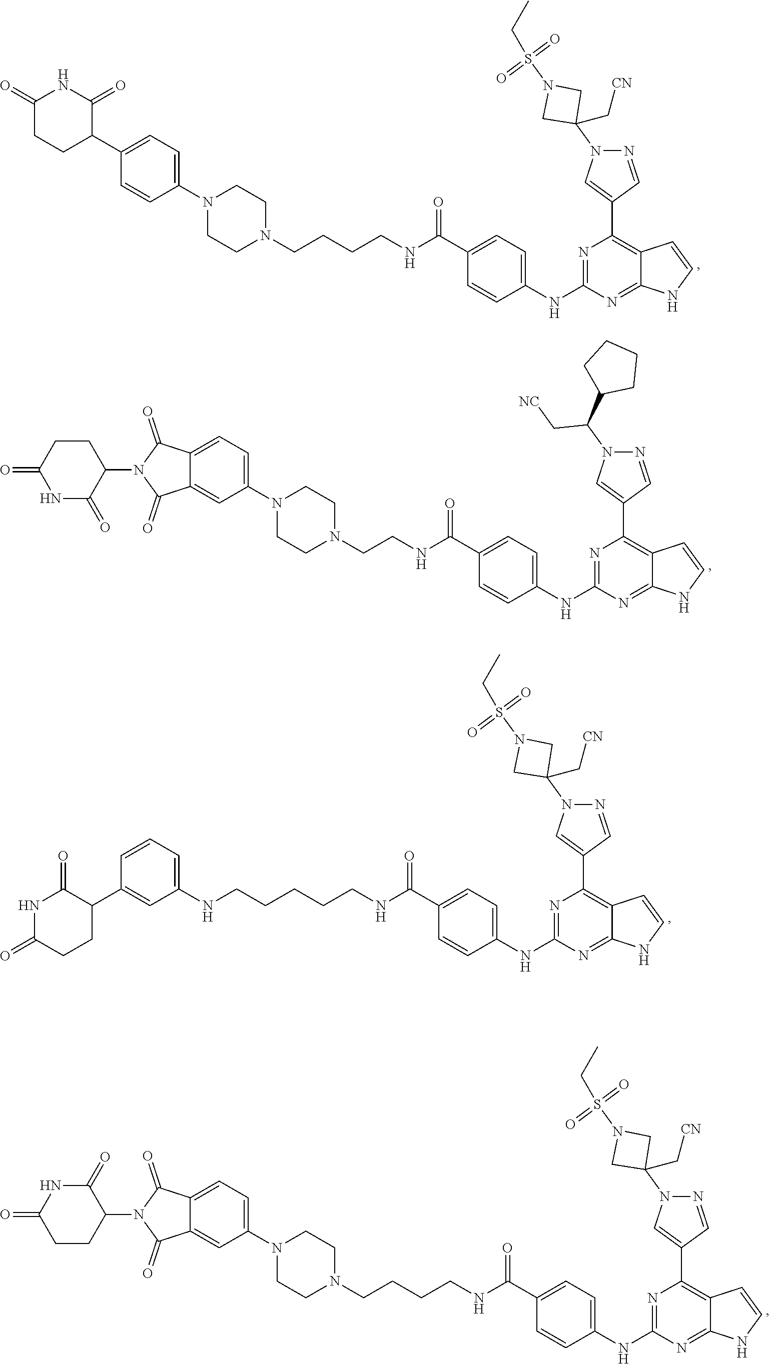

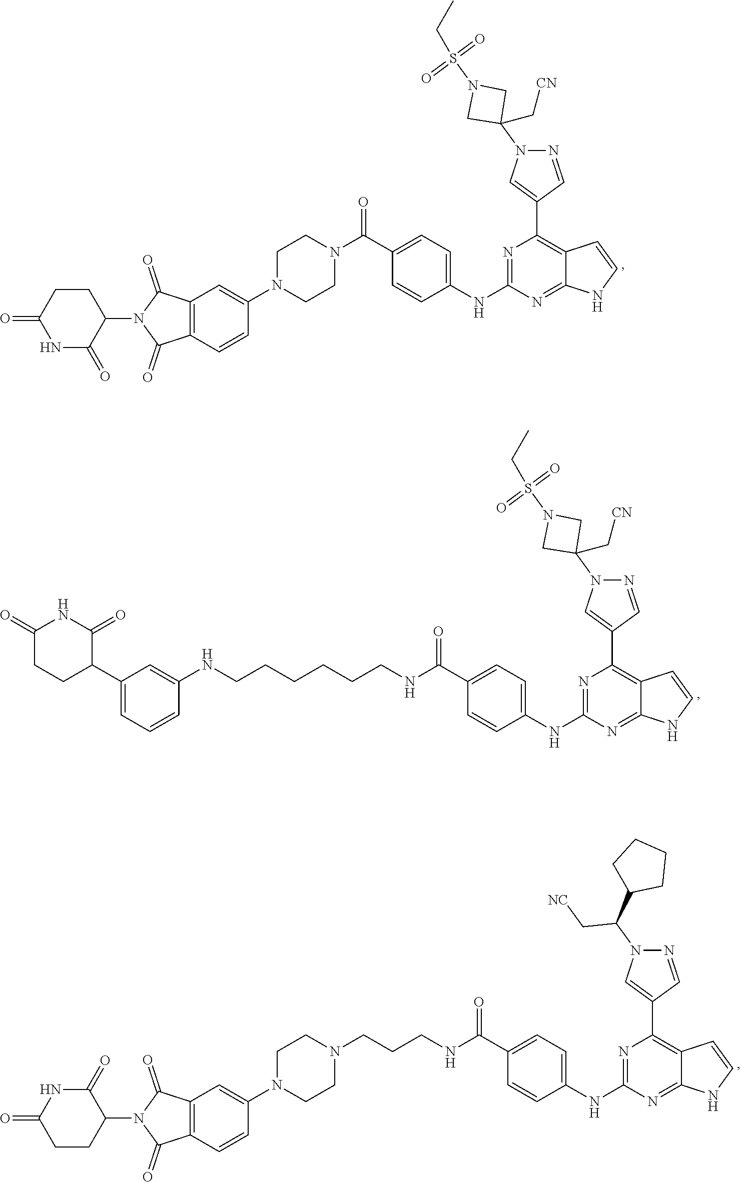 [SMILES: CCS(=O)(=O)N1CC(CC#N)(n2cc(-c3nc(Nc4ccc(C(=O)N5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCCCCNc5cccc(C6CCC(=O)NC6=O)c5)cc4)nc4[nH]ccc34)cn2)C1 ; N#CC[C@@H](C1CCCC1)n1cc(-c2nc(Nc3ccc(C(=O)NCCCN4CCN(c5ccc6c(c5)C(=O)N(C5CCC(=O)NC5=O)C6=O)CC4)cc3)nc3[nH]ccc23)cn1]

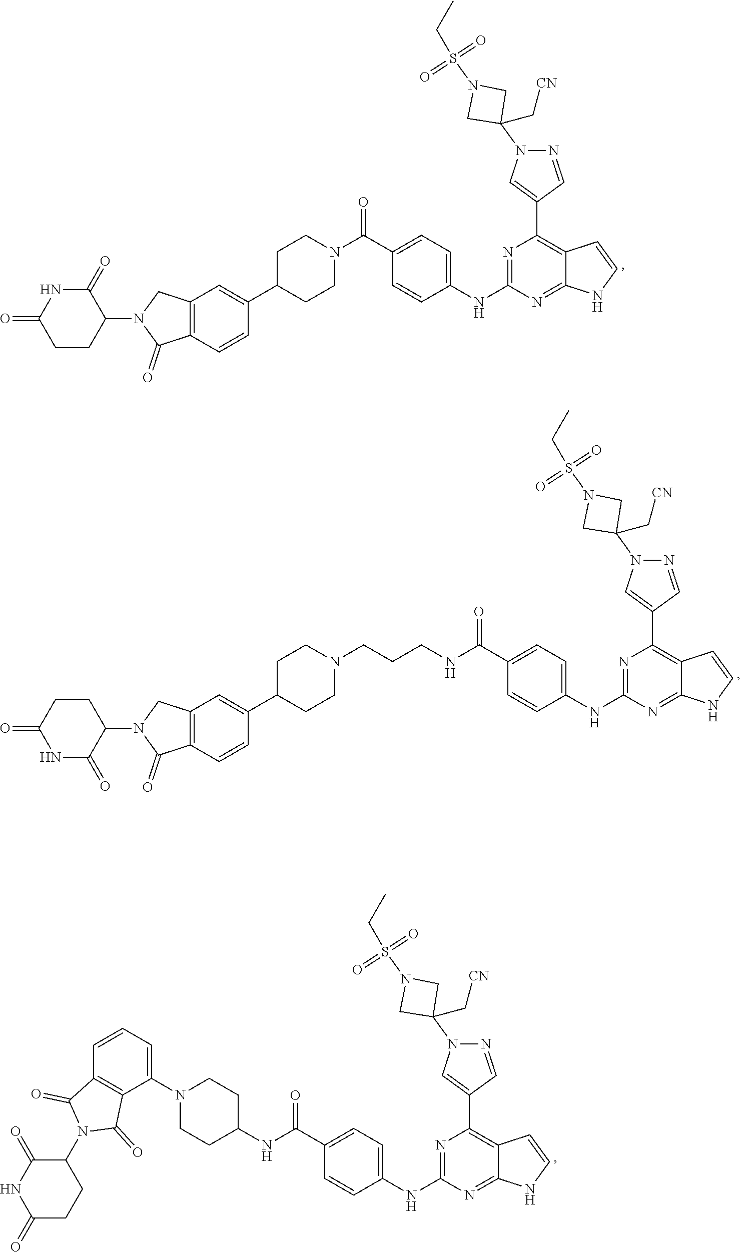 [SMILES: CCS(=O)(=O)N1CC(CC#N)(n2cc(-c3nc(Nc4ccc(C(=O)N5CCC(c6ccc7c(c6)CN(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCN5CCC(c6ccc7c(c6)CN(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NC5CCN(c6cccc7c6C(=O)N(C6CCC(=O)NC6=O)C7=O)CC5)cc4)nc4[nH]ccc34)cn2)C1]

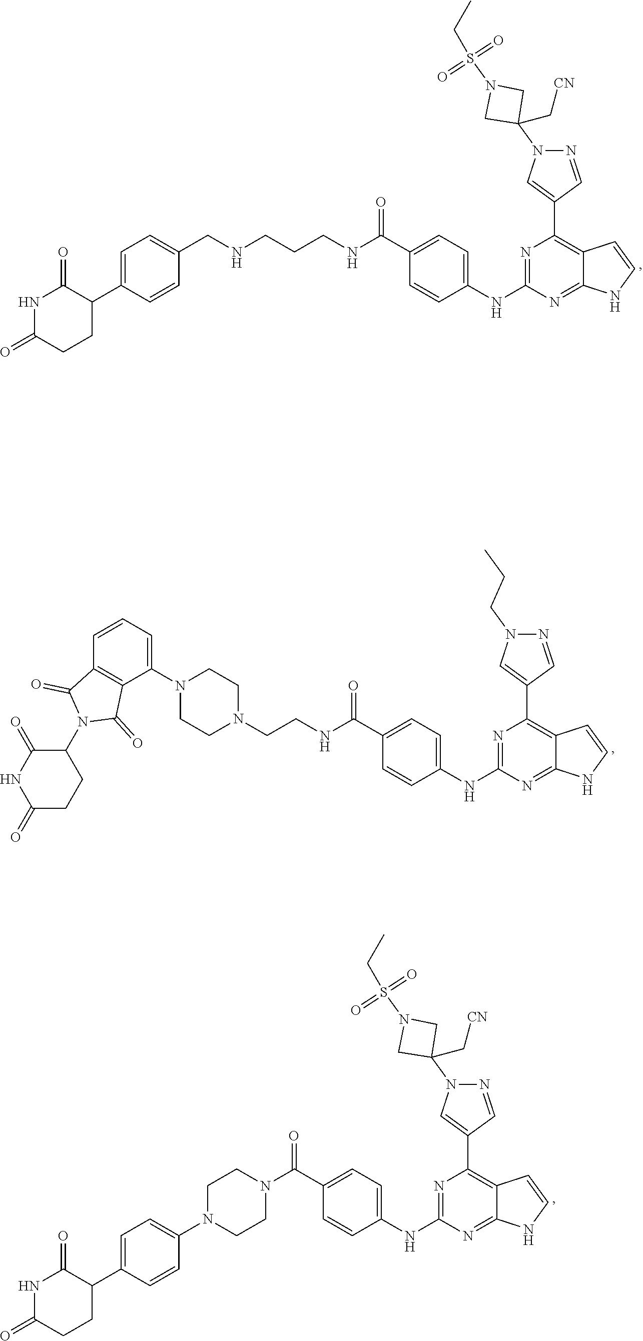 [SMILES: CCS(=O)(=O)N1CC(CC#N)(n2cc(-c3nc(Nc4ccc(C(=O)NCCCNCc5ccc(C6CCC(=O)NC6=O)cc5)cc4)nc4[nH]ccc34)cn2)C1 ; CCCn1cc(-c2nc(Nc3ccc(C(=O)NCCN4CCN(c5cccc6c5C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)N5CCN(c6ccc(C7CCC(=O)NC7=O)cc6)CC5)cc4)nc4[nH]ccc34)cn2)C1]

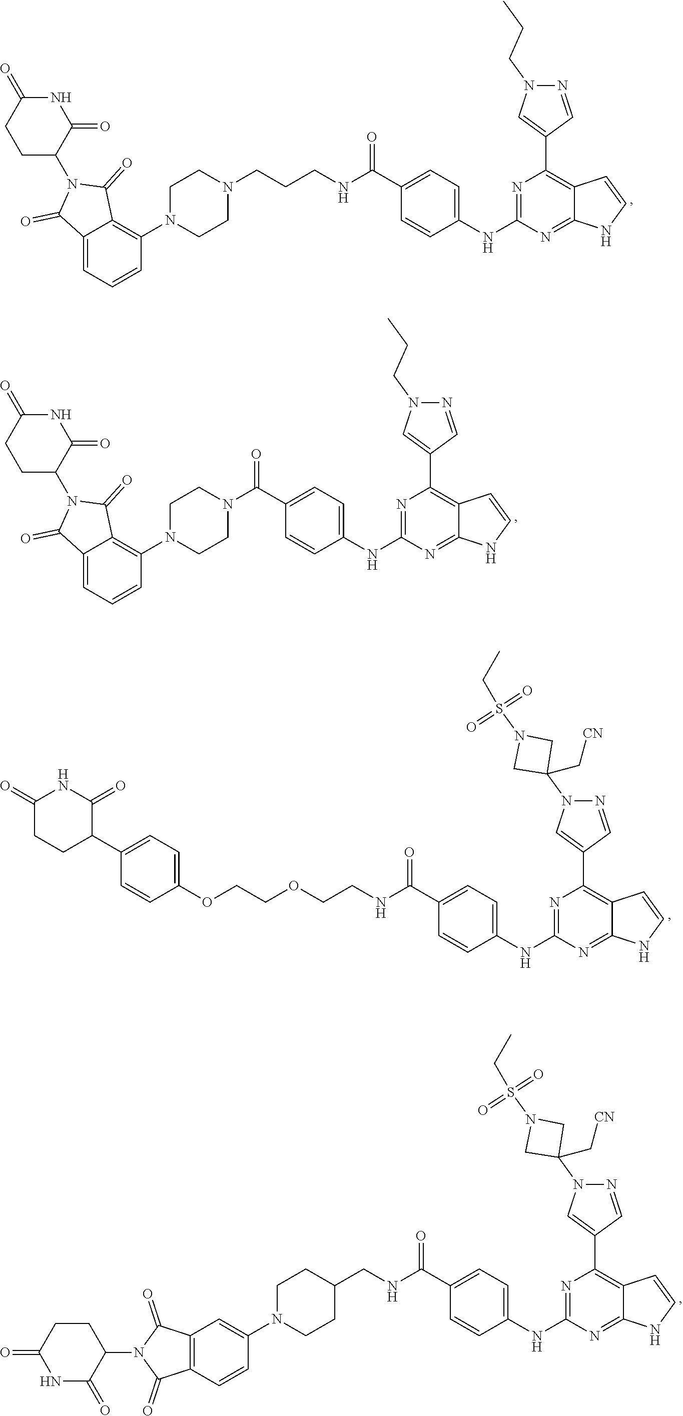 [SMILES: CCCn1cc(-c2nc(Nc3ccc(C(=O)NCCCN4CCN(c5cccc6c5C(=O)N(C5CCC(=O)NC5=O)C6=O)CC4)cc3)nc3[nH]ccc23)cn1 ; CCCn1cc(-c2nc(Nc3ccc(C(=O)N4CCN(c5cccc6c5C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCC5CCN(c6ccc7c(c6)C(=O)N(C6CCC(=O)NC6=O)C7=O)CC5)cc4)nc4[nH]ccc34)cn2)C1]

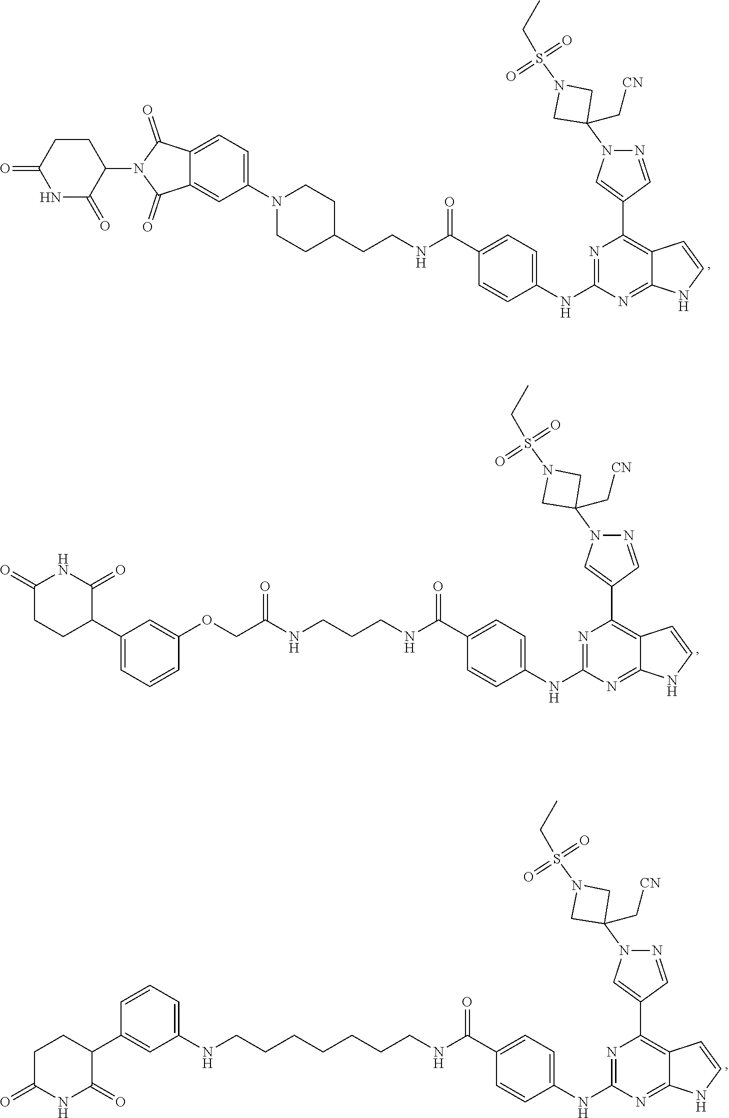 [SMILES: CCS(=O)(=O)N1CC(CC#N)(n2cc(-c3nc(Nc4ccc(C(=O)NCCC5CCN(c6ccc7c(c6)C(=O)N(C6CCC(=O)NC6=O)C7=O)CC5)cc4)nc4[nH]ccc34)cn2)C1 ; CCS(=O)(=O)N1CC(CC#N)(n2cc(-c3nc(Nc4ccc(C(=O)NCCCNC(=O)COc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCCCCCNc5cccc(C6CCC(=O)NC6=O)c5)cc4)nc4[nH]ccc34)cn2)C1]

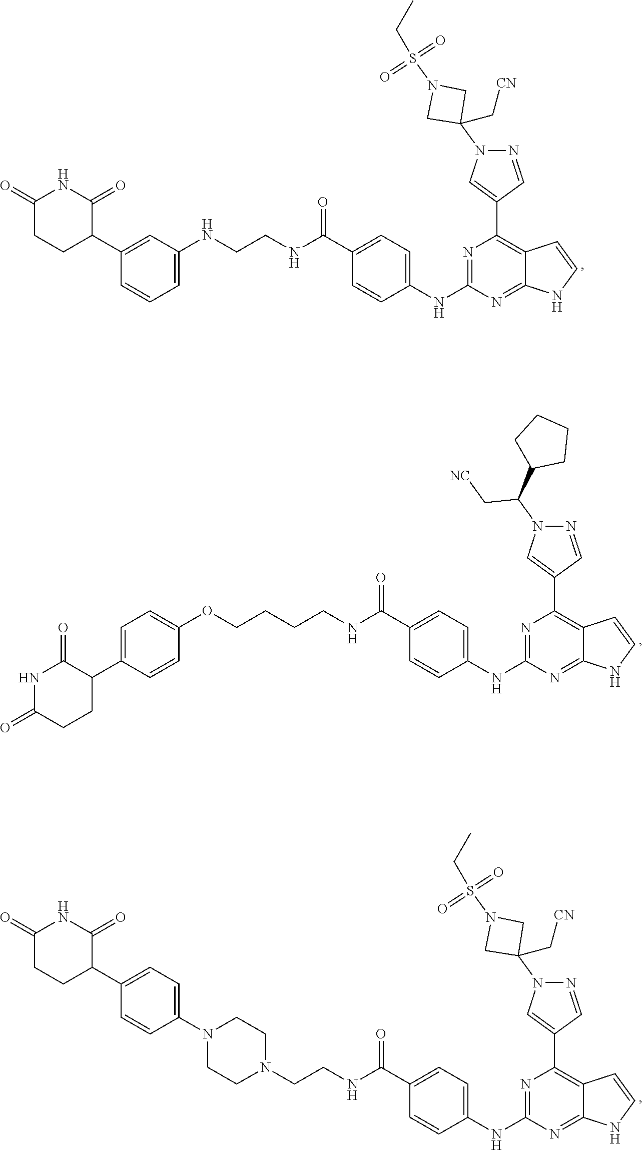 [SMILES: CCS(=O)(=O)N1CC(CC#N)(n2cc(-c3nc(Nc4ccc(C(=O)NCCNc5cccc(C6CCC(=O)NC6=O)c5)cc4)nc4[nH]ccc34)cn2)C1 ; N#CC[C@@H](C1CCCC1)n1cc(-c2nc(Nc3ccc(C(=O)NCCCCOc4ccc(C5CCC(=O)NC5=O)cc4)cc3)nc3[nH]ccc23)cn1 ; CCS(=O)(=O)N1CC(CC#N)(n2cc(-c3nc(Nc4ccc(C(=O)NCCN5CCN(c6ccc(C7CCC(=O)NC7=O)cc6)CC5)cc4)nc4[nH]ccc34)cn2)C1]

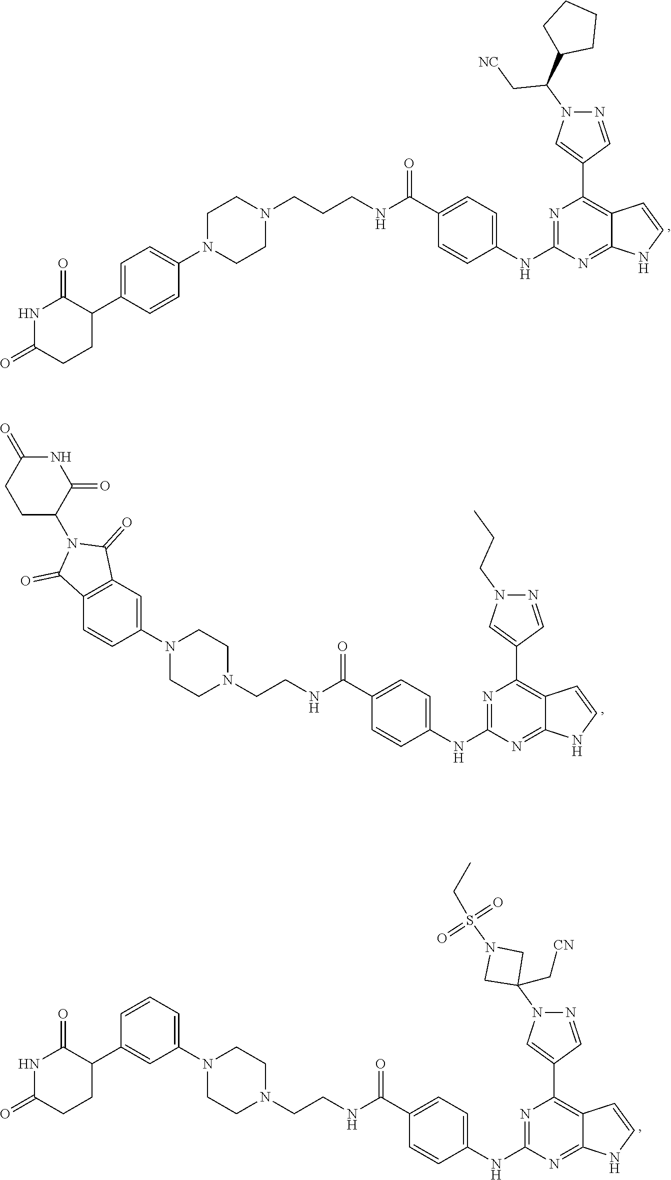 [SMILES: N#CC[C@@H](C1CCCC1)n1cc(-c2nc(Nc3ccc(C(=O)NCCCN4CCN(c5ccc(C6CCC(=O)NC6=O)cc5)CC4)cc3)nc3[nH]ccc23)cn1 ; CCCn1cc(-c2nc(Nc3ccc(C(=O)NCCN4CCN(c5ccc6c(c5)C(=O)N(C5CCC(=O)NC5=O)C6=O)CC4)cc3)nc3[nH]ccc23)cn1 ; CCS(=O)(=O)N1CC(CC#N)(n2cc(-c3nc(Nc4ccc(C(=O)NCCN5CCN(c6cccc(C7CCC(=O)NC7=O)c6)CC5)cc4)nc4[nH]ccc34)cn2)C1]

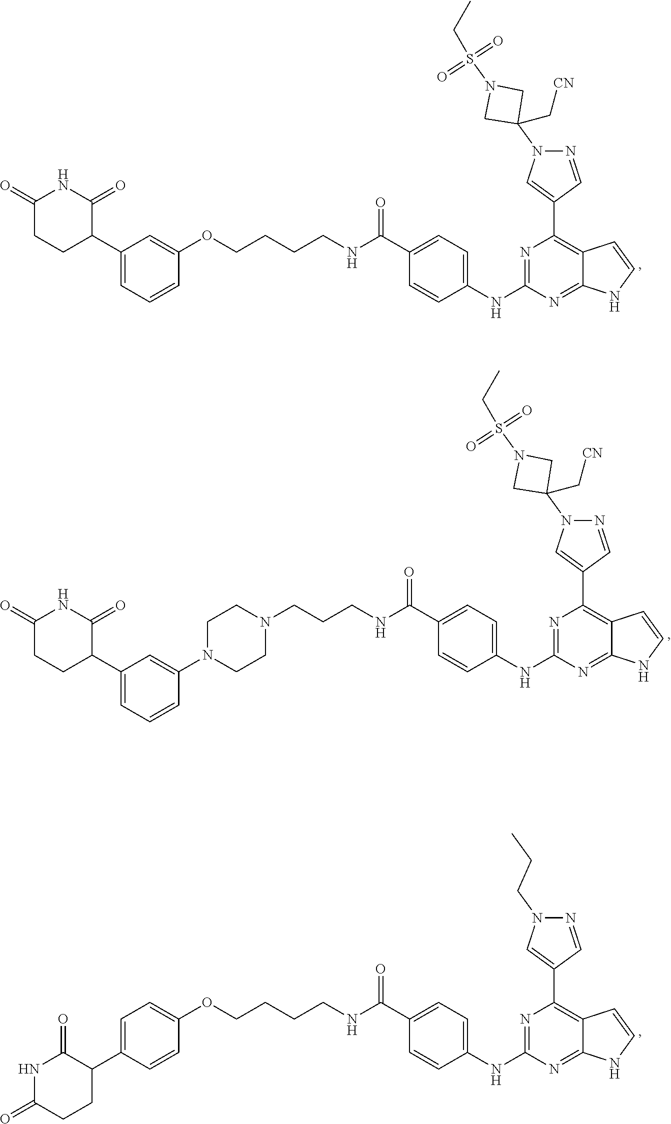 [SMILES: CCS(=O)(=O)N1CC(CC#N)(n2cc(-c3nc(Nc4ccc(C(=O)NCCCCOc5cccc(C6CCC(=O)NC6=O)c5)cc4)nc4[nH]ccc34)cn2)C1 ; CCS(=O)(=O)N1CC(CC#N)(n2cc(-c3nc(Nc4ccc(C(=O)NCCCN5CCN(c6cccc(C7CCC(=O)NC7=O)c6)CC5)cc4)nc4[nH]ccc34)cn2)C1 ; CCCn1cc(-c2nc(Nc3ccc(C(=O)NCCCCOc4ccc(C5CCC(=O)NC5=O)cc4)cc3)nc3[nH]ccc23)cn1]

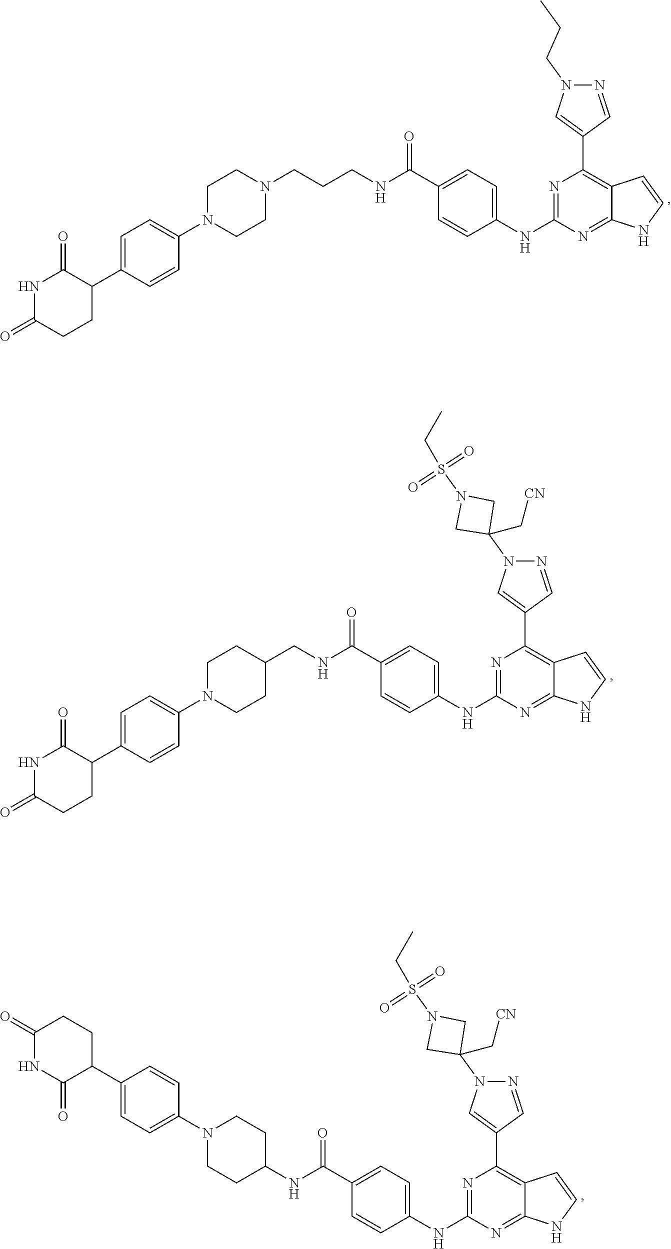 [SMILES: CCCn1cc(-c2nc(Nc3ccc(C(=O)NCCCN4CCN(c5ccc(C6CCC(=O)NC6=O)cc5)CC4)cc3)nc3[nH]ccc23)cn1 ; CCS(=O)(=O)N1CC(CC#N)(n2cc(-c3nc(Nc4ccc(C(=O)NCC5CCN(c6ccc(C7CCC(=O)NC7=O)cc6)CC5)cc4)nc4[nH]ccc34)cn2)C1 ; CCS(=O)(=O)N1CC(CC#N)(n2cc(-c3nc(Nc4ccc(C(=O)NC5CCN(c6ccc(C7CCC(=O)NC7=O)cc6)CC5)cc4)nc4[nH]ccc34)cn2)C1]

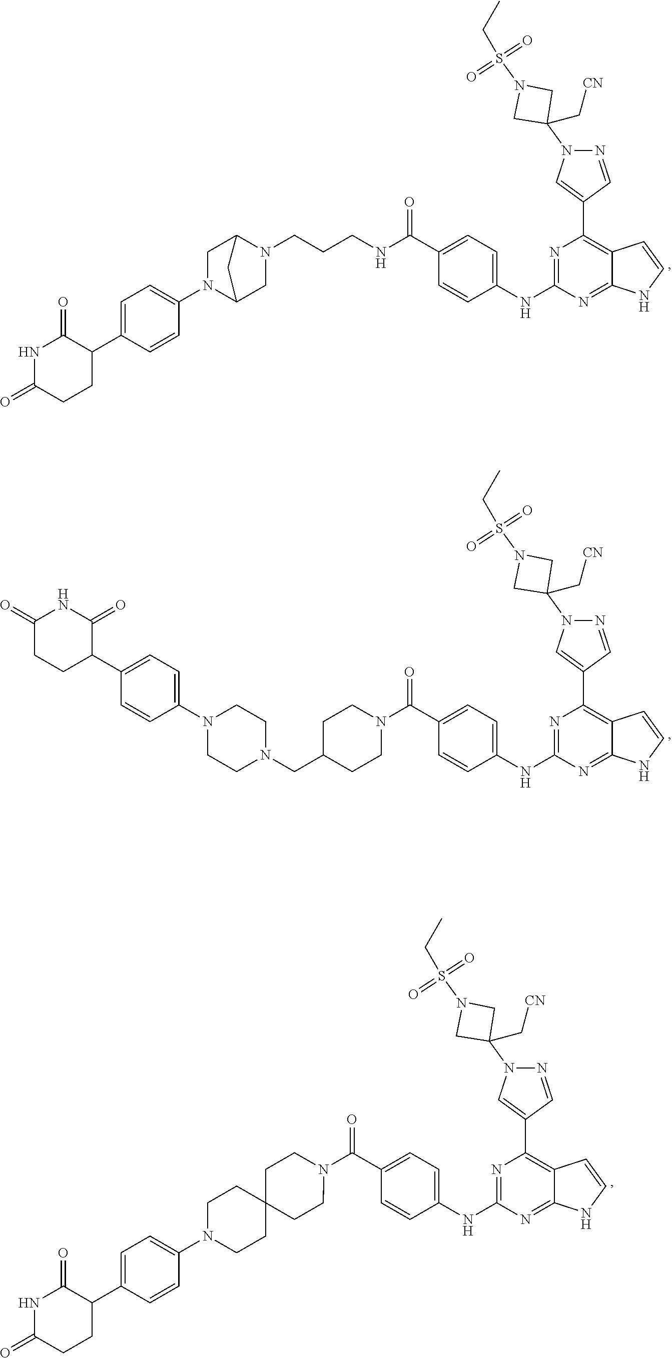 [SMILES: CCS(=O)(=O)N1CC(CC#N)(n2cc(-c3nc(Nc4ccc(C(=O)NCCCN5CC6CC5CN6c5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)N5CCC(CN6CCN(c7ccc(C8CCC(=O)NC8=O)cc7)CC6)CC5)cc4)nc4[nH]ccc34)cn2)C1 ; CCS(=O)(=O)N1CC(CC#N)(n2cc(-c3nc(Nc4ccc(C(=O)N5CCC6(CC5)CCN(c5ccc(C7CCC(=O)NC7=O)cc5)CC6)cc4)nc4[nH]ccc34)cn2)C1]

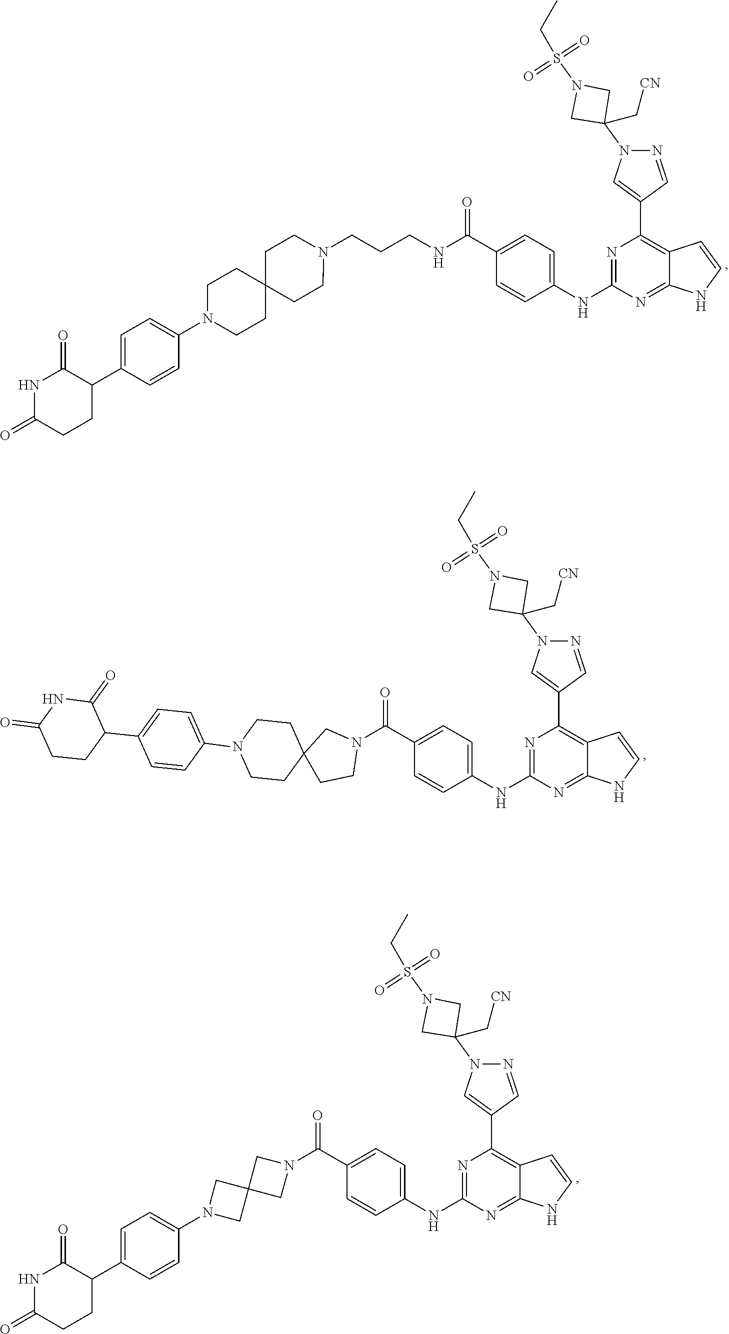 [SMILES: CCS(=O)(=O)N1CC(CC#N)(n2cc(-c3nc(Nc4ccc(C(=O)NCCCN5CCC6(CC5)CCN(c5ccc(C7CCC(=O)NC7=O)cc5)CC6)cc4)nc4[nH]ccc34)cn2)C1 ; CCS(=O)(=O)N1CC(CC#N)(n2cc(-c3nc(Nc4ccc(C(=O)N5CCC6(CCN(c7ccc(C8CCC(=O)NC8=O)cc7)CC6)C5)cc4)nc4[nH]ccc34)cn2)C1 ; CCS(=O)(=O)N1CC(CC#N)(n2cc(-c3nc(Nc4ccc(C(=O)N5CC6(C5)CN(c5ccc(C7CCC(=O)NC7=O)cc5)C6)cc4)nc4[nH]ccc34)cn2)C1]

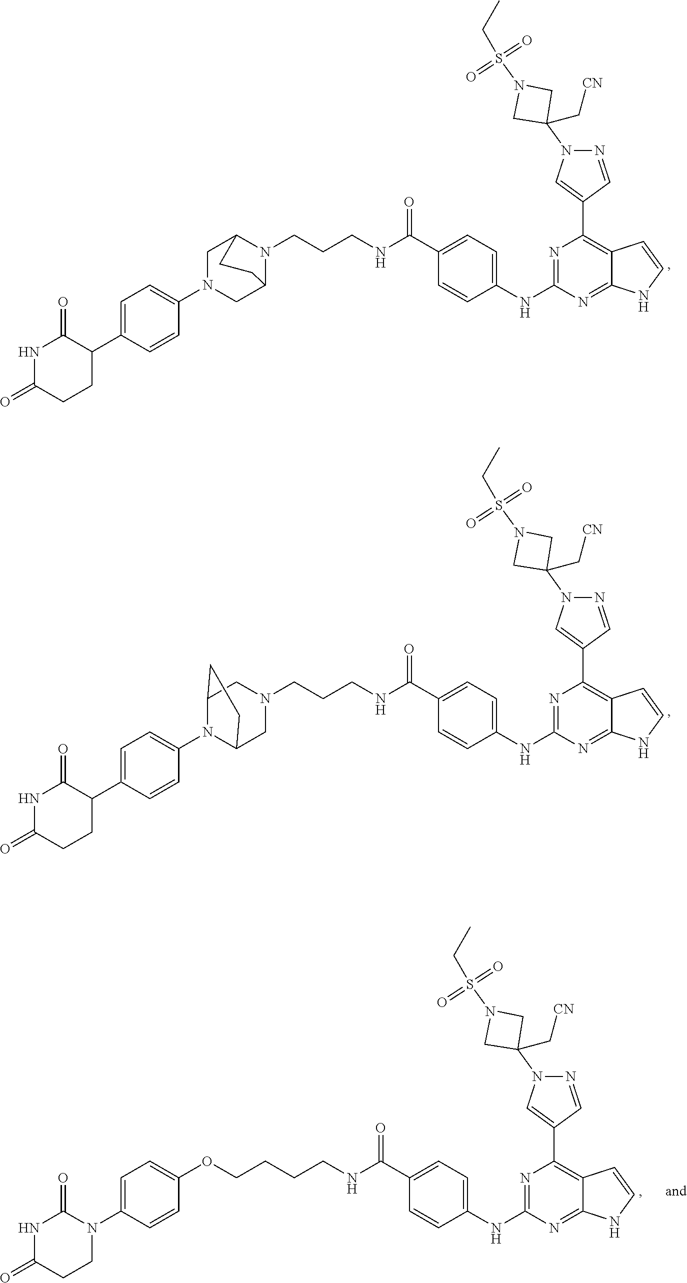 [SMILES: CCS(=O)(=O)N1CC(CC#N)(n2cc(-c3nc(Nc4ccc(C(=O)NCCCN5C6CCC5CN(c5ccc(C7CCC(=O)NC7=O)cc5)C6)cc4)nc4[nH]ccc34)cn2)C1 ; CCS(=O)(=O)N1CC(CC#N)(n2cc(-c3nc(Nc4ccc(C(=O)NCCCN5CC6CCC(C5)N6c5ccc(C6CCC(=O)NC6=O)cc5)cc4)nc4[nH]ccc34)cn2)C1 ; CCS(=O)(=O)N1CC(CC#N)(n2cc(-c3nc(Nc4ccc(C(=O)NCCCCOc5ccc(N6CCC(=O)NC6=O)cc5)cc4)nc4[nH]ccc34)cn2)C1]

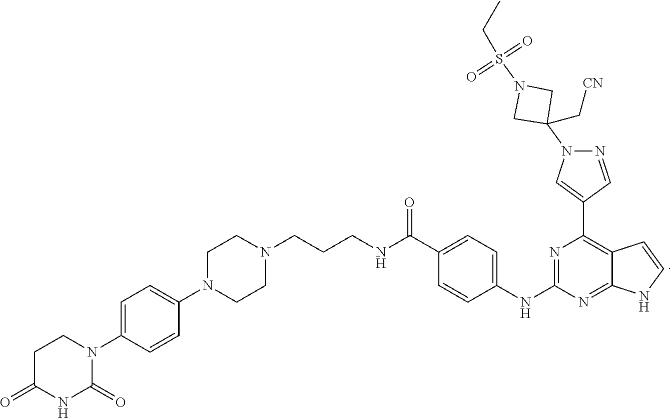 [SMILES: CCS(=O)(=O)N1CC(CC#N)(n2cc(-c3nc(Nc4ccc(C(=O)NCCCN5CCN(c6ccc(N7CCC(=O)NC7=O)cc6)CC5)cc4)nc4[nH]ccc34)cn2)C1]

33. The compound of claim 1, wherein the compound is not:

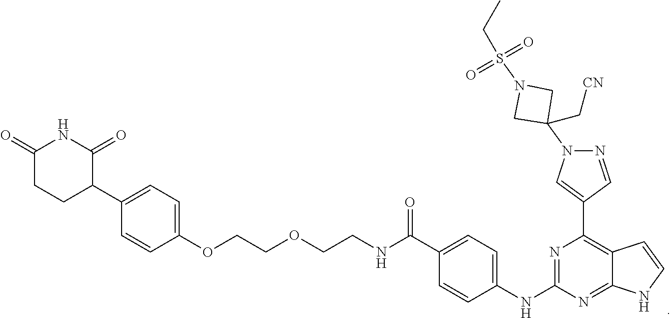 [SMILES: CCS(=O)(=O)N1CC(CC#N)(n2cc(-c3nc(Nc4ccc(C(=O)NCCOCCOc5ccc(C6CCC(=O)NC6=O)cc5)cc4)nc4[nH]ccc34)cn2)C1]

34. The compound of claim 1, wherein the compound is selected from:

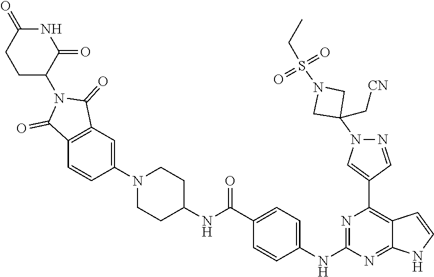 [SMILES: CCS(=O)(=O)N1CC(CC#N)(n2cc(-c3nc(Nc4ccc(C(=O)NC5CCN(c6ccc7c(c6)C(=O)N(C6CCC(=O)NC6=O)C7=O)CC5)cc4)nc4[nH]ccc34)cn2)C1]

35. A pharmaceutical composition comprising a therapeutically effective amount of the compound of claim 1 and a pharmaceutically acceptable carrier.

36. A method of treating a disorder of uncontrolled cellular proliferation in a subject, the method comprising administering to the subject an effective amount of the compound of claim 1.

37-42. (canceled)

43. The method of claim 36, wherein the disorder is a cancer.

44. (canceled)

45. The method of claim 43, wherein the cancer is acute lymphoblastic leukemia (ALL).

46-57. (canceled)
